(19) United States
(12) Patent Application Publication
(10) Pub. No.: US 2025/0292907 A1
(43) Pub. Date: Sep. 18, 2025

(54) SYSTEMS AND METHODS FOR PERFORMING GAIT AND SPEECH ASSESSMENT ASSOCIATED WITH A DISEASE STATE

(71) Applicant: Regeneron Pharmaceuticals, Inc., Tarrytown, NY (US)

(72) Inventors: Rolando J. ACOSTA, Cambridge, MA (US); Matthew F. Wipperman, Brooklyn, NY (US); Oren Levy, New York, NY (US); Bharatkumar Koyani, Cary, NC (US); Jacek K. Urbanek, Eastchester, NY (US)

(73) Assignee: Regeneron Pharmaceuticals, Inc., Tarrytown, NY (US)

(21) Appl. No.: 19/079,265

(22) Filed: Mar. 13, 2025

Related U.S. Application Data
(60) Provisional application No. 63/565,069, filed on Mar. 14, 2024.
(60) Provisional application No. 63/633,487, filed on Apr. 12, 2024.
(60) Provisional application No. 63/695,789, filed on Sep. 17, 2024.

(51) Int. Cl. G16H 50/20; A61B 5/00; A61B 5/11; G16H 10/20; G16H 20/10

(52) U.S. Cl. CPC G16H 50/20; A61B 5/112; A61B 5/4803; G16H 10/20; G16H 20/10

(57) ABSTRACT

Embodiments disclosed herein are directed to systems and processes that derive clinically meaningful data from a test device and a production device for assessing gait and speech in subjects having a neurological condition. The test device for gait analysis may be a simpler or more updated device in reference to a production device. Speech characteristics may be evaluated using a digital mobile speech-based platform. Other aspects are disclosed and claimed.

### CROSS-REFERENCE TO RELATED APPLICATIONS

This application claims priority to U.S. Provisional Patent Application No. 63/565,069, filed on Mar. 14, 2024, U.S. Provisional Patent Application No. 63/633,487, filed on Apr. 12, 2024, and U.S. Provisional Patent Application No. 63/695,789 filed on Sep. 17, 2024, the entireties of each of which are incorporated herein by reference.

The tables disclosed in Appendix A of U.S. Provisional Patent Application No. 63/695,789, filed on Sep. 17, 2024, and specifically including Tables 1-14, are hereby incorporated by reference.

### TECHNICAL FIELD

The present disclosure relates generally to the field of medical devices and digital health technology. More specifically, the embodiments disclosed herein are directed to systems and methods for the quantitative assessment of gait and speech in patients with neurological disorders. Aspects of the embodiments leverage wearable sensor technology and mobile applications to provide continuous, objective measurements of gait and speech parameters, facilitating both clinical evaluation and home-based monitoring of disease progression and symptom management.

### INTRODUCTION

Understanding human gait and speech is important for assessing the severity and progression of neurological conditions, such as Parkinson's Disease (PD). Gait abnormalities and speech difficulties are core features of PD that significantly affect patient's quality of life. Current clinical assessments for PD are largely qualitative and rater-dependent, limiting their sensitivity to detect changes in early-stage PD. Moreover, existing quantitative gait measurement systems are confined to clinical settings and are impractical for home use. One or more aspects of this disclosure may address one or more of the issues described above.

The introduction provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

Aspects of the present disclosure relate to validating test devices using a trained machine learning model generated based on a production device.

In one aspect, a method for triggering an automated action is disclosed. The method may include: receiving, at a computer system, a first set of gait characteristic data from a first device and a second set of gait characteristic data from a second device for an activity performed by a subject in an OFF-state at a first time; receiving, at the computer system, a third set of gait characteristic data from the first device and a fourth set of gait characteristic data from the second device for the activity performed by the subject in an ON-state at a second time, wherein the second time occurs after the first time; comparing, using a processor associated with the computer system, the first set of gait characteristic data, the second set of gait characteristic data, the third set of gait characteristic data, and the fourth set of gait characteristic data against a reference score scale associated with a disease condition; and triggering, at the computer system, the automated action based on a result of the comparing.

In another aspect, a method for triggering an automated action is disclosed. The method may include: receiving, at a computer system, a first set of speech characteristic data from a speech application platform of a mobile device for at least one activity performed by a subject in an OFF-state at a first time; receiving, at the computer system, a second set of speech characteristic data from the speech application platform for the at least one activity performed again by the subject in an ON-state at a second time, wherein the second time occurs after the first time; comparing, using a processor associated with the computer system, the first set of speech characteristic data and the second set of speech characteristic data against a reference score scale associated with a disease condition; and triggering, at the computer system, the automated action based on a result of the comparing.

In yet another aspect, a method for triggering an automated action is disclosed. The method may include: receiving, at a computer system, a first set of gait characteristic data from a first device and a second set of gait characteristic data from a second device for a first activity performed by a subject associated with an OFF-state at a first time; receiving, at the computer system, a first set of speech characteristic data from a speech application platform of a third device for at least one second activity performed by the subject associated with the OFF-state; receiving, at the computer system, a third set of gait characteristic data from the first device and a fourth set of gait characteristic data from the second device for the first activity performed again by the subject associated with an ON-state at a second time, wherein the second time occurs after the first time; receiving, at the computer system, a second set of speech characteristic data from the speech application platform for the at least one second activity performed again by the subject associated with the ON-state; analyzing, using a processor associated with the computer system, a data collection against a reference score scale associated with a disease condition, wherein the data collection include: the first set of gait characteristic data, the second set of gait characteristic data, the third set of gait characteristic data, the fourth set of gait characteristic data, the first set of speech characteristic data, and the second set of speech characteristic data; assessing, using the processor and based on the analyzing, a severity of the disease condition and a fluctuation between the ON-state and the OFF-state of the subject; and triggering, at the computer system, the automated action based on a result of the assessing.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1A is a system environment for validating a test device, in accordance with aspects of the present disclosure.

FIG. 1B is a flow chart for validating a test device, in accordance with aspects of the present disclosure.

FIG. 2A is a system block diagram for validating a machine learning model, in accordance with aspects of the present disclosure.

FIG. 2B is a flow chart for validating a machine learning model, in accordance with aspects of the present disclosure.

FIG. 3 is a flow chart for comparing data from subjects measured by a test and production device, in accordance with aspects of the present disclosure

FIG. 4 provides a diagram of a gait assessment activity, in accordance with aspects of the present disclosure.

FIGS. 5A-5C provide graphs that each illustrate the change in MDS-UPDRS III and LE subscores per subject between an ON-state and an OFF-state, in accordance with aspects of the present disclosure.

FIG. 6 provides a graph that illustrates the correlation between the MDS-UPDRS III score and the lower extremity (LE) subscore between OFF and ON states, in accordance with aspects of the present disclosure.

FIG. 7 provides a correlation heat map of gait features from the production device and the wearable insole test device, in accordance with aspects of the present disclosure.

FIG. 8A provides a scree plot presenting the percentage of variance of the gait characteristics between the production device and the test device, in accordance with aspects of the present disclosure.

FIG. 8B provides a scree plot presenting the cumulative percentage of variance of the gait characteristics between the production device and the test device, in accordance with aspects of the present disclosure.

FIG. 9A provides a scatter plot of overall data in which the first principal component (PC1) is plotted against the second principal component (PC2), in accordance with aspects of the present disclosure.

FIG. 9B provides a scatter plot of site data in which the PC1 is plotted against the PC2, in accordance with aspects of the present disclosure.

FIG. 9C provides a scatter plot of gender data in which the PC1 is plotted against the PC2, in accordance with aspects of the present disclosure.

FIG. 9D provides a scatter plot of subject data in which the PC1 is plotted against the PC2, in accordance with aspects of the present disclosure.

FIG. 10A provides a correlation heat map of speech characteristics for a paragraph reading task assessed using the digital mobile speech-based platform, in accordance with aspects of the present disclosure.

FIG. 10B provides a correlation heat map of speech characteristics for a picture description 1 task assessed using the digital mobile speech-based platform, in accordance with aspects of the present disclosure.

FIG. 10C provides a correlation heat map of speech characteristics for a picture description 2 task assessed using the digital mobile speech-based platform, in accordance with aspects of the present disclosure.

FIG. 10D provides a correlation heat map of speech characteristics for a diadochokinetic rate (DDK) task assessed using the digital mobile speech-based platform, in accordance with aspects of the present disclosure.

FIG. 10E provides a correlation heat map of speech characteristics for a sustained vowel phonation task assessed using the digital mobile speech-based platform, in accordance with aspects of the present disclosure.

FIG. 10F provides a correlation heat map of speech characteristics for a semantic fluency task assessed using the digital mobile speech-based platform, in accordance with aspects of the present disclosure.

FIG. 10G provides a correlation heat map of speech characteristics for a phonemic fluency task assessed using the digital mobile speech-based platform, in accordance with aspects of the present disclosure.

FIG. 11A provides a plurality of scree and scatter graphs that plot principal components 1 (PC1) against principal components (PC2) by a paragraph reading speech task, in accordance with aspects of the present disclosure.

FIG. 11B provides a plurality of scree and scatter graphs that plot PC1 against PC2 by a picture description 1 speech task, in accordance with aspects of the present disclosure.

FIG. 11C provides a plurality of scree and scatter graphs that plot PC1 against PC2 by a picture description 2 speech task, in accordance with aspects of the present disclosure.

FIG. 11D provides a plurality of scree and scatter graphs that plot PC1 against PC2 by a DDK speech task, in accordance with aspects of the present disclosure.

FIG. 11E provides a plurality of scree and scatter graphs that plot PC1 against PC2 by a sustained vowel phonation speech task, in accordance with aspects of the present disclosure.

FIG. 11F provides a plurality of scree and scatter graphs that plot PC1 against PC2 by a semantic fluency speech task, in accordance with aspects of the present disclosure.

FIG. 11G provides a plurality of scree and scatter graphs that plot PC1 against PC2 by a phonemic fluency speech task, in accordance with aspects of the present disclosure.

FIG. 12A provides a scatter plot for the test device in which the Spearman correlation between each gait characteristic and the Movement Disorder Society-Unified Parkinson's Disease Rating Scale III (MDS-UPDRS III) score is plotted against the percent difference in each gait characteristic between the ON-state and the OFF-state, in accordance with aspects of the present disclosure.

FIG. 12B provides a scatter plot for the production device in which the Spearman correlation between each gait characteristic and the MDS-UPDRS III score is plotted against the percent difference in each gait characteristic between the ON-state and the OFF-state, in accordance with aspects of the present disclosure.

FIG. 13 provides a heat map that presents a visualization of the scaled values of gait characteristics across different subjects, in accordance with aspects of the present disclosure.

FIG. 14A presents a scatter plot for a test device between Parkinson's Disease (PD) severity and a cadence gait characteristic, in accordance with aspects of the present disclosure.

FIG. 14B presents a scatter plot for the test device illustrating differences between levodopa states for the cadence gait characteristic, in accordance with aspects of the present disclosure.

FIG. 15A presents a scatter plot for the test device between PD severity and a delta of walk times gait characteristic, in accordance with aspects of the present disclosure.

FIG. 15B presents a scatter plot for the test device illustrating differences between levodopa states for the delta of walk times gait characteristic, in accordance with aspects of the present disclosure.

FIG. 16A presents a scatter plot for the test device between PD severity and a distance gait characteristic, in accordance with aspects of the present disclosure.

FIG. 16B presents a scatter plot for the test device illustrating differences between levodopa states for the distance gait characteristic, in accordance with aspects of the present disclosure.

FIG. 17A presents a scatter plot for the test device between PD severity and a double support time gait characteristic, in accordance with aspects of the present disclosure.

FIG. 17B presents a scatter plot for the test device illustrating differences between levodopa states for the double support time gait characteristic, in accordance with aspects of the present disclosure.

FIG. 18A presents a scatter plot for the test device between PD severity and a foot dynamics walk direction gait characteristic, in accordance with aspects of the present disclosure.

FIG. 18B presents a scatter plot for the test device illustrating differences between levodopa states for the foot dynamics walk direction gait characteristic, in accordance with aspects of the present disclosure.

FIG. 19A presents a scatter plot for the test device between PD severity and a force rising gait characteristic, in accordance with aspects of the present disclosure.

FIG. 19B presents a scatter plot for the test device illustrating differences between levodopa states for the force rising gait characteristic, in accordance with aspects of the present disclosure.

FIG. 20A presents a scatter plot for the test device between PD severity and a forefoot hindfoot dominance gait characteristic, in accordance with aspects of the present disclosure.

FIG. 20B presents a scatter plot for the test device illustrating differences between levodopa states for the forefoot hindfoot dominance gait characteristic, in accordance with aspects of the present disclosure.

FIG. 21A presents a scatter plot for the test device between PD severity and a gait line forefoot velocity gait characteristic, in accordance with aspects of the present disclosure.

FIG. 21B presents a scatter plot for the test device illustrating differences between levodopa states for the gait line forefoot velocity gait characteristic, in accordance with aspects of the present disclosure.

FIG. 22A presents a scatter plot for the test device between PD severity and a gait line hindfoot velocity gait characteristic, in accordance with aspects of the present disclosure.

FIG. 22B presents a scatter plot for the test device illustrating differences between levodopa states for the gait line hindfoot velocity gait characteristic, in accordance with aspects of the present disclosure.

FIG. 23A presents a scatter plot for the test device between PD severity and a gait line length gait characteristic, in accordance with aspects of the present disclosure.

FIG. 23B presents a scatter plot for the test device illustrating differences between levodopa states for the gait line length gait characteristic, in accordance with aspects of the present disclosure.

FIG. 24A presents a scatter plot for the test device between PD severity and a gait line midfoot velocity gait characteristic, in accordance with aspects of the present disclosure.

FIG. 24B presents a scatter plot for the test device illustrating differences between levodopa states for the gait line midfoot velocity gait characteristic, in accordance with aspects of the present disclosure.

FIG. 25A presents a scatter plot for the test device between PD severity and a gait line velocity gait characteristic, in accordance with aspects of the present disclosure.

FIG. 25B presents a scatter plot for the test device illustrating differences between levodopa states for the gait line velocity gait characteristic, in accordance with aspects of the present disclosure.

FIG. 26A presents a scatter plot for the test device between PD severity and a gait line width gait characteristic, in accordance with aspects of the present disclosure.

FIG. 26B presents a scatter plot for the test device illustrating differences between levodopa states for the gait line width gait characteristic, in accordance with aspects of the present disclosure.

FIG. 27A presents a scatter plot for the test device between PD severity and a load intensity gait characteristic, in accordance with aspects of the present disclosure.

FIG. 27B presents a scatter plot for the test device illustrating differences between levodopa states for the load intensity gait characteristic, in accordance with aspects of the present disclosure.

FIG. 28A presents a scatter plot for the test device between PD severity and a longitudinal final ground contact deviation gait characteristic, in accordance with aspects of the present disclosure.

FIG. 28B presents a scatter plot for the test device illustrating differences between levodopa states for the longitudinal final ground contact deviation gait characteristic, in accordance with aspects of the present disclosure.

FIG. 29A presents a scatter plot for the test device between PD severity and a longitudinal initial ground contact deviation gait characteristic, in accordance with aspects of the present disclosure.

FIG. 29B presents a scatter plot for the test device illustrating differences between levodopa states for the longitudinal initial ground contact deviation gait characteristic, in accordance with aspects of the present disclosure.

FIG. 30A presents a scatter plot for the test device between PD severity and a maximum force gait characteristic, in accordance with aspects of the present disclosure.

FIG. 30B presents a scatter plot for the test device illustrating differences between levodopa states for the maximum force gait characteristic, in accordance with aspects of the present disclosure.

FIG. 31A presents a scatter plot for the test device between PD severity and a mean force gait characteristic, in accordance with aspects of the present disclosure.

FIG. 31B presents a scatter plot for the test device illustrating differences between levodopa states for the mean force gait characteristic, in accordance with aspects of the present disclosure.

FIG. 32A presents a scatter plot for the test device between PD severity and a medial lateral dominance gait characteristic, in accordance with aspects of the present disclosure.

FIG. 32B presents a scatter plot for the test device illustrating differences between levodopa states for the medial lateral dominance gait characteristic, in accordance with aspects of the present disclosure.

FIG. 33A presents a scatter plot for the test device between PD severity and a speed gait characteristic, in accordance with aspects of the present disclosure.

FIG. 33B presents a scatter plot for the test device illustrating differences between levodopa states for the speed gait characteristic, in accordance with aspects of the present disclosure.

FIG. 34A presents a scatter plot for the test device between PD severity and a stance time gait characteristic, in accordance with aspects of the present disclosure.

FIG. 34B presents a scatter plot for the test device illustrating differences between levodopa states for the stance time gait characteristic, in accordance with aspects of the present disclosure.

FIG. 35A presents a scatter plot for the test device between PD severity and a steps gait characteristic, in accordance with aspects of the present disclosure.

FIG. 35B presents a scatter plot for the test device illustrating differences between levodopa states for the steps gait characteristic, in accordance with aspects of the present disclosure.

FIG. 36A presents a scatter plot for the test device between PD severity and a stride length gait characteristic, in accordance with aspects of the present disclosure.

FIG. 36B presents a scatter plot for the test device illustrating differences between levodopa states for the stride length gait characteristic, in accordance with aspects of the present disclosure.

FIG. 37A presents a scatter plot for the test device between PD severity and a swing time gait characteristic, in accordance with aspects of the present disclosure.

FIG. 37B presents a scatter plot for the test device illustrating differences between levodopa states for the swing time gait characteristic, in accordance with aspects of the present disclosure.

FIG. 38A presents a scatter plot for the test device between PD severity and a time walked gait characteristic, in accordance with aspects of the present disclosure.

FIG. 38B presents a scatter plot for the test device illustrating differences between levodopa states for the time walked gait characteristic, in accordance with aspects of the present disclosure.

FIG. 39A presents a scatter plot for the test device between PD severity and a transversal final ground gait characteristic, in accordance with aspects of the present disclosure.

FIG. 39B presents a scatter plot for the test device illustrating differences between levodopa states for the transversal final ground gait characteristic, in accordance with aspects of the present disclosure.

FIG. 40A presents a scatter plot for the test device between PD severity and a transversal initial ground contact deviation gait characteristic, in accordance with aspects of the present disclosure.

FIG. 40B presents a scatter plot for the test device illustrating differences between levodopa states for the transversal initial ground contact deviation gait characteristic, in accordance with aspects of the present disclosure.

FIG. 41A presents a scatter plot for the test device between PD severity and a turning steps gait characteristic, in accordance with aspects of the present disclosure.

FIG. 41B presents a scatter plot for the test device illustrating differences between levodopa states for the turning steps gait characteristic, in accordance with aspects of the present disclosure.

FIG. 42A presents a scatter plot for the test device between PD severity and a turning velocity gait characteristic, in accordance with aspects of the present disclosure.

FIG. 42B presents a scatter plot for the test device illustrating differences between levodopa states for the turning velocity gait characteristic, in accordance with aspects of the present disclosure.

FIG. 43A presents a scatter plot for the test device between PD severity and a walk time gait characteristic, in accordance with aspects of the present disclosure.

FIG. 43B presents a scatter plot for the test device illustrating differences between levodopa states for the walk time gait characteristic, in accordance with aspects of the present disclosure.

FIG. 44A presents a scatter plot for a production device between PD severity and a ambulation time gait characteristic, in accordance with aspects of the present disclosure.

FIG. 44B presents a scatter plot for the production device illustrating differences between levodopa states for the ambulation time gait characteristic, in accordance with aspects of the present disclosure.

FIG. 45A presents a scatter plot for the production device between PD severity and a cadence gait characteristic, in accordance with aspects of the present disclosure.

FIG. 45B presents a scatter plot for the production device illustrating differences between levodopa states for the cadence gait characteristic, in accordance with aspects of the present disclosure.

FIG. 46A presents a scatter plot for the production device between PD severity and a cycle time gait characteristic, in accordance with aspects of the present disclosure.

FIG. 46B presents a scatter plot for the production device illustrating differences between levodopa states for the cycle time gait characteristic, in accordance with aspects of the present disclosure.

FIG. 47A presents a scatter plot for the production device between PD severity and a cycle time differential gait characteristic, in accordance with aspects of the present disclosure.

FIG. 47B presents a scatter plot for the production device illustrating differences between levodopa states for the cycle time differential gait characteristic, in accordance with aspects of the present disclosure.

FIG. 48A presents a scatter plot for the production device between PD severity and a distance gait characteristic, in accordance with aspects of the present disclosure.

FIG. 48B presents a scatter plot for the production device illustrating differences between levodopa states for the distance gait characteristic, in accordance with aspects of the present disclosure.

FIG. 49A presents a scatter plot for the production device between PD severity and a double support percent cycle gait characteristic, in accordance with aspects of the present disclosure.

FIG. 49B presents a scatter plot for the production device illustrating differences between levodopa states for the double support percent cycle gait characteristic, in accordance with aspects of the present disclosure.

FIG. 50A presents a scatter plot for the production device between PD severity and a double support time gait characteristic, in accordance with aspects of the present disclosure.

FIG. 50B presents a scatter plot for the production device illustrating differences between levodopa states for the double support time gait characteristic, in accordance with aspects of the present disclosure.

FIG. 51A presents a scatter plot for the production device between PD severity and a double support time stand deviation gait characteristic, in accordance with aspects of the present disclosure.

FIG. 51B presents a scatter plot for the production device illustrating differences between levodopa states for the double support time stand deviation gait characteristic, in accordance with aspects of the present disclosure.

FIG. 52A presents a scatter plot for the production device between PD severity and a double support load percent GC gait characteristic, in accordance with aspects of the present disclosure.

FIG. 52B presents a scatter plot for the production device illustrating differences between levodopa states for the double support load percent GC gait characteristic, in accordance with aspects of the present disclosure.

FIG. 53A presents a scatter plot for the production device between PD severity and a double support load time gait characteristic, in accordance with aspects of the present disclosure.

FIG. 53B presents a scatter plot for the production device illustrating differences between levodopa states for the double support load time gait characteristic, in accordance with aspects of the present disclosure.

FIG. 54A presents a scatter plot for the production device between PD severity and a double support unload percent GC gait characteristic, in accordance with aspects of the present disclosure.

FIG. 54B presents a scatter plot for the production device illustrating differences between levodopa states for the double support unload percent GC gait characteristic, in accordance with aspects of the present disclosure.

FIG. 55A presents a scatter plot for the production device between PD severity and a double support unload time gait characteristic, in accordance with aspects of the present disclosure.

FIG. 55B presents a scatter plot for the production device illustrating differences between levodopa states for the double support unload time gait characteristic, in accordance with aspects of the present disclosure.

FIG. 56A presents a scatter plot for the production device between PD severity and a foot length gait characteristic, in accordance with aspects of the present disclosure.

FIG. 56B presents a scatter plot for the production device illustrating differences between levodopa states for the foot length gait characteristic, in accordance with aspects of the present disclosure.

FIG. 57A presents a scatter plot for the production device between PD severity and a foot width gait characteristic, in accordance with aspects of the present disclosure.

FIG. 57B presents a scatter plot for the production device illustrating differences between levodopa states for the foot width gait characteristic, in accordance with aspects of the present disclosure.

FIG. 58A presents a scatter plot for the production device between PD severity and a heel off on percent gait characteristic, in accordance with aspects of the present disclosure.

FIG. 58B presents a scatter plot for the production device illustrating differences between levodopa states for the heel off on percent gait characteristic, in accordance with aspects of the present disclosure.

FIG. 59A presents a scatter plot for the production device between PD severity and a heel off on standard deviation gait characteristic, in accordance with aspects of the present disclosure.

FIG. 59B presents a scatter plot for the production device illustrating differences between levodopa states for the heel off on standard deviation gait characteristic, in accordance with aspects of the present disclosure.

FIG. 60A presents a scatter plot for the production device between PD severity and a heel off on time gait characteristic, in accordance with aspects of the present disclosure.

FIG. 60B presents a scatter plot for the production device illustrating differences between levodopa states for the heel off on time gait characteristic, in accordance with aspects of the present disclosure.

FIG. 61A presents a scatter plot for the production device between PD severity and a HH base support gait characteristic, in accordance with aspects of the present disclosure.

FIG. 61B presents a scatter plot for the production device illustrating differences between levodopa states for a base support gait characteristic, in accordance with aspects of the present disclosure.

FIG. 62A presents a scatter plot for the production device between PD severity and a single support percent cycle gait characteristic, in accordance with aspects of the present disclosure.

FIG. 62B presents a scatter plot for the production device illustrating differences between levodopa states for the single support percent cycle gait characteristic, in accordance with aspects of the present disclosure.

FIG. 63A presents a scatter plot for the production device between PD severity and a single support time gait characteristic, in accordance with aspects of the present disclosure.

FIG. 63B presents a scatter plot for the production device illustrating differences between levodopa states for the single support time gait characteristic, in accordance with aspects of the present disclosure.

FIG. 64A presents a scatter plot for the production device between PD severity and a single support time standard deviation gait characteristic, in accordance with aspects of the present disclosure.

FIG. 64B presents a scatter plot for the production device illustrating differences between levodopa states for the single support time standard deviation gait characteristic, in accordance with aspects of the present disclosure.

FIG. 65A presents a scatter plot for the production device between PD severity and a stance percent of cycle gait characteristic, in accordance with aspects of the present disclosure.

FIG. 65B presents a scatter plot for the production device illustrating differences between levodopa states for the stance percent of cycle gait characteristic, in accordance with aspects of the present disclosure.

FIG. 66A presents a scatter plot for the production device between PD severity and a stance time gait characteristic, in accordance with aspects of the present disclosure.

FIG. 66B presents a scatter plot for the production device illustrating differences between levodopa states for the stance time gait characteristic, in accordance with aspects of the present disclosure.

FIG. 67A presents a scatter plot for the production device between PD severity and a stance time standard deviation gait characteristic, in accordance with aspects of the present disclosure.

FIG. 67B presents a scatter plot for the production device illustrating differences between levodopa states for the stance time standard deviation gait characteristic, in accordance with aspects of the present disclosure.

FIG. 68A presents a scatter plot for the production device between PD severity and a step count gait characteristic, in accordance with aspects of the present disclosure.

FIG. 68B presents a scatter plot for the production device illustrating differences between levodopa states for the step count gait characteristic, in accordance with aspects of the present disclosure.

FIG. 69A presents a scatter plot for the production device between PD severity and a step length standard deviation gait characteristic, in accordance with aspects of the present disclosure.

FIG. 69B presents a scatter plot for the production device illustrating differences between levodopa states for the step length standard deviation gait characteristic, in accordance with aspects of the present disclosure.

FIG. 70A presents a scatter plot for the production device between PD severity and a step length gait characteristic, in accordance with aspects of the present disclosure.

FIG. 70B presents a scatter plot for the production device illustrating differences between levodopa states for the step length gait characteristic, in accordance with aspects of the disclosure.

FIG. 71A presents a scatter plot for the production device between PD severity and a step length differential gait characteristic, in accordance with aspects of the present disclosure.

FIG. 71B presents a scatter plot for the production device illustrating differences between levodopa states for the step length differential gait characteristic, in accordance with aspects of the present disclosure.

FIG. 72A presents a scatter plot for the production device between PD severity and a step time gait characteristic, in accordance with aspects of the present disclosure.

FIG. 72B presents a scatter plot for the production device illustrating differences between levodopa states for the step time gait characteristic, in accordance with aspects of the present disclosure.

FIG. 73A presents a scatter plot for the production device between PD severity and a step time differential gait characteristic, in accordance with aspects of the present disclosure.

FIG. 73B presents a scatter plot for the production device illustrating differences between levodopa states for the step time differential gait characteristic, in accordance with aspects of the present disclosure.

FIG. 74A presents a scatter plot for the production device between PD severity and a step time standard deviation gait characteristic, in accordance with aspects of the present disclosure.

FIG. 74B presents a scatter plot for the production device illustrating differences between levodopa states for the step time standard deviation gait characteristic, in accordance with aspects of the present disclosure.

FIG. 75A presents a scatter plot for the production device between PD severity and a stride length gait characteristic, in accordance with aspects of the present disclosure.

FIG. 75B presents a scatter plot for the production device illustrating differences between levodopa states for the stride length gait characteristic, in accordance with aspects of the present disclosure.

FIG. 76A presents a scatter plot for the production device between PD severity and a stride length standard deviation gait characteristic, in accordance with aspects of the present disclosure.

FIG. 76B presents a scatter plot for the production device illustrating differences between levodopa states for the stride length standard deviation gait characteristic, in accordance with aspects of the present disclosure.

FIG. 77A presents a scatter plot for the production device between PD severity and a stride time standard deviation gait characteristic, in accordance with aspects of the present disclosure.

FIG. 77B presents a scatter plot for the production device illustrating differences between levodopa states for the stride time standard deviation gait characteristic, in accordance with aspects of the present disclosure.

FIG. 78A presents a scatter plot for the production device between PD severity and a stride velocity gait characteristic, in accordance with aspects of the present disclosure.

FIG. 78B presents a scatter plot for the production device illustrating differences between levodopa states for the stride velocity gait characteristic, in accordance with aspects of the present disclosure.

FIG. 79A presents a scatter plot for the production device between PD severity and a stride velocity standard deviation gait characteristic, in accordance with aspects of the present disclosure.

FIG. 79B presents a scatter plot for the production device illustrating differences between levodopa states for the stride velocity standard deviation gait characteristic, in accordance with aspects of the present disclosure.

FIG. 80A presents a scatter plot for the production device between PD severity and a support base standard deviation gait characteristic, in accordance with aspects of the present disclosure.

FIG. 80B presents a scatter plot for the production device illustrating differences between levodopa states for the support base standard deviation gait characteristic, in accordance with aspects of the present disclosure.

FIG. 81A presents a scatter plot for the production device between PD severity and a swing percent of cycle gait characteristic, in accordance with aspects of the present disclosure.

FIG. 81B presents a scatter plot for the production device illustrating differences between levodopa states for the swing percent of cycle gait characteristic, in accordance with aspects of the present disclosure.

FIG. 82A presents a scatter plot for the production device between PD severity and a swing time gait characteristic, in accordance with aspects of the present disclosure.

FIG. 82B presents a scatter plot for the production device illustrating differences between levodopa states for the swing time gait characteristic, in accordance with aspects of the present disclosure.

FIG. 83A presents a scatter plot for the production device between PD severity and a swing time standard deviation gait characteristic, in accordance with aspects of the present disclosure.

FIG. 83B presents a scatter plot for the production device illustrating differences between levodopa states for the swing time standard deviation gait characteristic, in accordance with aspects of the present disclosure.

FIG. 84A presents a scatter plot for the production device between PD severity and a toe in out gait characteristic, in accordance with aspects of the present disclosure.

FIG. 84B presents a scatter plot for the production device illustrating differences between levodopa states for the toe in out gait characteristic, in accordance with aspects of the present disclosure.

FIG. 85A presents a scatter plot for the production device between PD severity and a velocity gait characteristic, in accordance with aspects of the present disclosure.

FIG. 85B presents a scatter plot for the production device illustrating differences between levodopa states for the velocity gait characteristic, in accordance with aspects of the present disclosure.

FIG. 86 presents a scatter plot that provides a visual representation of both the strength of association with disease severity and the sensitivity to levodopa treatment, in accordance with aspects of the present disclosure.

FIG. 87 provides a volcano plot of wearable insole test device gait characteristic associations with MDS-UPDRS III scores, in accordance with aspects of the present disclosure.

FIG. 88 provides a volcano plot of production device gait characteristic associations with MDS-UPDRS III scores, in accordance with aspects of the present disclosure.

FIG. 89 provides a volcano plot of mean differences between the ON and OFF state per gait characteristic measured by the test device, in accordance with aspects of the present disclosure.

FIG. 90 provides a volcano plot of mean differences between the ON and OFF state per gait characteristic measured by the production device, in accordance with aspects of the present disclosure.

FIG. 91A provides a graph that presents the model estimated association between each gait characteristic measure by a test device, in accordance with aspects of the present disclosure.

FIG. 91B provides a graph that presents the model estimated association between each gait characteristic measure by a production device, in accordance with aspects of the present disclosure.

FIG. 92A provides a volcano plot corresponding to an acoustic speech characteristic association with the MDS-UPDRS III score, in accordance with aspects of the present disclosure.

FIG. 92B provides a volcano plot corresponding to a discourse speech characteristic association with the MDS-UPDRS III score, in accordance with aspects of the present disclosure.

FIG. 92C provides a volcano plot corresponding to a global coherence characteristic association with the MDS-UPDRS III score, in accordance with aspects of the present disclosure.

FIG. 92D provides a volcano plot corresponding to an information content characteristic association with the MDS-UPDRS III score, in accordance with aspects of the present disclosure.

FIG. 92E provides a volcano plot corresponding to a lexical speech characteristic association with the MDS-UPDRS III score, in accordance with aspects of the present disclosure.

FIG. 92F provides a volcano plot corresponding to a local coherence speech characteristic association with the MDS-UPDRS III score, in accordance with aspects of the present disclosure.

FIG. 92G provides a volcano plot corresponding to a morphological speech characteristic association with the MDS-UPDRS III score, in accordance with aspects of the present disclosure.

FIG. 92H provides a volcano plot corresponding to another speech characteristic association with the MDS-UPDRS III score, in accordance with aspects of the present disclosure.

FIG. 92I provides a volcano plot corresponding to a sentiment speech characteristic association with the MDS-UPDRS III score, in accordance with aspects of the present disclosure.

FIG. 92J provides a volcano plot corresponding to a syntactic speech characteristic association with the MDS-UPDRS III score, in accordance with aspects of the present disclosure.

FIG. 92K provides a volcano plot corresponding to a task score speech characteristic association with the MDS-UPDRS III score, in accordance with aspects of the present disclosure.

FIG. 92L provides a volcano plot corresponding to a timing speech characteristic association with the MDS-UPDRS III score, in accordance with aspects of the present disclosure.

FIG. 93A provides a volcano plot presenting a mean difference in an acoustic speech characteristic between an ON and OFF state, in accordance with aspects of the present disclosure.

FIG. 93B provides a volcano plot presenting a mean difference in a discourse speech characteristic between an ON and OFF state, in accordance with aspects of the present disclosure.

FIG. 93C provides a volcano plot presenting a mean difference in a global coherence speech characteristic between an ON and OFF state, in accordance with aspects of the present disclosure.

FIG. 93D provides a volcano plot presenting a mean difference in an information content speech characteristic between an ON and OFF state, in accordance with aspects of the present disclosure.

FIG. 93E provides a volcano plot presenting a mean difference in a lexical speech characteristic between an ON and OFF state, in accordance with aspects of the present disclosure.

FIG. 93F provides a volcano plot presenting a mean difference in a local coherence characteristic between an ON and OFF state, in accordance with aspects of the present disclosure.

FIG. 93G provides a volcano plot presenting a mean difference in a morphological speech characteristic between an ON and OFF state, in accordance with aspects of the present disclosure.

FIG. 93H provides a volcano plot presenting a mean difference in another speech characteristic between an ON and OFF state, in accordance with aspects of the present disclosure.

FIG. 931 provides a volcano plot presenting a mean difference in a sentiment speech characteristic between an ON and OFF state, in accordance with aspects of the present disclosure.

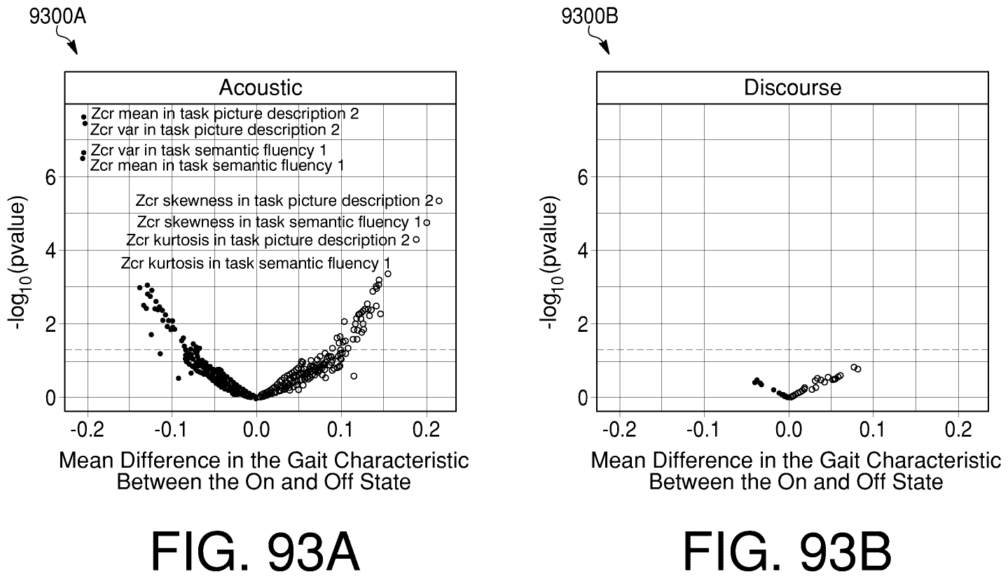

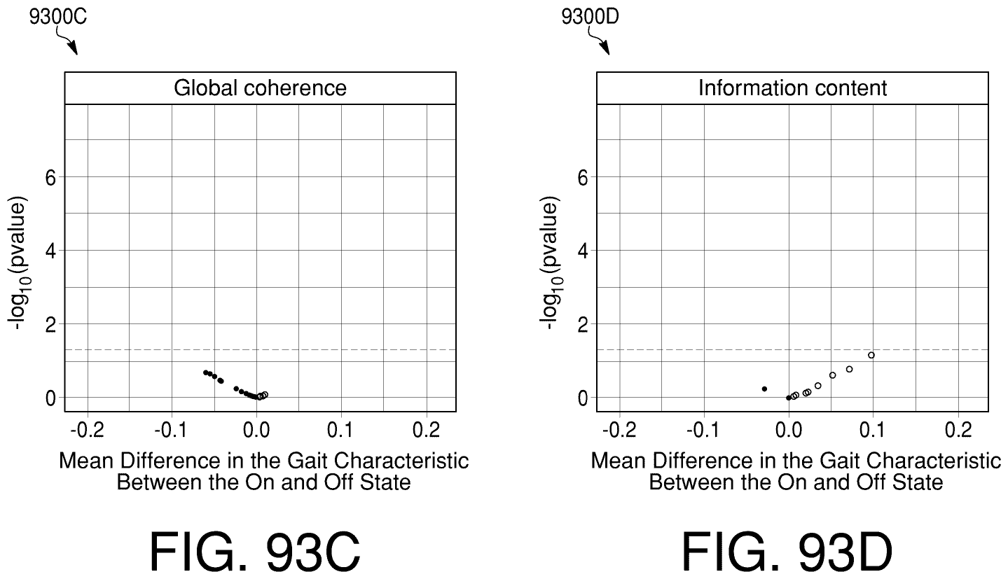

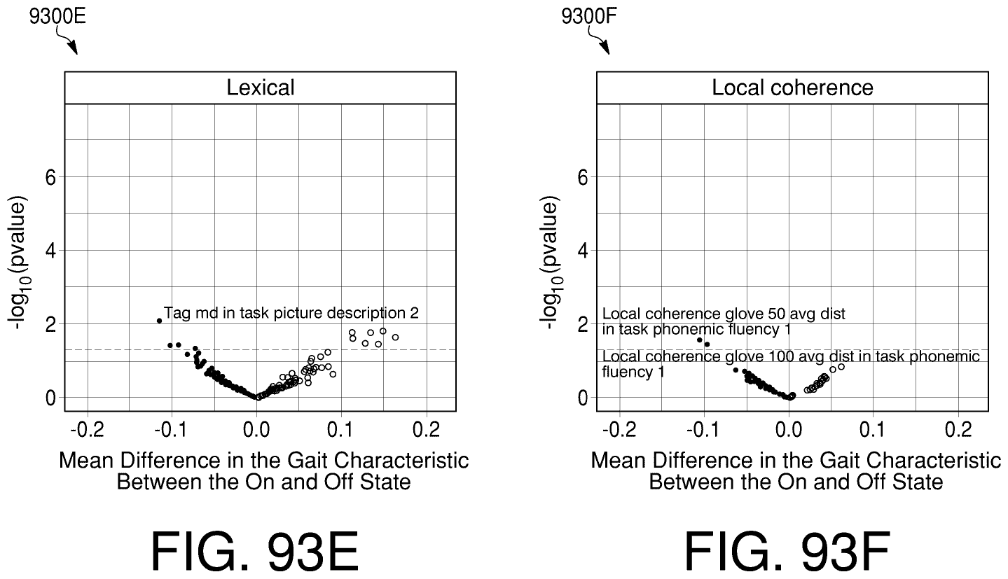

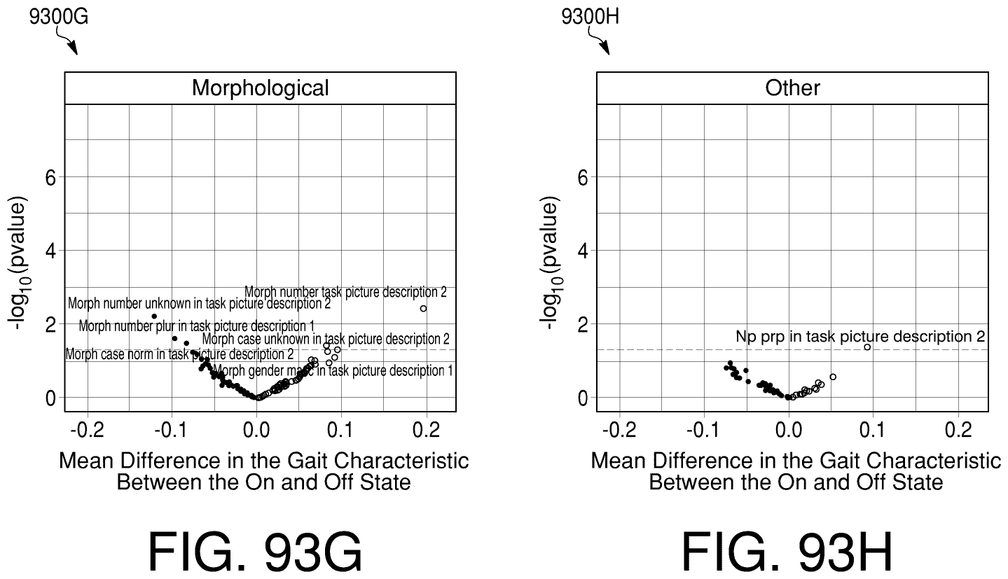

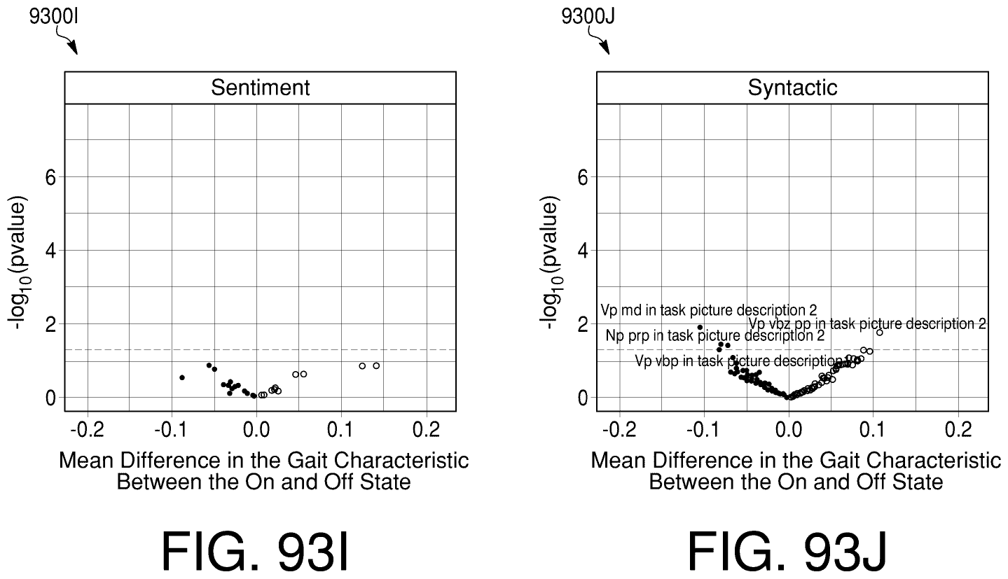

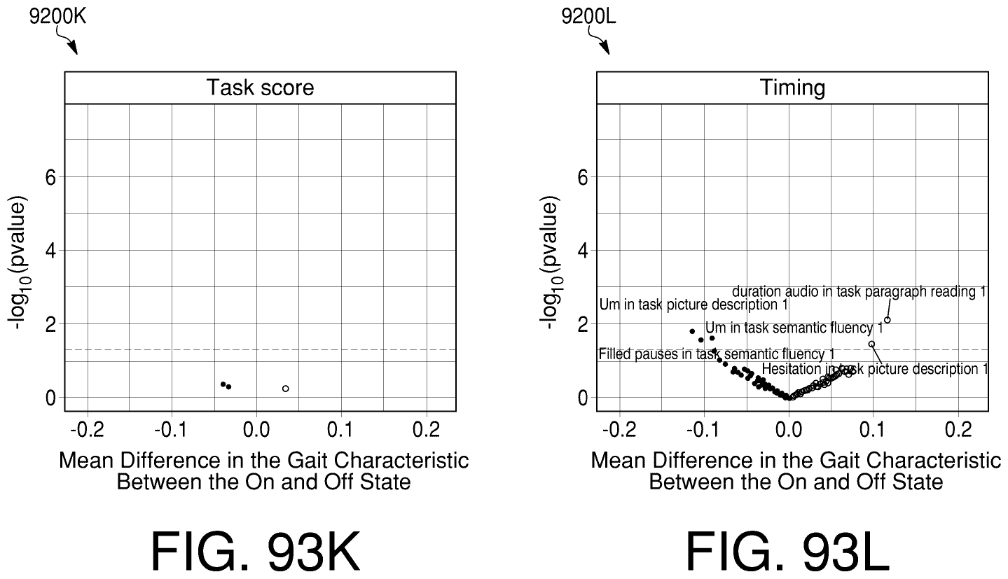

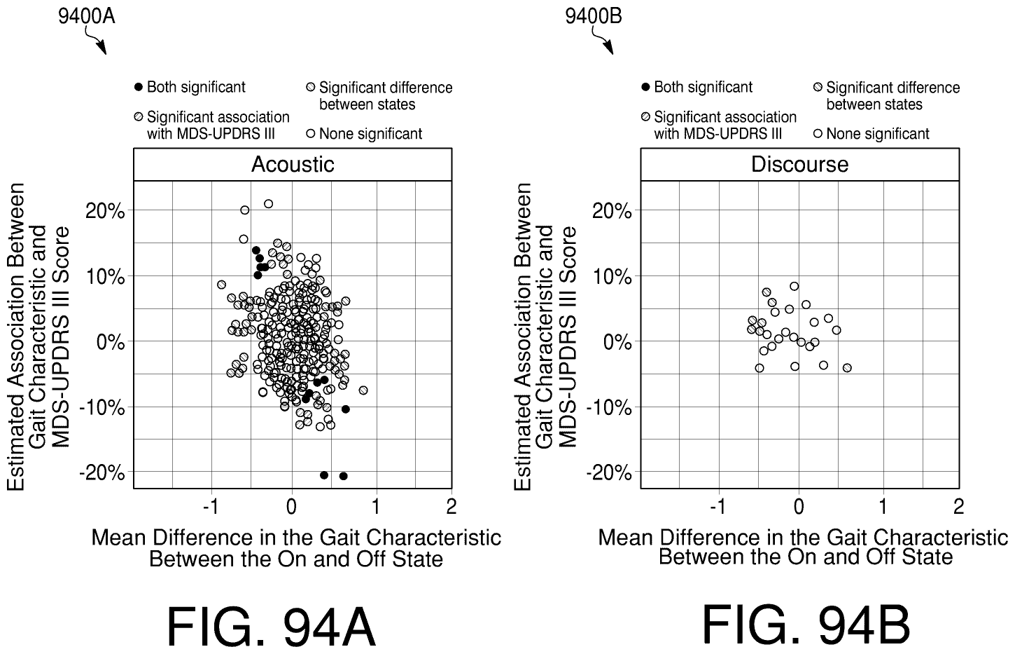

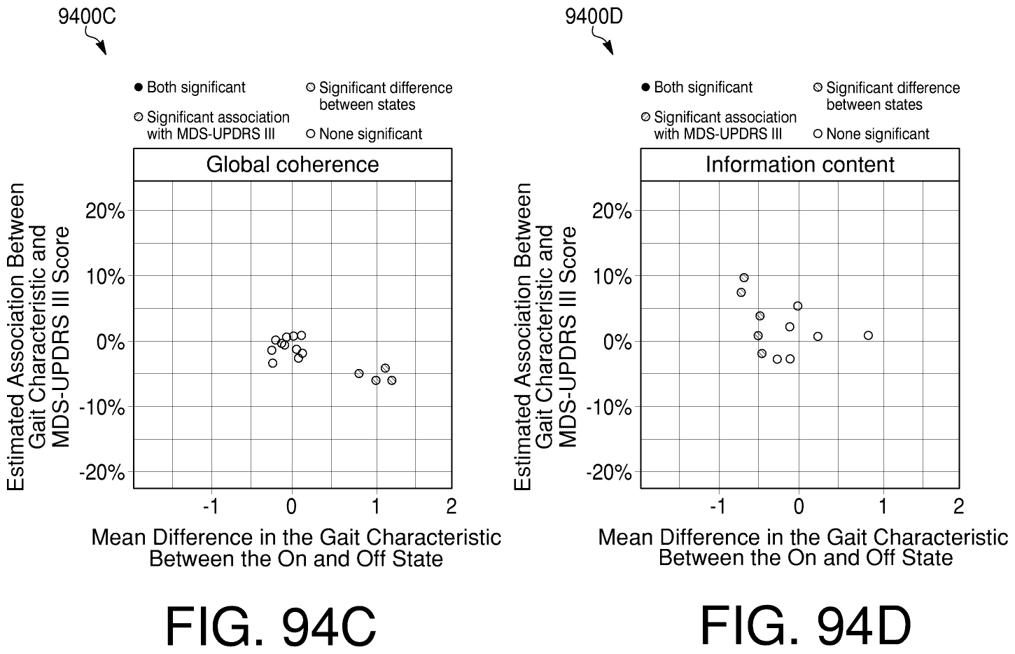

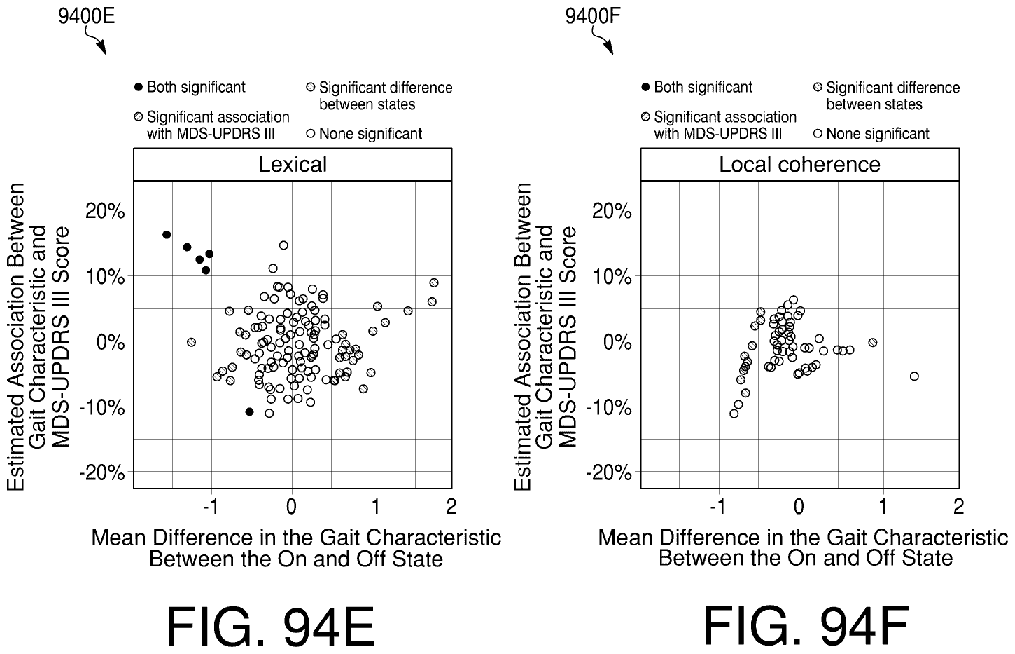

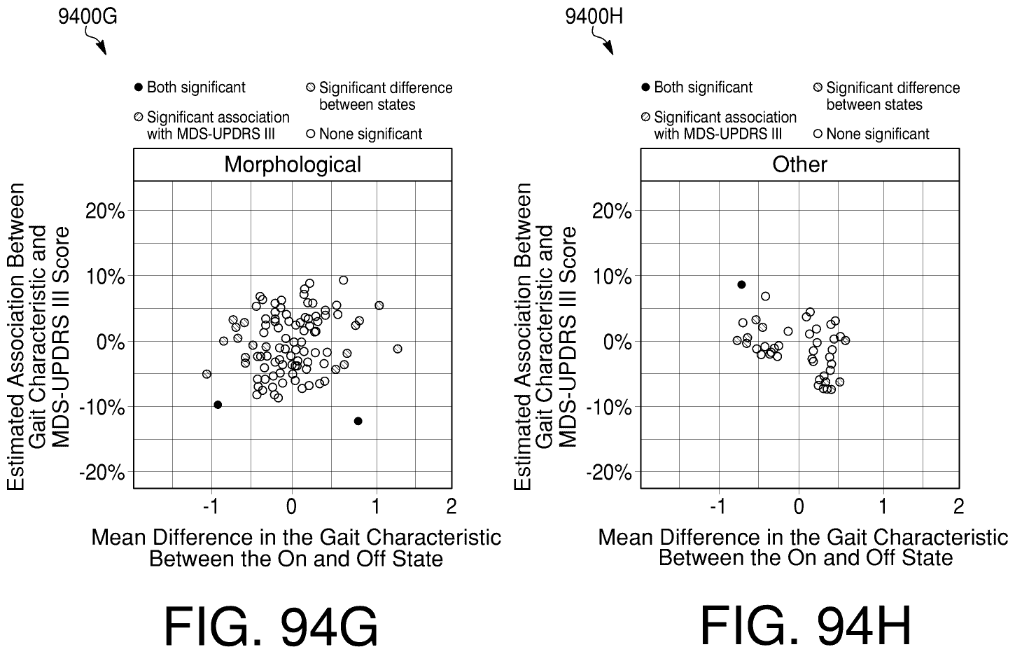

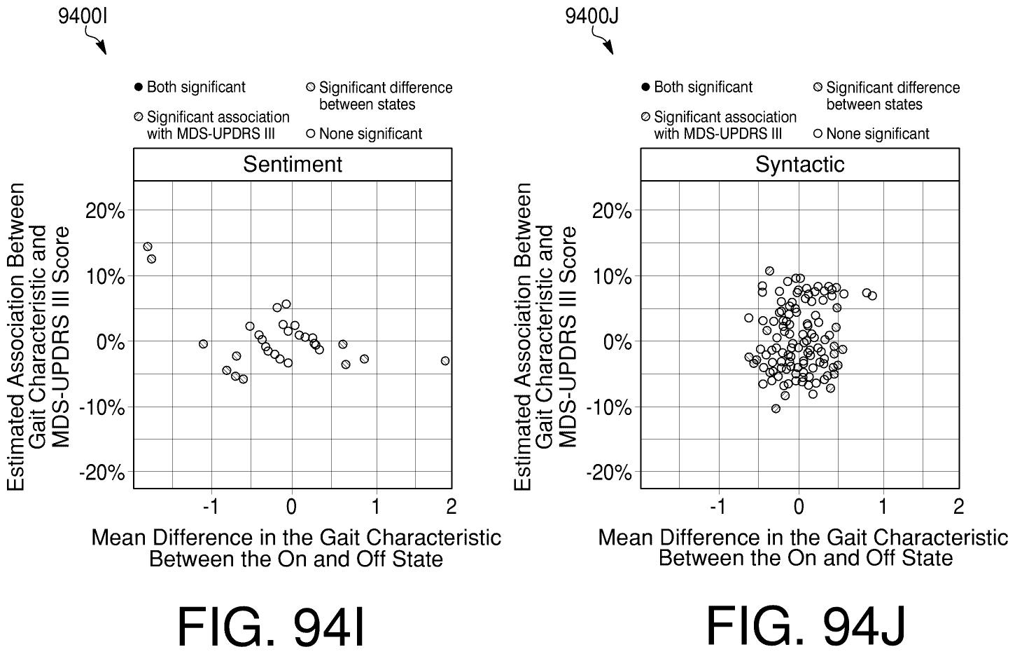

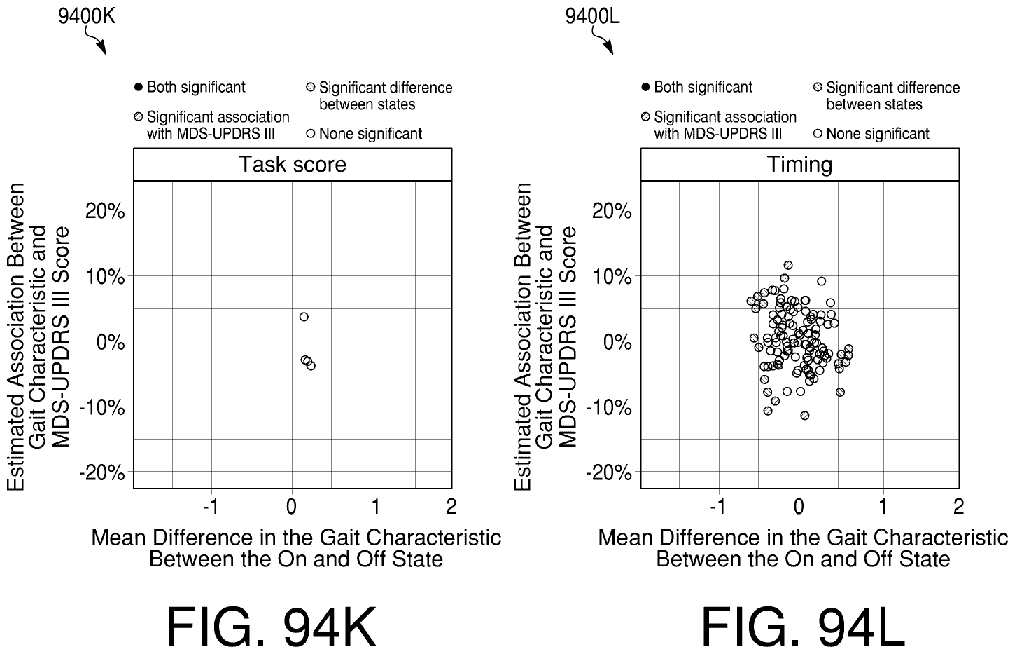

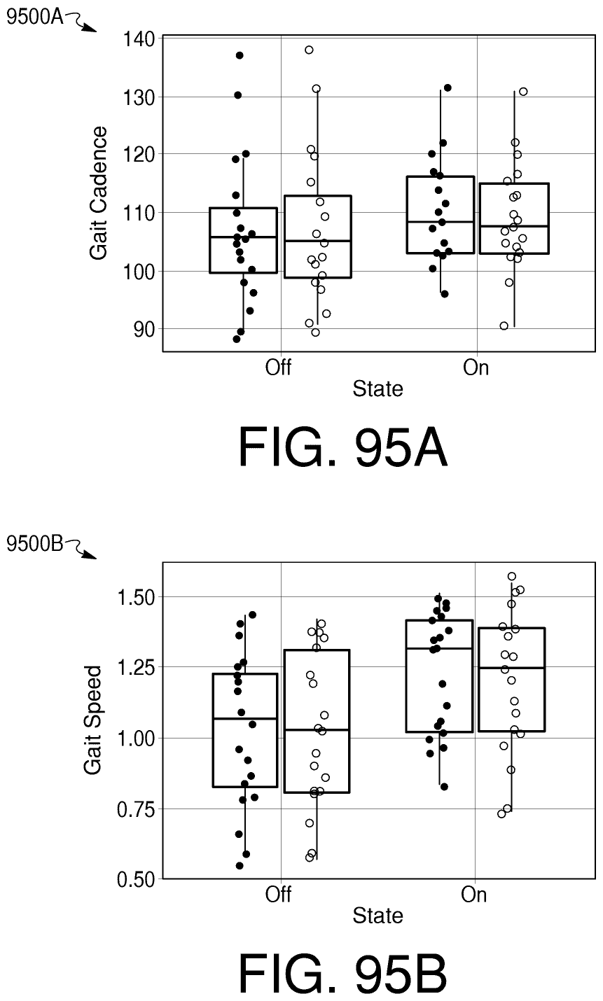

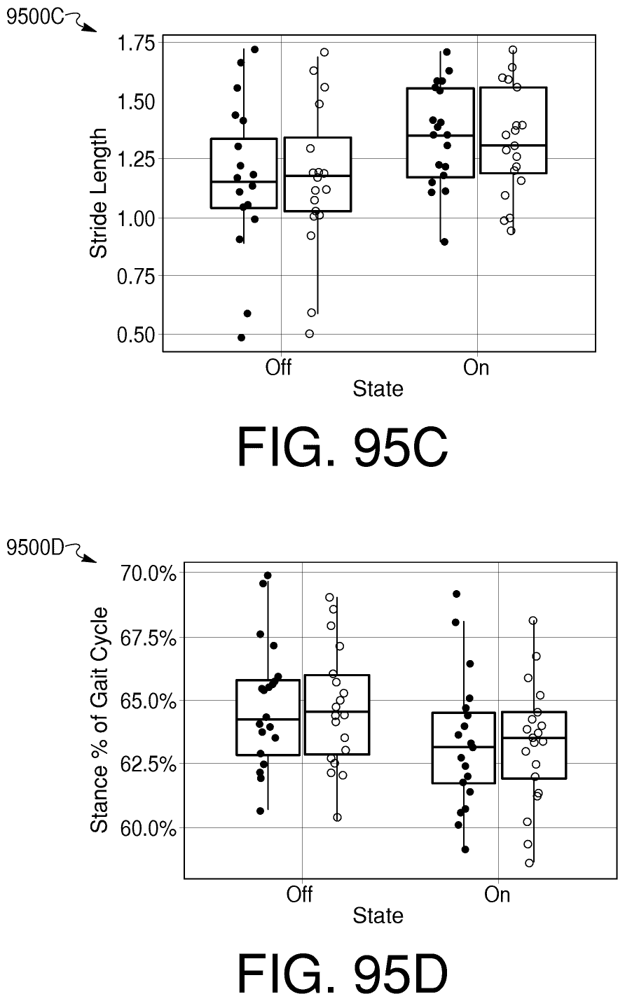

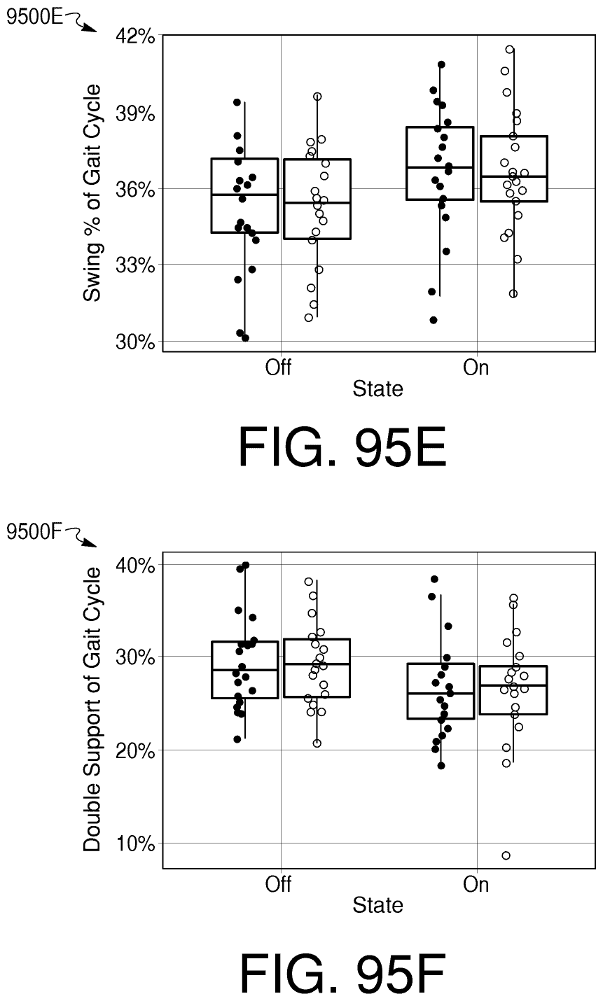

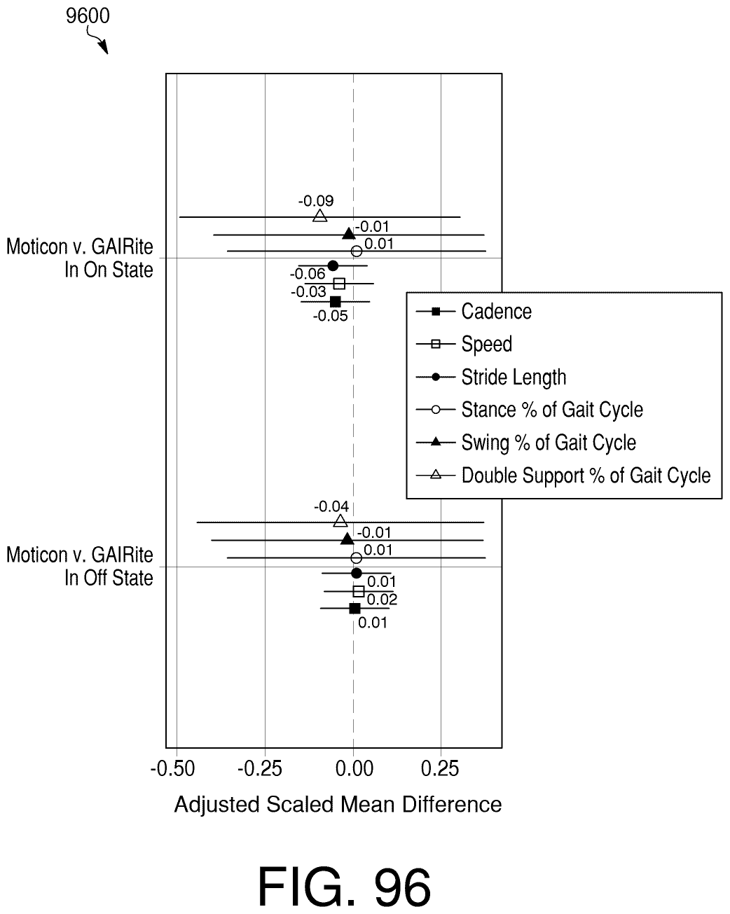

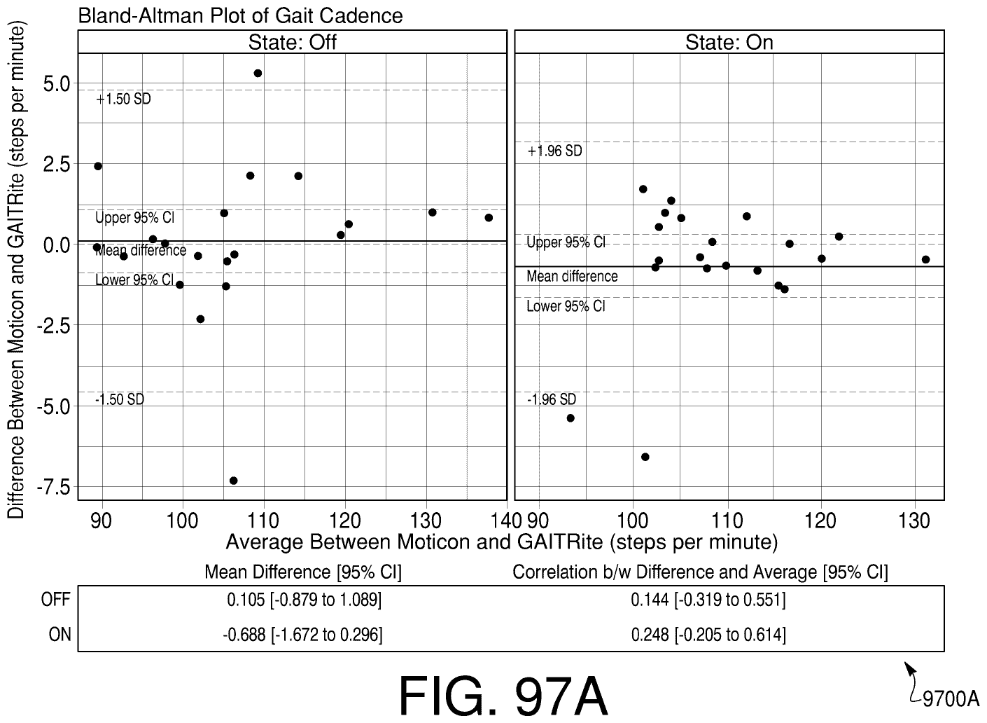

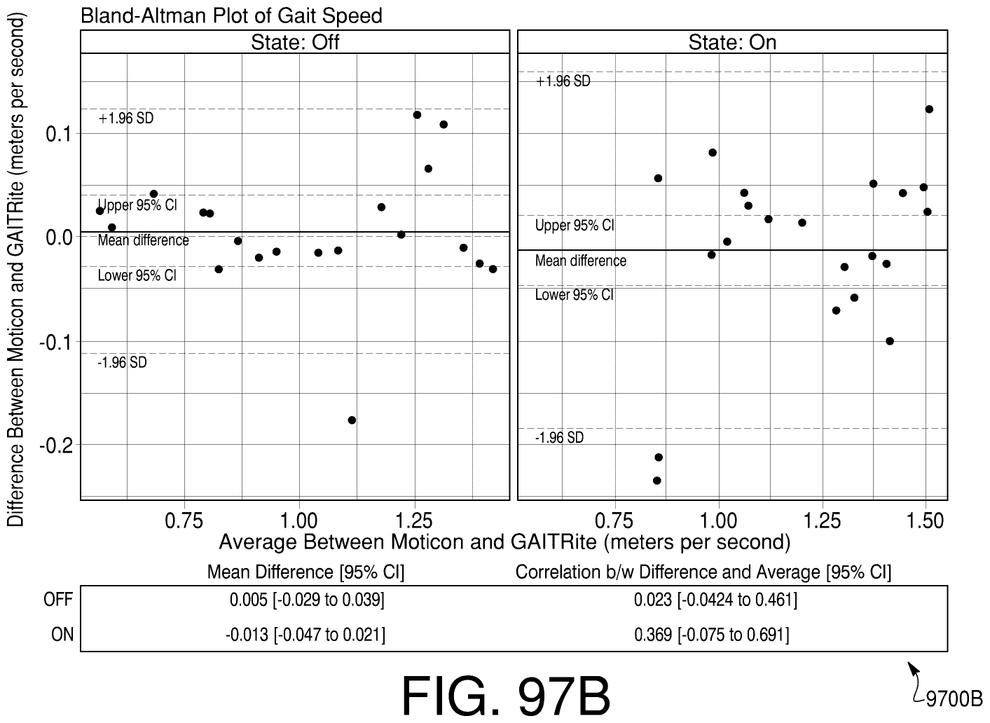

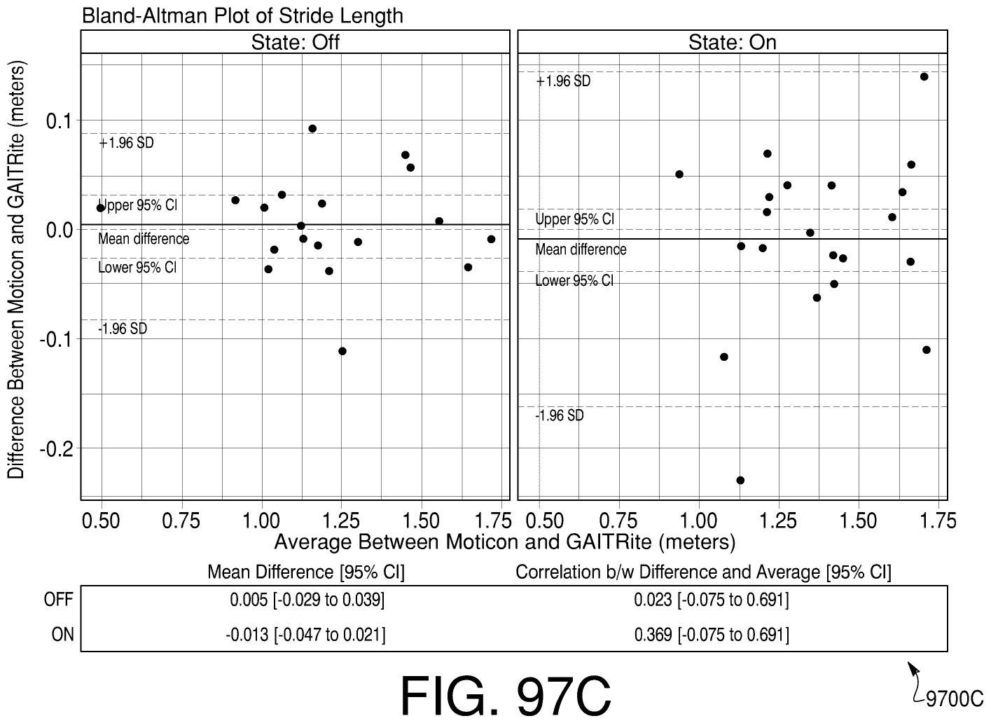

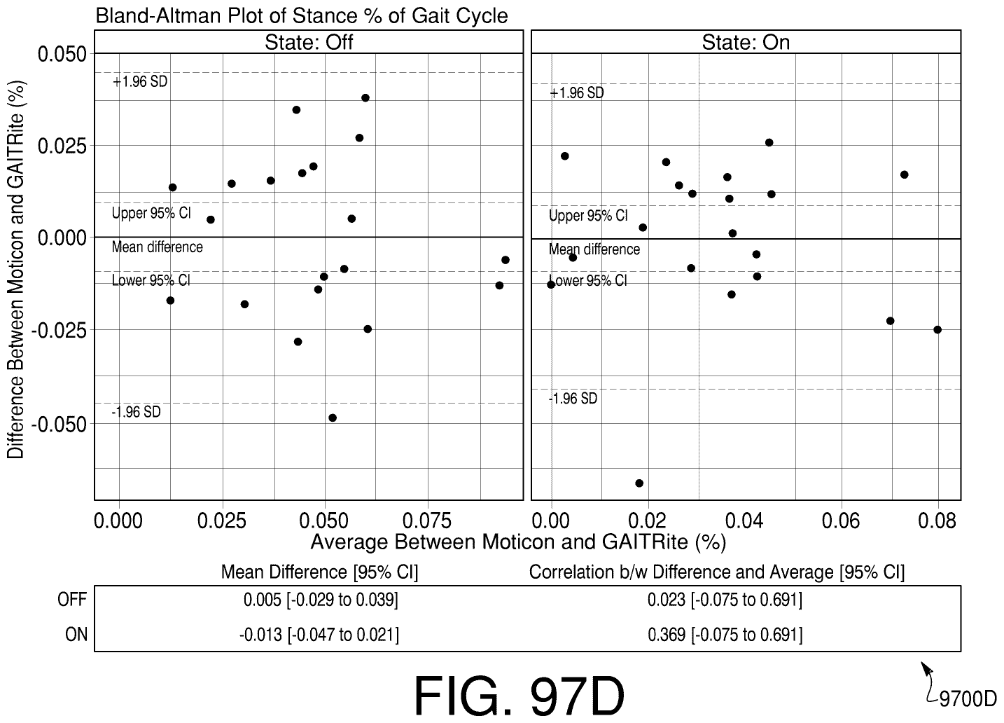

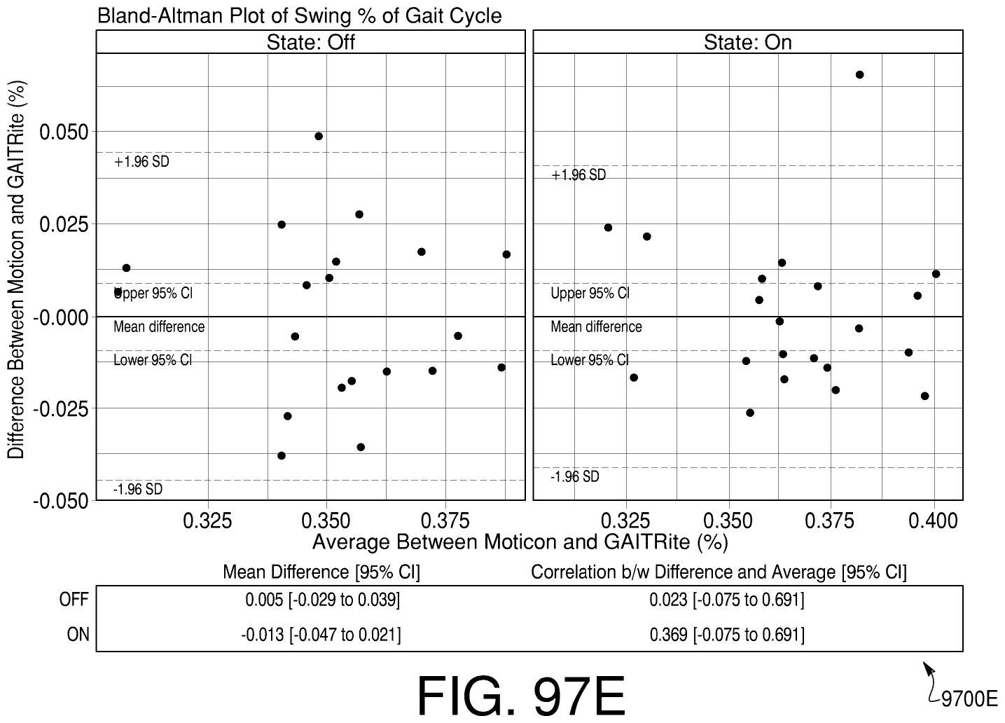

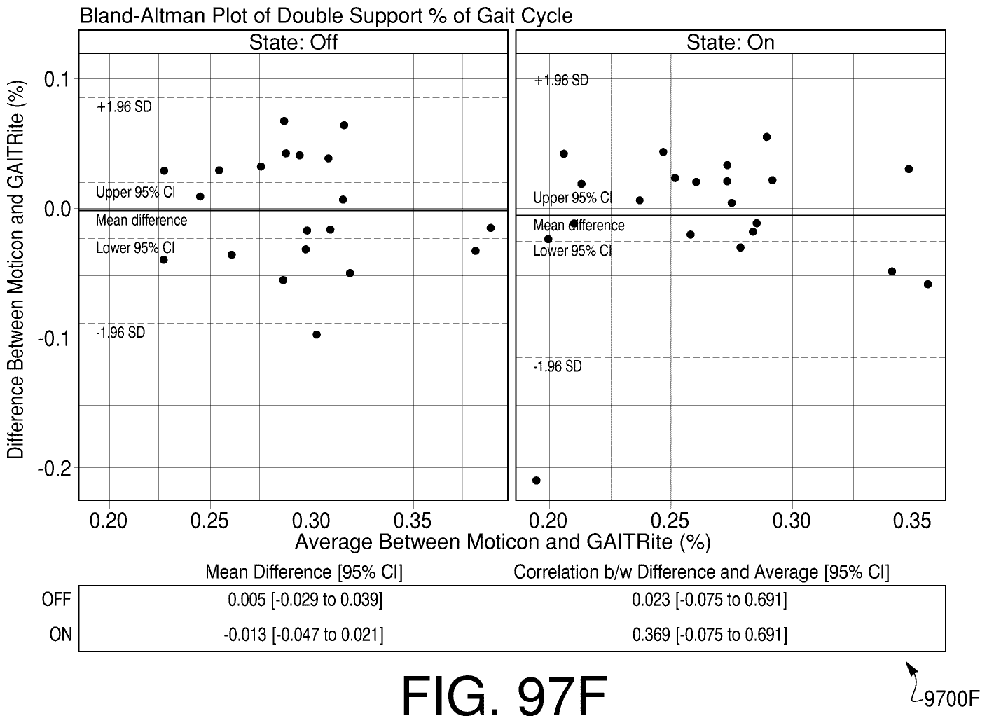

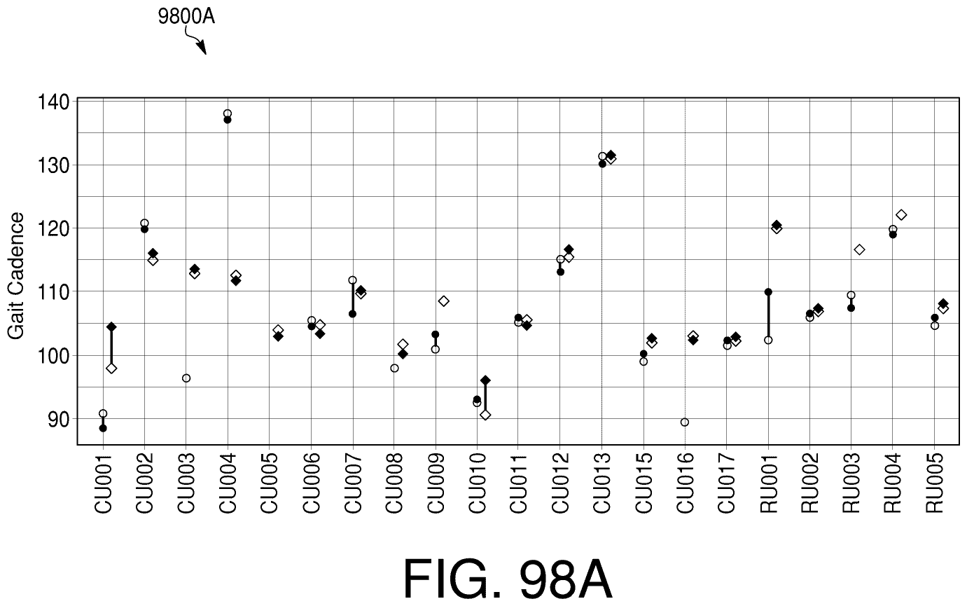

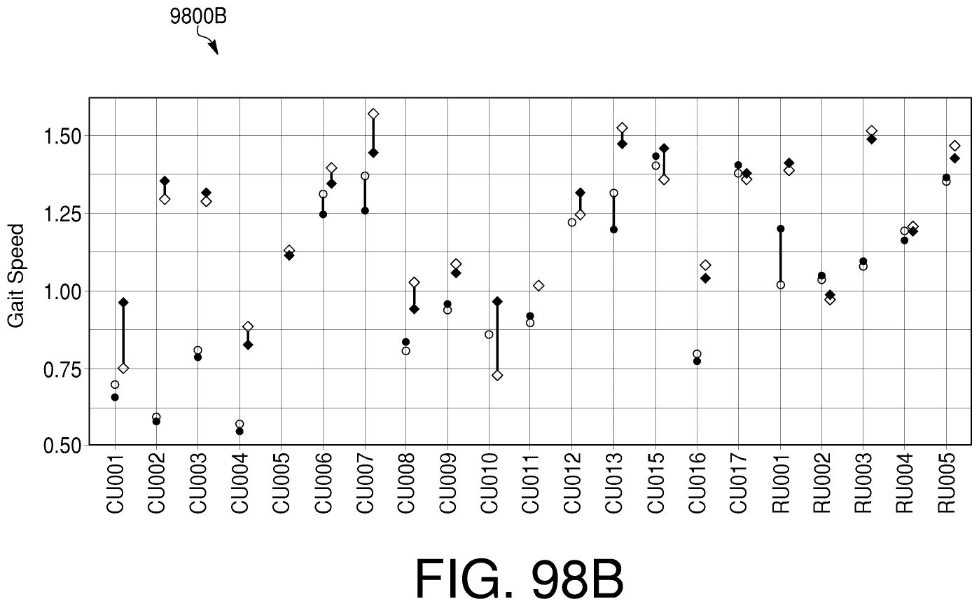

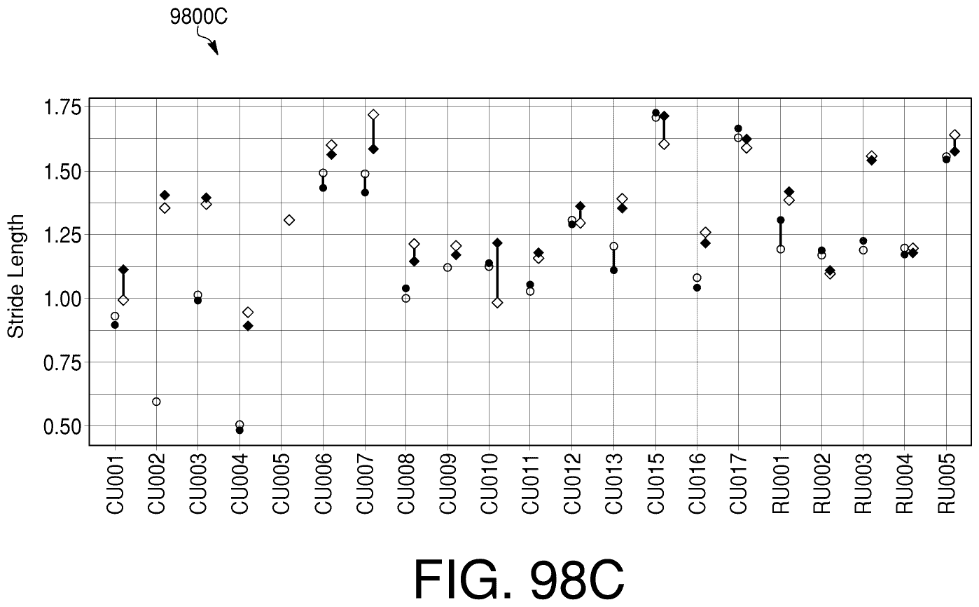

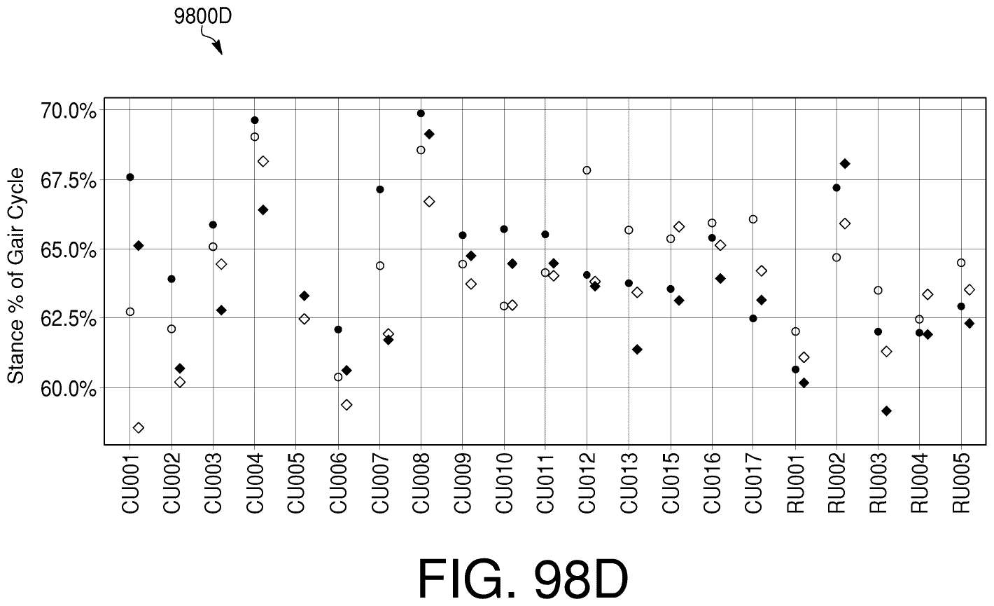

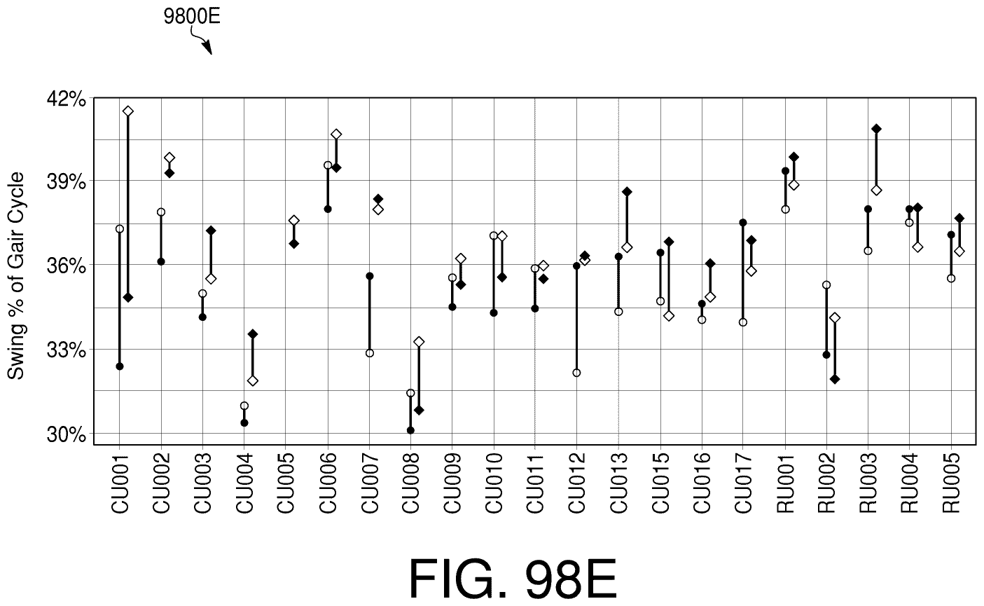

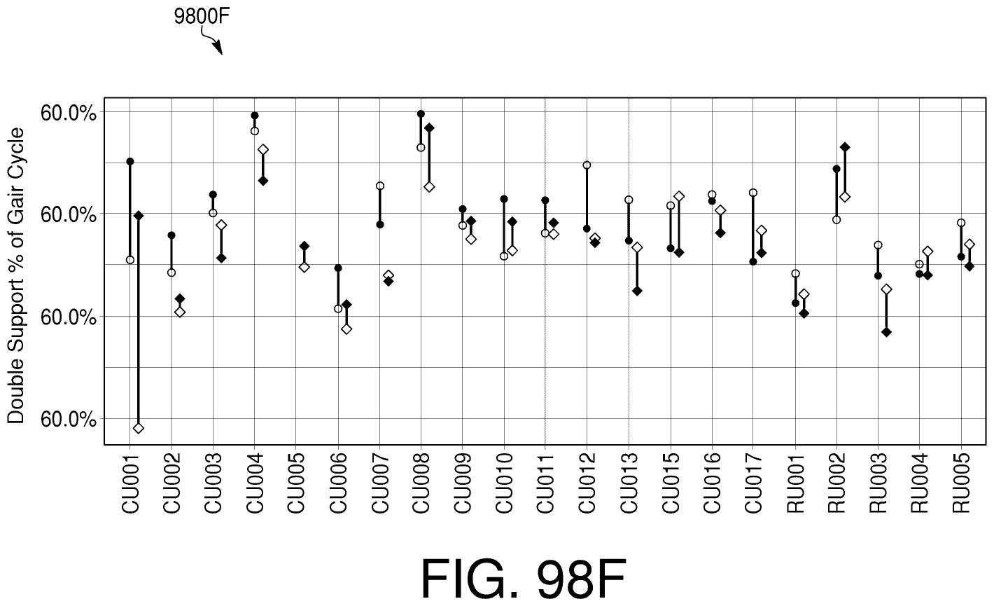

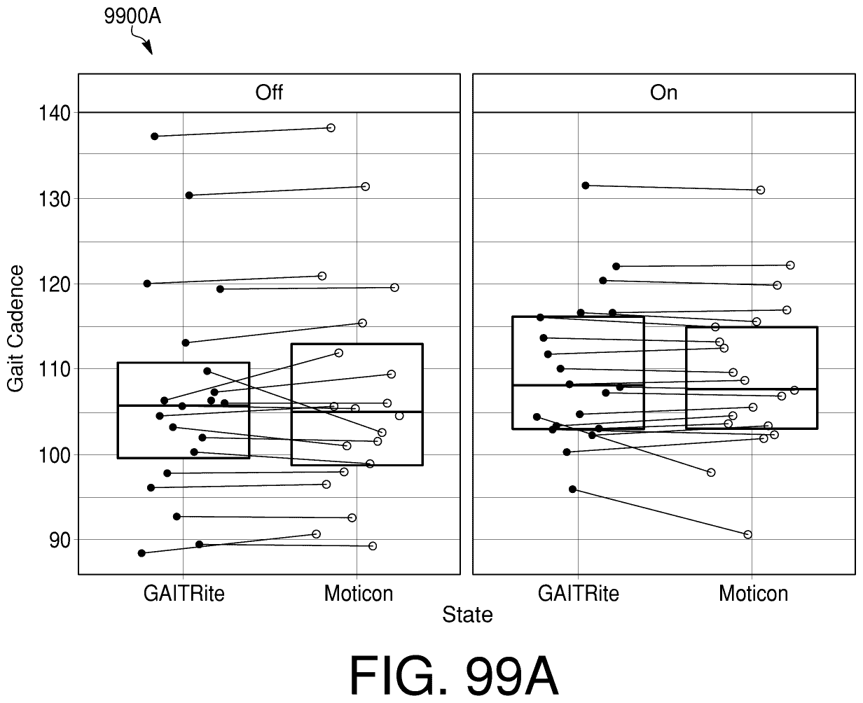

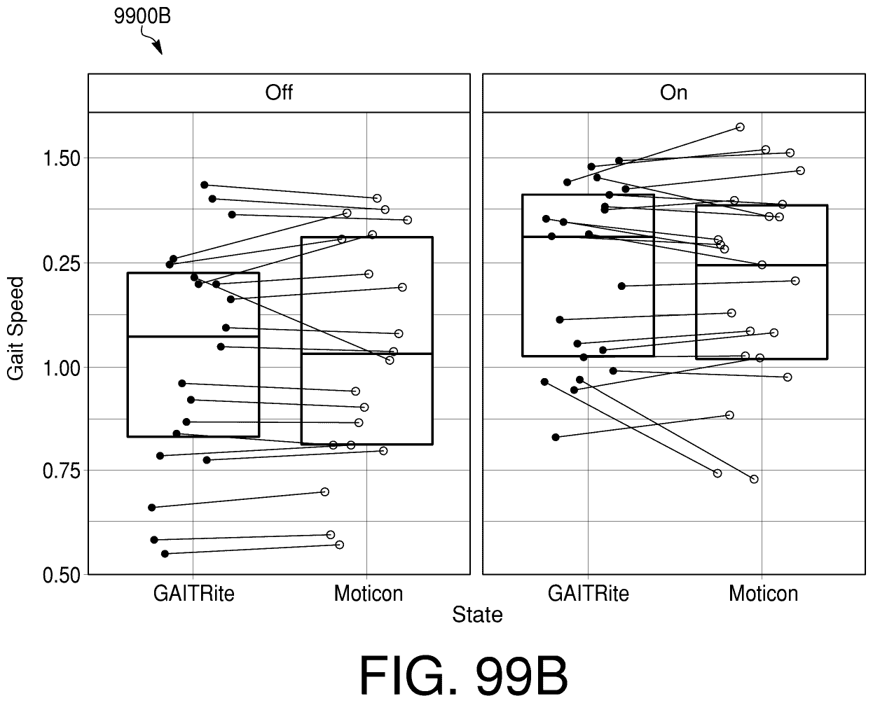

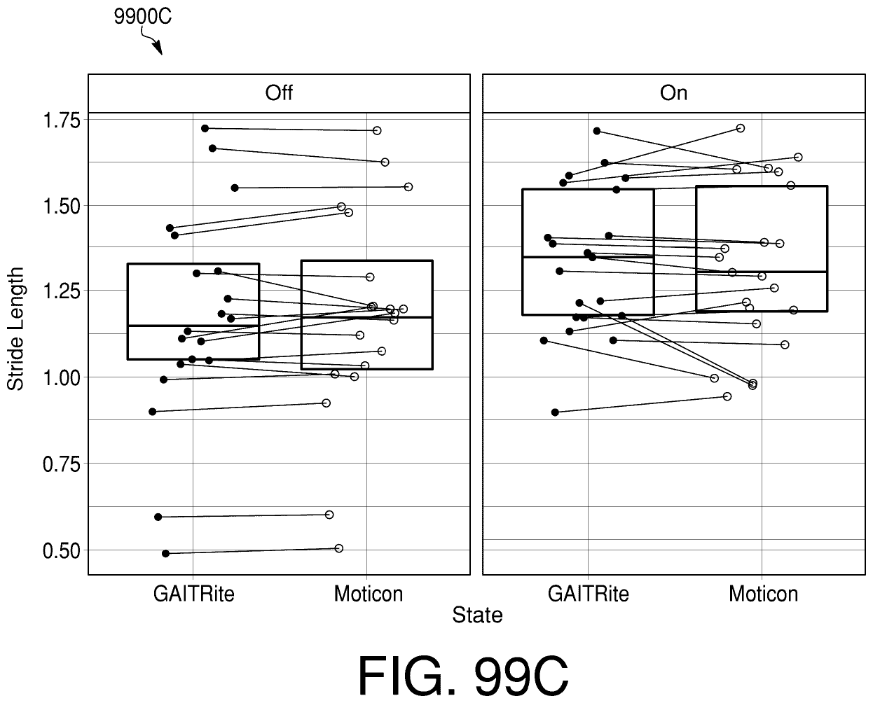

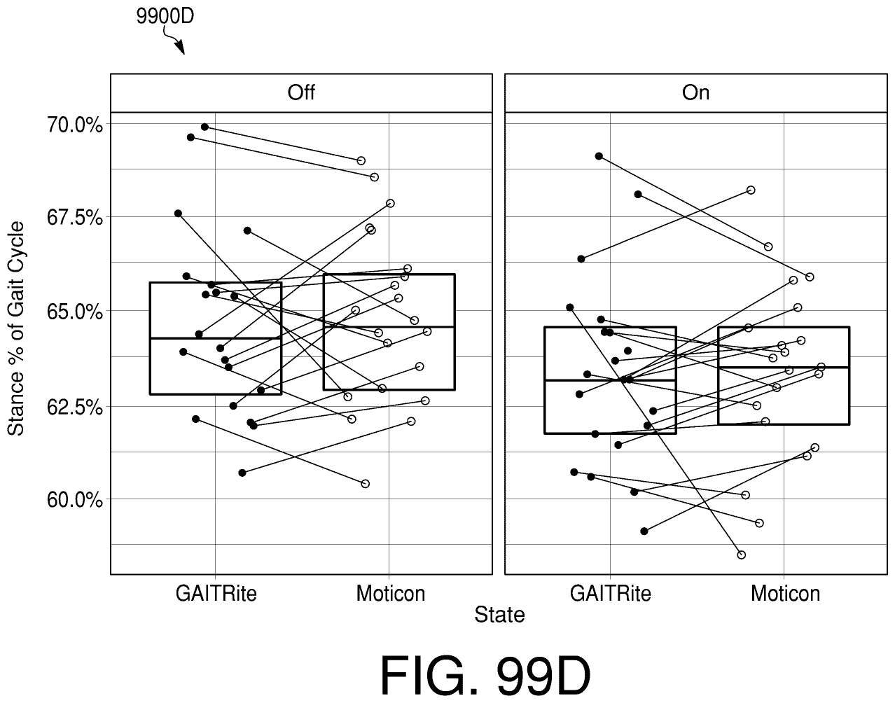

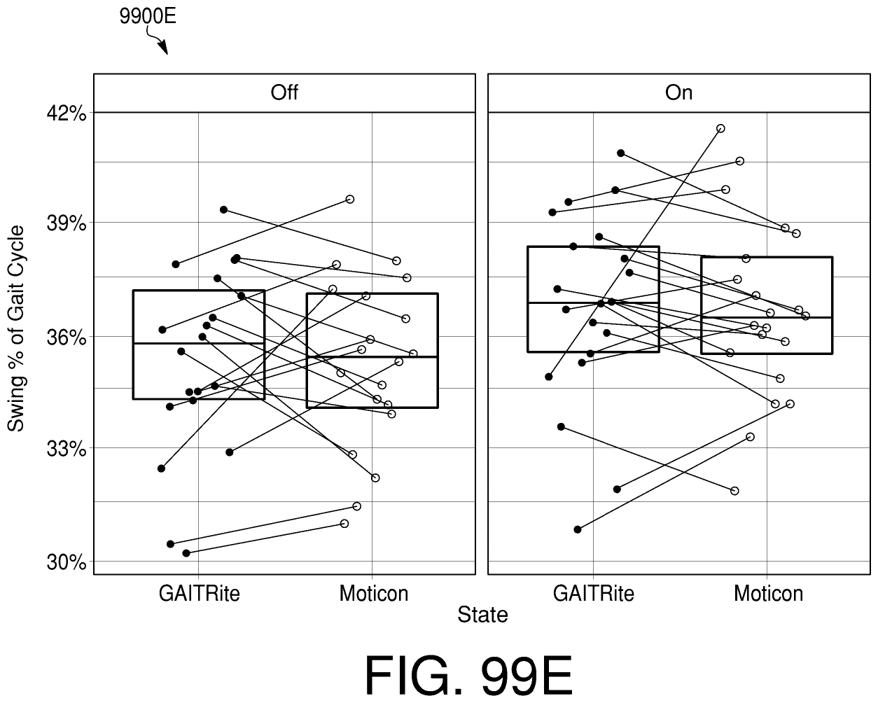

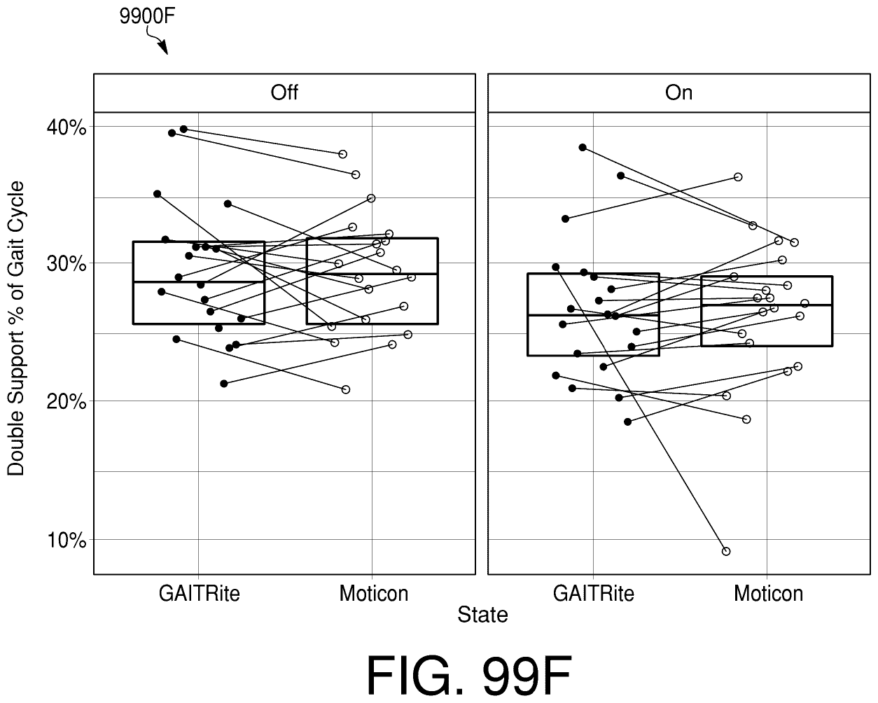

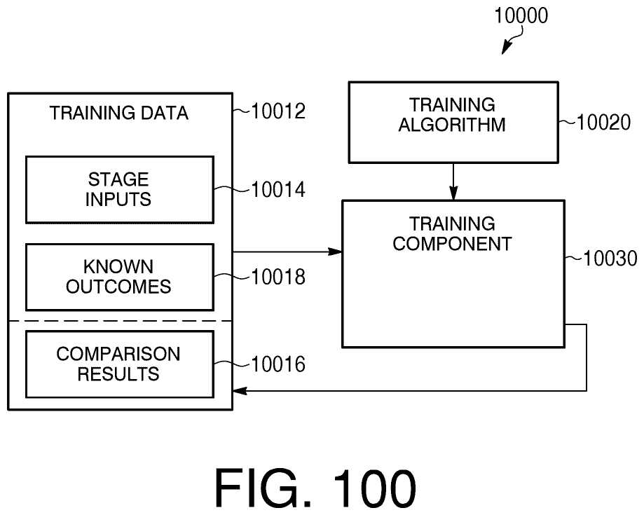

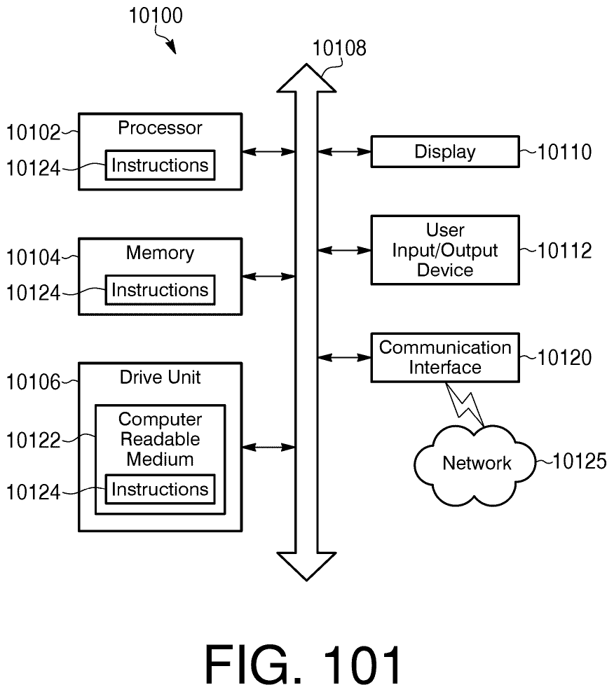

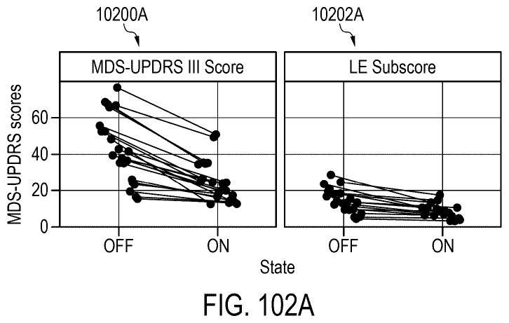

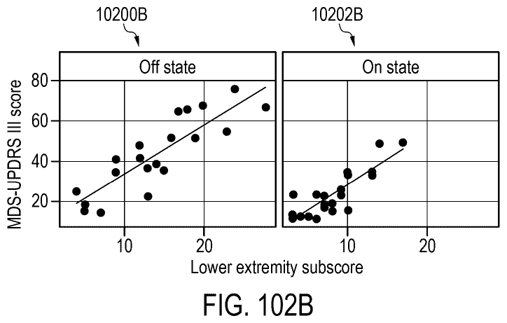

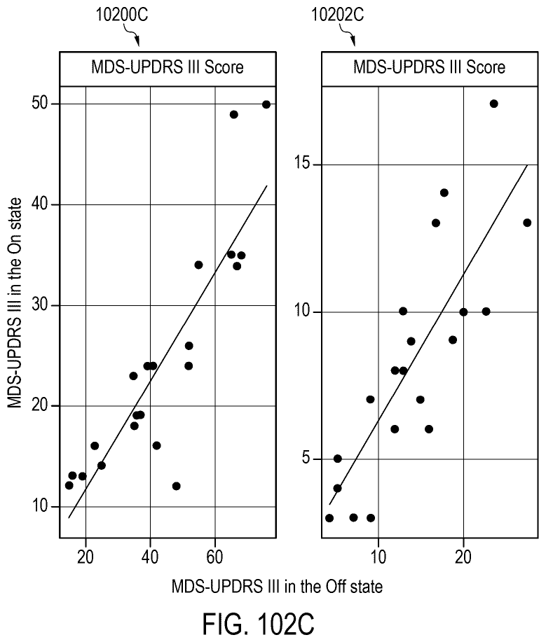

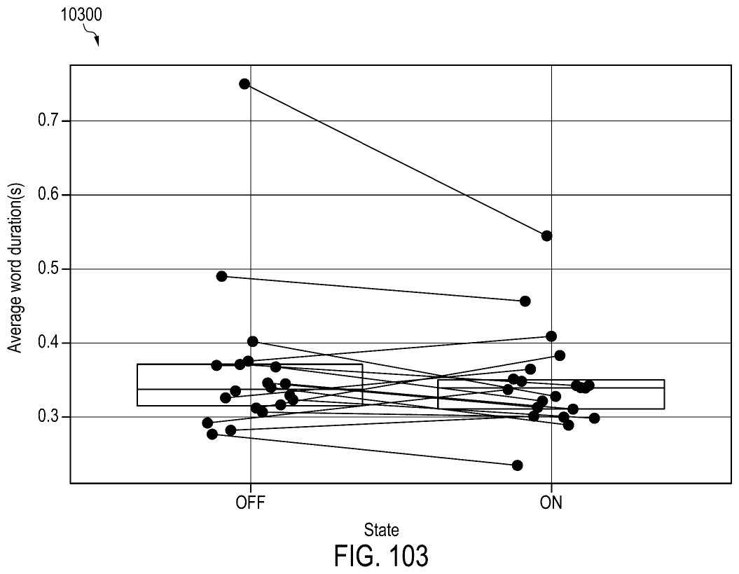

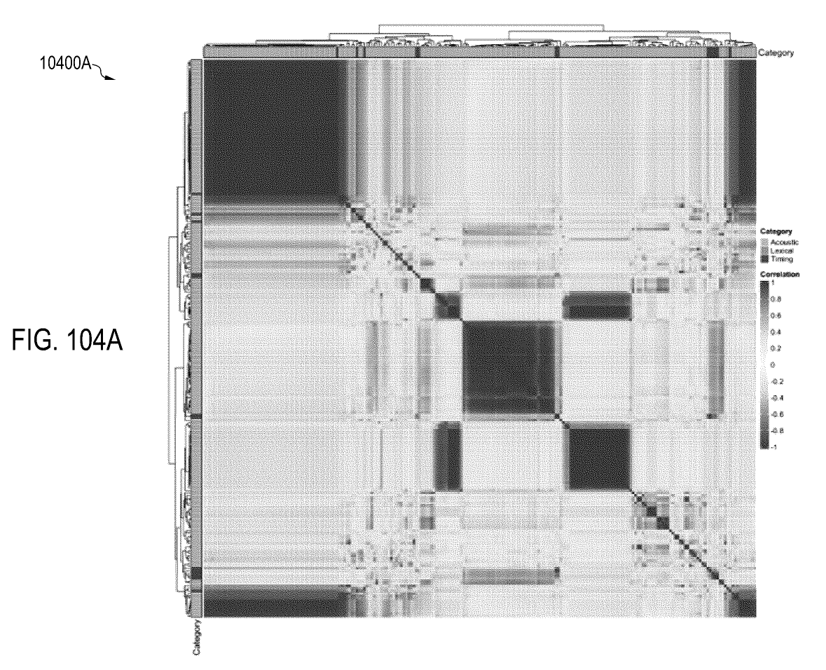

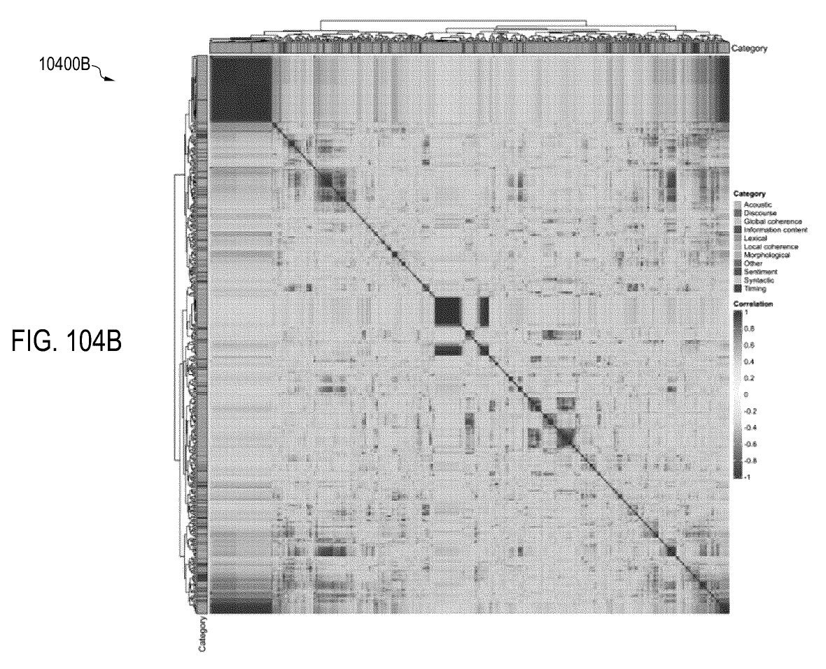

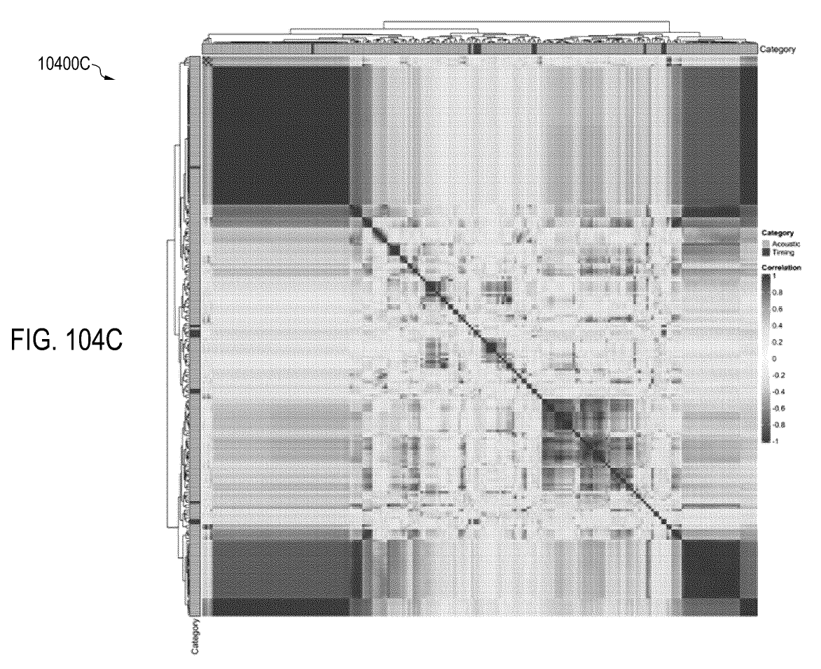

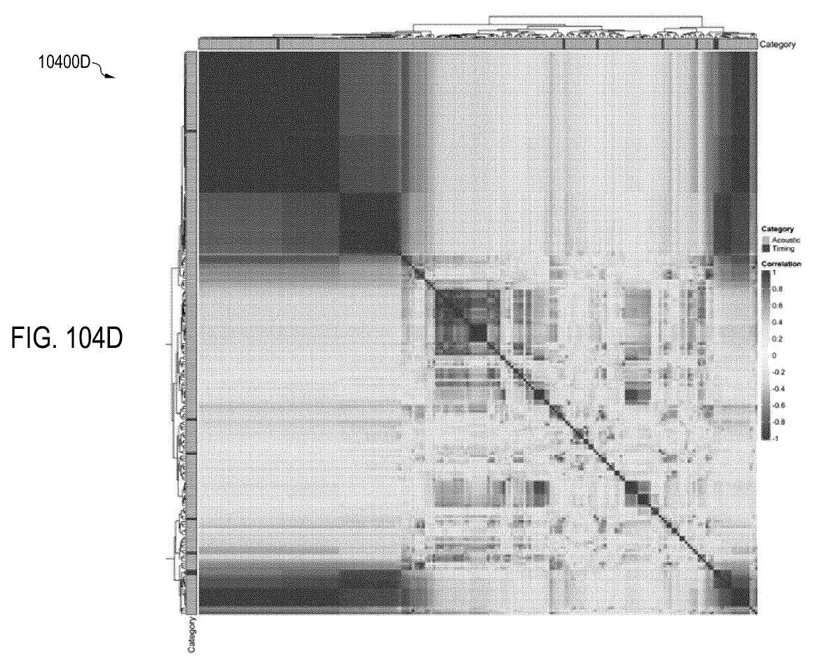

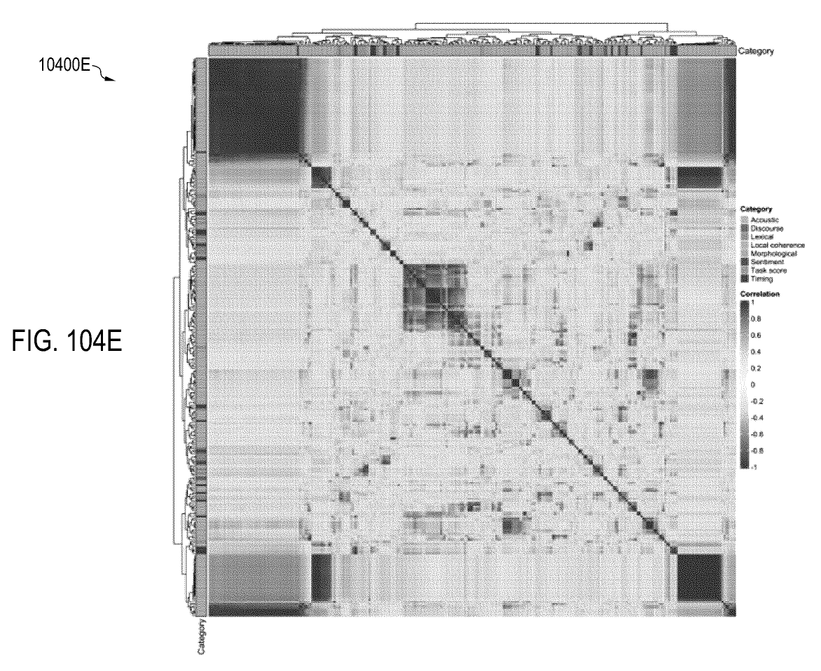

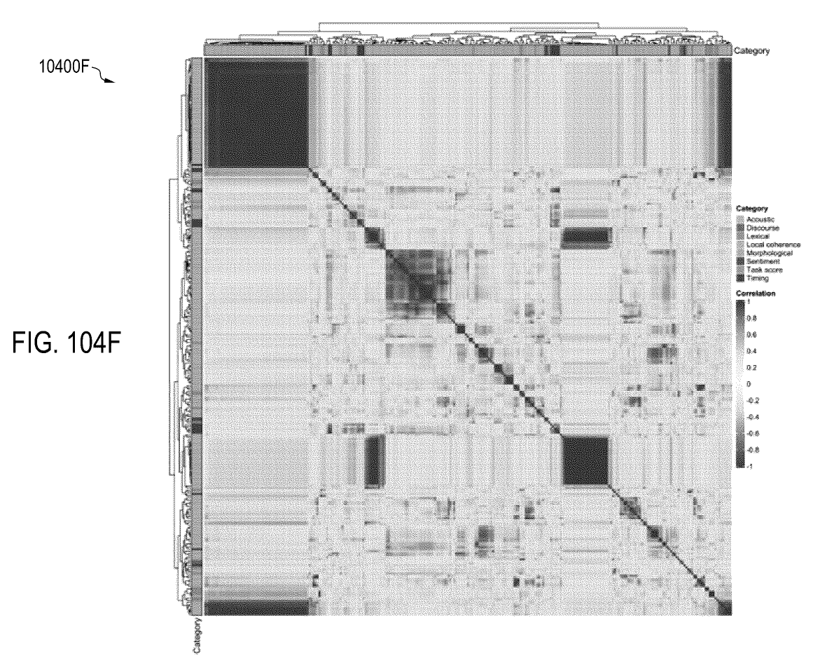

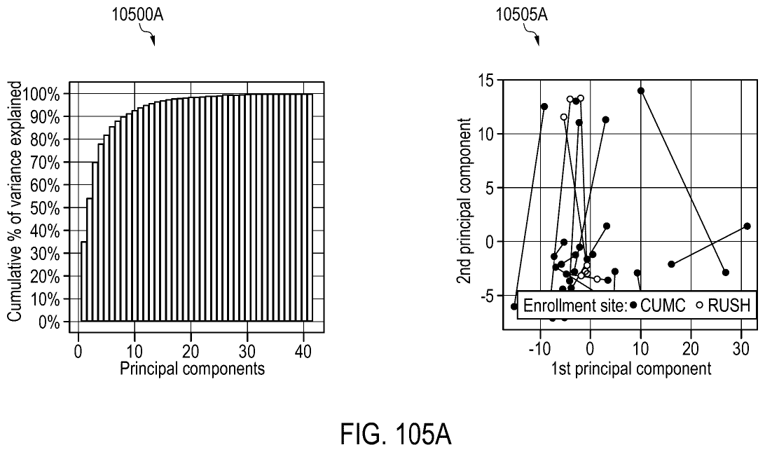

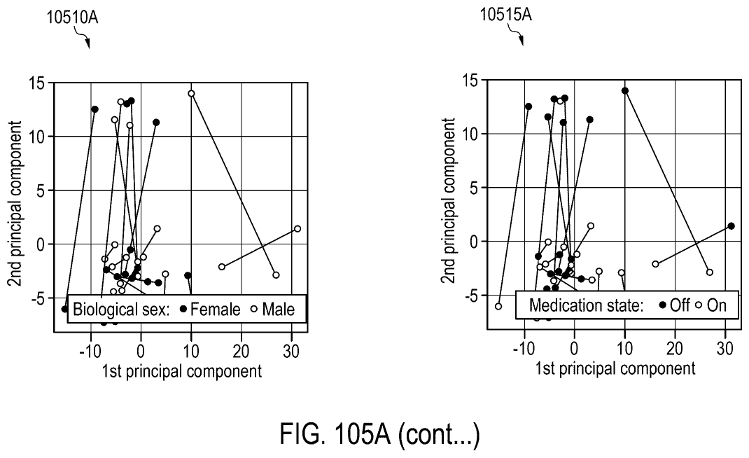

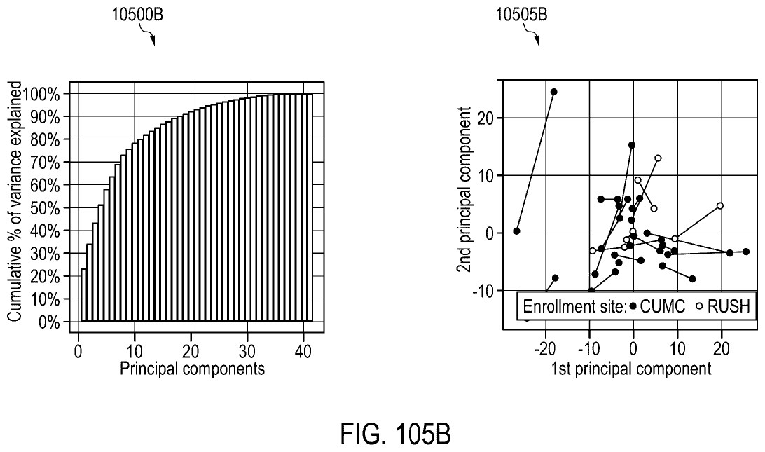

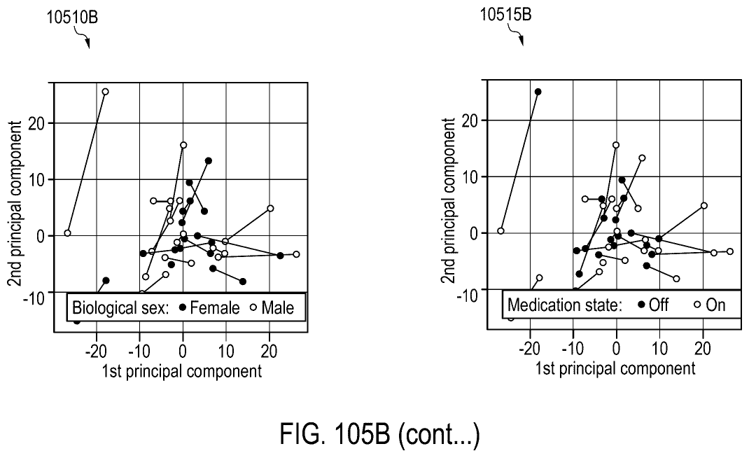

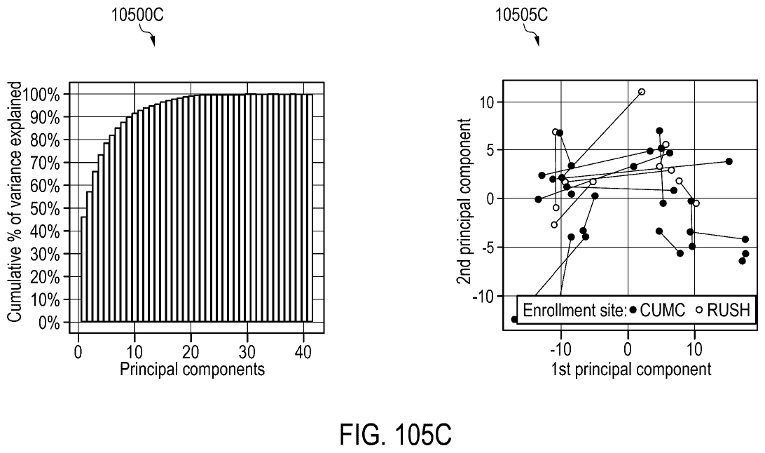

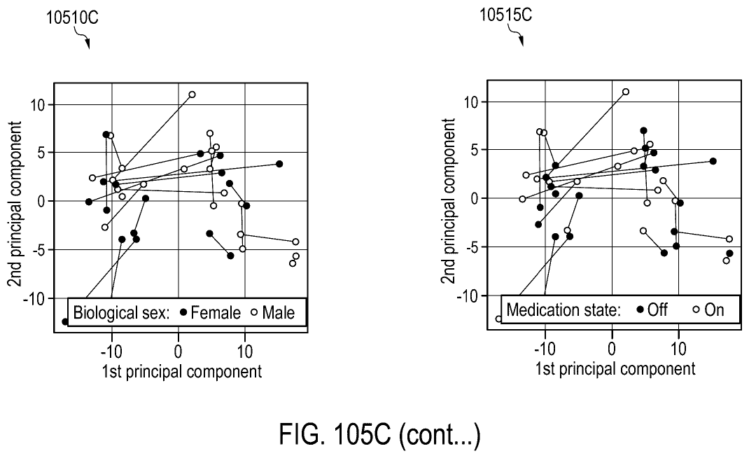

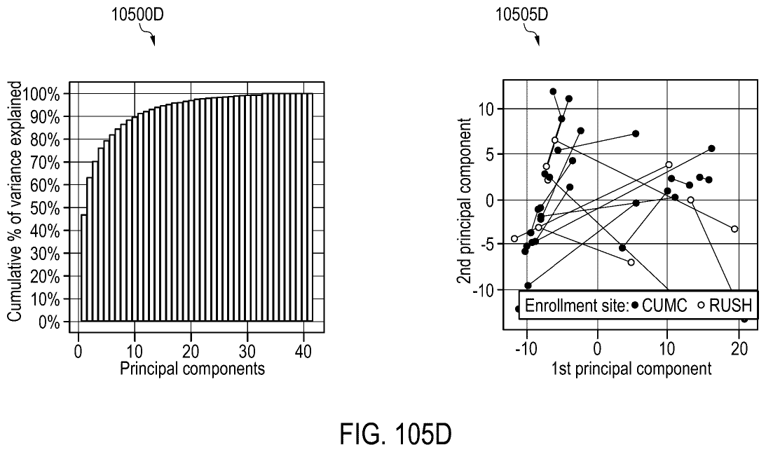

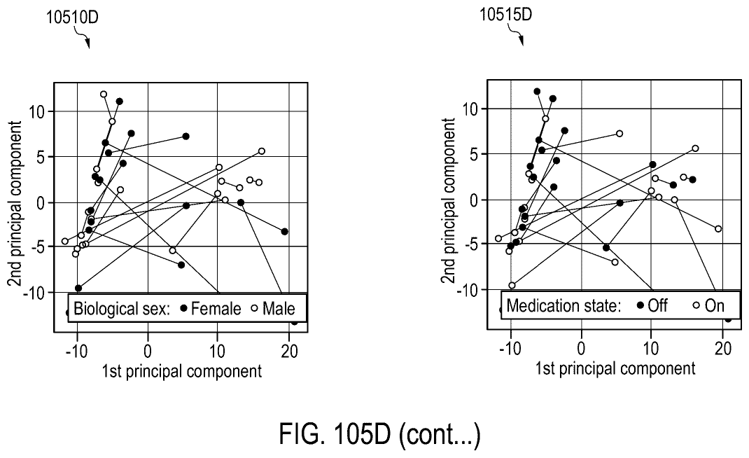

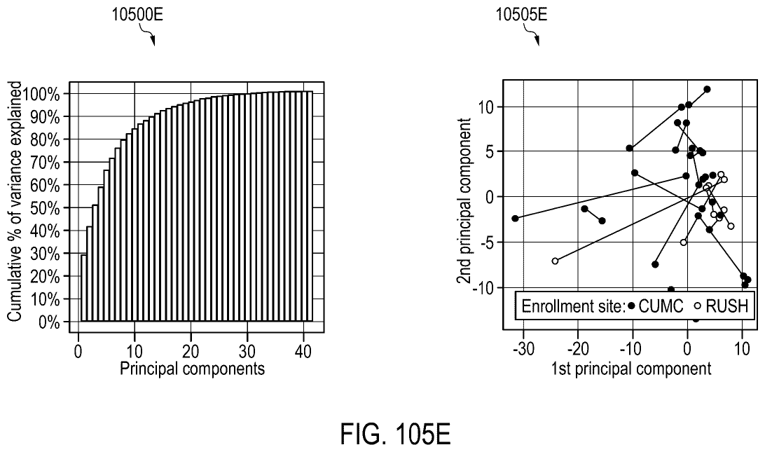

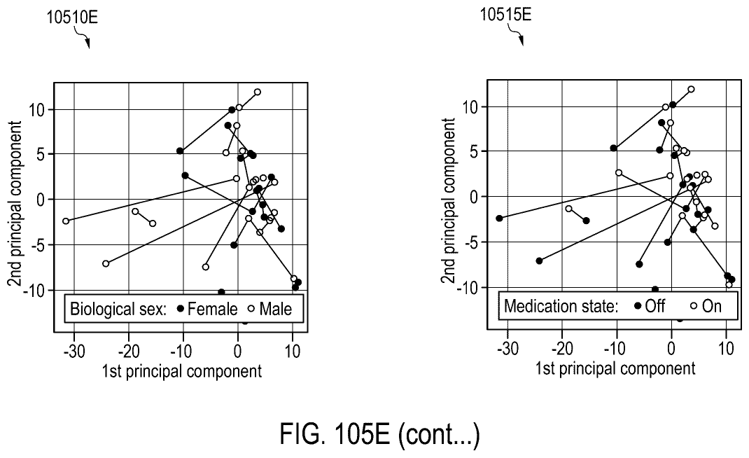

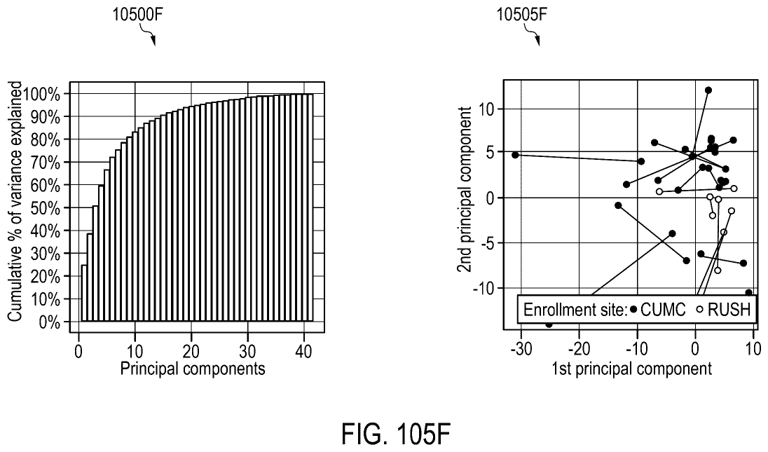

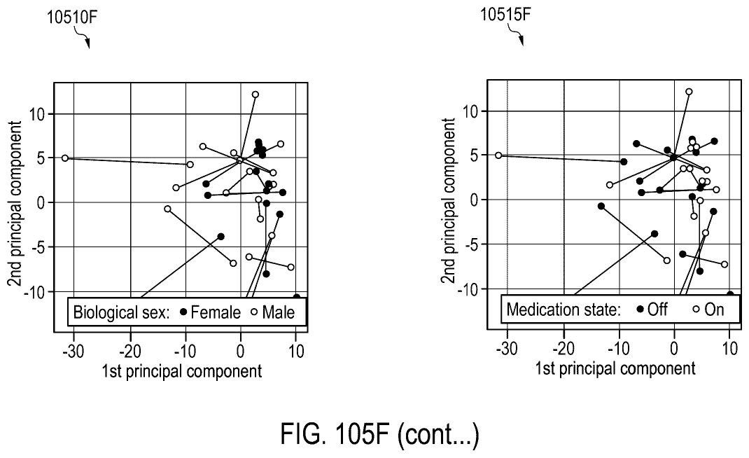

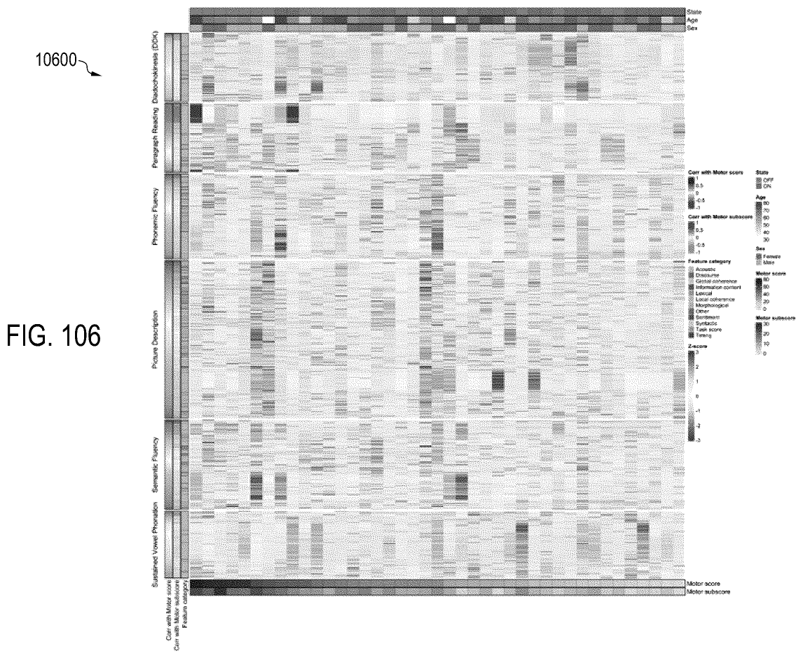

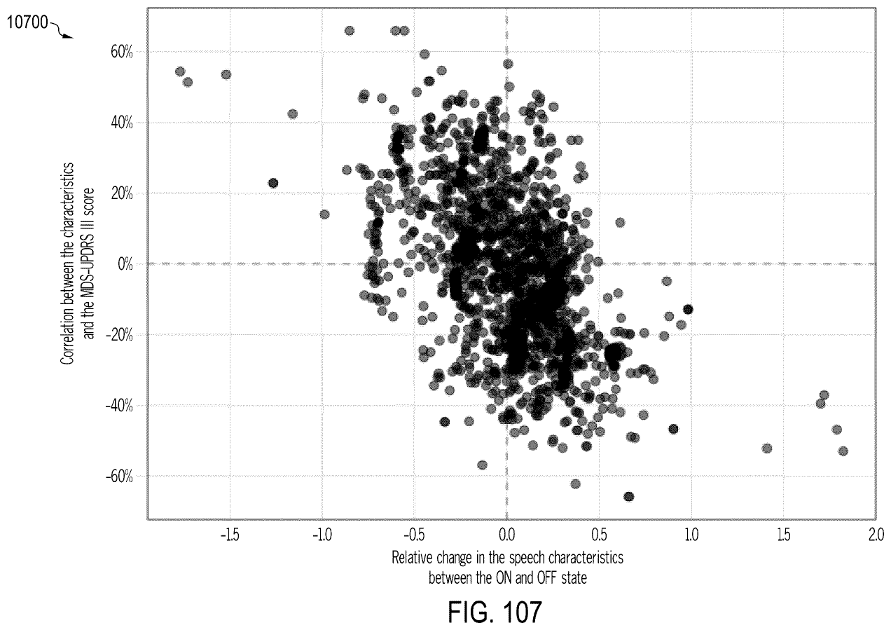

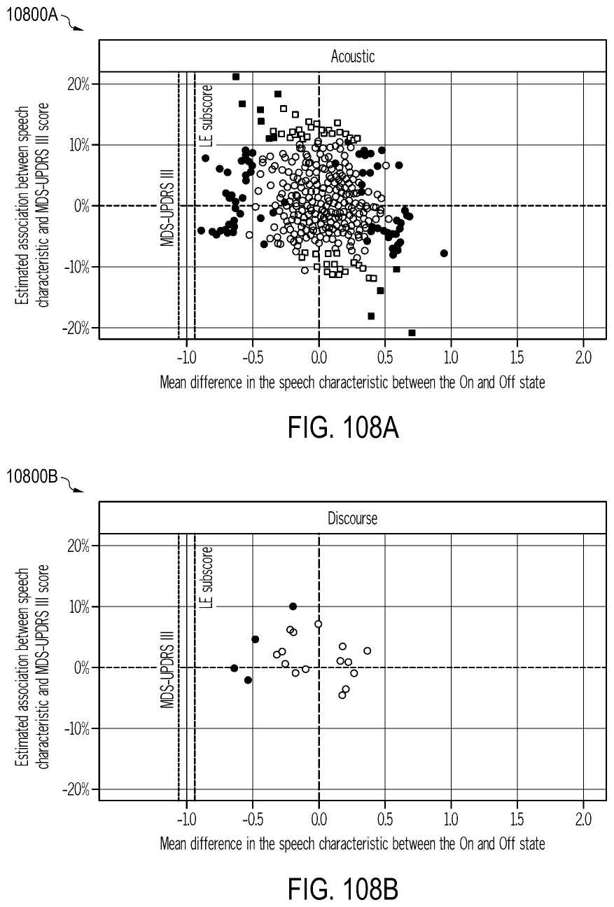

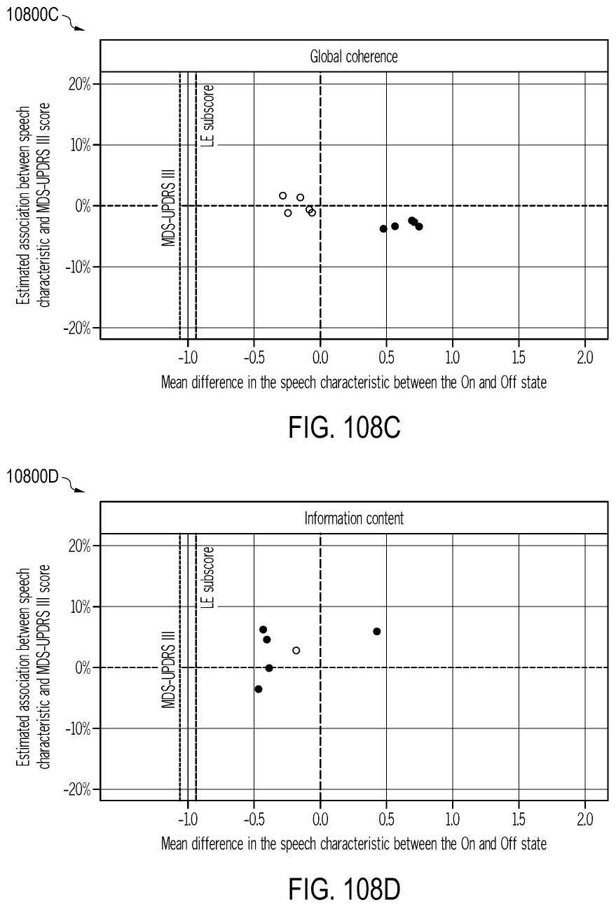

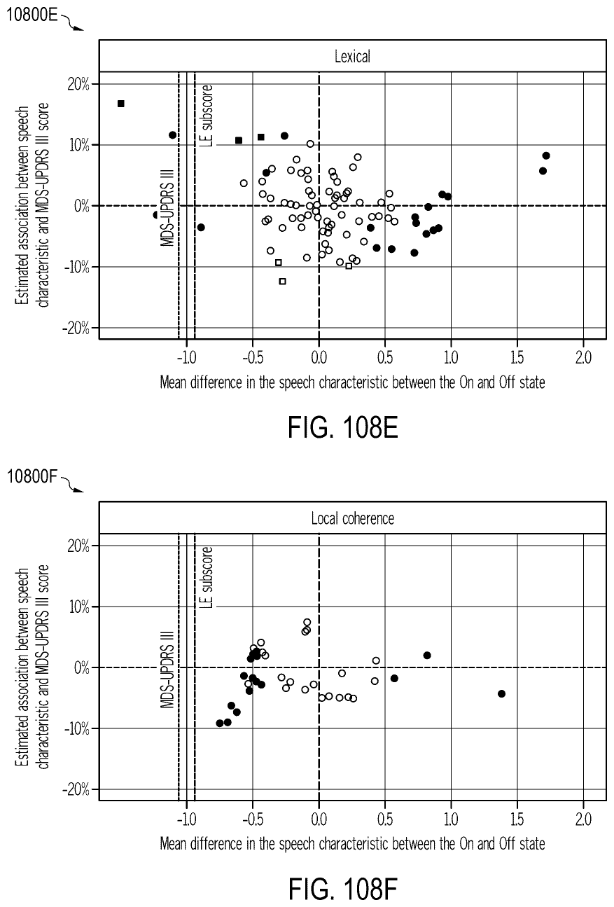

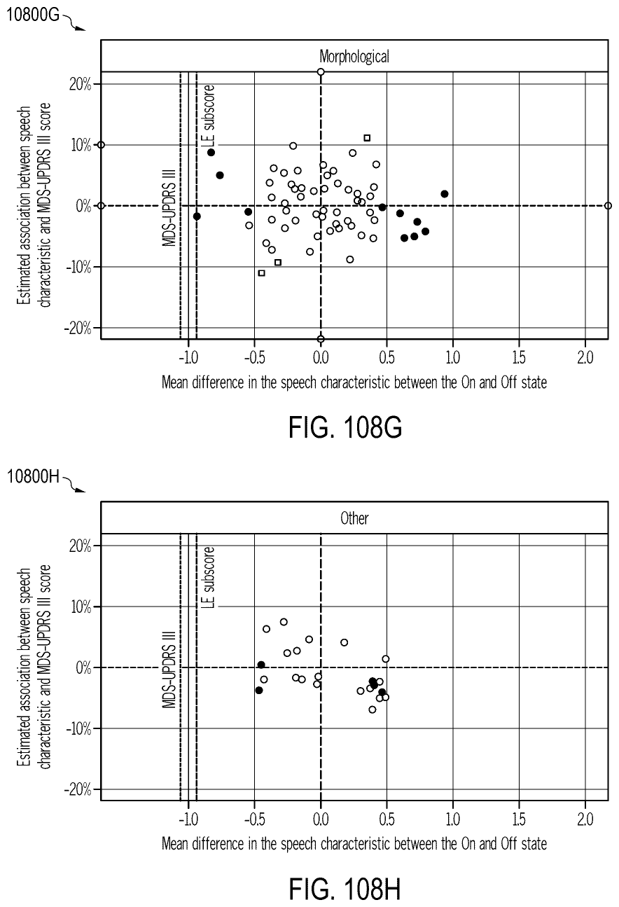

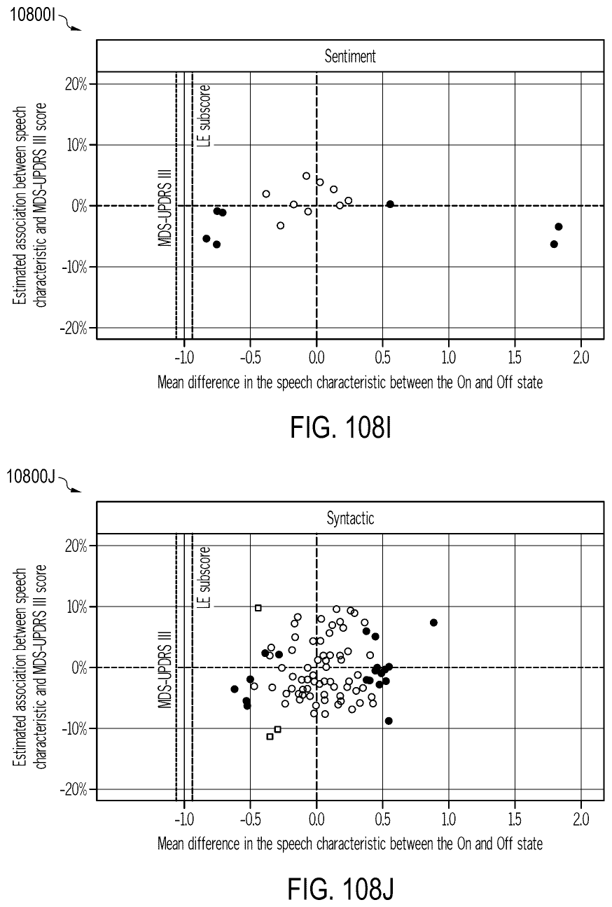

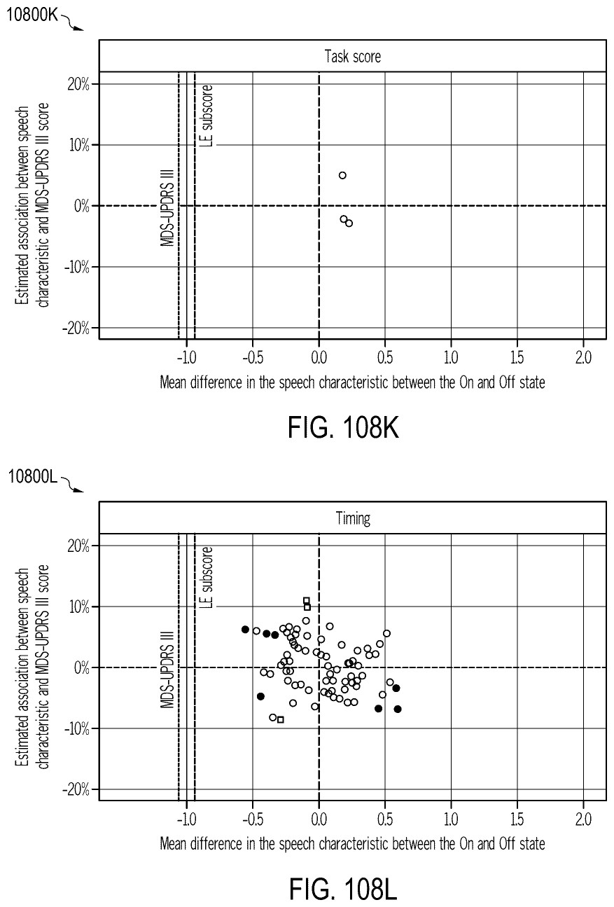

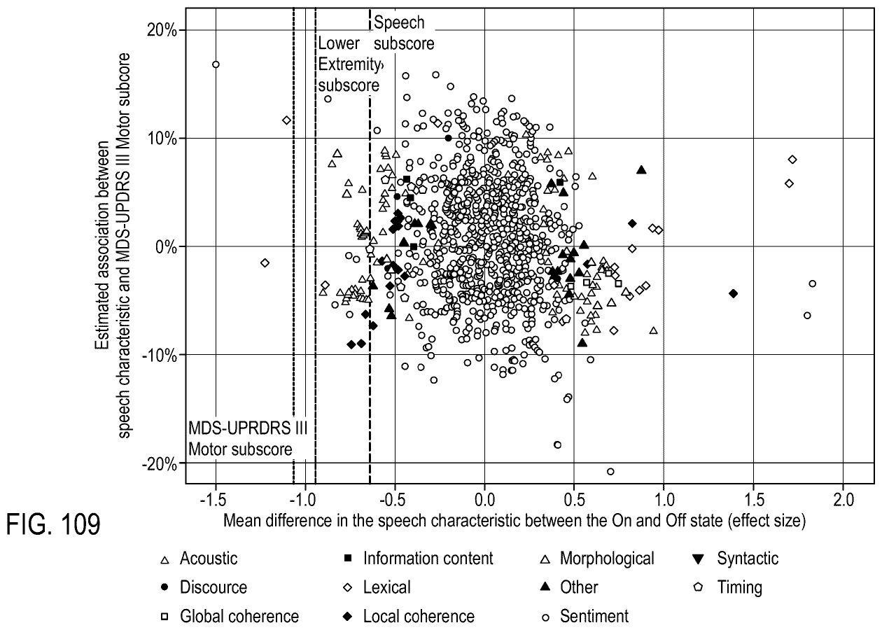

FIG. 93J provides a volcano plot presenting a mean difference in a syntactic speech characteristic between an ON and OFF state, in accordance with aspects of the present disclosure.

FIG. 93K provides a volcano plot presenting a mean difference in a task score speech characteristic between an ON and OFF state, in accordance with aspects of the present disclosure.

FIG. 93L provides a volcano plot presenting a mean difference in a timing speech characteristic between an ON and OFF state, in accordance with aspects of the present disclosure.

FIG. 94A provides a volcano plot, in which the model estimated association between an acoustic speech characteristic and the MDS-UPDRS III score is plotted against the mean difference in the acoustic speech characteristic between the ON and OFF state, in accordance with aspects of the present disclosure.

FIG. 94B provides a volcano plot, in which the model estimated association between a discourse speech characteristic and the MDS-UPDRS III score is plotted against the mean difference in the acoustic discourse characteristic between the ON and OFF state, in accordance with aspects of the present disclosure.

FIG. 94C provides a volcano plot, in which the model estimated association between a global coherence speech characteristic and the MDS-UPDRS III score is plotted against the mean difference in the global coherence speech characteristic between the ON and OFF state, in accordance with aspects of the present disclosure.

FIG. 94D provides volcano plot, in which the model estimated association between an information content speech characteristic and the MDS-UPDRS III score is plotted against the mean difference in the information content speech characteristic between the ON and OFF state, in accordance with aspects of the present disclosure.

FIG. 94E provides volcano plot, in which the model estimated association between a lexical speech characteristic and the MDS-UPDRS III score is plotted against the mean difference in the lexical speech characteristic between the ON and OFF state, in accordance with aspects of the present disclosure.

FIG. 94F provides volcano plot, in which the model estimated association between a local coherence speech characteristic and the MDS-UPDRS III score is plotted against the mean difference in the acoustic local coherence characteristic between the ON and OFF state, in accordance with aspects of the present disclosure.

FIG. 94G provides volcano plot, in which the model estimated association between a morphological speech characteristic and the MDS-UPDRS III score is plotted against the mean difference in the morphological speech characteristic between the ON and OFF state, in accordance with aspects of the present disclosure.

FIG. 94H provides volcano plot, in which the model estimated association between another speech characteristic and the MDS-UPDRS III score is plotted against the mean difference in the other speech characteristic between the ON and OFF state, in accordance with aspects of the present disclosure.

FIG. 941 provides volcano plot, in which the model estimated association between a sentiment speech characteristic and the MDS-UPDRS III score is plotted against the mean difference in the sentiment speech characteristic between the ON and OFF state, in accordance with aspects of the present disclosure.

FIG. 94J provides volcano plot, in which the model estimated association between a syntactic speech characteristic and the MDS-UPDRS III score is plotted against the mean difference in the syntactic speech characteristic between the ON and OFF state, in accordance with aspects of the present disclosure.

FIG. 94K provides volcano plot, in which the model estimated association between a task score speech characteristic and the MDS-UPDRS III score is plotted against the mean difference in the task score speech characteristic between the ON and OFF state, in accordance with aspects of the present disclosure.

FIG. 94L provides volcano plot, in which the model estimated association between a timing speech characteristic and the MDS-UPDRS III score is plotted against the mean difference in the timing speech characteristic between the ON and OFF state, in accordance with aspects of the present disclosure.

FIG. 95A provides a box and whisker plot that compares gait cadence measured by the test and production devices by levodopa state, in accordance with aspects of the present disclosure.

FIG. 95B provides a box and whisker plot that compares gait speed measured by the test and production devices by levodopa state, in accordance with aspects of the present disclosure.

FIG. 95C provides a box and whisker plot that compares stride length measured by the test and production devices by levodopa state, in accordance with aspects of the present disclosure.

FIG. 95D provides a box and whisker plot that compares stance percentage of the gait cycle measured by the test and production devices by levodopa state, in accordance with aspects of the present disclosure.

FIG. 95E provides a box and whisker plot that compares swing percentage of the gait cycle measured by the test and production devices by levodopa state, in accordance with aspects of the present disclosure.

FIG. 95F provides a box and whisker plot that compares double support percentage of the gait cycle measured by the test and production devices by levodopa state, in accordance with aspects of the present disclosure.

FIG. 96 provides a diagram of the analytical validation results that illustrate the comparison of the measured gait characteristics and levodopa state, in accordance with aspects of the present disclosure.

FIG. 97A provides a Bland-Altman plot that further assesses device equivalence for analytical validation for gait cadence, in accordance with aspects of the present disclosure.

FIG. 97B provides a Bland-Altman plot that further assesses device equivalence for analytical validation for gait speed, in accordance with aspects of the present disclosure.

FIG. 97C provides a Bland-Altman plot that further assesses device equivalence for analytical validation for stride length, in accordance with aspects of the present disclosure.

FIG. 97D provides a Bland-Altman plot that further assesses device equivalence for analytical validation for stance percentage of the gait cycle, in accordance with aspects of the FIG. 97E provides a Bland-Altman plot that further assesses device equivalence for analytical validation for swing percentage of the gait cycle, in accordance with aspects of the present disclosure.

FIG. 97F provides a Bland-Altman plot that further assesses device equivalence for analytical validation for double support percentage of the gait cycle, in accordance with aspects of the present disclosure.

FIG. 98A provides a graph that visually represents the reliability of gait cadence, in accordance with aspects of the present disclosure.

FIG. 98B provides a graph that visually represents the reliability of gait speed, in accordance with aspects of the present disclosure.

FIG. 98C provides a graph that visually represents the reliability of stride length, in accordance with aspects of the present disclosure.

FIG. 98D provides a graph that visually represents the reliability of stance percentage of the gait cycle, in accordance with aspects of the present disclosure.

FIG. 98E provides a graph that visually represents the reliability of swing percentage of the gait cycle, in accordance with aspects of the present disclosure.

FIG. 98F provides a graph that visually represents the reliability of double support percentage of the gait cycle, in accordance with aspects of the present disclosure.

FIG. 99A provides a plot that visually assess discrepancies between equivalency and reliability for the test and production devices in the varying OFF and ON levodopa states for gait cadence, in accordance with aspects of the present disclosure.

FIG. 99B provides a plot that visually assess discrepancies between equivalency and reliability for the test and production devices in the varying OFF and ON levodopa states for gait speed, in accordance with aspects of the present disclosure.

FIG. 99C provides a plot that visually assess discrepancies between equivalency and reliability for the test and production devices in the varying OFF and ON levodopa states for stride length, in accordance with aspects of the present disclosure.

FIG. 99D provides a plot that visually assess discrepancies between equivalency and reliability for the test and production devices in the varying OFF and ON levodopa states for stance percentage of the gait cycle, in accordance with aspects of the present disclosure.

FIG. 99E provides a plot that visually assess discrepancies between equivalency and reliability for the test and production devices in the varying OFF and ON levodopa states for swing percentage of the gait cycle, in accordance with aspects of the present disclosure.

FIG. 99F provides a plot that visually assess discrepancies between equivalency and reliability for the test and production devices in the varying OFF and ON levodopa states for double support percentage of the gait cycle, in accordance with aspects of the present disclosure.

FIG. 100 discloses an exemplary data flow for training a machine learning model, in accordance with aspects of the present disclosure.

FIG. 101 provides a simplified functional block diagram of a computer system that may be configured as a device for executing the techniques disclosed herein, in accordance with aspects of the present disclosure.

FIG. 102A provides a graph that illustrates the change in MDS-UPDRS III and LE scores per subject overall, in accordance with aspects of the present disclosure.

FIG. 102B provides a graph that illustrates the correlation between the MDS-UPDRS III score and the LE subscore, in accordance with aspects of the present disclosure.

FIG. 102C provides a graph that illustrates the effects on the MDS-UPDRS III score and the LE subscore when the subject is in the OFF state versus the ON state, in accordance with aspects of the present disclosure.

FIG. 103 provides a graph that identifies the change in average word duration spoken by subjects in different medication states, in accordance with aspects of the present disclosure.

FIG. 104A provides a correlation heat map of speech characteristics for a paragraph reading task assessed using the digital mobile speech-based platform, in accordance with aspects of the present disclosure.

FIG. 104B provides a correlation heat map of speech characteristics for a picture description task assessed using the digital mobile speech-based platform, in accordance with aspects of the present disclosure.

FIG. 104C provides a correlation heat map of speech characteristics for a DDK rate task assessed using the digital mobile speech-based platform, in accordance with aspects of the FIG. 104D provides a correlation heat map of speech characteristics for a sustained vowel phonation task assessed using the digital mobile speech-based platform, in accordance with aspects of the present disclosure.

FIG. 104E provides a correlation heat map of speech characteristics for a semantic fluency task assessed using the digital mobile speech-based platform, in accordance with aspects of the present disclosure.

FIG. 104F provides a correlation heat map of speech characteristics for a phonemic fluency task assessed using the digital mobile speech-based platform, in accordance with aspects of the present disclosure.

FIG. 105A provides a plurality of scree and scatter graphs that plot principal components 1 (PC1) against principal components (PC2) by a paragraph reading speech task, in accordance with aspects of the present disclosure.

FIG. 105B provides a plurality of scree and scatter graphs that plot principal components 1 (PC1) against principal components (PC2) by a picture description speech task, in accordance with aspects of the present disclosure.

FIG. 105C provides a plurality of scree and scatter graphs that plot principal components 1 (PC1) against principal components (PC2) by a DDK rate speech task, in accordance with aspects of the present disclosure.

FIG. 105D provides a plurality of scree and scatter graphs that plot principal components 1 (PC1) against principal components (PC2) by a sustained vowel phonation speech task, in accordance with aspects of the present disclosure.

FIG. 105E provides a plurality of scree and scatter graphs that plot principal components 1 (PC1) against principal components (PC2) by a semantic fluency speech task, in accordance with aspects of the present disclosure.

FIG. 105F provides a plurality of scree and scatter graphs that plot principal components 1 (PC1) against principal components (PC2) by a phonemic fluency speech task, in accordance with aspects of the present disclosure.

FIG. 106 provides a heat map of scaled speech characteristic values across subject, in accordance with aspects of the present disclosure.

FIG. 107 provides a plot representing the correlation of each speech characteristic with the MDS-UPDRS III score as a function of how much the corresponding characteristics change between the ON and OFF states, in accordance with aspects of the present disclosure.

FIG. 108A provides a volcano plot, in which the model estimated association between an acoustic speech characteristic and the MDS-UPDRS III score is plotted against the mean difference in the acoustic speech characteristic between the ON and OFF state, in accordance with aspects of the present disclosure.

FIG. 108B provides a volcano plot, in which the model estimated association between a discourse speech characteristic and the MDS-UPDRS III score is plotted against the mean difference in the acoustic discourse characteristic between the ON and OFF state, in accordance with aspects of the present disclosure.

FIG. 108C provides a volcano plot, in which the model estimated association between a global coherence speech characteristic and the MDS-UPDRS III score is plotted against the mean difference in the global coherence speech characteristic between the ON and OFF state, in accordance with aspects of the present disclosure.

FIG. 108D provides volcano plot, in which the model estimated association between an information content speech characteristic and the MDS-UPDRS III score is plotted against the mean difference in the information content speech characteristic between the ON and OFF state, in accordance with aspects of the present disclosure.

FIG. 108E provides volcano plot, in which the model estimated association between a lexical speech characteristic and the MDS-UPDRS III score is plotted against the mean difference in the lexical speech characteristic between the ON and OFF state, in accordance with aspects of the present disclosure.

FIG. 108F provides volcano plot, in which the model estimated association between a local coherence speech characteristic and the MDS-UPDRS III score is plotted against the mean difference in the acoustic local coherence characteristic between the ON and OFF state, in accordance with aspects of the present disclosure.

FIG. 108G provides volcano plot, in which the model estimated association between a morphological speech characteristic and the MDS-UPDRS III score is plotted against the mean difference in the morphological speech characteristic between the ON and OFF state, in accordance with aspects of the present disclosure.

FIG. 108H provides volcano plot, in which the model estimated association between another speech characteristic and the MDS-UPDRS III score is plotted against the mean difference in the other speech characteristic between the ON and OFF state, in accordance with aspects of the present disclosure.

FIG. 108I provides volcano plot, in which the model estimated association between a sentiment speech characteristic and the MDS-UPDRS III score is plotted against the mean difference in the sentiment speech characteristic between the ON and OFF state, in accordance with aspects of the present disclosure.

FIG. 108J provides volcano plot, in which the model estimated association between a syntactic speech characteristic and the MDS-UPDRS III score is plotted against the mean difference in the syntactic speech characteristic between the ON and OFF state, in accordance with aspects of the present disclosure.

FIG. 108K provides volcano plot, in which the model estimated association between a task score speech characteristic and the MDS-UPDRS III score is plotted against the mean difference in the task score speech characteristic between the ON and OFF state, in accordance with aspects of the present disclosure.

FIG. 108L provides volcano plot, in which the model estimated association between a timing speech characteristic and the MDS-UPDRS III score is plotted against the mean difference in the timing speech characteristic between the ON and OFF state, in accordance with aspects of the present disclosure.

FIG. 109 provides a plot that illustrates the estimated association between each speech characteristic and the MDS-UPDRS III score plotted against the estimated mean differences between the ON and OFF states, in accordance with aspects of the present disclosure.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

Table A below provides a set of abbreviations and corresponding definitions, as used herein.

TABLE A

| Abbreviation | Definition |
|---|---|
| BR | Biomarker Report |
| ECG | Electrocardiogram |
| ES | Enrichment Score |
| IV | Intravenous |
| NES | Normalized Enrichment Score |
| PK | Pharmacokinetic |
| SAP | Statistical Analysis Plan |
| SAS | Statistical Analysis System |
| SC | Subcutaneous |
| ADJP | Adjective Phrase Parameters |
| ADVP | Adverb Phrase Parameters |
| CI | Confidence Intervals |
| COP | Center of Pressure |
| FRAG | Fragment Parameter |
| HNR | Harmonic to Noise Ratio |
| INTJ | Interjection Parameters |
| MDS-UPDRS | Movement Disorder Society's Unified |
| | Parkinson's Disease Rating Scale |
| PD | Parkinson's Disease |

Gait abnormalities are one of the hallmark symptoms in a variety of neurological diseases, such as Parkinson's Disease (PD), and play a critical role in assessing the severity and progression of the condition. Gait is a complex motor activity regulated by multiple regions of the nervous system, and its impairment in PD may significantly impact patients' quality of life. There are various known gait characteristics exhibited by individuals afflicted with PD. For instance, in the early stages of PD, patients often exhibit subtle changes in their gait. These may include shorter steps, reduced arm swing, and difficulty with turns. As the disease progresses, these symptoms worsen. For instance, patients may develop a shuffling gait, where they have difficulty lifting their feet from the ground, leading to small, dragging steps. Additionally, gait festination, characterized by short, rapid steps that are difficult to control, and freezing of gait, is also common. Furthermore, PD patients often experience significant fluctuations in motor function, particularly in response to medication. Certain treatments such as, for example, amino acid based treatments (e.g., Levodopa) can be used to treat PD and may dramatically improve motor function, including gait, but these improvements are often temporary. Gait abnormalities increase the risk of falls, leading to injuries and a subsequent decline in physical health and independence. Impaired gait limits mobility, reducing patients' ability to perform daily activities and diminishing their overall quality of life. Accordingly, objective and precise measurement of gait may provide valuable insights into disease progression and the efficacy of treatments.

Speech difficulties are another significant aspect of PD, affecting communication and social interaction, which are crucial for maintaining a good quality of life. There are various known speech characteristics exhibited by individuals afflicted with PD. For instance, patients may exhibit reduced vocal volume, making it difficult to be heard. As another example, some patients exhibit rapid, stammering speech that may be challenging to understand. In yet another example, patients may exhibit slurred speech due to poor articulation and control of the muscles used in speaking. Speech impairments hinder effective communication, potentially leading to social isolation and emotional distress. Difficulties in speech may also significantly contribute to the overall reduction in quality of life for PD patients. Accordingly, changes in speech characteristics may serve as indicators of disease progression and response to treatment.

Current gait assessment methods for PD predominantly rely on qualitative evaluations performed by clinicians. These assessments, often subjective in nature, depend heavily on the observer's expertise and judgment, leading to considerable variability and potential bias. Standard clinical scales, such as the Movement Disorder Society-Unified Parkinson's Disease Rating Scale (MDS-UPDRS), include gait evaluation but provide limited quantitative detail and sensitivity, particularly in the early stages of PD. Additionally, traditional quantitative tools (e.g., the GAITRite mat), while providing accurate and reliable data, are confined to clinical settings due to their size, cost, and operational requirements. This limitation hinders continuous and naturalistic gait monitoring, crucial for capturing the day-to-day fluctuations in gait patterns typical in PD. Consequently, there is a need for portable, user-friendly, and precise gait assessment tools that may be used outside clinical environments to provide a more comprehensive understanding of gait impairments in PD patients.

Speech assessment in PD also faces significant challenges due to the qualitative nature of current evaluation techniques. Speech impairments, such as hypophonia, dysarthria, and tachyphemia, are commonly assessed through clinical observation and patient self-reporting; both of which are subject to variability and bias. The MDS-UPDRS includes components for speech evaluation, but these are limited in granularity and fail to capture subtle changes in speech that may indicate disease progression or treatment response. Furthermore, existing speech assessment tools are not extensively developed or validated for frequent, real-world use. More particularly, these tools often require specialized equipment and settings, making them impractical for regular monitoring. This gap underscores the need for robust, quantitative speech assessment methods that are easily deployable in everyday settings. Such tools would enable more accurate tacking of speech changes over time, providing deeper insights into the impact of PD on patients' communication abilities and overall quality of life.

Accordingly, the present disclosure proposes a novel approach to address the foregoing challenges by evaluating the use of a wearable insole for gait assessment and a mobile device-based speech program to monitor and analyze gait and speech characteristics in patients with PD. The assessment may be conducted both before and after the administration of a dopaminergic medication commonly prescribed to treat PD, such as an amino acid based treatment (e.g., Levodopa). This allows for a comprehensive analysis of how the medication affects motor and speech functions, using the patients' own OFF/ON state comparison as a control.

In some aspects, the wearable insole may be embedded with sensors that capture a range of gait parameters (e.g., step length, step time, stride length, stride time, pressure distribution, etc.). Unlike traditional gait assessment tools confined to clinical settings, these insoles may be used in everyday environments, providing continuous and naturalistic monitoring of gait patterns. This portability and convenience address the limitations of existing gait quantification tools, enabling real-world data collection that reflects the true variability and progression of gait impairments in PD patients.

In some aspects, the mobile device-based speech program facilitates the assessment of various speech tasks including paragraph reading, picture description, object naming, rapid syllable repetition, and sustained phonation. These tasks are designed to evaluate different aspects of speech affected by PD, such as volume, rate, articulation, and fluency. The use of a mobile platform allows for frequent and flexible data collection, overcoming the constraints of traditional speech assessment methods that require specialized equipment and settings. This frequent data collection can capture subtle changes in speech that may indicate disease progression or response to treatment.

By comparing gait and speech data collected in the OFF state (e.g., before intake treatment such as an intake of an amino acid based treatment) and the ON stage (e.g., after treatment), the aspects described herein aim to identify significant changes attributable to the medication. This within-patient comparison helps to control for individual variability and provides a clear picture of how dopaminergic treatment affects motor and speech functions. Additionally, correlating these digital biomarkers with clinical ratings from the MDS-UPDRS offers a more nuanced understanding of disease severity and progression.

The exploratory investigations described herein aim to validate the utility of these innovative digital measures in capturing clinically meaningful data. By establishing the reliability and accuracy of the wearable insoles and digital mobile speech platform, the aspects described herein aim to identify important digital biomarkers for PD. These biomarkers may enhance the monitoring of disease progression, improve the precision of clinical assessments, and inform treatment decisions. Ultimately, this approach has the potential to drive broader application of digital health technologies in the management of neurodegenerative diseases, offering more comprehensive and patient-centered care.

The concepts described herein represent significant improvements to computer technology, particularly in the realm of digital health monitoring and neurodegenerative disease management. For instance, one advancement is the integration of advanced wearable technology, specifically sensor-equipped insoles, for comprehensive gait analysis. These insoles utilize several integrated sensors to capture detailed gait parameters, allowing for continuous, real-world monitoring outside of clinical settings. Validation of the utilization of these insoles to effectively identify symptoms (e.g., gait characteristics) in individuals having PD overcomes the limitations of traditional gait assessment tools, offering a more naturalistic and frequent data collection method than has traditionally been available.

Accordingly, the concepts described herein are not directed to a specific application of technology aimed at improving the monitoring and management of Parkinson's disease. The use of wearable insoles with embedded sensors and a mobile speech analysis platform provides a tangible technological salutation that collects and analyzes data in real-time. This data may then be used to offer precise and actionable insights into a subject's condition, which directly impacts their treatment plan. Furthermore, the integration with clinical evaluation methods, such as MDS-UPDRS, further demonstrates that the concepts described herein represent a practical application with a clear technological benefit, rather than a mere abstract concept.

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

Aspects of the disclosed subject matter are directed to receiving signals generated based on a body component of an individual. The signals may be or may be generated based on electrical activity, physical activity, biometric data, movement data, or any attribute of an individual's body, an action associated with the individual's body, reaction of the individual's body, or the like. The signals may be generated by a production device that may capture the signals using one or more sensors. For example, aspects of the disclosed subject matter are directed to methods for conducting gait assessment using a gait lab, and specifically a dedicated gait mat that is configured to detect and/or generate, among other data, ground reaction force (GRF) data. As discussed herein, biosensor data collected by wearable devices (e.g., smart or digital insoles) may be comparable to lab-based clinical assessments and may be used to identify subject-specific gait patterns. In other examples, a lab-based gold standard may be used to identify subject-specific gait patterns.

Aspects of the disclosed subject matter are further directed to receiving signals generated using a test device. The signals generated using a test device may be similar to the signals generated using the production device, or may be signals generated to conduct analysis similar to analysis conducted using the production device. Analyzed data may be generated by applying a continuous function line based on sensed data and generating a stance phase based on the continuous function.

As used herein, a production device may be one or more devices or systems that are known in a given industry as a gold standard device. As discussed herein, a gold standard device may be a device used to conduct a gold standard test. A gold standard test may be a diagnostic test or benchmark that is the best available under reasonable conditions. A gold standard device may be one that has been tested and has a reputation in the field as a reliable method. For example, for gait analysis a gold standard may include, but is not limited to, a gait lab including one or more gait mats, force plates, sensors, cameras, or the like. A gait lab may use equipment that enables the creation of extensive models of human movement, including force plates to measure ground reaction force (GRF), video analysis to enable mapping of an individual skeletal architecture, and/or electromyography to measure muscle activation during movement. For simplicity purposes, the concepts described herein refer to a GAITRite mat as the production device.

A test device may be a non-gold standard device that may be used to generate results or analysis similar to a production device. A test device may be a simpler, newer, and/or unverified version of a production device. A test device may have a number of sensors. The number of sensors in or associated with the test device may be less than a corresponding production device. The sensors in or associated with the test device may be less dense than a corresponding production device. A test device may require validation to confirm that results provided by and/or analysis conducted using data output by the test device provides comparable performance (e.g., meets a threshold performance) to a production device. Test devices may be novel digital health technologies (DHTs) that require validation before being deployed. For example, for gait analysis, as discussed herein, a test device may be a wearable insole device that may be used to calculate vertical ground reaction forces (vGRF). As another example, for speech analysis, the test device may be a mobile device-based speech program that facilitates the assessment of various speech tasks.

As described herein, the test device for gait assessment may be a sensor insole designed to be worn inside a shoe and is configured to measure plantar pressure at the sole of the foot along with foot acceleration in three axes using an inertial measurement unit (IMU). This insole computes various parameters, including plantar pressure distribution, functional gait characteristics (such as balance and symmetry), and movement patterns. In some aspects, the insole may weigh approximately 80 grams. In some aspects, the insole may resemble and feel like a regular insole. The sensor insole may incorporate a plurality of capacitive pressure sensors (e.g., 16 capacitive pressure sensors), an IMU, and a temperature sensor. The sensor insole may measure peak pressures, pressure distribution, acceleration, motion sequences, and other gait patterns. The insole may estimate the vector of force using the distribution of force on the sole. In some aspects, the sensor insole may be wireless and may come with internal data storage, enabling the processing of raw sensor data from each physical component. In an aspect, derived gait characteristics from the sensor insoles include metrics related to activity, dynamics, coordination, flexibility, and speed. The advantages of utilizing these sensor insoles over other types of sensor insoles include wireless control, data export, data analysis and reporting capabilities, suitability for various shoe types due to its thin design, measurement of plantar pressure distribution using pluralities of sensors, and potential for home use (off-site measurements).

As described herein, the test device for speech assessment may be a digital mobile speech-based platform. This application platform may leverage machine learning and natural language processing (NLP) to analyze speech and language patterns. In some aspects, the application platform may generate over approximately 760 individual variables from any given speech recording, providing detailed insights into both acoustic and linguistic features. In some aspects, the platform's data processing and feature extraction may be powered by a combination of Python-based libraries, including spaCy, the Stanford parser, and Praat/Parselmouth, along with custom code. By integrating these tools, this platform may capture a broad and detailed view of speech's acoustic and linguistic characteristics.

In the systems and processes described herein, one or more mixed effects models may be employed to analyze and assess the data by incorporating both fixed effects (e.g., which account for population-level influences) and random effects (e.g., which account for individual variability among subjects). More particularly, with respect to the former, fixed effects may include factors such as the subject OFF and ON stages (as further described herein), age, sex, and other clinicodemographic covariates that are expected to influence the outcome across the entire population. With respect to the latter, random effects capture the variability between subjects, accounting for the fact that each subject may have unique baseline levels and trajectories of change in gait and speech characteristics. This approach may be useful for repeated measures data, where multiple observations are made on the same subjects over time, such as in an assessment of gait and speech characteristics in PD.

According to implementations of the disclosed subject matter a test device may be validated based on a machine learning model trained using a production device. For example, for gait analysis, a wearable insole device may be validated based on a machine learning model trained using data generated at or related to a gait lab. As another example, for speech analysis, a mobile device-based speech program may be validated based on a machine learning model trained using data generated from MDS-UPDRS scores. As discussed herein, sensed data for a control group may be received from or generated at a production device. The sensed data may be output by one or more sensors associated with the production device. For example, the production device may correspond to a gait lab having one or more force sensor plates, mats, cameras, etc. A user may use the gait lab and the force sensor plates, cameras, etc. may output sensed data.

The production device and test device may each be configured to output data that can be used to identify a given condition. The given condition may be a medical condition, a physical condition, or the like. For example, the given condition may be a disorder such as Parkinson's disease, progressive supranuclear palsy, multiple sclerosis, osteoarthritis (OA), or the like. The production device may be configured to sense data (e.g., vGRF data) that may be analyzed to determine whether a given individual has a given condition, based on the sensed data. The control group may include a group of individuals that are know not to have and/or exhibit the given condition.

Production sensed data for a target group with individuals having the given condition (e.g., a target condition) may be received from or generated at the production device. For example, the production device may first sense data for a control group of individuals. Accordingly, the production device may be used to generate or provide both sensed data for a control group and a target group, where the target group includes individuals having a given condition. Additionally, according to an implementation, production sensed data from a production device for the control group may be used to generate control analyzed data. Similarly, production sensed data from the production device for the target group may be used to generate target analyzed data. Production sensed data may be data detected by one or more sensors of the production device (e.g., a production system such as a gait lab). The one or more sensors may include, but are not limited to, pressure sensors, motion sensors, cameras, biometric sensors, environment sensors, weight sensors, accelerometers, gyroscopes, or the like. Additionally, according to an implementation, production sensed data from a production device for the target group may be used to generate target analyzed data.

A machine learning model may be trained to identify a difference between the sensed data for the control group and the sensed data for the target group. A trained machine learning model may be generated based on the training. Different techniques to train machine learning models are disclosed herein. For example, supervised machine learning may be used to train the machine learning model such that, during training, the sensed data for the control group is marked as such and sensed data for the target group is marked as such. Accordingly, the machine learning model may be trained to identify the differences between the sensed data for the control group verses the sensed data for the target group, based on the markings.

Test sensed data sensed using a test device may be generated. The test data may be sensed for a test group of individuals that includes both individuals without the given condition and users that have the given condition. For example, the test device may be different than the production device and may be used by a group of individuals to generate the test sensed data. Whether an individual in the test group has the given condition or does not have the given condition may be known, though the test sensed data may not be marked to indicate whether a given user has or does not have the condition.

The test sensed data may be provided to the trained machine learning model, trained using the production sensed data. The trained machine learning model may receive the test sensed data and may generate a machine learning output based on the test sensed data. The machine learning output may categorize each or a subset of individuals in the test group as either having the given condition or as not having the given condition. The machine learning output categorizations may be compared to the known categorization of each respective individual. The comparison may be a determination of whether the individuals categorized by the machine learning model as having the given condition are known to have the given condition and/or whether the individuals categorized by the machine learning model as not having the given condition are known to not have the given condition. A match value may be determined based on the comparison and may quantify or qualify the degree to which the machine learning model correctly categorizes the test group. The match value may be compared to a match threshold, and if the match value meets or exceeds the match threshold, then the test device may be validated. Validation may mean that the test device performs at least as well as the production device to categorize individuals, as dictated by the match threshold.

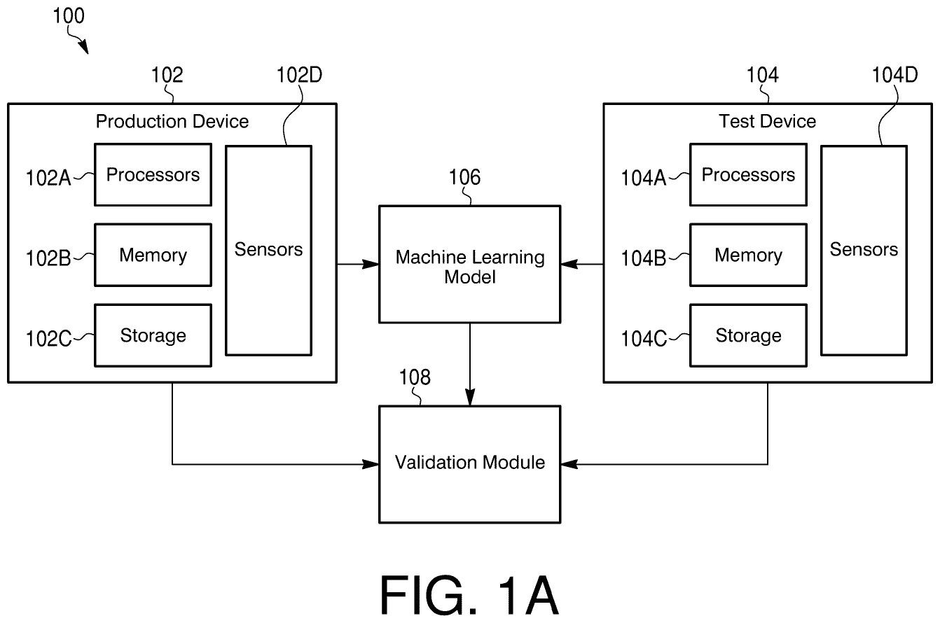

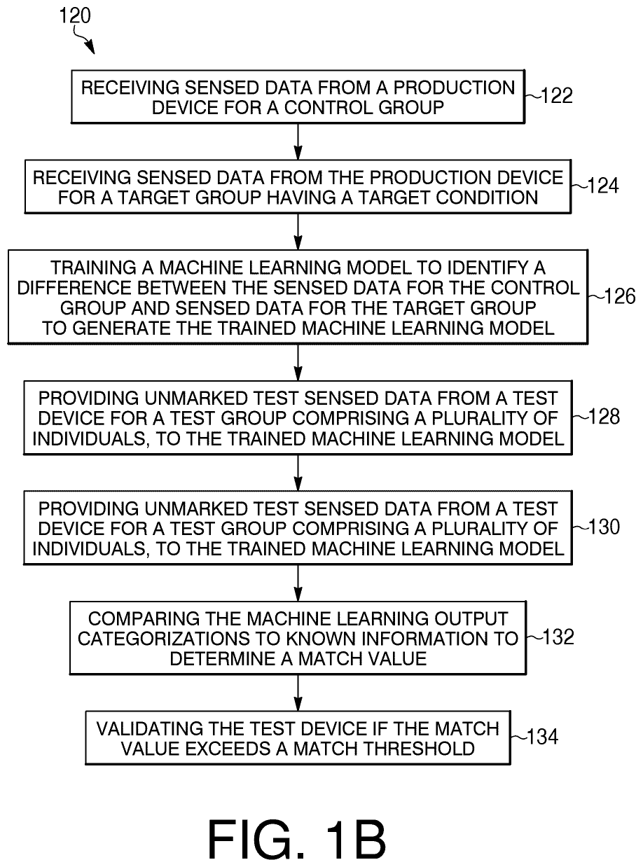

FIG. 1A shows a system environment 100 for validating a test device in accordance with the subject matter disclosed herein. As shown, a production device 102 may include one or more processors 102A, memories 102B, storage 102C, and/or sensors 102D. In some implementations, processors 102A may include one or more microprocessors, microchips, or application-specific integrated circuits. Memory 102B may include one or more types of random-access memory (RAM), read-only memory (ROM), and cache memory employed during execution of program instructions. Storage 102C may include one or more databases, cloud components, servers, or the like. Storage 102C may include a computer-readable, non-volatile hardware storage device that stores information and program instructions. Sensors 102D may be any sensors applicable to production device 102 and may include, but are not limited to, pressure sensors, motion sensors, cameras, biometric sensors, environment sensors, weight sensors, accelerometers, gyroscopes, or the like. Processors 102A may use data buses to communicate with memory 102B, storage 102C, and/or sensors 102D.

As also shown, a test device 104 may include one or more processors 104A, memories 104B, storage 104C, and/or sensors 104D. In some implementations, processors 104A may include one or more microprocessors, microchips, or application-specific integrated circuits. Memory 104B may include one or more types of random-access memory (RAM), read-only memory (ROM), and cache memory employed during execution of program instructions. Storage 104C may include one or more databases, cloud components, servers, or the like. Storage 104C may include a computer-readable, non-volatile hardware storage device that stores information and program instructions. Sensors 104D may be any sensors applicable to production device 102 and may include, but are not limited to, pressure sensors, motion sensors, cameras, biometric sensors, environment sensors, weight sensors, accelerometers, gyroscopes, or the like. Processors 104A may use data buses to communicate with memory 104B, storage 104C, and/or sensors 104D.

As shown in system environment 100, production device 102 and/or test device 104 may communicate with a machine learning model 106. Machine learning model 106 may be a standalone component or may be a part of production device 102 and/or test device 104. For example, production device 102 and/or test device 104 may communicate with machine learning model 106 over a network such that machine learning model 106 is a cloud component or stored at a cloud component. Machine learning model 106 may be implemented using one or more processors, memory, storage, or the like. According to an implementation, machine learning model 106 may receive data generated using sensors 102D and/or sensors 104D. Machine learning model 106 may receive the data directly from production device 102 and/or test device 104 (e.g., over a network) or may receive the data through a different component that receives the data from production device 102 and/or test device 104.

Validation module 108 may communicate with machine learning model 106, production device 102, and/or test device 104. Validation module 108 may receive a machine learning output (e.g., categorizations) from machine learning model 106 and may compare the output to known information (e.g., from production device 102 and/or test device 104). Validation module 108 may generate a match value based on the comparison and may compare the match value against a match threshold to validate test device 104.

FIG. 1B shows a flowchart 120 for validating a test device, in accordance with the subject matter disclosed herein. At step 122, sensed data from a production device for a control group may be received. The sensed data may be generated at one or more sensors 102D that may be part of a device or a system. The sensed data may be provided by processors 102A and may be stored at memory 102B and/or storage 102C such that processors 102A may retrieve the sensed data from memory 102B and/or storage 102C. The sensed data may be in the format output by one or more sensors 102D or may be in a different format. For example, processors 102A may receive the sensed data in a first format and may convert the sensed data to a second format. As further discussed herein processors 102A and/or one or more other components may generate analyzed data based on the sensed data. The control group may include individuals that are known not to have a given condition, as disclosed herein.

At step 124, sensed data from the production device for a target group may be received. The sensed data may be generated, provided, and/or formatted as disclosed in reference to the sensed data at step 122. The target group may include individuals that are known to have the given condition, as disclosed herein.

At step 126, a machine learning model may be trained to identify one or more differences between the sensed data for the control group (step 122) and the sensed data for the target group (step 124). A trained machine learning model may be generated based on the training. Training the machine learning model may include modifying one or more weights, biases, layers, nodes, or the like of the machine learning model based on the sensed data and/or differences in the sensed data for the control group and target group. Accordingly, the trained machine learning model may be configured to receive new sensed data (e.g., test sensed data as further discussed herein) to categorize an individual, to whom the new sensed data corresponds to, as either having the given condition or as not having the given condition. Techniques for training the machine learning model are further disclosed herein.

At step 128, unmarked test data from a test device for a test group may be provided to the trained machine learning model. The test group may include some individuals known to have the given condition and some individuals known not to have the given condition. The test sensed data may be unmarked such that the unmarked test data input to the machine learning model may not include a marker or other indication of whether a given individual who is part of the test group has or does not have the given condition. For example, the test group may include first individuals known to have the target condition and second individuals known to not have the target condition. Accordingly, the trained machine learning model may not receive a marker or other indication about whether each individual in the test group has or does not have the given condition. The unmarked test data may be processed by the trained machine learning model based on one or more of the weights, biases, layers, nodes, or the like of the trained machine learning model.

At step 130, a machine learning output may be received from the trained machine learning model. The machine learning model may categorize each of the plurality of individuals in the test group as respectively either having the given condition or not having the given condition. Accordingly, the trained machine learning model may independently determine whether a given individual is categorized as having the given condition or as not having the given condition, without prior input or knowledge of the same. For example, the machine learning output may categorize some of the individuals in the input test group as third individuals having the given condition or fourth individuals not having the given condition. It will be understood that some individuals from the test group may not be categorized as having the given condition or not having the given condition. For example, the unmarked test data for a given individual may be ambiguous such that the trained machine learning model may not categorize that given individual with a required level of certainty.

At step 132, the machine learning output categorizations may be compared to the known information about each individual in the test group, to determine a match value. For example, the first individuals (known to have the given condition) may be compared to the third individuals categorized by the machine learning output as having the given condition. Alternatively, or in addition, second individuals (known to not have the given condition) may be compared to the fourth individuals categorized by the machine learning output as not having the given condition. Accordingly, at step 132, a comparison of the known information for each respective individual may be compared to the categorization determined by the machine learning model.

A match value may be determine based on the comparison at step 132. The match value may be a quantitative or qualitative comparison and may indicate the degree to which the machine learning output correctly categorized individuals in the test group as either having or not having the given condition. The match value may be a numerical score, a correlation value, an overlap value, a percentage, a tier, or the like that indicates the success of the machine learning output in correctly categorizing the individuals in the test group as having or not having the given condition.

At step 134, a validation component (e.g., validation module 208) and/or the test device may determine if the match value meets or exceeds a match threshold. If the match value meets or exceeds the match threshold, then the test device may be validated. A match threshold format may be comparable to the match value format such that the match value can be compared to the match threshold. The match threshold may be predetermined, may be set (e.g., via user input), or may be dynamically determined. A dynamically determined match threshold may be dynamically determined using a match threshold machine learning model and/or an algorithm or other computation mechanism. A dynamic match threshold may be determined based on the given condition, the production device, the test device, the test group, or the like.

Accordingly, a validated test device may be a device that can be used to categorize individuals as having a given condition or not having a given condition, and/or to accurately determine whether characteristics associated with a given condition are present in an individual, as tested against a production device. A validated test device may be approved for use to determine presence of the given condition, or symptoms associated therewith, in a manner similar to determining the presence of the given condition using the production device. However, it will be understood that the test device may use different components (e.g., sensors) than the production device. For example, the test device may include simpler or different components than the production device, yet may be validated to perform the same test(s) as the production device.

The production device and/or the test device may each generate sensed data based on respective components (e.g., sensors). Accordingly, the sensed data from the production device may be in a different format, may be calibrated differently, may be categorized and/or stored differently, or the like, than the sensed device from the test device. For example, sensed data from a gait lab may include force plate data for number of sensors in one or more force plates at the gait lab and may also include camera data, motion data, etc. Sensed data from a wearable insole device may be pressure data detected by sensors included within the insole. Sensed data from a digital mobile speed-based platform may include characteristics of audible input provided by a user. Accordingly, the sensed data from a production device may not provide a one-to-one comparison to sensed data from a test device. As a result, a machine learning model trained using production sensed data may not be configured to provide an applicable machine learning output based on test sensed data.

According to implementations of the disclosed subject matter, control analyzed data may be generated based on the control group production sensed data and target analyzed data may be generated based on the target group production sensed data. Similarly, test analyzed data may be generated based on the test sensed data. For example, production sensed data from a gait lab may be used to determine a control vGRF for each individual in the control group. Production sensed data from the gait lab may be used to determine a target vGRF for each individual in the target group. Similarly, test sensed data from the wearable insole device may be used to determine a test vGRF for each individual in the test group. Accordingly, each of the control analyzed data, the target analyzed data, and the test analyzed data may have a one-to-one correlation such that although the underlying sensed data may be incomparable for each of the control, target, and test groups, the analyzed data may be comparable.

The machine learning model may be trained based on the control analyzed data and the target analyzed data. Subsequently, test analyzed data may be provided to the trained machine learning model and a machine learning output may be generated based on the test analyzed data. In this implementation, the machine learning model may be trained using the same format or type of data as the machine learning model uses to generate a machine learning output. The machine learning output may categorize each or a subset of individuals in the test group as either having the given condition or as not having the given condition, based on their respective test analyzed data (e.g., test vGRF plots). The machine learning output categorizations may be compared to the known categorization of each respective individual in the test group. The comparison may be a determination of whether the individuals categorized by the machine learning model as having the given condition are known to have the given condition and/or whether the individuals categorized by the machine learning model as not having the given condition are known to not have the given condition. A match value may be determined based on the comparison and may quantify or qualify the degree to which the machine learning model correctly categorizes the test group. The match value may be compared to a match threshold, and if the match value meets or exceeds the match threshold, then the test device may be validated. Validation may mean that the test device performs at least as well as the production device to categorize individuals, as dictated by the match threshold.

According to an implementation of the disclosed subject matter, a trained machine learning model may be validated based on a control group. Production sensed data for a first subset of a control group with individuals not having a given condition (e.g., a target condition) may be received from or generated at the production device. Similarly, production sensed data for a first subset of a target group with individuals having the given condition may be received from or generated at the production device. Accordingly, the production device may be used to generate or provided both sensed data for a first subset of the control group and a first subset of the target group, where the target group includes individuals having a given condition. Production sensed data may be data detected by one or more sensors of the production device (e.g., a production system such as a gait lab).

A machine learning model may be trained to identify a difference between the sensed data for the first subset of the control group and the sensed data for the first subset of the target group. A trained machine learning model may be generated based on the training. Different techniques to train machine learning models are disclosed herein. For example, supervised machine learning may be used to train the machine learning model such that, during training, the sensed data for the first subset of the control group is marked as such and sensed data for the first subset of the target group is marked as such. Accordingly, the machine learning model may be trained to identify the differences between the sensed data for the first subset of the control group verses the sensed data for the first subset of the target group, based on the markings.

A verification group may include a second subset of the control group with individuals known to not have the given condition and also a second subset of the target group with individuals known to not have the given condition. Production sensed data for the second subset of the control group with individuals known to not have the given condition may be received from or generated at the production device. Similarly, production sensed data for the second subset of the target group with individuals known to have the given condition may be received from or generated at the production device. The sensed data for the second subset of the control group and the second subset of the target group may not be marked. Unmarked verification sensed data may correspond to the sensed data for the second subset of the control group and the second subset of the target group (the verification group).

The unmarked verification sensed data for the verification group may be provided to the trained machine learning model. The trained machine learning model may receive the unmarked verification sensed data and may generate a machine learning output based on the same. The machine learning output may categorize each or a subset of individuals in the unmarked verification sensed data as either having the given condition or as not having the given condition. The machine learning output categorizations may be compared to the known categorization of each respective individual. The comparison may be a determination of whether the individuals categorized by the trained machine learning model as having the given condition are known to have the given condition (i.e., are part of the second subset of the control group) and/or whether the individuals categorized by the machine learning model as not having the given condition are known to not have the given condition (i.e., are part of the second subset of the target group). A match value may be determined based on the comparison and may quantify or qualify the degree to which the trained machine learning model correctly categorizes the test group. The match value may be compared to a match threshold, and if the match value meets or exceeds the match threshold, then the trained machine learning model may be validated. Validation may mean that the trained machine learning model is configured to categorize individuals as having or not having the given condition with a level of certainty, as dictated by the match threshold.

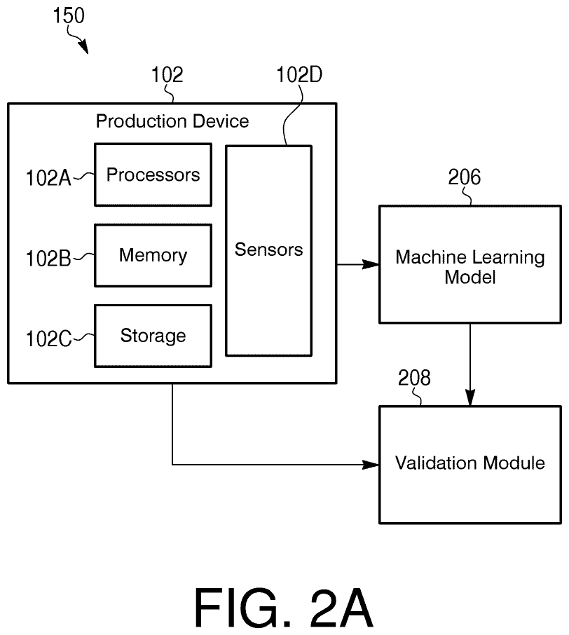

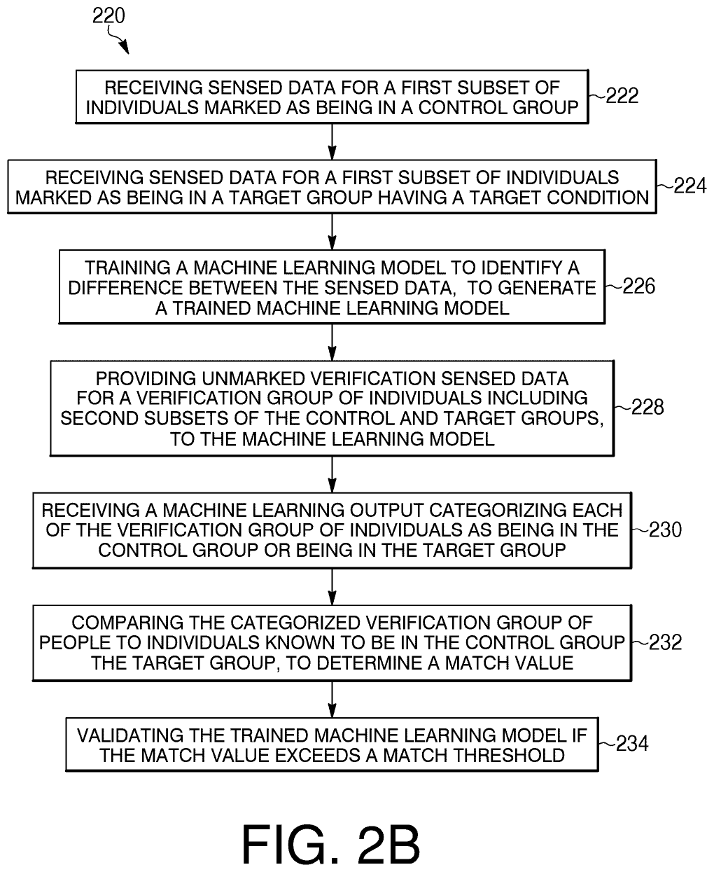

FIG. 2A shows a system environment 150 for validating a machine learning model in accordance with the subject matter disclosed herein. As shown, system environment 150 may include some components that are the same as or similar to the components of system environment 100 of FIG. 1A. Accordingly, such components are not described again for brevity. As shown, system environment 150 may include production device 102, processors 102A, memory 102B, storage 102C, and sensors 102D. Production device 102 and/or one or more of its components may communicate with machine learning model 206 which may be similar to or different than machine learning model 106 of FIG. 1A. Machine learning model 206 is further discussed herein.

Validation module 208 may communicate with machine learning model 206 and/or production device 102. Validation module 208 may receive a machine learning output (e.g., categorizations) from machine learning model 206 and may compare the output to known information (e.g., from production device 102). Validation module 208 may generate a match value based on the comparison and may compare the match value against a match threshold to validate test device 104.

FIG. 2B shows a flowchart 220 for validating a machine learning, in accordance with the subject matter disclosed herein. At step 222 sensed data from a production device for a first set of individuals marked as being in a control group may be received. The sensed data may be generated at one or more sensors 102D that may be part of a device or a system. The sensed data may be provided by processors 102A and may be stored at memory 102B and/or storage 102C such that processors 102A may retrieve the sensed data from memory 102B and/or storage 102C. The sensed data may be in the format output by one or more sensors 102D or may be in a different format. For example, processors 102A may receive the sensed data in a first format and may convert the sensed data to a second format. As further discussed herein processors 102A and/or one or more other components may generate analyzed data based on the sensed data. The control group may include individuals that are known not to have a given condition, as disclosed herein.

At step 224, sensed data from the production device for a first set of individuals marked as being in a target group may be received in a manner similar to that discussed for step 222. The target group may include individuals that are known to have the given condition, as disclosed herein.

At step 226, a machine learning model may be trained to identify one or more differences between the sensed data for the first subset of the control group (step 222) and the sensed data for the first subset of the target group (step 224). A trained machine learning model may be generated based on the training. Training the machine learning model may include modifying one or more weights, biases, layers, nodes, or the like of the machine learning model based on the sensed data and/or differences in the sensed data for the first subset of the control group and the first subset of the target group. Accordingly, the trained machine learning model may be configured to receive verification sensed data (e.g., sensed data for a verification group having second subsets of the control group and/or the target group, as further discussed herein) to categorize an individual, to whom the verification sensed data corresponds to, as either having the given condition or as not having the given condition. Techniques for training the machine learning model are further disclosed herein.

At step 228, unmarked verification sensed data from the production device for a verification group may be provided to the trained machine learning model. The verification group may include a second subset of the control group not having the given condition and a second subset of the target group having the given condition. The verification sensed data may be unmarked such that the unmarked verification sensed data input to the machine learning model may not include a marker or other indication of whether a given individual who is part of the verification group has or does not have the given condition. Accordingly, the trained machine learning model may not receive a marker or other indication about whether each individual in the verification group has or does not have the given condition. The unmarked verification sensed data may be processed by the trained machine learning model based on one or more of the weights, biases, layers, nodes, or the like of the trained machine learning model.

At step 230, a machine learning output may be received from the trained machine learning model. The machine learning model may categorize each of the plurality of individuals in the verification group as respectively either having the given condition or not having the given condition. Accordingly, the trained machine learning model may independently determine whether a given individual in the verification group is categorized as having the given condition or as not having the given condition, without prior input or knowledge of the same. It will be understood that some individuals from the verification group may not be categorized as having the given condition or not having the given condition. For example, the unmarked verification data for a given individual may be ambiguous such that the trained machine learning model may not categorize that given individual with a required level of certainty.

At step 232, the machine learning output categorizations may be compared to the known information about each individual in the verification group, to determine a match value. For example, the individuals in the second subset of the control group (known to have the given condition) may be compared to the individuals categorized by the machine learning output as having the given condition. Alternatively, or in addition, individuals in the second subset of the target group (known to not have the given condition) may be compared to the individuals categorized by the machine learning output as not having the given condition. Accordingly, at step 232, a comparison of the known information for each respective individual may be compared to the categorization determined by the machine learning model.

A match value may be determine based on the comparison at step 232. The match value may be a quantitative or qualitative comparison and may indicate the degree to which the machine learning output correctly categorized individuals in the verification group as either having or not having the given condition. The match value may be a numerical score, a correlation value, an overlap value, a percentage, a tier, or the like that indicates the success of the machine learning output in correctly categorizing the individuals in the verification group as having or not having the given condition.

At step 234, a validation component (e.g., validation module 208) may determine if the match value meets or exceeds a match threshold. If the match value meets or exceeds the match threshold, then the machine learning model trained at step 226 may be validated. A match threshold format may be comparable to the match value format such that the match value can be compared to the match threshold. The match threshold may be predetermined, may be set (e.g., via user input), or may be dynamically determined. A dynamically determined match threshold may be dynamically determined using a match threshold machine learning model and/or an algorithm or other computation mechanism. A dynamic match threshold may be determined based on the given condition, the production device, the test device, the test group, or the like.

Accordingly, a validated machine learning model may be a model that can be used to categorize individuals as having a given condition or not having a given condition, as tested against subsets of control and target individuals. A validated machine learning model may be approved for use to, for example, determine if a test device (e.g., as described in FIGS. 1A and 1B) is validated. For example, an untrained or previously trained version of a validated machine learning model may be trained at step 126 of FIG. 1B.

In a manner similar to that described above, the machine learning model may be trained based on control analyzed data and target analyzed data. The trained machine learning model may receive verification analyzed data and may generate a machine learning output based on the verification analyzed data.

Each block in the flow diagram of FIG. 1A or 2A, or flowcharts of FIGS. 1B, 2B can represent a module, segment, or portion of program instructions, which includes one or more computer executable instructions for implementing the illustrated functions and operations. In some alternative implementations, the functions and/or operations illustrated in a particular block of a flow diagram or flowchart can occur out of the order shown in the respective figure.

For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

In various implementations disclosed herein, systems and methods are described for using machine learning to validate a test device and/or for validation of a machine learning model. By training a machine learning model, e.g., via supervised or semi-supervised learning, to learn associations between training data and ground truth data, the trained machine learning model may be used to validate one or more test devices.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, extreme gradient boosting (XGBoost), random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

As discussed herein, machine learning techniques adapted to validate a model and/or validate a test device, may include one or more aspects according to this disclosure, e.g., a particular selection of training data, a particular training process for the machine learning model, operation of a particular device suitable for use with the trained machine learning model, operation of the machine learning model in conjunction with particular data, modification of such particular data by the machine learning model, etc., and/or other aspects that may be apparent to one of ordinary skill in the art based on this disclosure.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine learning model, e.g., compare the output of the trained model with the ground truth for that portion of the training data to evaluate an accuracy of the trained model. The training of the machine learning model may be configured to cause the machine learning model to learn associations between training data and ground truth data, such that the trained machine learning model is configured to determine an output in response to the input data based on the learned associations.

In various implementations, the variables of a machine learning model may be interrelated in any suitable arrangement in order to generate the output. For example, in some embodiments, the machine learning model may include image-processing architecture that is configured to identify, isolate, and/or extract features, geometry, and or structure in one or more of the medical imaging data and/or the non-optical in vivo image data. For example, the machine learning model may include one or more convolutional neural network (“CNN”) configured to identify features in data, and may include further architecture, e.g., a connected layer, neural network, etc., configured to determine a relationship between the identified features in order to determine a location in the data.

In some instances, different samples of training data and/or input data may not be independent. Thus, in some embodiments, the machine learning model may be configured to account for and/or determine relationships between multiple samples.

For example, in some embodiments, the machine learning models described in FIGS. 1A-2B may include a Recurrent Neural Network (“RNN”). Generally, RNNs are a class of feed-forward neural networks that may be well adapted to processing a sequence of inputs. In some embodiments, the machine learning model may include a Long Shor Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of non-optical in vivo images as input, and generate a sequence of locations, e.g., a path, in the medical imaging data as output.

Implementations of the disclosed subject matter are disclosed herein with references to one or more examples. It will be understood that the implementations disclosed herein are not limited only to the data, orders, or specifics disclosed in the examples. It will be further understood that the example includes implementations that can be applied generally to the subject matter disclosed herein.

### Objectives for Assessing the Feasibility of Gait and Speech Analysis Using Test Devices

Objectives of this disclosure focus on evaluating the effectiveness and correlation of a test device configured for gait analysis (e.g., a wearable insole) in assessing gait parameters and motor symptoms associated with PD. First, the disclosure aims to assess the ability of wearable insoles to detect changes in gait and motor function parameters, as recorded by the production device and MDS-UPDRS III, from the OFF state (prior to treatment administration such as an amino acid based treatment) to the ON state (post-treatment administration). Second, the disclosure determines relationships between gait parameters measured by the test device and those measured by the production device. Lastly, the disclosure evaluates the relationship between gait parameters measured by the test device and applicable scores (e.g., scores based on MDS-UPDRS III).

To achieve these objectives, several endpoints were investigated. These include the change in gait from OFF to ON state, as measured by the test and production devices and specific items of MDS-UPDRS III related to lower extremity and axial function. Additionally, the disclosure examines the concordance of gait parameters derived from the test and production devices, as well as the concordance between gait parameters from the test device and scores (e.g., MDS-UPDRS III scores) for items related to lower extremity rigidity, toe tapping, leg agility, gait, freezing of gait, postural instability, and posture.

The disclosure additionally aims to assess changes in voice performance and their relationship with motor function in PD. Specifically, the disclosure documents the evaluation of the change in voice performance from the OFF state to the ON state using a digital mobile speech-based platform, which is configured to perform quantitative speech analysis. The disclosure further aims to establish the relationship between quantitative speech analysis and applicable scores (e.g., scores based on MDS-UPDRS III). To address these objectives, the disclosure investigated endpoints such as the change in speech performance from OFF to ON state as measured by the digital mobile speech-based platform, and the change in voice and cranial function scores from OFF to ON state as measured by MDS-UPDRS III.

The disclosure further aims to understand specific aspects of motor function and the usability of the gait analysis test devices in PD patients. These exploratory objectives include establishing the relationship between changes in toe tapping parameters and changes in MDS-UPDRS Part III scores, as well as the relationship between changes in leg agility parameters and changes in MDS-UPDRS Part III scores. To explore these objectives, the disclosure investigated endpoints such as the change in toe tapping parameters from OFF to ON state, as measured by the wearable insoles, and the change in leg agility parameters from OFF to ON state, as measured by the wearable insoles.

The disclosure further aims to evaluate the effectiveness of specialized test devices in assessing the general performance of subjects diagnosed with medical conditions (e.g., neurological conditions) to complete certain tasks. The objectives include measuring changes in the subject's task performance capabilities across different treatment states (e.g., OFF and ON states) using both test devices and establish clinical scales, such as the MDS-UPDRS III. Additionally, the disclosure seeks to establish the correlation between measurements obtained from test devices and those from traditional production devices, aiming to validate their clinical utility. Exploratory goals involve examining specific aspects of how a subject is able to perform a designated task, which may provide valuable insights into the potential benefits of these technologies for monitoring and managing symptoms effectively.

### Definitions and Derived Variables

Provided below are lists of various terms, and definitions associated therewith, that may be relevant to some or all aspects of the disclosure described herein.

Movement Disorder Society's Unified Parkinson's Disease Rating Scale (MDS-UPDRS) Overall Score: This is a comprehensive tool used for monitoring the progression of Parkinson's Disease (PD) and assessing the severity of symptoms. It includes a combination of self-reported and clinician-scored sections that evaluate non-motor and motor experiences of daily living, motor examination, and motor complications. The MDS-UPDRS includes four parts:

Part I corresponds to Non-Motor Experiences of Daily Living: This part is focused on the non-motor symptoms often associated with PD. It may further be divided into two subsections, one for patient-reported experiences and another for caregiver- or clinician-reported experiences. Areas of assessment may include cognitive impairment, mood, sleep, and autonomic function.

Part II corresponds to Motor Experiences of Daily Living: This part assess the subject's self-perception of motor function in daily life. It may include questions related to speech, salivation, eating, dressing, hygiene, handwriting, doing hobbies and other daily activities, and how the disease might be impacting these tasks.

Part III corresponds to Motor Examination: This section may be evaluated by a clinician and focuses on the motor symptoms of PD. It may involve assessments of speech, facial expression, tremors, rigidity, posture, gait, and bradykinesia (slowness of movement), among other things.

Part IV corresponds to Motor Complications: This part is designed to evaluate the presence and extent of motor complications often associated with long-term PD or its treatment. It may cover dyskinesias (involuntary movements), motor fluctuations (e.g., ‘on-off’ phenomenon), and other related complications.

Some or all of the following demographic variables were measured and considered as covariates in downstream analysis, including: age at screening (year), race (e.g., American Indian/Alaskan Native, Asian, Black/African American, Native Hawaiian/Other Pacific Islander, White and Other), baseline weight, baseline height, concomitant medications, baseline BMI, and leg length (if available).

Some or all of the following gait variables may be considered when assessing the production device, including: stride length (e.g., the distance between successive points of initial contact of the same foot), step length (e.g., the distance between successive points of initial contact of the opposite foot), walking speed/velocity (e.g., the speed at which the individual is walking, typically measured in meters per second), cadence (e.g., the number of steps taken per minute), step time (e.g., the time it takes for a step, from footfall to footfall), stride time (e.g., the time for a full cycle of gait, usually measured from the time of initial contact of one foot to the time of the next initial contact of the same foot), stance time (e.g., the duration of time that a foot remains on the ground during a gait cycle), swing time (e.g., the time period when the foot is in the air during the gait cycle), single support time (e.g., the duration when only one foot is in contact with the ground), double support time (e.g., the duration when both feet are in contact with the ground), foot angle (e.g., the angle of foot placement relative to the line of progression), and base of support/step width (e.g., the lateral distance between the line of the two feet during the gait cycle).

Some or all of the following digital insole variables may be considered when assessing the test device, including: mean gait line length (e.g., average length of the gait line for both left and right feet (mm)), mean gait line width (e.g., average width of the gait line for both left and right feet (mm)), mean center of pressure (COP) in X and Y directions (e.g., average location of COP in both the X and Y axes for both left and right feet (mm)), standard deviation (SD) of COP in X and Y directions (e.g., the variability of COP in both the X and Y axes for both left and right feet (mm)), dimensions of bounding box of COP (e.g., length and width of the bounding box containing all COP points for both left and right feet (mm)), mean COP velocity (e.g., average speed of the COP traveling across the sensor insole surface for both left and right feet (mm/s)), COP trace length (e.g., overall travel of the COP across the sensor insole surface for both left and right feet (m)), force during stance phase (e.g., mean and maximum force during stance phase for both left and right feet (N)), mean of all maximum total force during stance phase (e.g., average of the maximum total force for all steps for both left and right feet (N)), mean gait cycle time (e.g., time from one heel strike to the next of the same foot, averaged over all steps(s)), and mean gait cadence (e.g., number of strides per minute (Strides/min)). Mean double support time and fraction (e.g., time during which both feet are on the ground, averaged over all steps, and the relative mean double support time to the mean gait cycle time (s, %)), side-specific mean double support time (e.g., time starting from one foot's heel strike to the other foot's toe off, for both left and right feet(s)), mean step duration (e.g., average time between consecutive heel strikes for both left and right feet(s)), mean and SD stance duration (e.g., time from heel strike to toe off, averaged over all steps, and its standard deviation for both left and right feet(s)), mean and SD swing duration (e.g., time from toe off to heel strike, averaged over all steps, and its standard deviation for both left and right feet(s)), mean and SD fraction of stance phase (e.g., mean stance duration relative to the mean gait cycle time and its standard deviation for both left and right feet (%)), mean and SD fraction of swing duration (e.g., mean swing duration relative to the mean gait cycle time and its standard deviation for both left and right feet (%)), force raise (e.g., time to first force peak after initial ground contact for both left and right feet(s)), takeoff dynamics (e.g., ratio of force value of force minimum in stance phase and 2nd force peak (takeoff) for both left and right feet), gait direction dynamics (e.g., amplitude of foot acceleration in the direction of walking for both left and right feet (g)), mean stride length (e.g., displacement of the same foot in the walking direction averaged over all detected steps (m)), foot flexibility (e.g., amplitude of foot acceleration in the body's vertical direction for both left and right feet (g)), walking distance (e.g., the total distance traveled over the entire measurement (m)), mean walking speed (e.g., the mean stride length divided by the mean gait cycle time (m/s)).

Some or all of the following speech variables may be considered when assessing the digital mobile speech-based platform, including: ADJP, ADVP, FRAG, INTJ Parameters (e.g., these parameters are related to the parts of speech in the English language. They help measure the usage frequency of these parts in the speech, including adjectives, adverbs, fragments, and interjections), articulation rate (e.g., this measurement indicates the speed at which the speaker pronounces words), average word duration and length (e.g., these parameters measure the average duration and length (in number of syllables or letters) of words in the speech), familiarity and frequency (e.g., these metrics assess how common or familiar words used by the speaker are in a certain language or context), fundamental frequency parameters (e.g., these relate to the pitch of the speaker's voice, including maximum, mean, median, minimum, range, and variance of the fundamental frequency), global and local coherence parameters (e.g., these features measure the semantic coherence or continuity of speech at both global (whole text) and local (sentence-level) scales, using word embedding models like GloVe and Google's word2vec), pause duration and count (e.g., these metrics measure the length and frequency of pauses in the speech, which could be indicative of hesitations or thought processing speed), morphological parameters (e.g., these parameters measure different morphological aspects of the language used, like the tense, number, person, voice, mood, etc. of the verbs, or the case, gender, number, etc. of the nouns), mel-frequency cepstral coefficients (MFCCs) (e.g., these are coefficients that collectively make up an MFC. They are derived from a type of cepstral representation of the audio clip (a nonlinear “spectrum-of-a-spectrum”)), intensity parameters (e.g., these parameters measure the loudness or intensity of the speaker's voice, including maximum, mean, median, minimum, range, and variance of the intensity), jitter and harmonic to noise ratio (HNR) (e.g., these parameters measure the stability and quality of the speaker's voice. Jitter relates to the frequency instability, while HNR contrasts the harmonic (periodic) and noisy parts of the speech signal), graph parameters (e.g., these features represent the speech as a graph (network of words or sentences) and measure different graph properties like density, diameter, total degree, etc.), and semantic and sentiment parameters (e.g., these metrics measure the meanings or sentiments conveyed by the words or sentences in the speech, including sentiment arousal, dominance, and valence).

### Participant Subject Characteristics

The subjects involved in the studies discussed in this disclosure were required to satisfy a variety of inclusion criteria to be eligible for study participation and simultaneously could not be associated with any exclusion criteria.

To participate in the study, participants were required to meet several inclusion criteria. First, they needed a clinical diagnosis of PD confirmed by a treating movement disorders neurologist. Participants had to be between the ages of 40 and 70. They should be taking standing levodopa as part of their PD regimen and exhibit motor fluctuations by clinical history, specifically with mild to moderate severity, as defined by a score of 2 (mild) or 3 (moderate) on the MDS-UPDRS item 4.4 (Functional impact of fluctuations). Participants had to be willing to undergo a levodopa challenge test, which involves coming to an assessment site for evaluation in the morning after not taking levodopa since the night before (e.g., at least 12 hours). They would then take levodopa at the site, wait for it to take effect (achieving the “ON” state), and repeat the motor evaluation. Additionally, participants needed to be ambulatory in the “OFF” state, meaning they could walk safely without a walking aid on a flat surface at the start of the levodopa challenge test. Further, participants had to be willing and able to comply with clinic visits and study-related procedures, provide informed consent either signed by themselves or a legally authorized representative, or be able to understand and complete study-related procedures. Lastly, they must be fluent in either English or Spanish.

Participants were excluded from participating in the study if they had a clinical history of dementia, significant leg or back arthritis, or any signs or symptoms suggesting atypical parkinsonism. Additionally, those who have had a previous stroke or recent orthopedic surgery (within the past year) were not be eligible. Members of the clinical site study team and their immediate family members were also excluded from participation, unless prior approval was granted.

The study included multiple primary endpoints and within these endpoints, multiple parameters that were be tested. The strength of the correlation between different wearable technologies has not been shown in prior studies, and the correlation may differ in strength depending on the parameter tested. Furthermore, in some aspects, it is possible that a strong correlation with other wearable technologies may be achieved, but the study may not also demonstrate a strong correlation with clinical evaluation. An aim of this study is to obtain estimates of these parameters and perform a preliminary comparison to help power future trials. The sample size for this study is a convenience sample. The interim analysis may be comprised of N=10 patients and estimates of the precision around the mean parameters are listed below.

The mean and standard deviation of Gait characteristics from a reference PD population of N=119 are listed below. Gait was quantified using an industry standard production device, from which these 16 gait characteristics were derived and assessed. The reference PD population were 119 people with early PD diagnosed according to the UK Parkinson's Disease Brain Bank criteria by a movement disorder specialist. The 95% confidence intervals (CI) of the 16 Gait parameters were calculated using the expected interim sample size of N=10.

Table B below provides the mean and standard deviation of gait characteristics from a reference PD population

TABLE B

| Gait Model | Gait | | |
|---|---|---|---|
| Domain | Characteristics | Mean (SD) | 95% CI |
| Pace | Step Velocity (m/s) | 1.125 (0.213) | (0.973, 1.277) |
| | Step Length (m) | 0.623 (0.101) | (0.551, 0.695) |
| | Swing Time | 0.018 (0.006) | (0.014, 0.022) |
| | Variability (s) | | |
| Rhythm | Step Time (m) | 0.560 (0.049) | (0.525, 0.595) |
| | Swing Time (s) | 0.392 (0.033) | (0.368, 0.416) |
| | Stance Time (s) | 0.728 (0.077) | (0.673, 0.783) |
| Variability | Step Velocity | 0.054 (0.017) | (0.042, 0.066) |
| | Variability (m/s) | | |
| | Step Length | 0.023 (0.008) | (0.017, 0.029) |
| | Variability (m) | | |
| | Step Time | 0.019 (0.006) | (0.015, 0.023) |
| | Variability (s) | | |
| | Stance Time | 0.023 (0.009) | (0.017, 0.029) |
| | Variability (s) | | |
| Asymmetry | Step Time | 0.023 (0.028) | (0.003, 0.043) |
| | Asymmetry (s) | | |
| | Swing Time | 0.017 (0.020) | (0.003, 0.031) |
| | Asymmetry (s) | | |
| | Stance Time | 0.017 (0.019) | (0.003, 0.031) |
| | Asymmetry (s) | | |
| Postural | Step Width (m) | 0.093 (0.031) | (0.071, 0.115) |
| Control | Step Width | 0.019 (0.006) | (0.015, 0.023) |
| | Variability (m) | | |
| | Step Length | 0.026 (0.022) | (0.010, 0.042) |
| | Asymmetry (m) | | |

### General Procedure

An aim of this disclosure is to comprehensively evaluate the effectiveness of test devices such as the gait and speech analysis test devices (e.g., the wearable insoles and the mobile-speech based platform, respectively) in patients diagnosed with PD. For the gait analysis test device, the evaluation encompasses both analytical and clinical aspects. Analytically, patients with PD undertook a walking task, during which gait parameters were measured concurrently using the wearable insoles test device. These insoles are equipped with advanced technology, including accelerometers, gyroscopes, and pressure sensors. Simultaneously, gait parameters were also recorded using the production device, renowned as the reference method for such assessments.

The clinical evaluation of both the wearable insoles and the mobile-speech based platform involves assessing patients in two distinct states: the OFF state, characterized by the absence of the effects of a treatment such as dopaminergic medication, and the ON state, when medication is optimally effective. In both the OFF and ON states, several assessments were conducted, including gait assessment using both the gait analysis test and production devices, quantitative speech and language analysis utilizing the digital mobile speech-based platform on a tablet or mobile device, and administration of the MDS-UPDRS, the established clinical rating scale for PD.

This comprehensive approach aims to provide a thorough understanding of the efficacy of the wearable insoles and the mobile speech-based platform in improving motor function and speech performance in patients with PD across varying medication states. By conducting assessments in both OFF and ON states and comparing results obtained from different evaluation methods, this disclosure seeks to determine the potential benefits of these technologies for PD management and treatment.

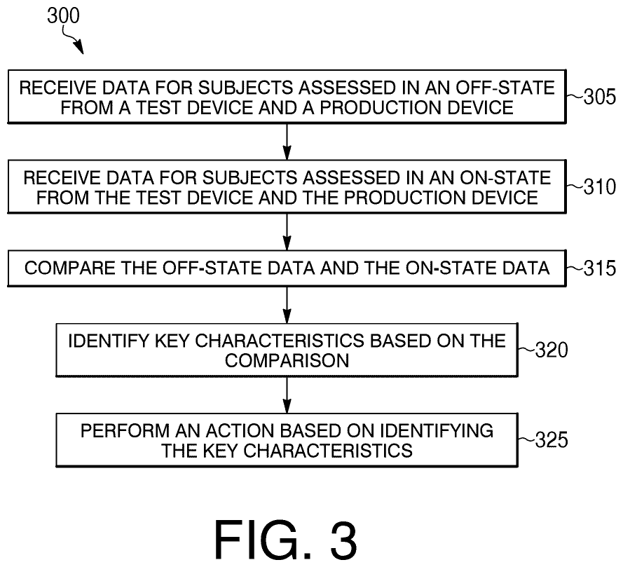

Referring now to FIG. 3, an exemplary workflow 300 is provided for comparing the characteristics of test and production devices in the accurate detection of gait and speech parameters associated with PD. Aspects of the exemplary workflow 300 may be performed in accordance with some or all components and processes described in FIGS. 1-2.

At step 305, system 100 may receive data from subjects that were assessed in an OFF-state. In the context of this application, the OFF-state may be characterized as a subject state when the subject is not under an effect of a treatment such as a dopaminergic medication (e.g., levodopa), a subject state when the subject is under the effect of the treatment such as the dopaminergic medication at a first dosage (e.g., a dosage under a threshold dosage at which drug effects are known to occur in a subject), and/or a subject state when the subject is under the effect of the treatment such as the dopaminergic medication after a threshold duration of time (e.g., where the threshold duration of time is a time at which most or all substantive drug effects are known to have worn off in the subject). In an aspect, the off-state assessments were designed to evaluate the baseline motor and functional abilities of subjects with PD without the influence of a treatment such as dopaminergic medication, e.g., levodopa. To achieve this, subjects were required to refrain from undertaking the treatment (e.g., taking dopaminergic medication for at least 12 hours prior to the assessment. This may typically involve subjects taking their last dose of medication the night before the assessment and then arriving at the assessment site the next morning without having taken their morning dose. This ensures that the medication has worn off, thereby placing the subject in the OFF state. In some aspects, upon arrival at the study site, subjects may be subjectively questioned to confirm that they are indeed in the OFF state (e.g., by confirming that their last dose was taken more than 12 hours ago and assessing their current symptoms and motor function). Although this is a subjective confirmation, it is important to ensure that the baseline measurements are accurate and reflective of the medication-free state. Once confirmed to be in the OFF state, the subjects underwent a series of assessments.

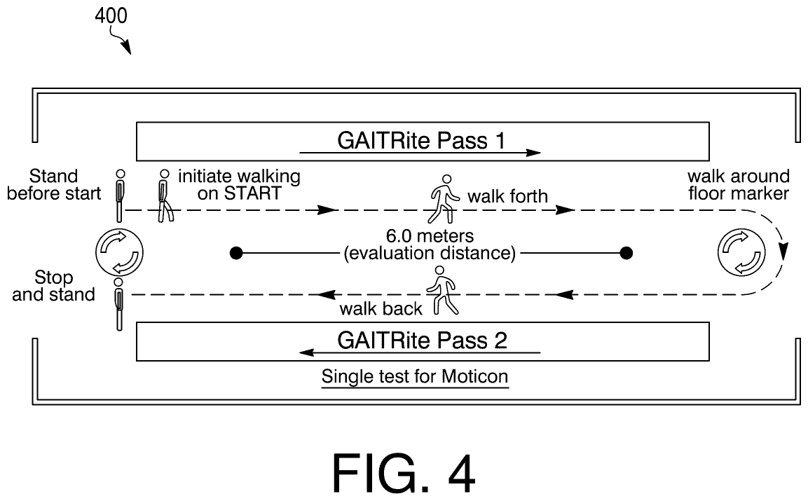

The first test is a gait assessment, where subjects were asked to perform a 10-meter walking task. This task involves starting from a standing position, walking 10 meters at a comfortable speed, turning around, and walking back 10 meters to the starting point. This test may be performed up to two times in both the ON and OFF states of PD treatment. Measurements during this test typically focus on the middle 6 meters of the walk to avoid the acceleration and deceleration phases, providing a more accurate assessment of the subject's gait. The task not only includes straight walking, but also evaluates turning, which is associated with gait quality, functional mobility, and is a potential predictor of disability, falls, and freezing of gait in PD. This 10-meter walking task has established age-specific normative values for several parameters, including walking speed. It has been extensively studied in various neurological diseases and is recommend for gait assessment in PD. In some aspects, during this walking task, gait parameters were simultaneously measured using the wearable insole test device, which may be equipped with one or more of: an accelerometer, gyroscope, and pressure sensors, and the production device, e.g., a walking mat embedded with pressure sensors. This dual measurement allows for a comprehensive analysis of gait characteristics in the OFF state. An exemplary illustration of the process described is provided in diagram 400 in FIG. 4.

Additionally, subjects were also tasked with performing voice and speech assessments using the digital mobile speech-based platform. These speech and language tasks were designed to evaluate different aspects of verbal ability and cognitive function in participants. In an aspect, these tasks were organized to be conducted in a quiet environment to ensure accurate measurement of speech parameters. In a picture description task, participants are shown a static image depicting an event and asked to describe it in their own words. This task serves as a proxy for spontaneous discourse, providing insights into natural language use. In a diadochokinetic (DDK) tasks, participants were required to produce a series of rapid, alternating sounds, such as “puh-tuh-kuh.” These tasks help clinicians assess speech disorders and motor speech difficulties by analyzing the speed and clarity of sound production, which can reveal issues with articulatory agility, speech motor control, or neurological problems affecting speech. In a paragraph reading task, participants were required to read one of three standard paragraphs designed to include all English language phonemes and an equal number of details and information units. This task evaluates reading fluency and pronunciation. In a syllable repetition task, participants were asked to repeat the sounds “pa-ta-ka” as quickly as possible for 15 seconds, assessing their ability to produce rapid sequences of sounds. In a sustained vowel phonation task, participants were required to take a deep breath and sustain the vowel sound/ah/as in ‘bat’ for 15 seconds, measuring their breath control and vocal strength. In a semantic fluency task, participants were asked to name as many different animals or household objects as they could within one minute, which tests their ability to retrieve and produce words within a specific category under time constraints. In a phonemic fluency task, participants were required to name as many unique words as possible that either start with or contain a specific letter (e.g., ‘f’ or ‘s’) within one minute. This task evaluates the participant's ability to access and generate words based on phonemic cues. Collectively, these tasks provide a comprehensive assessment of various speech and language functions, helping clinicians to diagnose and understand speech disorders and cognitive impairments.

In some aspects, the site investigator, who is a movement disorders specialist, was tasked with performing the MDS-UPDRS Part III motor examination. This involved a detailed clinical assessment of the subject's motor function, focusing on various aspects such as rigidity, bradykinesia, and tremors, among others. Specific tasks, including toe tapping and leg agility, were performed using the production device while wearing the wearable insole tests device to enable a quantitative analysis of these movements.

Overall, the OFF state assessments provide a detailed and comprehensive evaluation of the patients' motor and functional abilities without the influence of medication, serving as a critical baseline for subsequent comparisons with the ON state assessments.

At step 310, system 100 may receive data from the subjects that were assessed in the ON-state. In the context of this application, the ON-state may be characterized as a subject state when the subject is under an effect of a treatment such as a dopaminergic medication at a first dosage, a subject state when the subject is under the effect of the treatment such as the dopaminergic medication at a second dosage (e.g., a dosage above a threshold dosage at which drug effects are known to occur in a subject), and/or a subject state when the subject is under the effect of the treatment such as the dopaminergic medication under a threshold duration of time (e.g., where the threshold duration of time is a time at which most or all substantive drug effects are known to have worn off in the subject). More particularly, in an aspect, after completing the activities in the OFF-state, the subjects were administered their conventional dose of dopaminergic medication to transition the subjects from the OFF state to the ON state. In an aspect, following medication administration, there was a waiting period of 30 to 60 minutes to allow the medication to take effect fully. During this time, the subjects were monitored, and subjective reports were utilized to confirm that they transitioned to the ON state.

Once the ON state was confirmed, the same series of assessments conducted in the OFF state were repeated. For instance, subjects were again requested to perform the 10-meter walking task, where they start from a standing position, walk 10 meters at a comfortable speed, turn around, and walk back 10 meters to the starting point. During this task, gait parameters were measured simultaneously using the wearable insoles test device and the production device. The dual measurement approach ensures comprehensive data collection on gait characteristics, allowing for a detailed comparison of gait parameters between the OFF and ON states. Following the gait assessment, subjects again underwent voice and speech assessments using the digital mobile speech-based platform. This involved repeating the same set of voice-related activities performed in the OFF state, including paragraph reading, picture description, object naming, rapid syllable repetition, and sustained phonation. Conducting these tasks in a quiet environment ensured that the speech parameters were accurately measured without external distractions.

In an aspect, the site investigator, a movement disorders specialist, performed the MDS-UPDRS Part III motor examination in the ON state. This detailed clinical assessment focused on the same aspects of motor function evaluated in the OFF state, such as rigidity, bradykinesia, and tremors. Specific tasks, including toe tapping and leg agility, were performed using the production device while wearing the wearable insoles test device, enabling a quantitative analysis of these movements in the ON state.

The comprehensive assessment in the ON state, including gait analysis, voice and speech evaluation, and detailed motor examination, provides important data on the efficacy of dopaminergic medication. By comparing the results from the OFF and ON state assessments, e.g., at step 315, the improvements in motor and functional abilities due to medication and the effectiveness of the wearable insoles test device and the digital mobile speech-based platform in detecting these changes could be evaluated. This comparison is important for understanding the potential of these technologies in monitoring and managing PD symptoms. Specific results of the comparison are described throughout this disclosure.

At step 320, one or more key characteristics may be identified based on the comparison at step 315. To facilitate this process, one or more algorithms or data analytics tools may be leveraged to identify patterns in the collected data. In an aspect, system 100 may focus on one or more key data parameters, including changes in gait dynamics, variations in speech performance, and/or alterations in motor functions as measured by the speech and gait test devices. By comparing these parameters with the clinical MDS-UPDRS scores, system 100 may pinpoint significant correlations and/or deviations that indicate the efficacy of the treatment and the progression of the diseases. In an aspect, the key characteristics may include: an identification of significant improvements or deteriorations in one or more gait characteristics (e.g., gait speed, stride length, balance, etc.) or speech characteristics (e.g., clarity, fluency, phonation, etc.), variations in motor control (e.g., such as increased or decreased rigidity, etc.), correlation patterns between the test device measurements and clinical scores, highlighting specific motor and/or speech symptoms that respond well to treatment.

At step 325, system 100 may utilize the key characteristics identified in step 320 to initiate one or more automated actions that, for example, are aimed at optimizing patient care and treatment strategies for PD. For instance, in one aspect, system 100 may suggest personalized adjustments to a subject's medication regimen. For example, if the comparative analysis reveals significant improvements in gait stability and reduced tremors in the ON state, system 100 may recommend maintaining the current dosage of dopaminergic medication. Conversely, if improvements are minimal, system 100 may suggest increasing the dosage or introducing a complimentary therapy. In another aspect, system 100 may dynamically recommend one or more therapy regiments that are tailored specifically for the subject's condition, as assessed by the workflow outlined above. For instance, for subjects showing specific deficits in motor functions, such as reduced leg agility or toe tapping performance, system 100 may recommend a customized physical therapy regiment that focuses on exercises that enhance lower extremity strength and coordination. This may include balance training, gait exercises, strength conditioning, and the like. As another example, if the speech analysis identifies issues like decreased articulation rate, system 100 may recommend certain targeted speech therapy sessions that are focused on exercises to improve speech aspects.

In another example, based on the subject's specific motor and speech challenges, system 100 may provide personalized lifestyle recommendations. For instance, if the subject has difficulty with balance, system 100 may provide one or more home modifications to prevent falls, such as installing grab bars and/or using non-slip mats. These modifications may be general in nature or, if system 100 has knowledge of the subject's specific home characteristic (e.g., home size, layout details, etc.), may be more granular and tailored to the subject's situation.

In another example, system 100 may automatically generate an updated assessment if the data collected was insufficient to draw definitive conclusions about the subject's motor and speech functions. For instance, if the 10-meter walking task did not capture sufficient data, the system may trigger a 20-meter walking task or a task having additional activities, e.g., stair climbing or obstacle navigation. As another example, if the speech analysis did not provide sufficient information, system 100 may trigger one or more additional tasks that may be employed, e.g., extending reading passages or introducing additional phonemic fluency exercises. In an aspect, the tasks triggered by system 100 may be static or dynamic. For instance, with respect to the former, if system 100 identifies an insufficient data context, it may i) access a database containing one or more data-expanding tasks, and ii) select one or more of the data-expanding tasks to provide to the user. With respect to the latter, system 100 may identify the degree to which the collected data was insufficient and may then dynamically predict the types and numbers of additional activities that a subject could perform in order to meet a sufficiency threshold. In an aspect, the tasks for the revised test may be automatically sent to the user's device and/or another designated device.

In another example, system 100 may automatically generate an updated test if the test results identified key characteristics associated with and/or exhibited by the subject during initial test performance. For instance, during an initial gait assessment, a subject may be found to exhibit reduced stride length and poor balance. In such a circumstance, system 100 may automatically generate an updated test that may include balance-focused activities such as single-leg stands, tandem walking, or dynamic balance tasks. As another example, during an initial speech assessment, a subject may be found to have a reduced articulate rate. In such a circumstance, system 100 may automatically generate an updated test that may include exercises specifically targeting articulation, such as tongue twisters or complex syllable repetition tasks. In an aspect, the updated test may be automatically sent to the user's device and/or another designated device.

In another example, for a subject undergoing treatment such as with a dopaminergic medication, system 100 may analyze the data collected during gait and/or speech assessment and determine that the current treatment dosage is not sufficiently effective in improving the subject's gait and/or speech functions. Accordingly, system 100 may suggest a specific increase in the medication dosage or a change in the type of medication. In an aspect, system 100 may automatically implement the modification in dosage and/or medication to the subject's treatment plan. In another aspect, the proposed adjustment may be sent to a clinician for approval. If the clinician approves, the new dosage and/or medication type is updated in the subject's treatment plan. Once the modification is made, system 100 may monitor the subject's response to the new regimen, ensuring continuous optimization of treatment based on real-time data (e.g., collected via updated or renewed tests).

In an aspect, the continuous nature of the data collection may enable substantially real-time monitoring of the subject's condition. For instance, system 100 may establish alerts to notify healthcare providers if there are significant deviations from expected patterns (e.g., such as a sudden decline in gait performance or speech clarity), prompting timely interventions. Additionally or alternatively, for subjects whose data shows inconsistent or “concerning” patterns, system 100 may dynamically suggest additional diagnostic tests, such as brain imaging or electrophysiological studies, to further investigate the underlying causes and refine the treatment plan. Additionally or alternatively, system 100 may have knowledge of various current or upcoming clinical trials and may dynamically i) determine whether the subject is eligible to participate in any of these trials; and ii) transmit details associated with trial enrollment to the subject if they are eligible.

### Statistical Analysis Overview

### MDS-UPDRS Scores and Gait Characteristics

The statistical analysis for the study involves several detailed methods to represent and interpret the data. Scatter plots may be created to visualize MDS-UPDRS scores as they relate to various clinicodemographic covariates, such as age and levodopa state, as well as different gait characteristics. These scatter plots may aid in understanding the relationships between clinical scores and patient demographics.

In an aspect, the initial stage of analysis may involve computing correlation estimates between each gait characteristic and the MDS-UPDRS scores. These correlations are ranked from 1 to −1 based on the strength and direction of their association with the clinical outcomes. Additionally, differences in means for each gait characteristic may be assessed between the ON and OFF states to identify any significant changes. These analyses may be performed separately for each device used, e.g., the test device and the production device. In an aspect, given that the sample is not a random selection of PD subjects, it is important to control for potential biases by controlling for covariates. Therefore, in an aspect, some or all models may be adjusted for age and biological sex. Further adjustments may be made for treatment state (e.g., whether the patient is in the ON or OFF state) and other relevant clinicodemographic covariates.

In an aspect, a modeling scheme may involve performing multiple linear regression analyses separately for each device. The base model may adjust for age, sex, treatment state, and each gait characteristic independently. In an aspect, let Yᵢ be the MDS-UPDRS clinical outcome for the ith subject. Assume there are K gait characteristics in total, and let Xᵢⱼₖ be the jth observation of the kth gait characteristic for the ith subject. The model is defined as:

$${Y}_{i} = {β}_{0} + {β}_{{1}^{}} {Age}_{i} + {β}_{2} {Sex}_{i} + {β}_{3} {State}_{i} + {β}_{4} {X}_{ijk} + {ε}_{ijk} ,$$

where Ageᵢ and Xᵢⱼₖ are mean centered, Sexᵢ=1 for males and 0 otherwise, Stateᵢ=1 for the ON state and 0 otherwise, and εᵢⱼₖ˜N(0, σ²).

The parameter interpretations are:
β₀: Mean UPDRS score for a female in the OFF state with mean age and Xᵢⱼₖ value.
β₁: Mean difference in UPDRS score between two subjects who are one year apart in age with the same biological sex, levodopa state, and Xᵢⱼₖ value.
β₂: Mean difference in UPDRS score between a male and a female of the same age, in the same levodopa state, and with the same Xᵢⱼₖ value.
β₃: Mean difference in UPDRS score between a subject in the ON and OFF state who share the same age, sex, and Xᵢⱼₖ value.
β₄: Mean difference in UPDRS score between two subjects who are one unit apart in the Xᵢⱼₖ value and share the same, levodopa state, and age.
εᵢⱼ: Error term (also known as the residual) in the model. It represents the difference between the observed values and the values predicted by the model.

In an aspect, the association between Xᵢⱼₖ and Yᵢ is captured by β₄. In an aspect, the base model may assume that this association does not change with levodopa state. The model may further be expanded to allow the association between Xᵢⱼₖ and Yᵢ to differ by levodopa state:

$${Y}_{i} = \text{} {β}_{0} * + {β}_{1} * {Age}_{i} + {β}_{2} * {Sex}_{i} + {β}_{3} * {State}_{i} + {β}_{4} * {X}_{ijk} + {β}_{5} * {State}_{i} * {X}_{ijk} + {ε}_{ijk}$$

This last model allows the association between Xᵢⱼₖ and Yᵢ to differ by treatment (e.g., levodopa) state. The two models above may be compared with a likelihood ratio test for each of the K gait characteristics. Further, the two models above may be compared with a more parsimonious model:

$${Y}_{i} = {β}_{0}^{′} + {β}_{1}^{′} {X}_{ijk} + {ε}_{i}$$

This simpler model may also be nested within the previous model, allowing for comparison using a likelihood ratio test. In an aspect, all three models assume homoscedasticity (constant variance of residuals). However, they do not account for multiple observations per subject. To address this, the models may be expanded by introducing random intercepts for each subject. This approach allows for the estimation of variability in gait characteristics both within and between individuals, improving the robustness and accuracy of the analysis.

In an aspect, the system may be configured to generate a graphical user interface (GUI) that may display a visual representation of sensed gait characteristics. For instance, the visual representation may take the form of a heatmap, scatterplot, boxplot, and the like, which are organized based on the sensed gait characteristics. In an aspect, heatmaps may be used to display gait characteristics (organized as rows) against subject observations (organized as columns). The rows may be ordered by the strength of their association with MDS-UPDRS III scores, while the columns will be arranged by the MDS-UPDRS III scores. Two versions of the heatmap may be created: one where the color hue represents Z-scores computed across all subjects and another where the color hue represents Z-scores computed within individual subjects. These heatmaps may also be annotated with clinicodemographic information for additional context.

Scatter plots may be created that illustrate the estimated association between each gait characteristic and the MDS-UPDRS III score on the x-axis and the LE subscore on the y-axis. This will create a visual representation of data points for both the test and production devices within a [−1, 1]² limit, with four quadrants to aid interpretation.

Bar plots may be created that may show gait characteristics on the x-axis and the difference in association between each gait characteristic with the MDS-UPDRS III score and the LE subscore. These plots may highlight whether gait characteristics capture different information within the UPDRS scores.

Box plots of all gait characteristics and relevant summary statistics, such as mean and median values, may be created. These plots may include paired data connected with lines between the OFF and ON PD states, providing a clear visual representation of changes in gait characteristics due to levodopa administration.

### Gait Summary Metrics (Dimension Reduction Analysis)

According to embodiments of the disclosed subject matter, high-dimensional gait data produced by test and/or production devices may be simplified and/or interpreted using summarized gait metrics. Further, the strength of associations between summarized gait metrics and the MDS-UPDRS score or subcomponents thereof may be determined.

Principal component analysis (PCA) may be employed to simplify and interpret the high-dimensional gait data collected in the study. Let X_{G} represent the data matrix for gait characteristics measured by the production device, where the rows correspond to subject observations and the columns to various gait characteristics. Similarly, X_{M} denotes the data matrix for gait characteristics measured by the wearable insole test device. Here, N, K_{G}, and K_{M} stand for the total number of observations, the total number of gait characteristics measured by the production device, and the total number of gait characteristics measured by the test device, respectively.

For PCA, the first step may involve centering and scaling the data matrices X_{G} and Xv. The centered and scaled data matrices are denoted as X′_{G} and X′_{M}. The PCA process is detailed using singular value decomposition (SVD). Specifically, for X′_{G}, SVD is represented as X′_{G}=U_{G}D_{G}V^{T}_{G}, where U_{G} and V_{G} are orthonormal matrices, and D_{G} is a diagonal matrix containing singular values. Principal components may then be derived using P_{G}=X′_{G}′V_{G}, where the j-th principal component corresponds to the j-th column of P_{G}. The variance explained by the j-th principal component is given by d²ⱼ/(N−1), with dⱼ being the j-th diagonal element of D_{G}.

This entire process is similarly applied to X′_{M}, and the resulting principal components are referred to as summarized gait metrics. The SVD approach to PCA is particularly robust, as it may be applied to any data matrix, regardless of its dimensions. The number of summarized gait metrics for the production device will be equal to min (N, K_{G}), and for the test device, it will be min (N, K_{M}). This PCA process enables the reduction of complex gait data into a set of principal components that capture the most significant variance, facilitating easier interpretation and analysis of the gait characteristics in relation to Parkinson's disease severity and treatment effects.

Bar plots may be created with summarized gait metrics in the x-axis and percentage of variance explained in the y-axis for both test and production devices. Bar plots may be created with summarized gait metrics in the x-axis and cumulative percentage of variance explained in the y-axis for both test and production devices. Scatter plots may be created to visually compare the summarized gait metrics. The data points may be highlighted by different clinicodemographic variables, as needed. Scatter plots may be created between MDS-UPDRS scores and each summarized gait metrics. The data points may be highlighted by different clinicodemographic variables, as needed.

### Gait Summary Metrics (Enrichment Analysis)

According to embodiments of the disclosed subject matter, the overrepresentation of specific groups of gait features (e.g., spatial, temporal, kinematic) in PD OFF and ON states may be determined.

The Normalized Enrichment Score (NES) is an exploratory statistical measure used in the analysis of gait features. The NES reflects the extent to which a set of gait features is overrepresented at the extremes (either top or bottom) of the entire ranked list of gait features. These features are ranked based on their association strength with the PD OFF and ON states, which is derived from the previously discussed models.

Gait characteristics are categorized based on their nature and the specific aspects of gait they represent. For example, spatial gait features might include stride length, step length, and step width. These features provide information about the distance covered in each step or stride and the space between the feet during walking, which can be significantly affected in conditions like PD.

By grouping gait features in this way and using the NES to identify overrepresented groups, researchers may gain a more nuanced understanding of the changes in gait associated with PD. The Enrichment Score(ES) is computed using the Kolmogorov-Smirnov (K-S) statistic, a non-parametric test that compares the cumulative distribution function of the ranked list of gait features to a reference distribution. The ES is determined by identifying the maximum deviation from zero of this cumulative sum, which indicates the degree to which gait features are overrepresented at the extremes of the ranked list.

To derive the NES, the ES is normalized for the size of the set of gait features. The statistical significance of the observed NES is assessed using permutation testing to obtain the null distribution for the K-S statistic. This robust method provides a reliable way to evaluate the statistical significance of the NES, offering insights into which specific gait features or groups of features are most indicative of changes in PD states.

### Adjustment for Multiple Comparisons

In the analysis of gait characteristics, let K_{G} represent the total number of gait characteristics measured by the production device and K_{M} represent those measured by test device. Together, these yield a total of K_{G}+K_{M} association estimates. Since this analysis is exploratory, an a spending procedure is not used to adjust for multiple testing.

Given this exploratory approach, no specific thresholds for the statistical tests were pre-specified for the initial analysis. Instead, the Benjamini-Hochberg procedure was applied to control the false discovery rate (FDR). This method may help manage the proportion of false positives among the significant findings.

Additionally, results may be ranked by effect sizes and the absolute values of the test statistics. For each unadjusted p-value, a q-value is calculated. The q-value indicates the proportion of false positives expected when that particular test is deemed significant, providing a measure of FDR for each test. This approach may allow for a more nuanced understanding of the statistical significance of the findings while acknowledging the exploratory nature and potential for false positives.

### Analysis Conventions

In an aspect, the following analysis conventions may be used in the statistical analysis:
Analysis on all observed data regardless of study dropout use may be performed as supportive analysis (i.e., subject did not complete both timepoints of a visit).
All screening data should be included (including change and % change from baseline).
All baseline data for change and % change should be calculated (e.g., may be 0) rather than left as blank/missing in the tables, where applicable.
For data points above ULQ (e.g., for results reported as >XX), the ULQ may be used (where relevant).
For data points below LLQ (e.g., for results reported as <XX), the ½ LLQ may be used where relevant).

### Gait Platform Concordance

In an aspect, the implementation of early analytical validation by comparing identical derived gait characteristics between the production device, the industry standard, and the test device provides an important opportunity to affirm the accuracy and reliability of the test device in measuring key gait parameters. These parameters include cadence, speed, stride length, time walked, double support time, steps, and swing time, among others. Ensuring the accuracy of these measurements is essential for evaluating motor function and detecting gait abnormalities in patients with Parkinson's disease. Validating the test device against the established production device ensures that this newer technology can produce comparable results, thereby establishing its efficacy as a tool for gait analysis.

Early analytical validation plays a critical role in identifying potential discrepancies and technical issues, which can be addressed promptly before the technology is deployed on a larger scale. By comparing the outcomes from both devices, researchers may assess the degree of alignment and pinpoint any variations that may arise from differences in measurement methods. This comparative approach not only confirms the test device's ability to deliver consistent and reliable data in real-world conditions but also underscores its robustness and clinical utility. Ultimately, validating the test device against the production device ensures that this advanced technology can effectively track and assess motor function in patients with Parkinson's disease, supporting its adoption in clinical settings for ongoing patient monitoring and management.

According to embodiments of the disclosed subject matter, the congruency of gait characteristics as measured by test and/or production devices may be assessed. Differences in congruent gait characteristics when measured with a test versus production devices may be determined. Differences in congruent gait characteristics when measured with test versus production devices, as a function of a treatment state, may be determined. Relevant demographic features may be used as covariates in the analysis or in associated visualizations of the gait data.

In an aspect, for this analysis, the focus is on variables that are measured by the production device and/or the test device. To ensure accurate comparison and consistency, a thorough review of the gait characteristic names is conducted to verify that identical characteristics are mapped between the two devices. For example, terms like “gait speed” and “gait velocity” need to be confirmed as equivalent. Additionally, it is crucial to standardize the units of measurement across both modalities, such as ensuring that speed is consistently measured in meters per second (m/s).

The specific gait characteristics analyzed include cadence (steps per minute), speed (meters per second), stride length (meters), time walked (seconds), double support time (seconds), steps (count), and swing time (seconds). These variables are essential for evaluating gait and motor function, particularly in patients with Parkinson's disease. The standardization process ensures that these characteristics are directly comparable, facilitating accurate and reliable analysis.

To explore the inter-device performance, the data may be summarized using number of patients with data available the following example standard descriptive statistics: mean measured value for each device; median measured value for each device; mean change between test and production deice; mean % change between test and production deice; median change between test and production deice; median % change between test and production deice; Q1, Q3, and IQR of measured values for each device; Q1, Q3, and IQR of change between test and production deice; Q1, Q3, and IQR of % change between test and production deice; SD, SEM and N's for all of the above. For the descriptive statistics above the production device may be treated as the baseline.

In an aspect, to begin the analysis correlation estimates may be computed for each gait characteristic measured by the production device and the test device. These correlation estimates range from 1 to −1 and are ranked accordingly. Additionally, difference in means estimates are used to assess the differences in each gait characteristic between the two devices. This comparative analysis is conducted separately for each treatment (e.g., levodopa) state (e.g., the OFF and ON states). In an aspect, to mitigate potential sources of bias, models may control for covariates such as age and biological sex, with the possibility of including other clinicodemographic covariates. Additionally, all models may adjust for the treatment (e.g., levodopa) state of the subjects.

In an aspect, for analytical validation, a multiple linear regression model may be utilized. This model may adjust for age, sex, levodopa state, and/or the measurement device for each gait characteristic separately. Let Yᵢⱼₖ represent the j-th observation of the k-th gait characteristic for the i-th subject. Suppose there are K′ gait characteristics in common between the devices. The model is structured as follows:

$${Y}_{ijk} = {β}_{0} + {β}_{1} {Age}_{i} + {β}_{2} {Sex}_{i} + {β}_{3} {State}_{ij} + {β}_{4} {Device}_{ij} + {ε}_{ijk} ,$$

where Ageᵢ is mean centered, Sexᵢ=1 for males and 0 otherwise, Stateᵢⱼ=1 for the ON state and 0 otherwise, Deviceᵢⱼ=1 for Moticon and 0 otherwise, and εijk˜(0, σ²).

The parameter interpretations are:
β₀: Mean outcome for a female in the OFF state with mean age when measured using the production device.
β₁: Mean outcome difference between two subjects who are one year apart in age with when the gait characteristic is measured with the same device and the subjects share the same biological sex, and levodopa state.
β₂: Mean outcome difference between a male and a female of the same age and in the same levodopa state when the gait characteristic is measured with the same device.
β₃: Mean outcome difference between a subject in the ON state and another in the OFF state of the same age and in the same levodopa state when the gait characteristic is measured with the same device.
β₄: Mean outcome difference between a subject measured using a test device and another measured using ae production device of the same age and in the same levodopa state when the gait characteristic is measured with the same device.
εᵢⱼ: Error term (also known as the residual) in the model. It represents the difference between the observed values and the values predicted by the model.

The second aim may be addressed by β₄. The model above may assume that the mean difference in gait characteristics between the test and production devices are the same in both levodopa states. To address the third aim we expand the model above with the following:

$${Y}_{ijk} = \text{} {β}_{0} * + {β}_{1} * {Age}_{i} + {β}_{2} * Sex + {β}_{3} * {State}_{i} + {β}_{4} * {Device}_{ij} + {β}_{5} * {State}_{ij} * {Device}_{ij} + {ε}_{ijk}$$

This model allows the mean difference between the test device and the production device to vary based on the levodopa state. A likelihood ratio test is used to compare this model with the simpler model without the interaction term for each of the K′ gait characteristics. In an aspect, the most parsimonious model, which only includes the device variable, is also compared with the two previous models:

$${Y}_{ijk} = {β}_{0}^{′} + {β}_{{1}^{}}^{′} {Device}_{ij} + {ε}_{ijk}$$

This model may be nested within the previous models, allowing for a likelihood ratio test to assess model fit. All three models may assume homoscedasticity and do not initially account for the multiplicity of observations per subject.

To effectively compare and analyze the gait characteristics measured by the test device and the production device, several types of visualizations may be employed.

Scatter plots may be created for each concordant gait characteristic to visualize the association between measurements taken from the test device and the production device. Data points may be highlighted based on clinicodemographic variables, providing insights into how these factors influence the measurements.

Bland-Altman plots may be generated for each concordant gait characteristic to assess the agreement between measurements taken from the test device and the production device. These plots will help identify any systematic biases or limits of agreement between the two devices. Similar to the scatter plots, data points in the Bland-Altman plots may be highlighted by clinicodemographic variables to offer a clearer understanding of potential biases introduced by these factors.

Heatmaps may be created with concordant gait characteristics as rows and subject observations as columns. The rows will be organized by the strength of the association between the gait characteristic and the MDS-UPDRS III score, while the columns may be arranged by the MDS-UPDRS III score. Three versions of the heatmaps will be produced: (i) the first version may use color hue to represent scaled differences in means between the test device and the production device, assuming the same variance; (ii) the second version may map color hue to Z-scores of scaled differences in means computed across all subject observations; and (iii) the third version may map color hue to Z-scores of scaled differences in means computed within subjects. These heatmaps may be annotated with clinicodemographic information, providing a comprehensive visualization of the data.

Scatter plots may be created to show the estimated mean difference between devices for each gait characteristic. One set of plots will display these differences in the OFF state on the x-axis and the ON state on the y-axis, resulting in K′ data points. This visualization will help identify if device agreements vary by levodopa state.

Bar plots may be used to show the mean difference between devices for each gait characteristic on the x-axis. A second set of bar plots will illustrate the difference in differences between the ON and OFF states, providing an intuitive visualization of whether device agreements differ by levodopa state.

Scatter plots may also be created with the MDS-UPDRS III score on the x-axis and the scaled mean difference on the y-axis for each gait characteristic. Additionally, a comprehensive scatter plot will combine all concordant gait characteristics, with data smoothed using a LOESS curve. This approach will help assess if device agreement changes as a function of UPDRS scores, offering deeper insights into the relationship between clinical severity and device measurements.

### Adjustment for Multiple Comparisons

Let K′ be the total number of concordant gait characteristics-measured both by the test device and the production device, then we may have K′ association estimates in total. In some aspects, due to the exploratory nature of the analysis, no threshold for the statistical tests may be pre-specified for the first pass analysis. Instead, the Benjamini-Hochberg procedure may be applied to control the false discovery rate (FDR). Results may also be sorted by the effect sizes and absolute value of the test statistic. Q-values may be calculated for each unadjusted p-value. The q-value of a test measures the proportion of false positives incurred (FDR) when that test is called significant.

### Analysis Conventions

In an aspect, the following analysis conventions may be used in the statistical analysis:
Analysis on all observed data regardless of study dropout use may be performed as supportive analysis (i.e., subject did not complete both timepoints of a visit).
All screening data should be included (including change and % change from baseline).
All baseline data for change and % change should be calculated (e.g., may be 0) rather than left as blank/missing in the tables, where applicable.
For data points above ULQ (e.g., for results reported as >XX), the ULQ may be used (where relevant).
For data points below LLQ (e.g., for results reported as <XX), the ½ LLQ may be used where relevant).

### Vocal Profiling

### BACKGROUND

Disease monitoring in neuroscience is hampered by insensitive clinical scales which may not detect subtle changes in symptoms. By extracting hundreds of features of speech and language, Winterlight Labs' technology may measure fine-grained changes that traditional assessments cannot. Winterlight features and algorithms may be used in health research to help identify disease, track change over time, monitor disease severity, and detect response to treatment. Winterlight's technology has been used to study neurodegenerative and psychiatric diseases and disorders, including Alzheimer's Disease, Mild Cognitive Impairment, Frontotemporal Dementia, Parkinson's Disease, and others.

The Winterlight platform, developed by Winterlight Labs, is a tool that utilizes machine learning and natural language processing techniques to generate >760 individual variables that detail the speech and language patterns observed in any given speech recording. The platform relies on a pipeline that performs data processing and feature extraction using various Python-based acoustic and language processing libraries such as spaCy, Stanford parser, and Praat/Parselmouth, as well as custom code. This extensive catalog of variables supports the analysis of a wide array of speech characteristics, ranging from vocal pitch and tempo to more complex language features such as syntax and semantic content. spaCy is an open-source Python library for Natural Language Processing (NLP). It is widely recognized for its comprehensive approach to processing and understanding text. By performing tokenization, part-of-speech tagging, named entity recognition, dependency parsing, sentence recognition, and word-to-vector transformations, it provides a foundation for further analysis. Within the Winterlight platform, spaCy supports the linguistic analysis of the speech data, helping extract and identify the necessary textual and linguistic features from the spoken input.

The Stanford Parser offers grammar-based dependency parsing, which can be essential for understanding syntactic relationships within speech data. Neural network classifiers are trained to make parsing decisions within a transition-based dependency parser. This results in a fast, compact classifier that uses only a few hundred learned dense features, yielding significant improvements in parsing accuracy and speed. The parser's capabilities ensure that specific linguistic patterns in the analyzed speech data are captured. Praat, and its Python library Parselmouth, are used to analyze and manipulate speech at the acoustic level. Praat allows for detailed analysis of phonetic aspects of speech, including pitch, loudness, and formant frequencies, among others. It can also produce spectrograms and perform voice breaks analysis, thereby providing a rich set of acoustic features. Within the Winterlight platform, Praat and Parselmouth serve to convert audio clips into meaningful acoustic features. Through their combined use, the platform can capture a broad and detailed view of both the acoustic and linguistic characteristics of speech, providing a robust basis for the platform's subsequent machine learning and data analysis processes.

Table C below depicts a task assessment schedule for this study

TABLE C

| | |
|---|---|
| Visit | Visit 1 |

| | | |
|---|---|---|
| Assessment | OFF State | ON State |
| Study Day | 0 | 0 |
| Study Window | 0 | 0 |
| Paragraph Reading | Paragraph 1 | Paragraph 1 |
| Picture Description | Family in Kitchen | Family in Kitchen |
| Picture Description | Living Room | Romantic Dinner |
| Syllable Repetition | pataka | pataka |
| Sustained Phonation | a | a |
| Semantic Fluency | Animals | Household |
| Phonemic Fluency | F | S |

Subjects were also tasked with performing voice and speech assessments using the Winterlight application platform. These speech and language tasks were designed to evaluate different aspects of verbal ability and cognitive function in participants. In an aspect, these tasks were organized to be conducted in a quiet environment to ensure accurate measurement of speech parameters. In a picture description task, participants are shown a static image depicting an event and asked to describe it in their own words. This task serves as a proxy for spontaneous discourse, providing insights into natural language use. In a diadochokinetic (DDK) tasks, participants were required to produce a series of rapid, alternating sounds, such as “puh-tuh-kuh.” These tasks help clinicians assess speech disorders and motor speech difficulties by analyzing the speed and clarity of sound production, which can reveal issues with articulatory agility, speech motor control, or neurological problems affecting speech. In a paragraph reading task, participants were required to read one of three standard paragraphs designed to include all English language phonemes and an equal number of details and information units. This task evaluates reading fluency and pronunciation. In a syllable repetition task, participants were asked to repeat the sounds “pa-ta-ka” as quickly as possible for 15 seconds, assessing their ability to produce rapid sequences of sounds. In a sustained vowel phonation task, participants were required to take a deep breath and sustain the vowel sound/ah/as in ‘bat’ for 15 seconds, measuring their breath control and vocal strength. In a semantic fluency task, participants were asked to name as many different animals or household objects as they could within one minute, which tests their ability to retrieve and produce words within a specific category under time constraints. In a phonemic fluency task, participants were required to name as many unique words as possible that either start with or contain a specific letter (e.g., ‘f’ or ‘s’) within one minute. This task evaluates the participant's ability to access and generate words based on phonemic cues. Collectively, these tasks provide a comprehensive assessment of various speech and language functions, helping clinicians to diagnose and understand speech disorders and cognitive impairments.

### Specific Aims, Variables, and Descriptions of Data

All relevant demographic features may be used as covariates in the analysis or in associated visualizations of the speech data. The clinical variables include PD disease severity variables (e.g., ON and OFF state) and MDS-UPDRS scores and the vocal biomarkers include the vocal biomarker variables collected by the digital mobile speech-based platform.

Standard descriptive statistics may include, for example:
Mean measured value for each Parkinson's state.
Median measured value for each Parkinson's state.
Mean change between the ON and OFF state.
Mean % change between the ON and OFF state.
Median change between the ON and OFF state
Q1, Q3, and IQR of measured values for each Parkinson's state.
Q1, Q3, and IQR of change between the ON and OFF state.
Q1, Q3, and IQR of % change between the ON and OFF state.
SD, SEM and N's for all of the above.
SD, SEM and N's for all of the above.

### MDS-UPDRS Scores and Speech Characteristics

In an aspect, the initial stage of analysis may involve computing correlation estimates between each gait characteristic and the MDS-UPDRS scores. These correlations are ranked from 1 to −1 based on the strength and direction of their association with the clinical outcomes. Additionally, differences in means for each gait characteristic may be assessed between the ON and OFF states to identify any significant changes. These analyses may control for covariates and be performed separately for each task described herein. In an aspect, given that the sample is not a random selection of PD subjects, it is important to control for potential biases. Therefore, in an aspect, some or all models may be adjusted for age and biological sex. Further adjustments may be made for a treatment (e.g., levodopa) state (e.g., whether the patient is in the ON or OFF state) and other relevant clinicodemographic covariates.

In an aspect, a modeling scheme may involve performing multiple linear regression analyses separately for each task and/or device. The base model may adjust for age, sex, levodopa state, and each gait characteristic independently. In an aspect, let Yᵢ be the MDS-UPDRS clinical outcome for the ith subject. Assume there are K speech characteristics in total, and let Xᵢₖ be the value of the kth speech characteristic for the ith subject. The model may be defined as:

$${Y}_{i} = {β}_{0} + {β}_{{1}^{}} {Age}_{i} + {β}_{2} {Sex}_{i} + {β}_{3} {State}_{i} + {β}_{4} {X}_{ik} + {ε}_{ik}$$

where Ageᵢ and Xᵢₖ are mean centered, Sexᵢ=1 for males and 0 otherwise, Stateᵢ=1 for the ON state and 0 otherwise, and εᵢₖ˜N(0, σ²).

The parameter interpretations are:
β₀: Mean UPDRS score for a female in the OFF state with mean age and Xᵢₖ value.
β₁: Mean difference in UPDRS score between two subjects who are one year apart in age with the same biological sex, levodopa state, and Xᵢₖ value.
β₂: Mean difference in UPDRS score between a male and a female of the same age, in the same levodopa state, and with the same Xᵢₖ value.
β₃: Mean difference in UPDRS score between a subject in the ON and OFF state who share the same age, sex, and Xᵢₖ value.
β₄: Mean difference in UPDRS score between two subjects who are one unit apart in the Xᵢₖ value and share the same, levodopa state, and age.
εᵢ: Error term (also known as the residual) in the model. It represents the difference between the observed values and the values predicted by the model.

In an aspect, the association between Xᵢₖ and Yi does not change with levodopa state. The model may further be expanded with the following:

$${Y}_{i} = \text{} {β}_{0} * + {β}_{1} * {Age}_{i} + {β}_{2} * {Sex}_{i} + {β}_{3} * {State}_{i} + {β}_{4} * {X}_{i j k} + {β}_{5} * State * {X}_{k} + {ε}_{ik}$$

This last model allows the association between Xᵢₖ and Yᵢ to differ by levodopa state. The two models above may be compared with a likelihood ratio test for each of the K speech characteristics. Further, the two models above may be compared with a more parsimonious model:

$$Y = {β}_{0}^{′} + {β}_{1}^{′} {X}_{i k} + {ε}_{i}$$

This simpler model may also be nested within the previous model, allowing for comparison using a likelihood ratio test. In an aspect, all three models assume homoscedasticity (constant variance of residuals). However, they do not account for multiple observations per subject. To address this, the models may be expanded by introducing random intercepts for each subject. This approach allows for the estimation of variability in speech characteristics both within and between individuals, improving the robustness and accuracy of the analysis.

Scatter plots may be created to visualize the MDS-UPDRS scores as a function of different clinicodemographic covariates (e.g., age and levodopa state) and speech characteristics, as needed. Heatmaps may be used to display speech characteristics (organized as rows) against subject observations (organized as columns). The rows may be ordered by the strength of their association with MDS-UPDRS III scores, while the columns may be arranged by the MDS-UPDRS III scores. Two versions of the heatmap may be created: one where the color hue represents Z-scores computed across all subjects and another where the color hue represents Z-scores computed within individual subjects. These heatmaps may also be annotated with clinicodemographic information for additional context.

Scatter plots may be created that illustrate the estimated association between each speech characteristic and the MDS-UPDRS III score on the x-axis and the estimated association between each speech characteristic and the LE subscore on the y-axis. This may create a visual representation of data points for both the test and production devices within a [−1, 1]² limit, with four quadrants to aid interpretation.

Bar plots may be created that may show speech characteristics on the x-axis and the difference in association between each speech characteristics with the MDS-UPDRS III score and the LE subscore. These plots may highlight whether speech characteristics capture different information within the UPDRS scores.

Box plots of all speech characteristics and relevant summary statistics, such as mean and median values, may be created. These plots may include paired data connected with lines between the OFF and ON PD states, providing a clear visual representation of changes in speech characteristics due to levodopa administration.

### Speech Summary Metrics (Dimension Reduction Analysis)

According to embodiments of the disclosed subject matter, high-dimensional speech data produced by a mobile speech application in summarized speech metrics may be simplified and interpreted. The strength of associations between summarized speech metrics and the MDS-UPDRS score or subcomponents thereof may be determined. The overrepresentation of specific groups of speech features (e.g., articulatory, phonatory, prosodic) in PD OFF and ON states may be determined.

Principal component analysis (PCA) may be employed to simplify and interpret the high-dimensional speech data collected in the study. Let X represent the data matrix for speech characteristics as measured by the digital mobile speech-based platform, where the rows correspond to subject observations and the columns to various speech characteristics for a specific task. Matrices for other tasks may be similarly denoted. Furthermore, an aspect may denote N and K as the total number of observations and the total number of speech characteristics, respectively. For PCA, the data matrix may first be centered and scaled. Let X′ be the center and scaled data matrix. Simple derivation steps for X′ may also be applied to data matrices from other tasks. Via singular value decomposition (SVD), it is shown that X′=UDV^{T}, where U and V are orthonormal matrices, and D is a diagonal matrix of singular values. Then, principal components may be obtained with P=X′V′. That is, the jth principal component corresponds to the jth column of P. The variance explained by the jth principal component has the form: d²ⱼ/(N−1), where dⱼ is the jth diagonal element of D. This exact process may be replicated for data matrices from other tasks. The principal components may be referred to as summarized speech metrics. The SVD approach to PCA is robust to the dimension of the data matrix since an SVD exists for any data matrix.

Bar plots may be created with summarized speech metrics in the x-axis and percentage of variance explained in the y-axis for the digital mobile speech-based platform. Bar plots may be created with summarized speech metrics in the x-axis and cumulative percentage of variance explained in the y-axis for the digital mobile speech-based platform. Scatter plots may be created to visually compare the summarized speech metrics. The data points may be highlighted by different clinicodemographic variables, as needed. Scatter plots may be created between MDS-UPDRS scores and each summarized speech metric. The data points may be highlighted by different clinicodemographic variables, as needed.

### Speech Summary Metrics (Enrichment Analysis)

According to embodiments of the disclosed subject matter, the overrepresentation of specific groups of speech characteristics in PD OFF and ON states may be determined.

The Normalized Enrichment Score (NES) is an exploratory statistical measure used in the analysis of speech features. The NES reflects the extent to which a set of speech features is overrepresented at the extremes (either top or bottom) of the entire ranked list of speech features. These features may be ranked based on their association strength with the OFF and ON states, which is derived from the previously discussed models.

Speech characteristics may be categorized based on their nature and the specific aspects of speech they represent. For instance, temporal speech features from the digital mobile speech-based platform may include speech rate, pause duration, and utterance length. These features provide information about the speed of speech, the length of pauses between words, and the length of spoken phrases, which can be particularly affected in conditions like PD. By categorizing speech features in this way and using the digital mobile speech-based platform to identify overrepresented groups, a more nuanced understanding of the changes in speech associated with PD may be evident.

The Enrichment Score(ES) may be computed using the Kolmogorov-Smirnov (K-S) statistic, a non-parametric test that compares the cumulative distribution function of the ranked list of speech features to a reference distribution. The ES is determined by identifying the maximum deviation from zero of this cumulative sum, which indicates the degree to which speech features are overrepresented at the extremes of the ranked list.

To derive the NES, the ES may be normalized for the size of the set of speech features. The statistical significance of the observed NES is assessed using permutation testing to obtain the null distribution for the K-S statistic. This robust method provides a reliable way to evaluate the statistical significance of the NES, offering insights into which specific speech features or groups of features are most indicative of changes in PD states.

### Adjustment for Multiple Comparisons

In the analysis of speech characteristics, let K represent the total number of speech characteristics measured by the digital mobile speech-based platform. Due to the exploratory nature of the approach, no specific thresholds for the statistical tests were pre-specified for the initial analysis. Instead, the Benjamini-Hochberg procedure was applied to control the false discovery rate (FDR). This method may help manage the proportion of false positives among the significant findings. Additionally, results may be ranked by effect sizes and the absolute values of the test statistics. For each unadjusted p-value, a q-value is calculated. The q-value indicates the proportion of false positives expected when that particular test is deemed significant, providing a measure of FDR for each test. This approach may allow for a more nuanced understanding of the statistical significance of the findings while acknowledging the exploratory nature and potential for false positives.

### Analysis Conventions

In an aspect, the following analysis conventions may be used in the statistical analysis:
Analysis on all observed data regardless of study dropout use may be performed as supportive analysis (i.e., subject did not complete both timepoints of a visit).
All screening data should be included (including change and % change from baseline).
All baseline data for change and % change should be calculated (e.g., may be 0) rather than left as blank/missing in the tables, where applicable.
For data points above ULQ (e.g., for results reported as >XX), the ULQ may be used (where relevant).
For data points below LLQ (e.g., for results reported as <XX), the ½ LLQ may be used where relevant).

### Findings

### Findings for Clinical Validation for Gait and Speech

In the clinical validation of gait, 30 gait characteristics were measured using the wearable insoles test device and 42 using the production device. Of the test device characteristics, five (corresponding to 17%) showed a nominally statistically significant association with disease severity. These included gait speed, stride length, walk time, maximum force, and mean force, with the latter two being pressure-based characteristics. Thirteen (corresponding to 43%) of the test device characteristics demonstrated nominally statistically significant mean differences between the ON and OFF states. For the production device, nine (corresponding to 21%) gait characteristics were nominally statistically significantly associated with disease severity as measured by the MDS-UPDRS III. These characteristics included gait speed, stride length, stride velocity, step length, walk time, and several distributional characteristics like double support time standard deviation, single support time standard deviation, swing time standard deviation, and step time standard deviation. Additionally, 29 (corresponding to 69%) of the production device characteristics showed nominally statistically significant mean differences between the ON and OFF states. Notably, mean force was the only gait characteristic that was not sensitive to symptomatic treatment but was nominally associated with disease severity.

In the clinical validation of speech, over 2,000 speech characteristics were measured using the digital mobile speech-based platform. Of these characteristics, 166 (corresponding to 8%) were found to have a nominally statistically significant association with disease severity as measured by the MDS-UPDRS III. Additionally, 449 (corresponding to 20%) of the speech characteristics exhibited nominally statistically significant mean differences between the ON and OFF states. Furthermore, 134 (corresponding to 6%) of the speech characteristics were nominally statistically significantly associated with the MDS-UPDRS III but did not show substantial mean differences between the ON and OFF states. These findings highlight the potential of specific speech characteristics to reflect changes in disease severity and treatment effects in patients with Parkinson's disease.

### Findings for Analytical Validation for Gait

In the gait analytical validation study, six congruent gait characteristics were identified between the GAITRite and Moticon systems: gait cadence, gait speed, stride length, stance time, swing time, and double support time. The mean differences between the measurements from both devices were minimal and not significantly different from zero for all characteristics in both the OFF and ON states. For instance, the mean difference in gait cadence was 0.10 steps per minute in the OFF state and −0.69 steps per minute in the ON state, both with confidence intervals indicating no significant difference. Similarly, gait speed and stride length showed negligible differences, with zero mean differences and confidence intervals that included zero in both states. The mean difference finding data is provided below:
Gait cadence (steps per minute): 0.10 [95% CI: −0.88, 1.09] in the Off state and −0.69 [95% CI: −1.67, 0.30] in the On state.
Gait speed (meters/second): 0 [95% CI: −0.03, 0.04] in the Off state and −0.01 [95% CI: −0.05, 0.02] in the On state.
Stride length (meters): 0 [95% CI: −0.03, 0.03] in the Off state and −0.01 [95% CI: −0.04, 0.02] in the On state.
Stance time (percent): 0 [95% CI: −0.01, 0.01] in the Off state and 0 [95% CI: −0.01, 0.01] in the On state.
Swing time (percent): 0 [95% CI: −0.01, 0.01] in the Off state and 0 [95% CI: −0.01, 0.01] in the On state.
Double support time (percent): 0 [95% CI: −0.01, 0.01] in the Off state and 0 [95% CI: −0.01, 0.01] in the On state.

The reliability between the two devices was found to be moderate to excellent for most gait characteristics. Gait cadence exhibited very high reliability with intraclass correlation coefficients (ICCs) of 0.98 in the OFF state and 0.97 in the ON state. Gait speed and stride length also demonstrated high reliability, with ICCs of 0.98 and 0.99 in the OFF state, respectively, and slightly lower but still strong reliability in the ON state. Stance time, swing time, and double support time showed moderate reliability, with ICCs ranging from 0.56 to 0.65 in the OFF state and 0.48 to 0.64 in the ON state. The reliability data is provided below:
Gait cadence (steps per minute): 0.98 [95% CI: 0.96, 0.99] in the Off state and 0.97 [95% CI: 0.93, 0.99] in the On state.
Gait speed (meters/second): 0.98 [95% CI: 0.94, 0.99] in the Off state and 0.93 [95% CI: 0.84, 0.97] in the On state.
Stride length (meters): 0.99 [95% CI: 0.98, 1.00] in the Off state and 0.94 [95% CI: 0.85, 0.98] in the On state.
Stance time (percent): 0.56 [95% CI: 0.16, 0.80] in the Off state and 0.64 [95% CI: 0.29, 0.84] in the On state.
Swing time (percent): 0.57 [95% CI: 0.17, 0.80] in the Off state and 0.65 [95% CI: 0.29, 0.84] in the On state.
Double support time (percent): 0.58 [95% CI: 0.18, 0.81] in the Off state and 0.48 [95% CI: 0.06, 0.76] in the On state.

Ultimately, these findings indicate that the test device wearable insoles provide comparable measurements to the production device, supporting their use for reliable gait analysis for at least a subset of gait characteristics in PD patients.

The findings and embodiments disclosed herein, may be used to determine which body function (e.g., gait or speech) characteristics associate with an applicable score (e.g., MDS-UPDRS III score), which body function characteristics have substantial changes between an ON and OFF treatment state, and/or which body function characteristics are measured equivalently between test and production devices.

### Exploratory Data Analysis

### Clinicodemographic Information

Ultimately, after accounting for the inclusion and exclusion criteria, 22 subjects were chosen to participate in the study. The clinicodemographic information for the subjects involved in the study is provided in Table 1, at page 1 of Appendix A of U.S. Provisional Patent Application No. 63/695,789, which is incorporated by reference herein.

Table 2, from page 2 of Appendix A of U.S. Provisional Patent Application No. 63/695,789, which is incorporated by reference herein, further summarizes the clinicodemographic information across all subjects and by recruitment site. The demographic information is substantially balanced across the two testing sites.

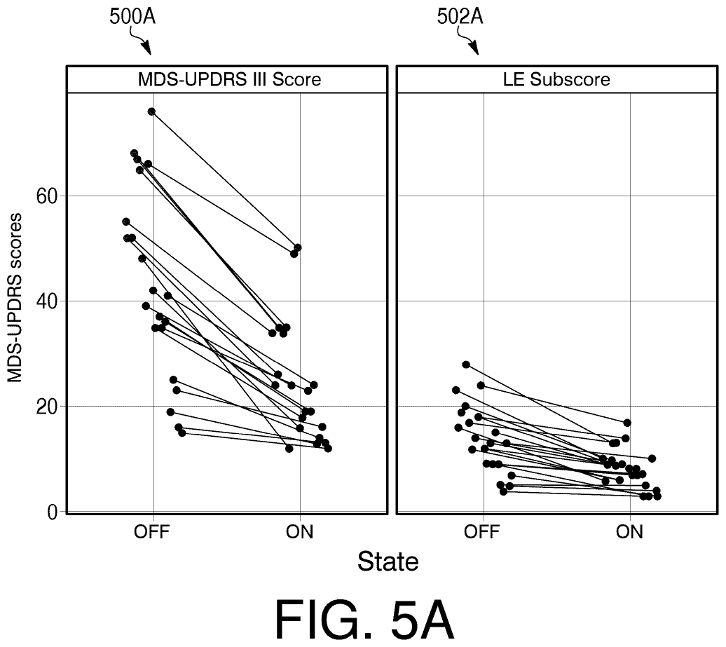

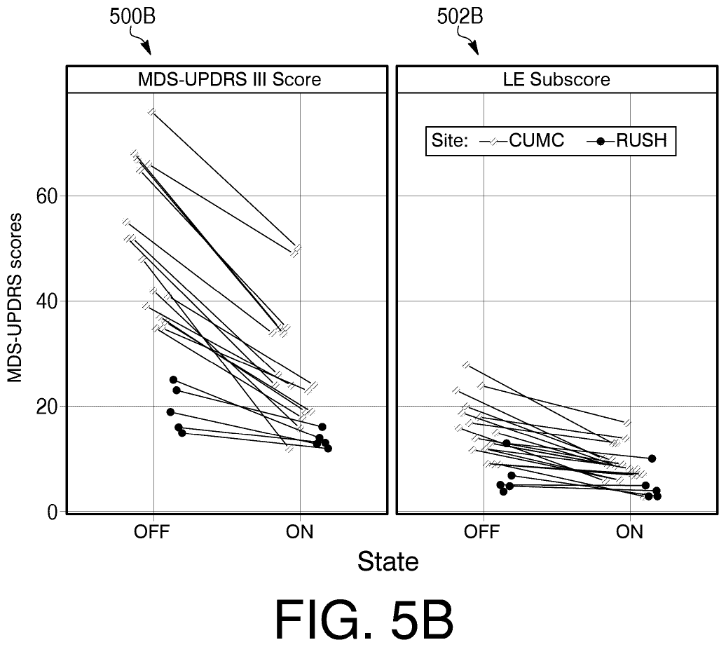

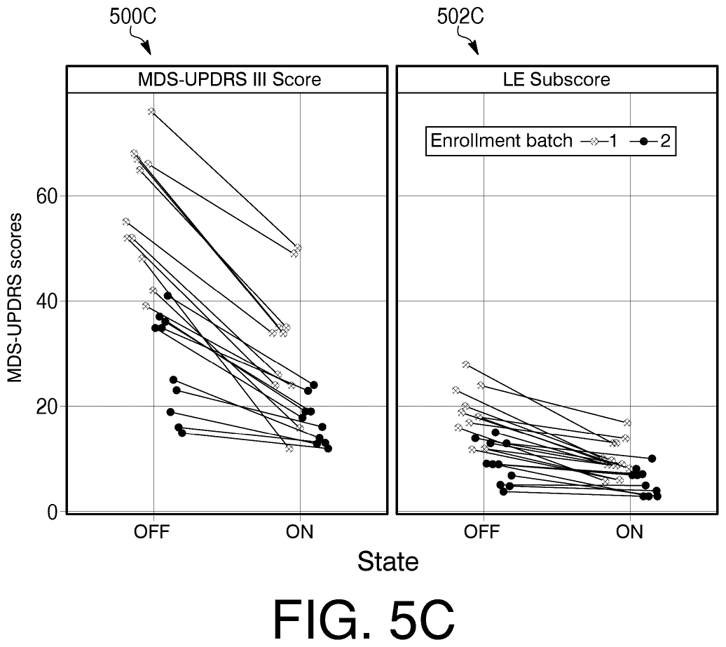

FIGS. 5A-5C provide graphs 500A-500C that collectively illustrate the change in MDS-UPDRS III scores per subject overall (e.g., in FIG. 5A), by site (e.g., in FIG. 5B), and by enrollment batch (e.g., in FIG. 5C). Each graph provides an indication of subject MDS-UPDRS III scores (500A, 500B, and 500C) and subject lower extremity (LE) subscores (502A, 502B, and 502C) before and after levodopa administration (e.g., when the subject is in the OFF state and again when they are in the ON state). In an aspect, MDS-UPDRS III refers to the third part of the MDS-UPDRS, which is focused on motor examination. The LE subscore may correspond to an aggregation of the scores from items specifically related to the lower extremities to give a focused measure of how PD affects this part of the body.

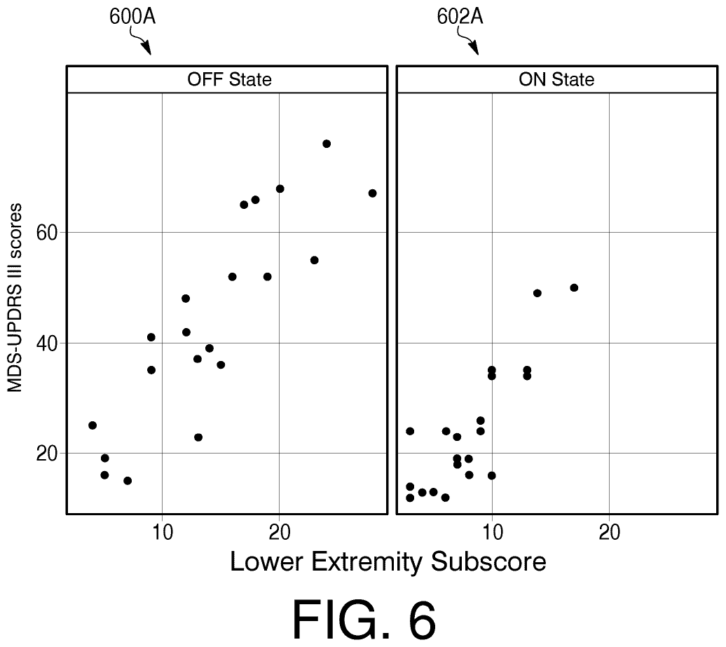

FIG. 6 provides graph 600 that illustrates the correlation between the MDS-UPDRS III score (plotted on the y-axis) and the LE subscore (plotted on the x-axis). In general, a strong correlation was found between these two score types in both OFF-state subjects and ON-state subjects. Specifically, the overall correlation was determined to be 0.87, the correlation in the OFF-state was determined to be 0.88, and the correlation in the ON-state was determined to be 0.78.

### Gait Characteristics

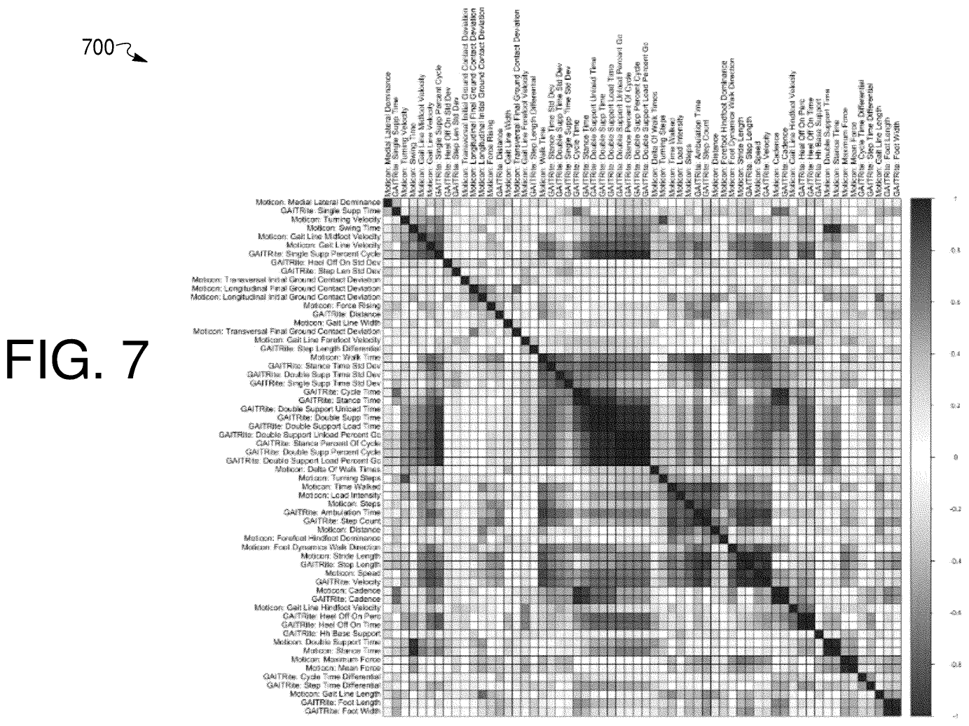

FIG. 7 provides correlation heat map 700 of all gait features from the production device and the wearable insole test device. The rows and columns are ordered using hierarchical clustering to better assess characteristics that are similar to each other. Upon examination, a subset of clusters provide interesting insights. For example, the test device “Walk Time” characteristic is positively correlated with several measured characteristics of the production device. Furthermore, important clinical characteristics like gait speed and gait cadence are clustered together even when measured by the different devices.

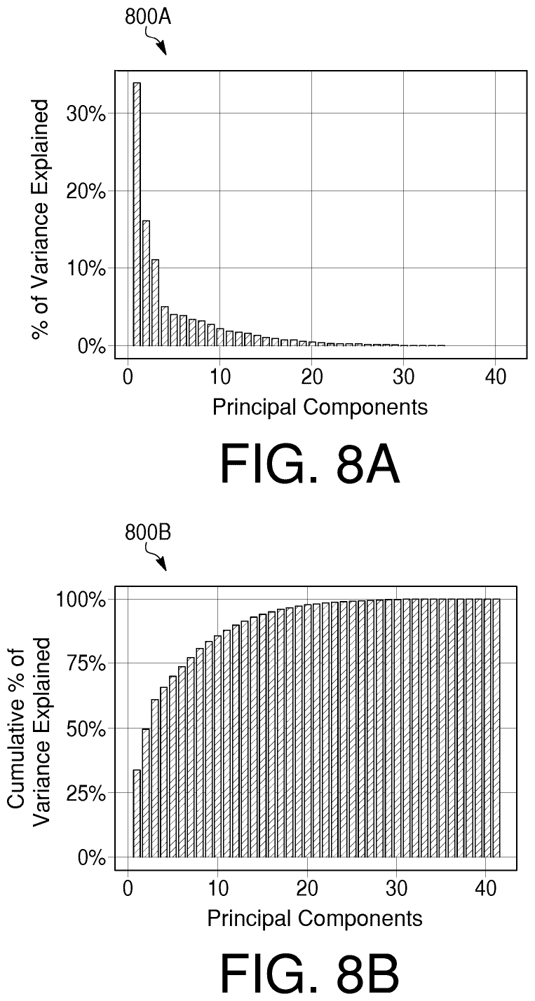

The amount of variation exhibited in correlation heat map 700 of FIG. 700 may be further investigated by performing principal component analysis (PCA). More particularly, the scree plots 800A and 800B illustrated in FIGS. 8A and 8B, respectively, present the percentage of variance and the cumulative percentage of variance, respectively, of the gait characteristics between the production device and the test device. Upon examination, the elbow in each scree plot appears to occur at the third principal component, where about 61% of the variation in the data is explained. By the eighth principal component, around 80% of the variation in the data is explained.

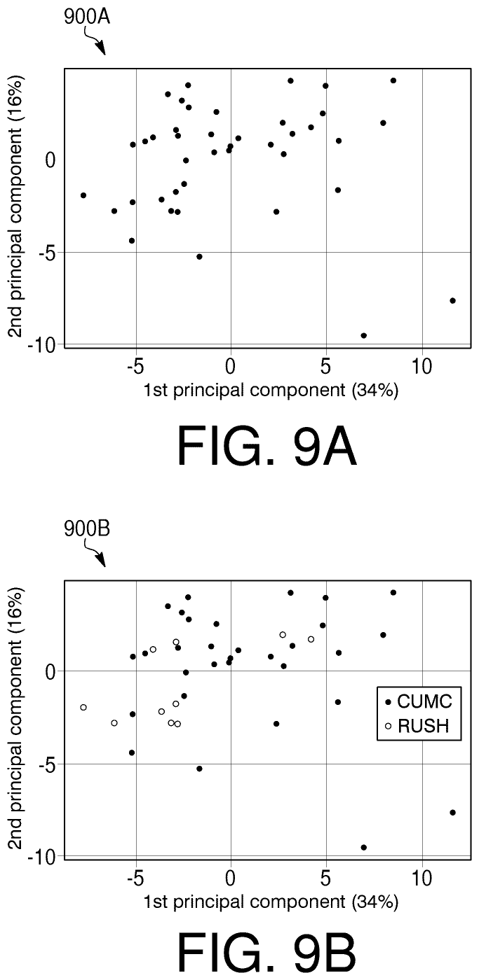

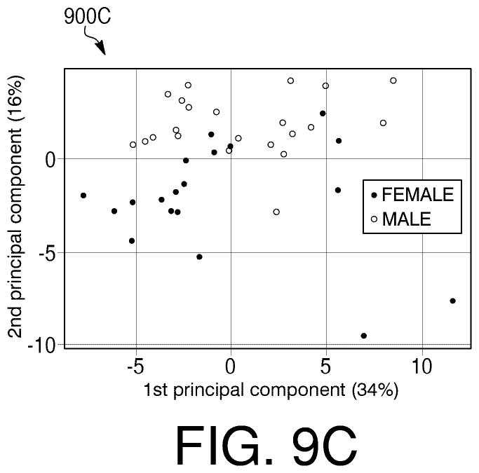

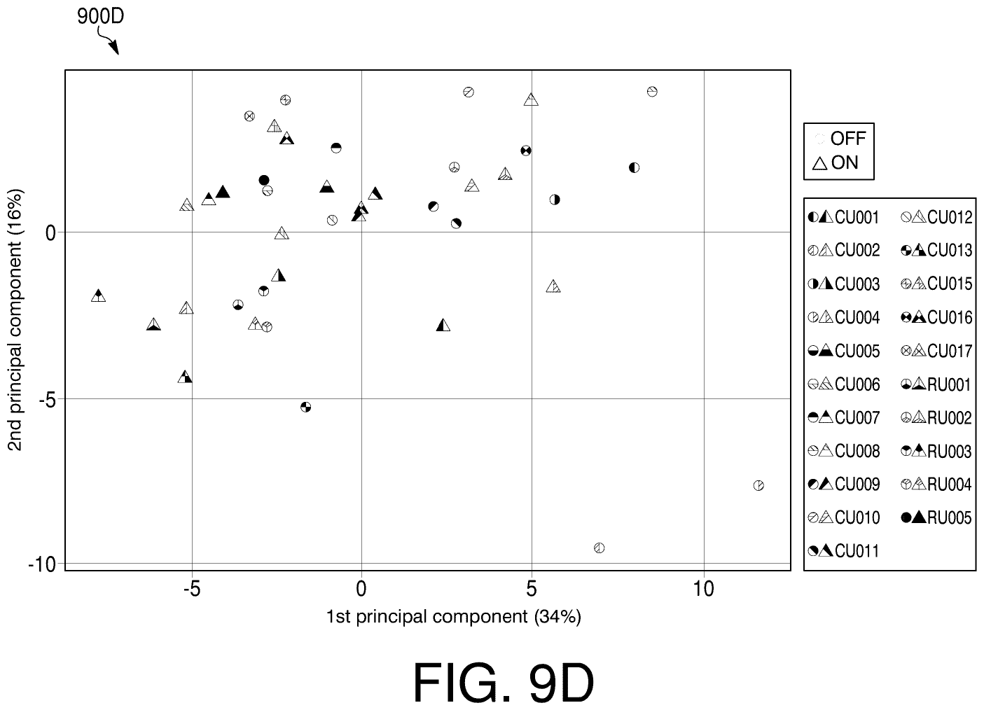

FIGS. 9A-9D provide scatter plots 900A-900D arranged by overall data (e.g., in FIG. 9A), by site (e.g., in FIG. 9B), by sex (e.g., in FIG. 9C), and by subject (e.g., in FIG. 9D), respectively, in which the first principal component (PC1) is plotted against the second principal component (PC2). The clustering and separation along PC1 suggests that the primary variability captured by PC1 is related to the individual response to Levodopa. For example, turning to FIG. 9D, in which PC1 is plotted against PC2 by individual subjects, the different shapes (e.g., circles and triangles) represent different conditions or states for each subject, (e.g., pre- and post-medication or treatment). For most subjects, the pattern where circles are to the right of triangles suggests a shift or change in the measured variables post-levodopa treatment that is captured by PC1. However, subjects CU010, RU002, and CU017 do not show this pattern, indicating a different or more complex response to levodopa that is not as easily captured by PC1. PC2 appears to be associated with biological sex, which highlights an important demographic factor that may influence the data. This may be important for ensuring that analyses and interpretations account for sex different, which also may affect treatment outcomes or other variables of interest.

### Speech Characteristics

As previously alluded to above, the digital mobile speech-based platform features are not uniformly relevant across all tasks. Given the variation in feature relevance, the analysis was conducted separately for each task. This allows for a more precise understanding of which features are relevant for each specific type of speech task, which may help in tailoring speech analysis tools and improving the accuracy of speech assessments.

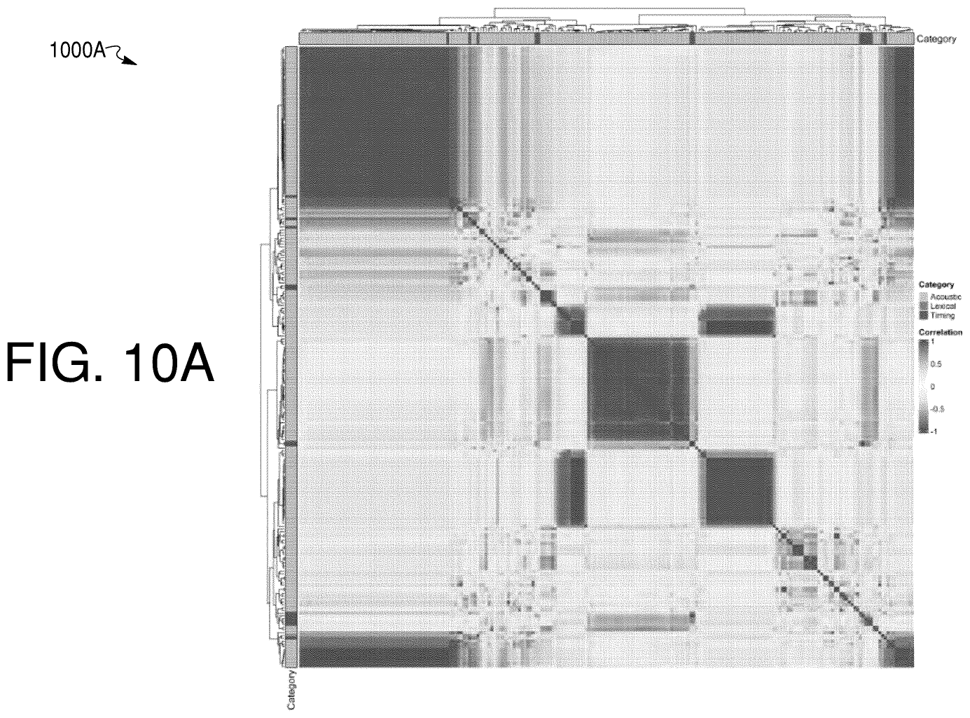

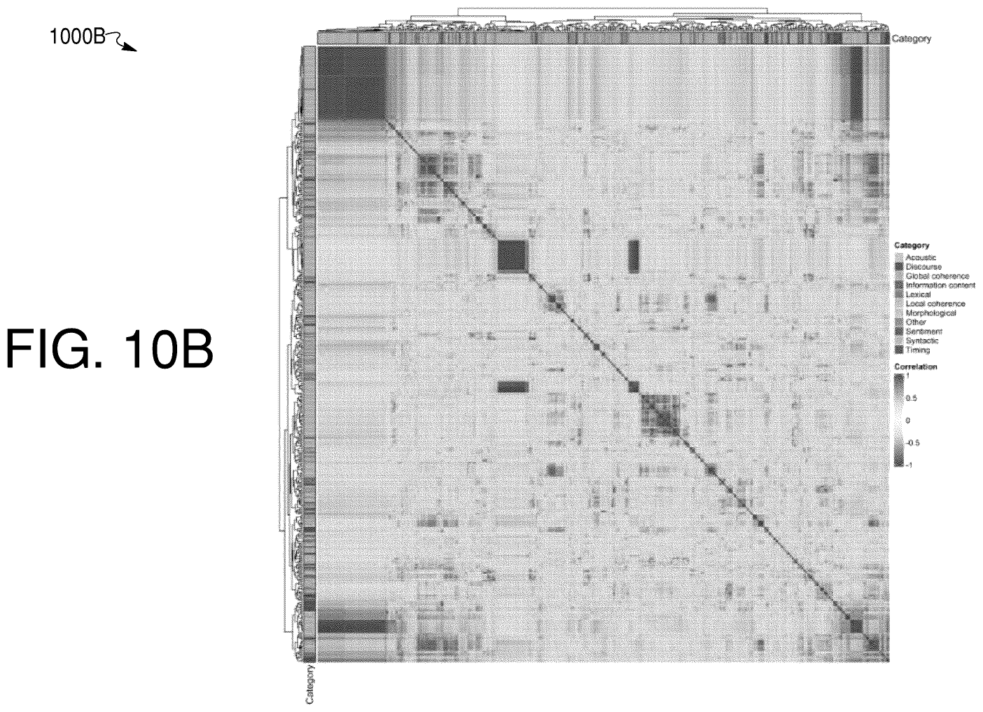

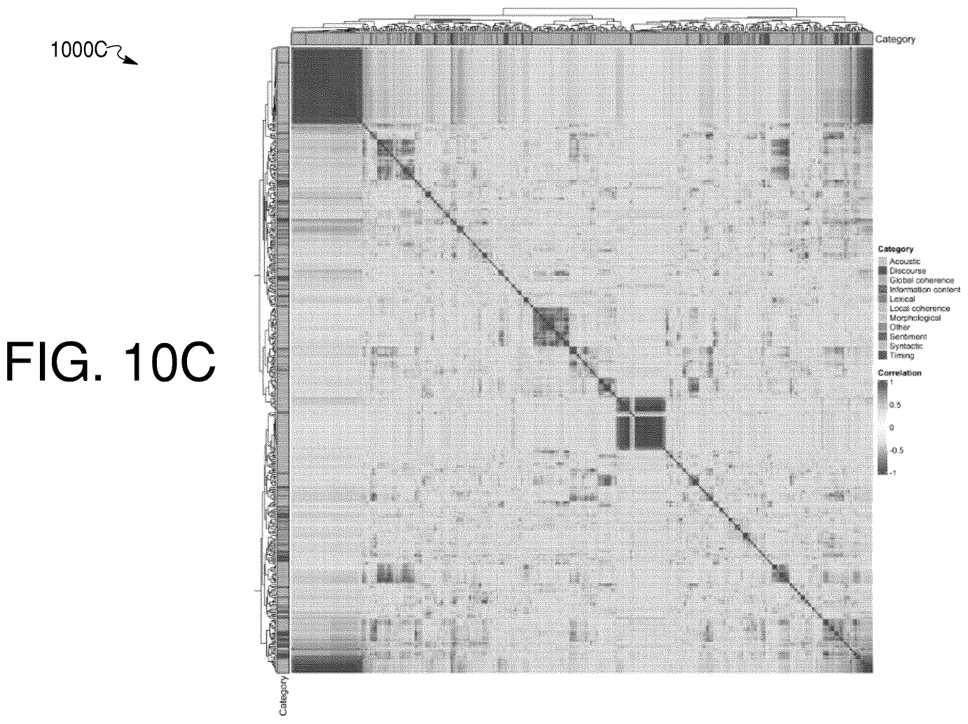

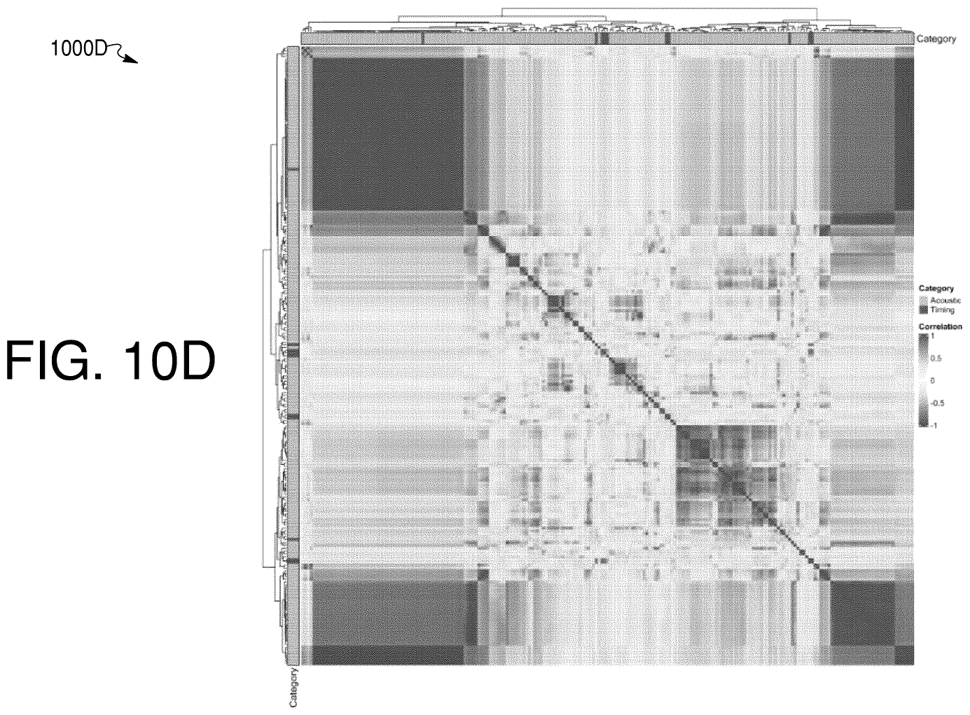

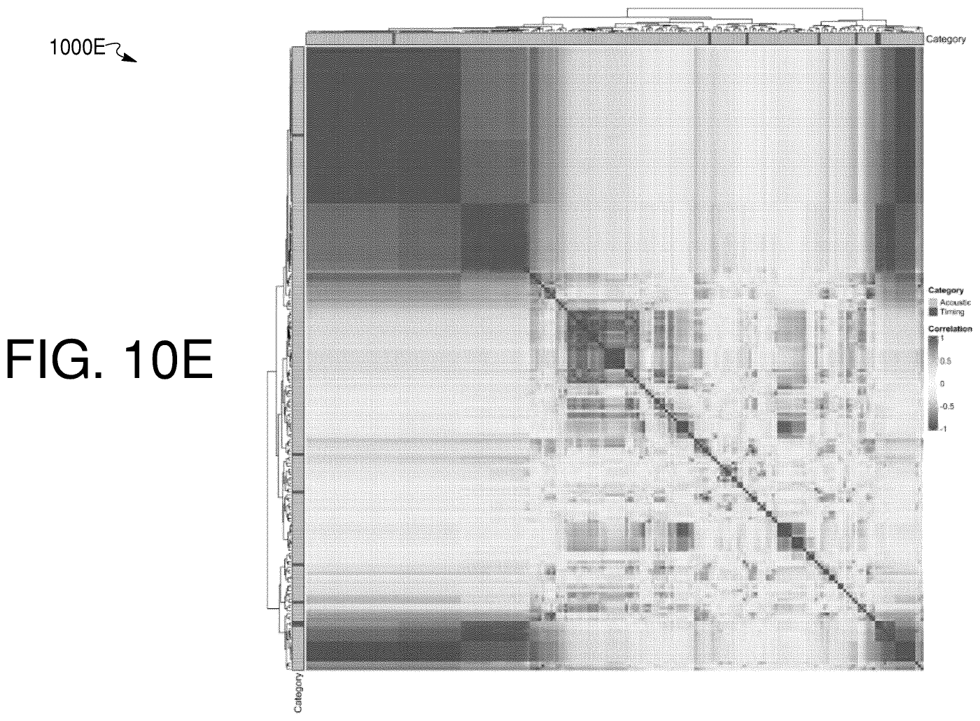

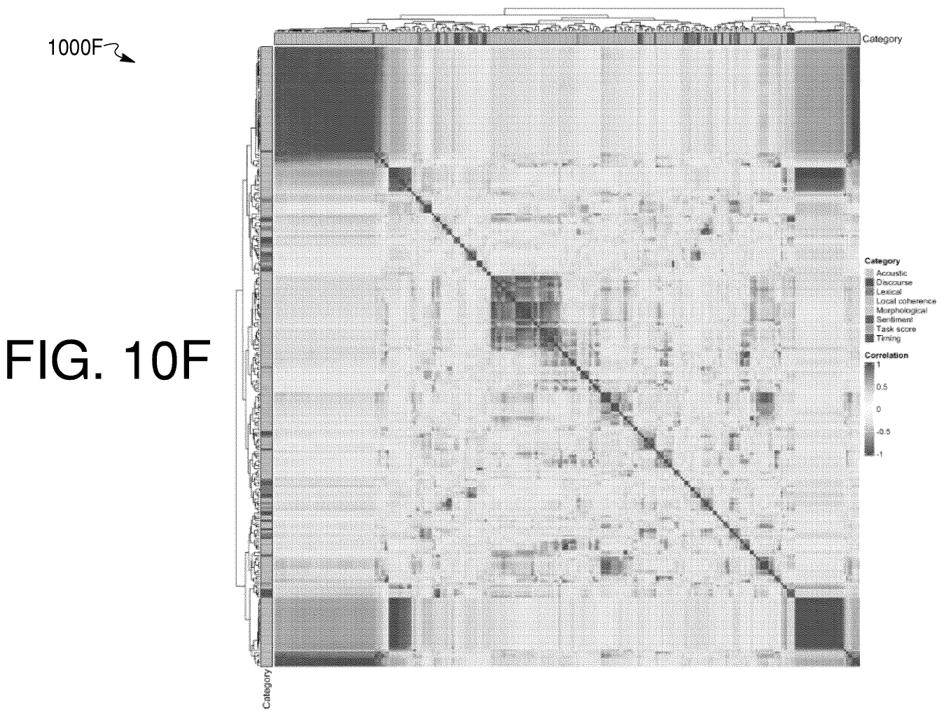

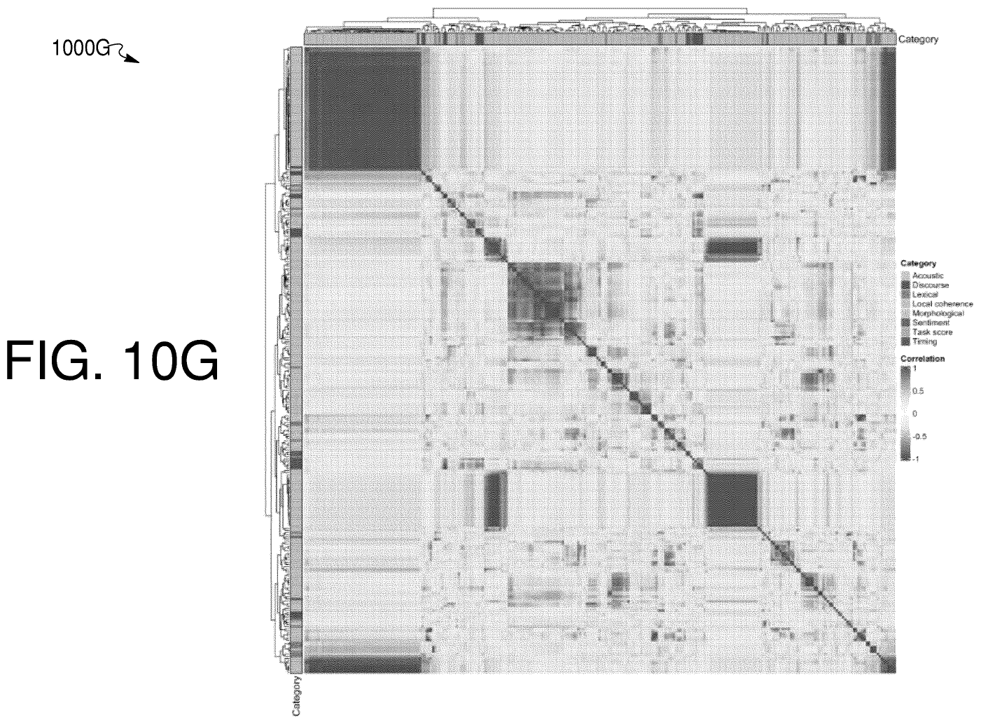

FIGS. 10A-10G provide a series of correlation heat maps 1000A-1000G of speech characteristics across tasks assessed using the digital mobile speech-based platform (e.g., paragraph reading in FIG. 10A, picture description 1 in FIG. 10B, picture description 2 in FIG. 10C, diadochokinetic rate (DDK) in FIG. 10D, sustained vowel phonation in FIG. 10E, semantic fluency in FIG. 10F, and phonemic fluency in FIG. 10G). These plots visualize the correlations between different features within each task. Most of the features across all tasks are acoustic in nature. Acoustic features include aspects like pitch, volume, and speech rate. Upon examination, the heat maps show large clusters of highly correlated features, indicating that many acoustic features tend to vary together. Furthermore, for tasks that involve describing a paragraph, the correlation heat maps show similarities both in the magnitude of correlations and in the overall matrix structure. This implies that the relationships between features in these tasks are consistent. In addition to acoustic features, some clusters in the heat maps are composed of syntactic features, which relate to the structure of the spoken language, such as grammar and sentence construction. The presence of these syntactic feature clusters highlight the importance of considering linguistic aspects of speech, not just acoustic ones.

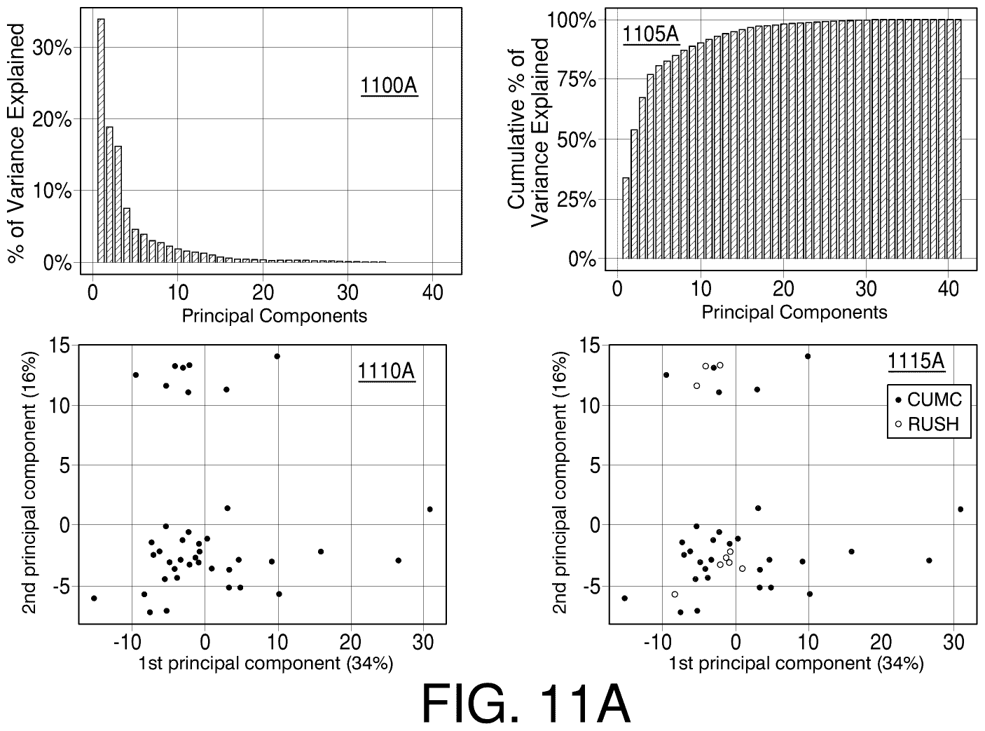

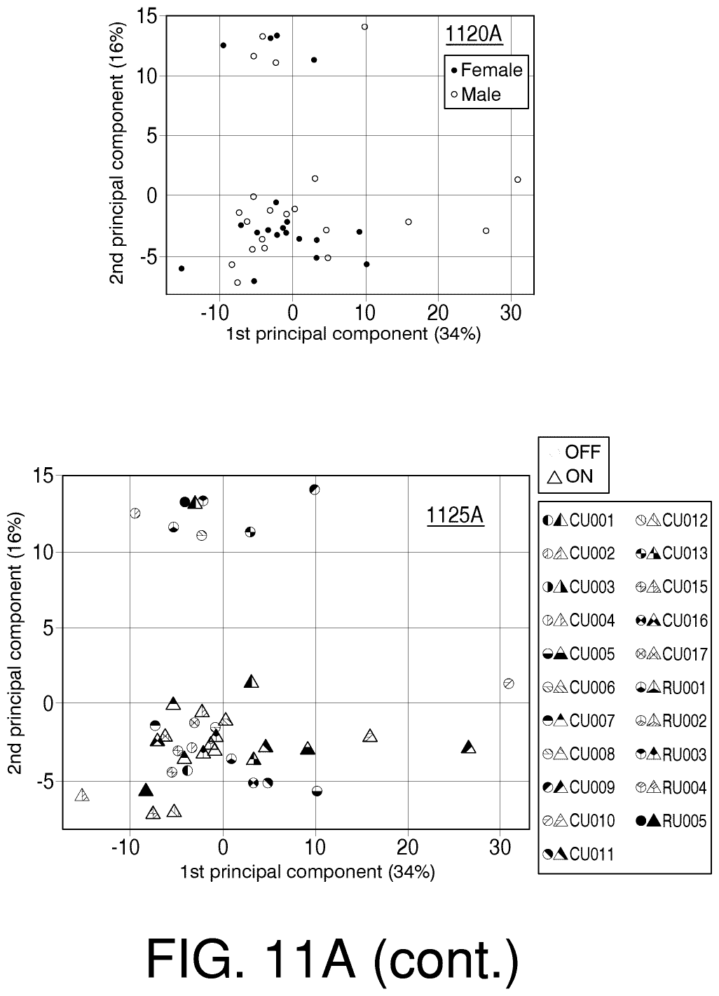

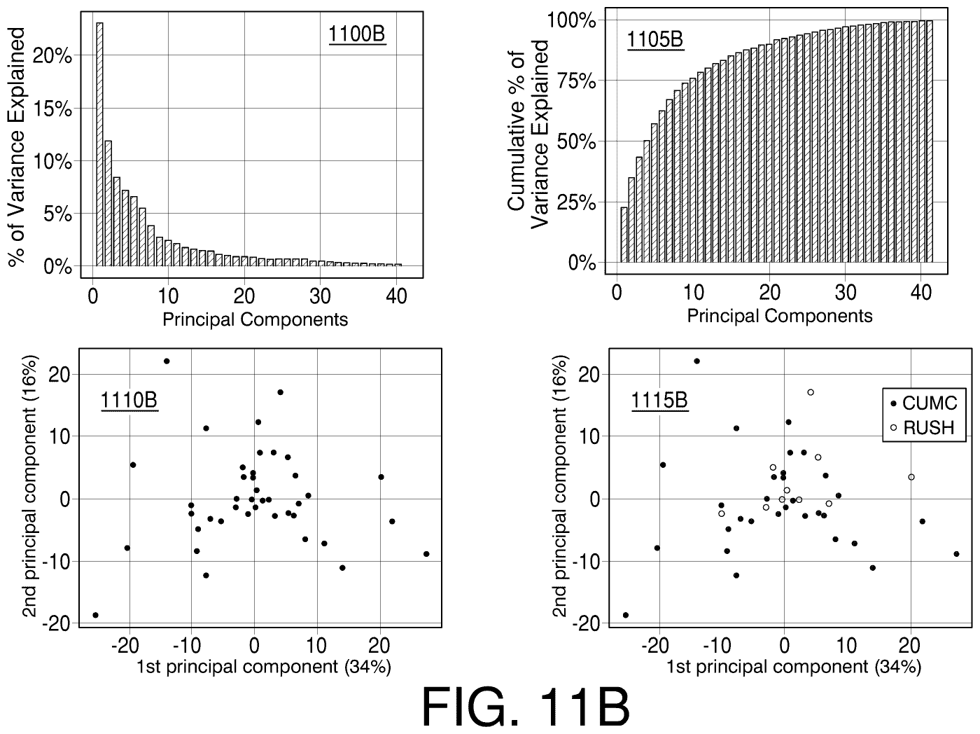

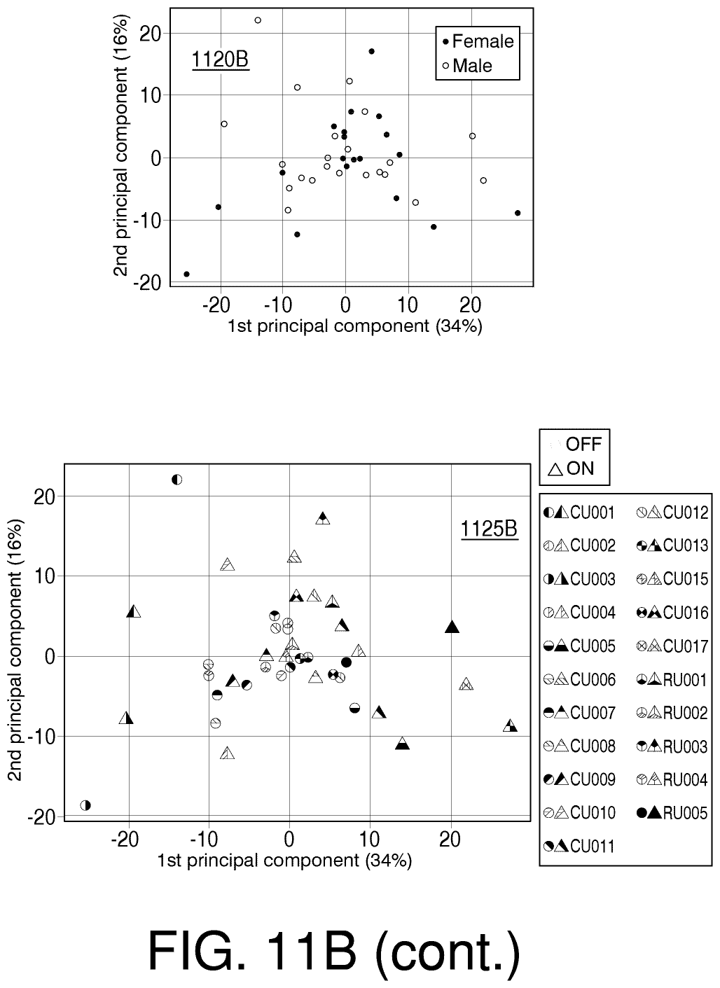

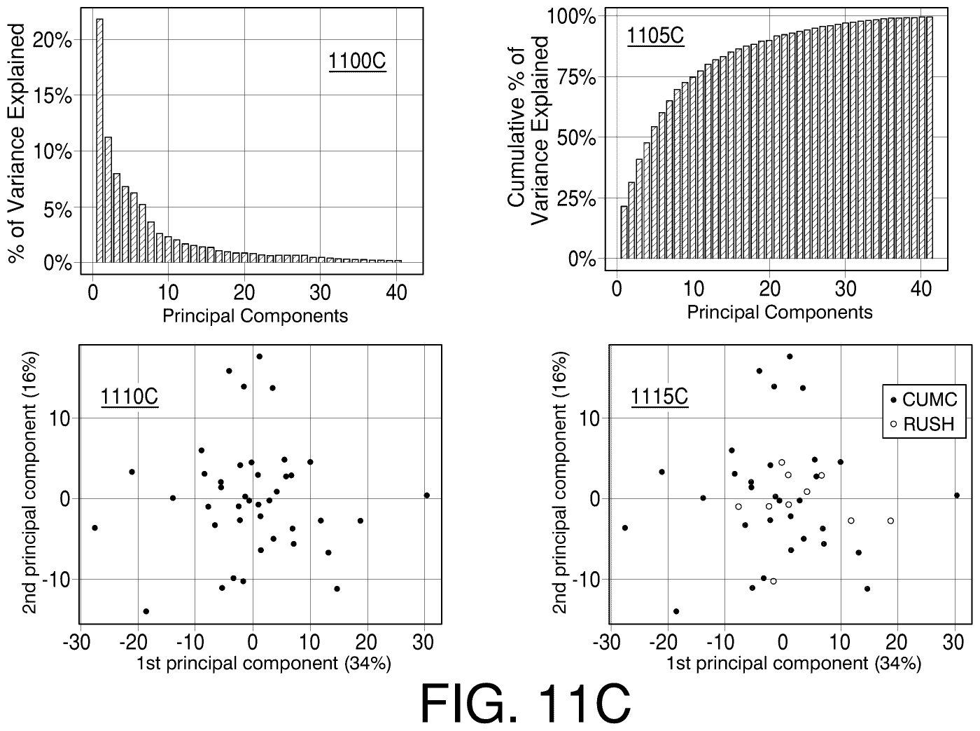

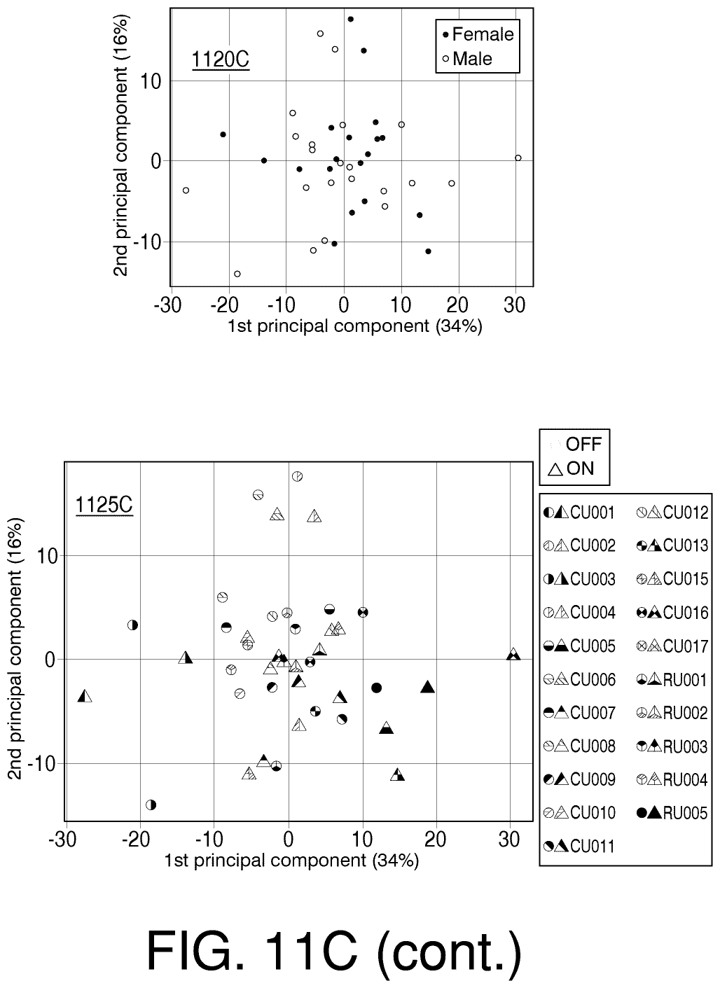

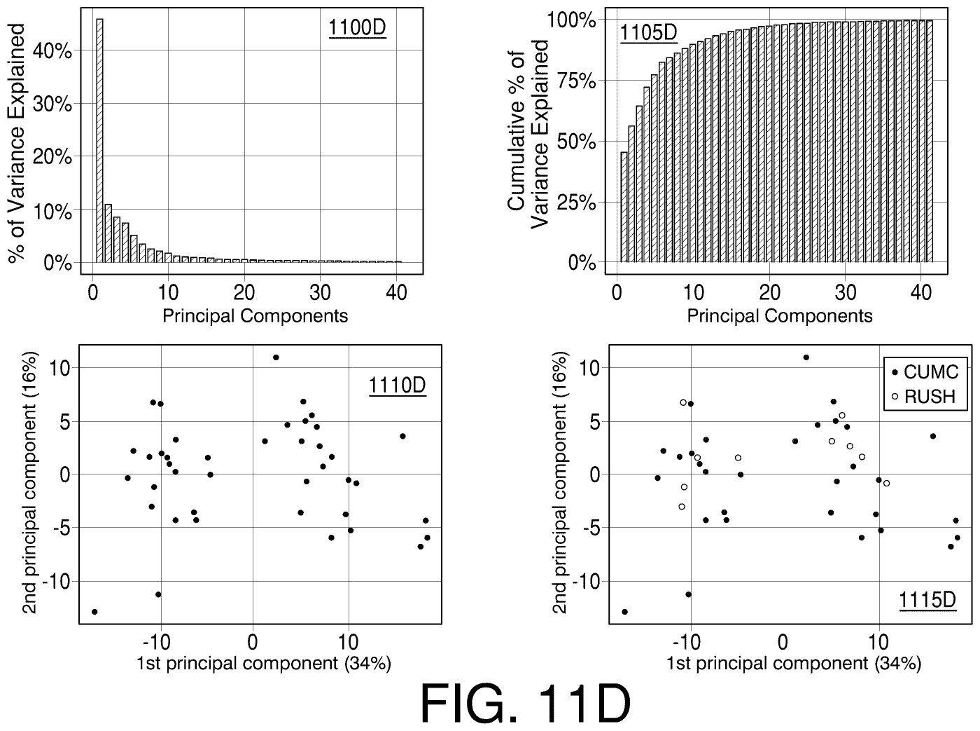

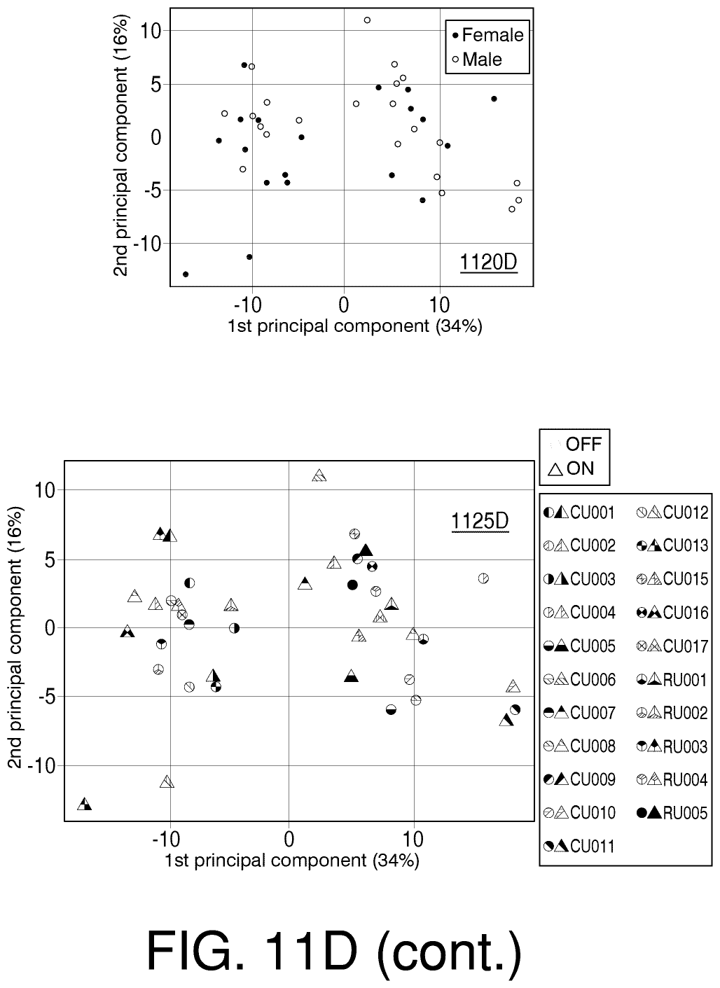

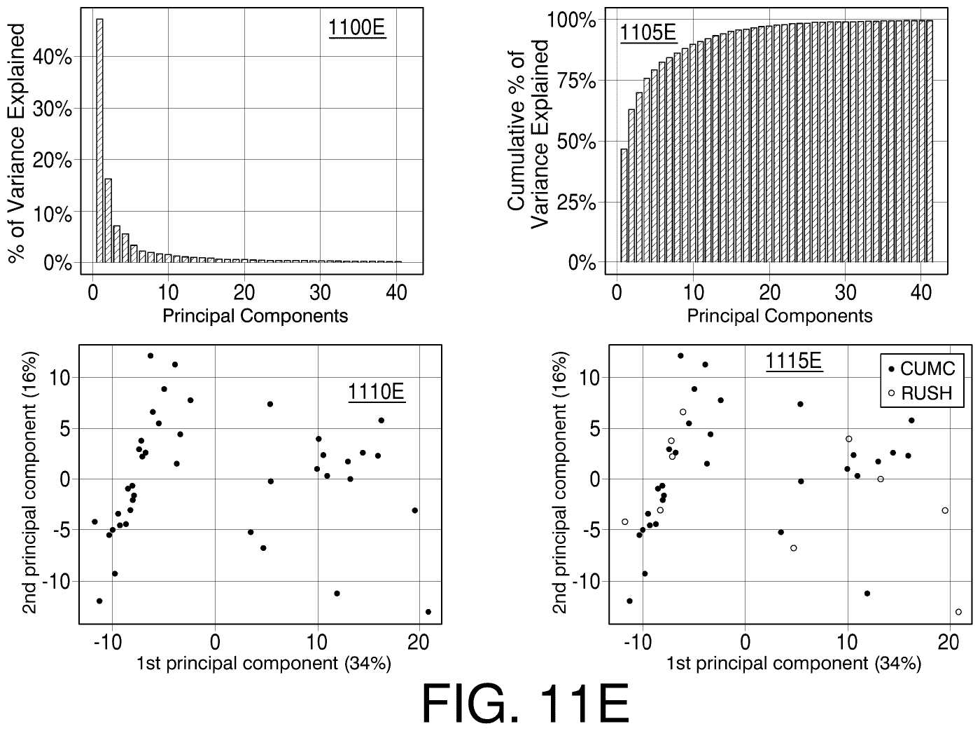

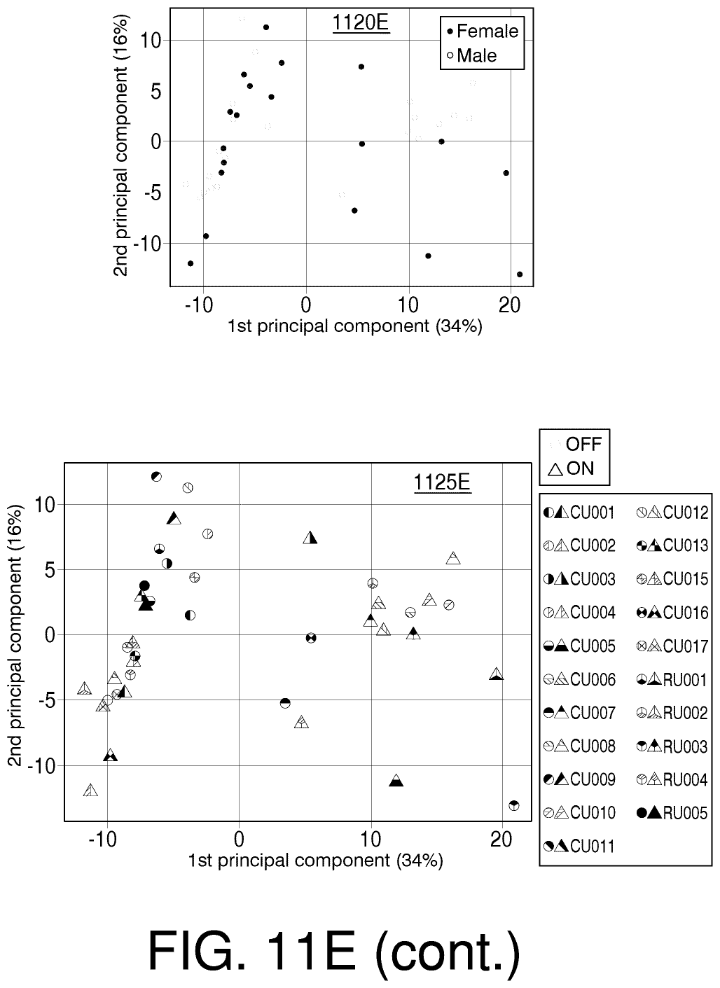

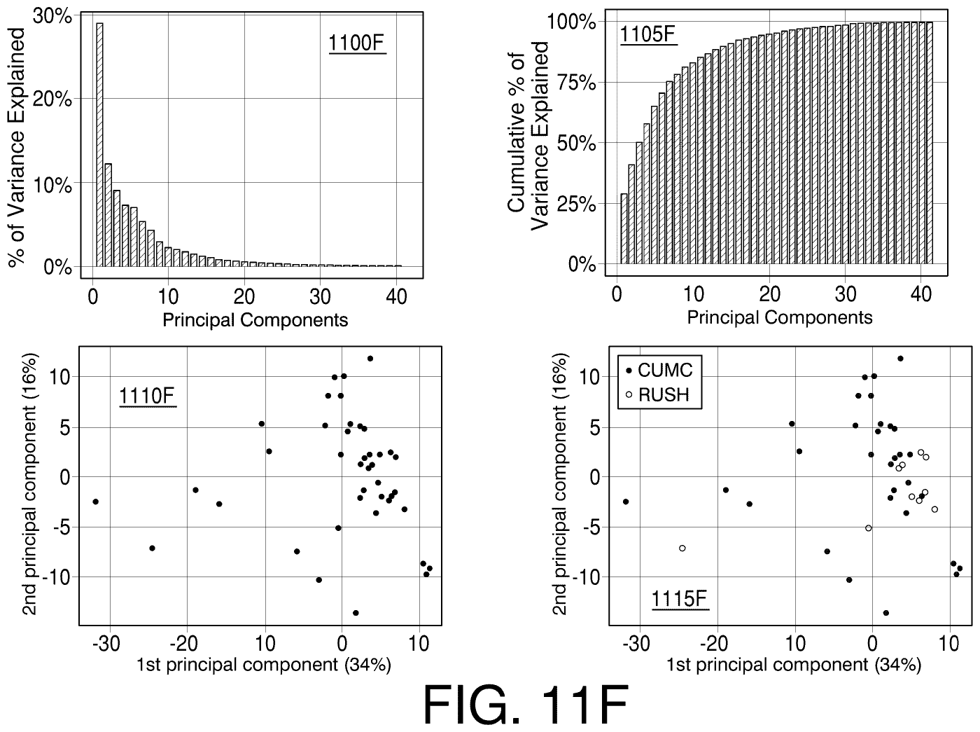

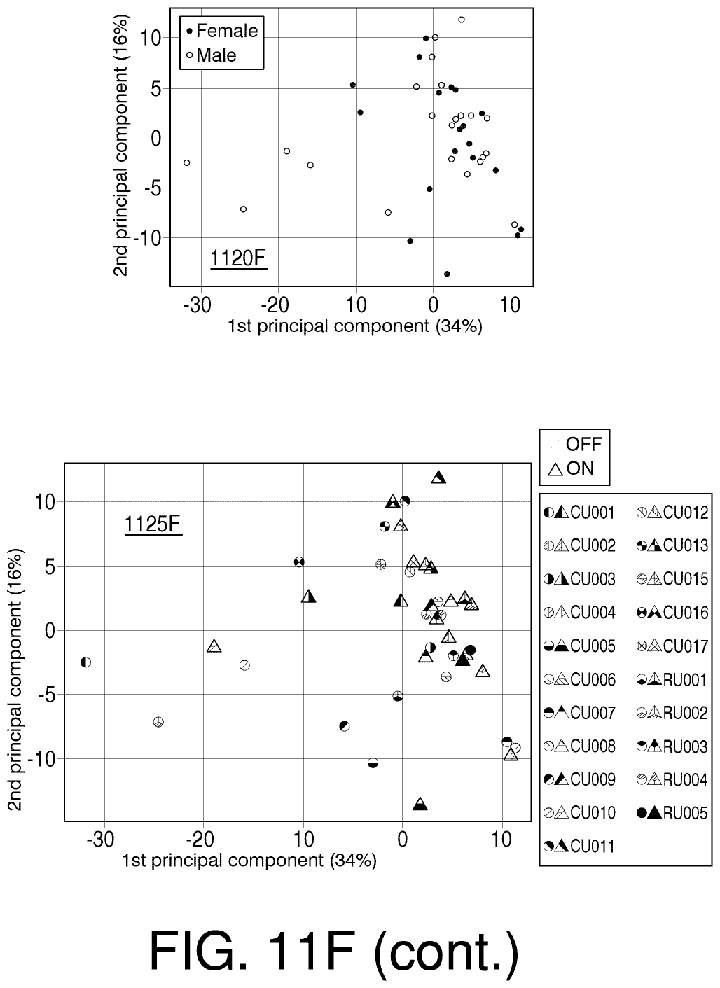

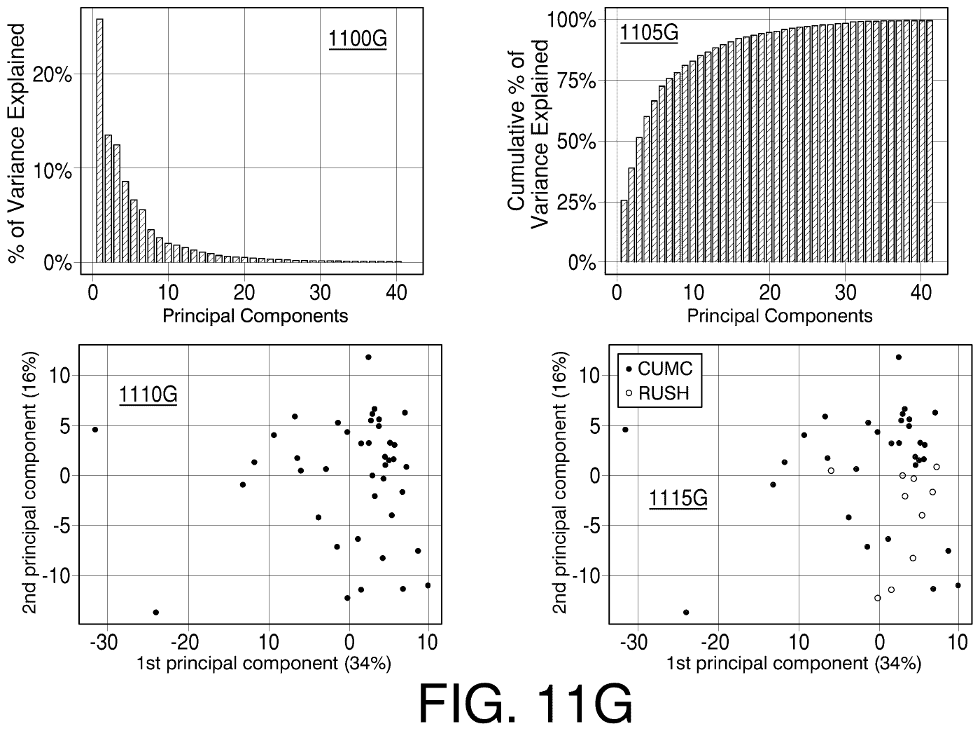

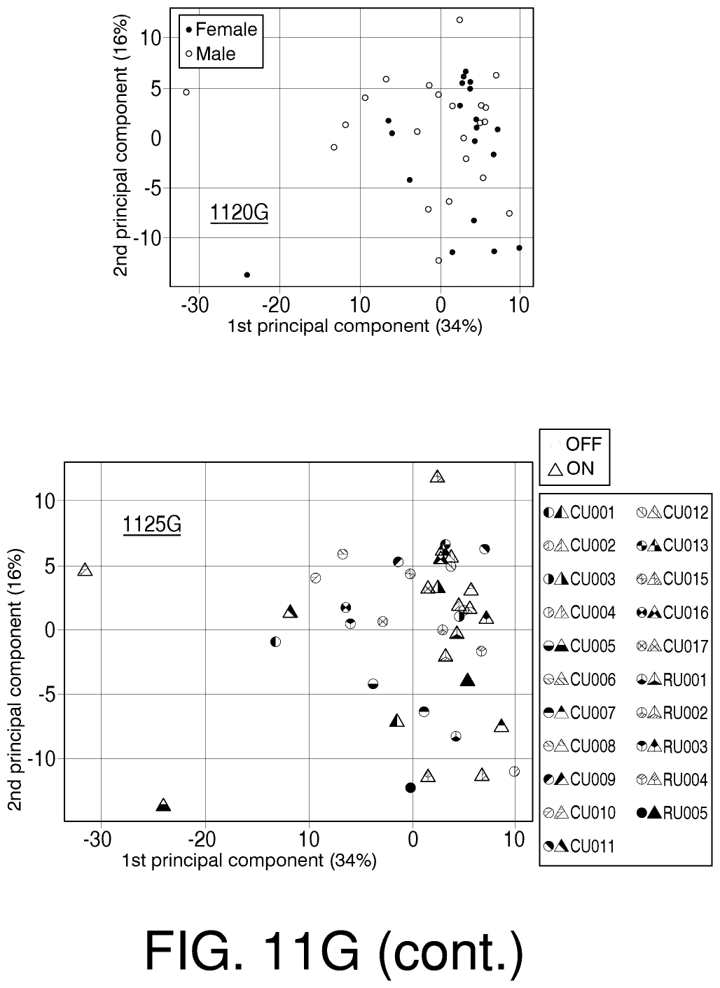

The amount of variation exhibited in the correlation heat maps of FIGS. 10A-10G may be further investigated by performing PCA of the speech characteristics by task. More particularly, scree and scatter plots illustrated in FIGS. 11A-FIG. 11G plot the first principal component (PC1) against the second principal component (PC2) by speech task (e.g., paragraph reading in plots 1100A-1125A in FIG. 11A, picture description 1 in plots 1100B-1125B in FIG. 11B, picture description 2 in plots 1100C-1125C in FIG. 11C, diadochokinetic rate (DDK) in plots 1100D-1125D in FIG. 11D, sustained vowel phonation in plots 1100E-1125E in FIG. 11E, semantic fluency in plots 1100F-1125F in FIG. 11F, and phonemic fluency in plots 1100G-1125G in FIG. 11G). Examination of these scree and scatter plots may reveal important information about various speech tasks. For instance, the clustering and separation along PC1 may explain the variance in the DDK and sustained vowel phonation tasks (e.g., FIG. 11D and FIG. 11E, respectively). Additional evidence of clustering using PC1 and PC2 was found in the paragraph reading, DDK, and sustained vowel phonation tasks (e.g., FIG. 11A, FIG. 11D and FIG. 11E, respectively).

### MDS-UPDRS III Score and Gait Characteristics

Gait characteristics were found to change by clinical status and disease severity, particularly in the context of PD. More particularly, clinical data (e.g., MDS-UPDRS scores) were integrated with the gait characteristic data to compute the correlation between each gait characteristic and the MDS-UPDRS III score. In an aspect, the Spearman correlation is used to measure the strength and direction of the association between gait characteristics and the MDS-UPDRS III score. This non-parametric measure may be useful for detecting monotonic relationships. Furthermore, the mean relative change in each gait characteristic between the ON-state and the OFF-state was calculated. This metric helps to understand how each gait characteristic is affected by the medication.

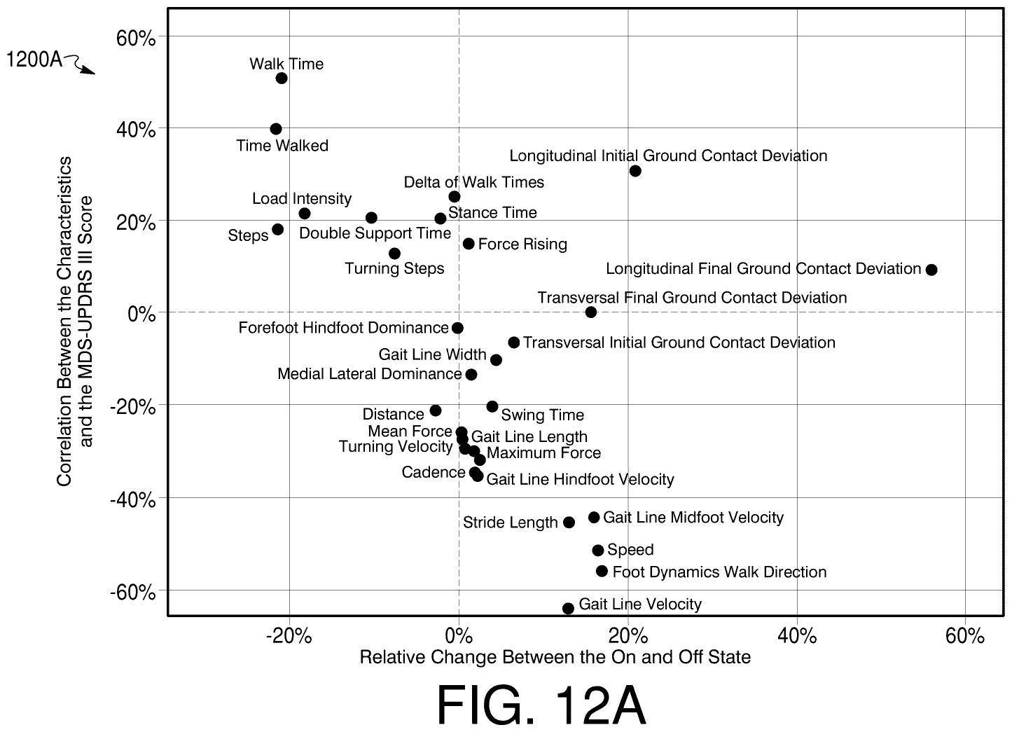

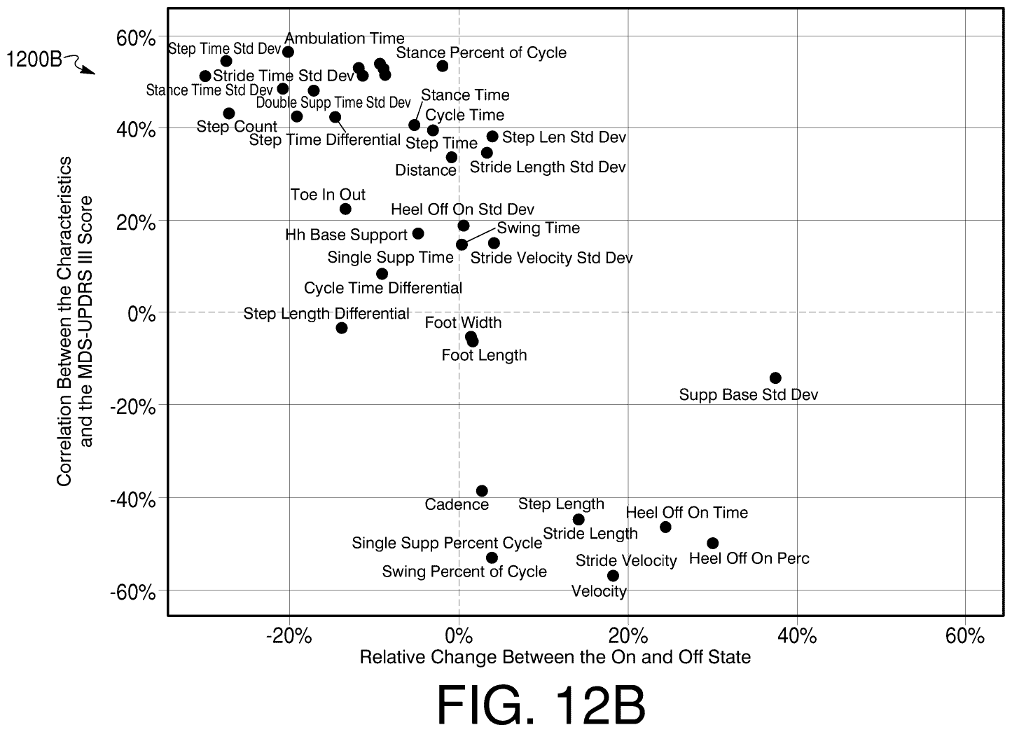

FIGS. 12A and 12B provide scatter plots 1200A and 1200B for the test and production devices, respectively, in which the Spearman correlation between each gait characteristic and the MDS-UPDRS III score is contained on the y-axis and the percent difference in each gait characteristic between the ON-state and the OFF-state is contained on the x-axis. The y-axis therefore represents how strongly each gait characteristic is associated with disease severity and the x-axis indicates the magnitude of change in gait characteristics due to levodopa. Examination of these scatter plots reveals some interesting trends. For instance, some gait characteristics, such as gait line velocity, show a moderately strong correlation with the MDS-UPDRS III score but exhibit relatively small changes between the ON-state and the OFF-state. This suggests that these characteristics are consistently indicative of disease severity, regardless of the levodopa state. As another example, other characteristics (e.g., longitudinal final ground contact deviation) show significant changes between the ON-state and the OFF-state but have weaker associations with the MDS-UPDRS III score. This implies that these characteristics are more sensitive to levodopa treatment but are less reliable as indicators of overall disease severity. Accordingly, the foregoing analysis helps identify which gait characteristics could serve as reliable biomarkers for assessing disease severity and treatment effects.

FIG. 13 presents heat map 1300 that visualizes the scaled values of gait characteristics across different subjects, particularly in relation to their clinical status as measured by the MDS-UPDRS III scores. The heat map provides a comprehensive view of the relationship between gait characteristics and disease severity. More particularly, the subjects on the x-axis are ordered based on their MDS-UPDRS III scores, with higher scores indicating more severe motor symptoms. Moving from left to right on the x-axis, there is a transition from subjects with lower MDS-UPDRS III scores (i.e., less severe symptoms) to those with higher scores (i.e., more severe symptoms). Gait characteristics on the y-axis are ordered based on their correlation with the MDS-UPDRS III score. Specifically, characteristics with the strongest positive or negative correlations are likely positioned at the top or bottom, respectively. This ordering helps in identifying which gait characteristics are most strongly associated with disease severity. In an aspect, each cell in the heat map represents the scaled value of a gait characteristic for a given subject. The scaling standardizes the values to a common range, allowing for meaningful comparison. In an aspect, the color of each cell reflects the magnitude of the scaled value.

At the top of the heat map, characteristics of the data are represented in various shades of gray, and a left-to-right gradient from lighter shading to darker shading is generally observed, which indicates that characteristics with darker shading are associated with lower MDS-UPDRS III scores (i.e., less severe symptoms) and lighter shading as the disease severity increases. At the bottom of the heat map, characteristics of the data are also represented in various shades of gray, and a left-to-right gradient from darker shading to lighter shading is generally observed, which indicates that gait characteristics with lower values are associated with lower MDS-UPDRS III scores and increase as the disease severity increases. Accordingly, the heat map generally exhibits the expected gradient behavior, confirming that the gait characteristics are correlated with disease severity in a predictable manner.

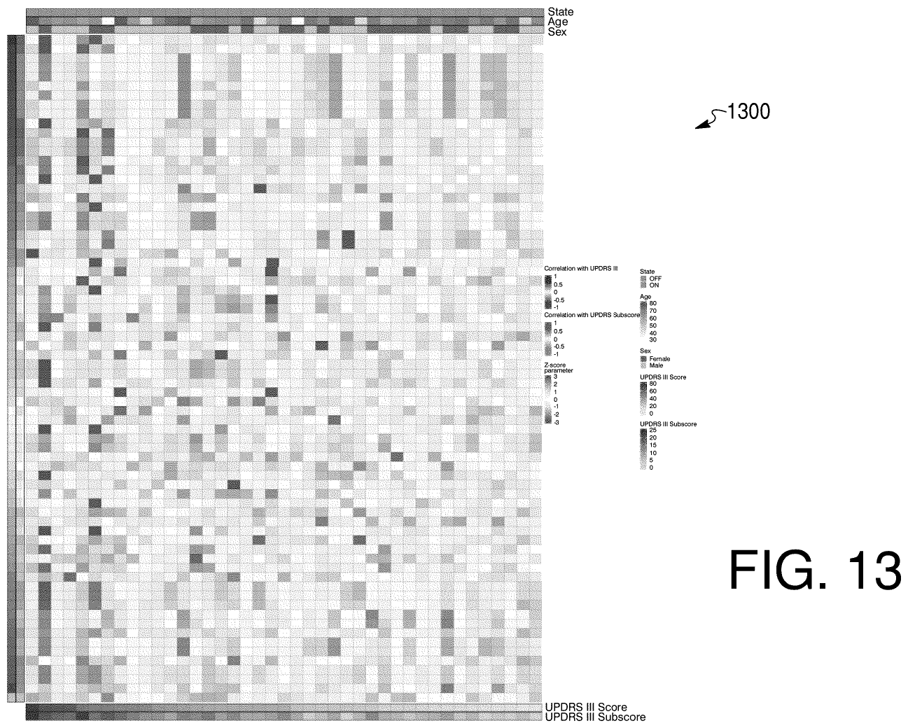

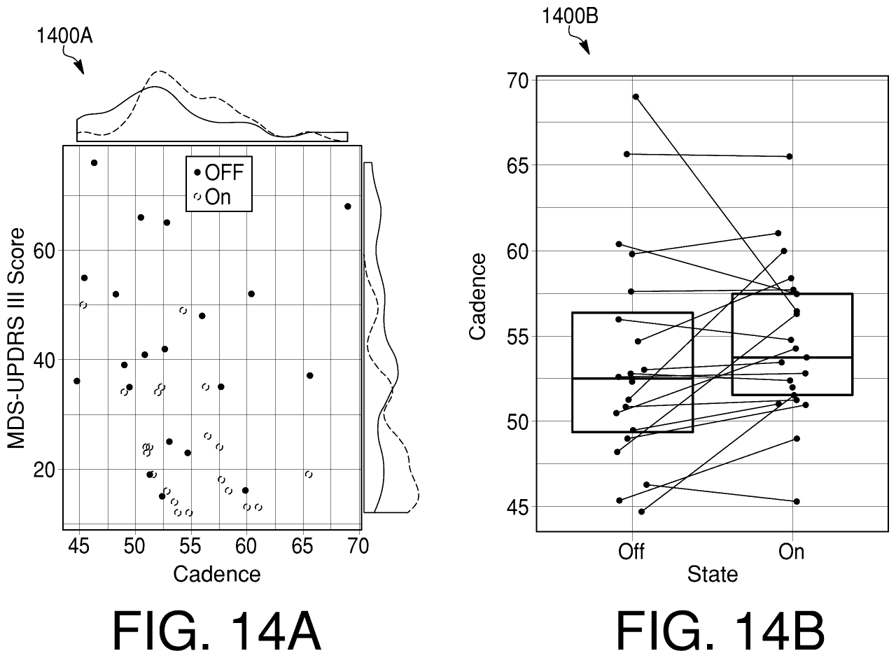

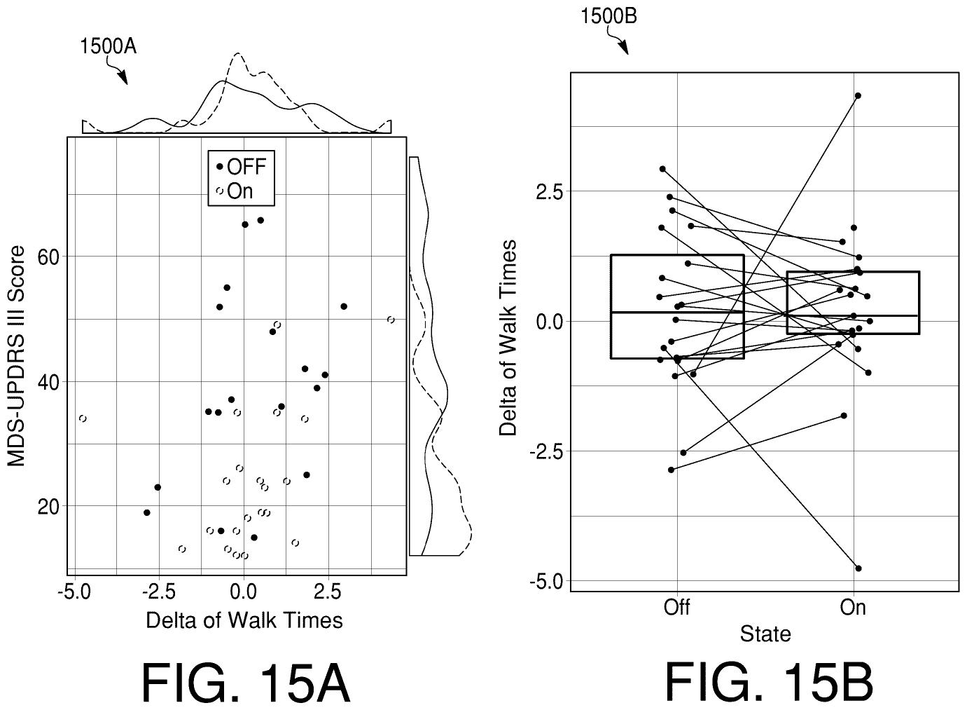

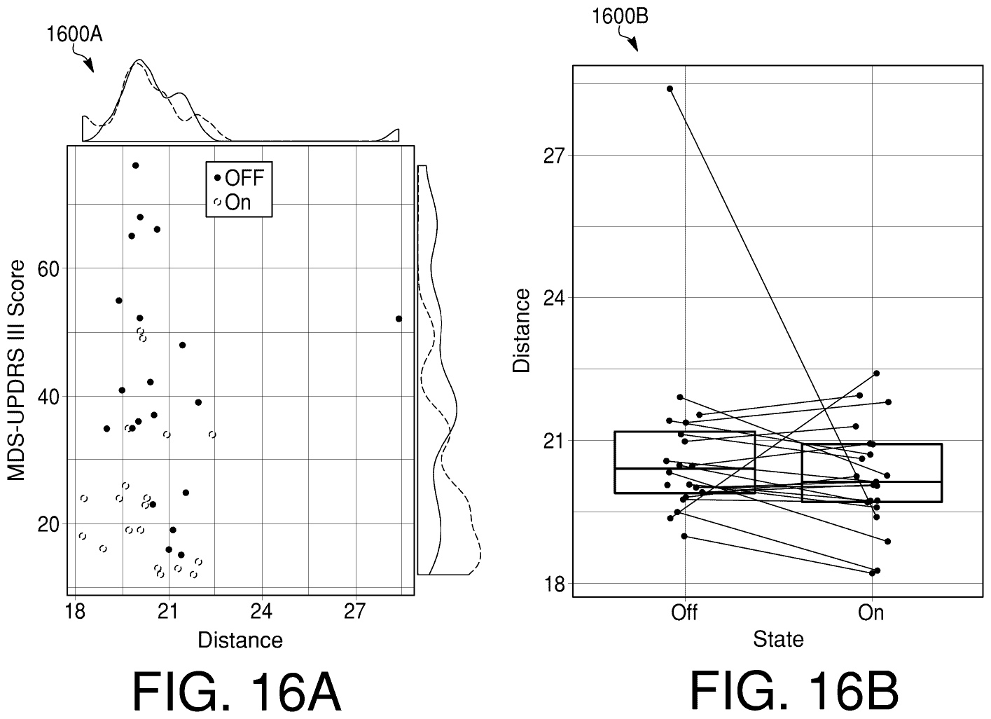

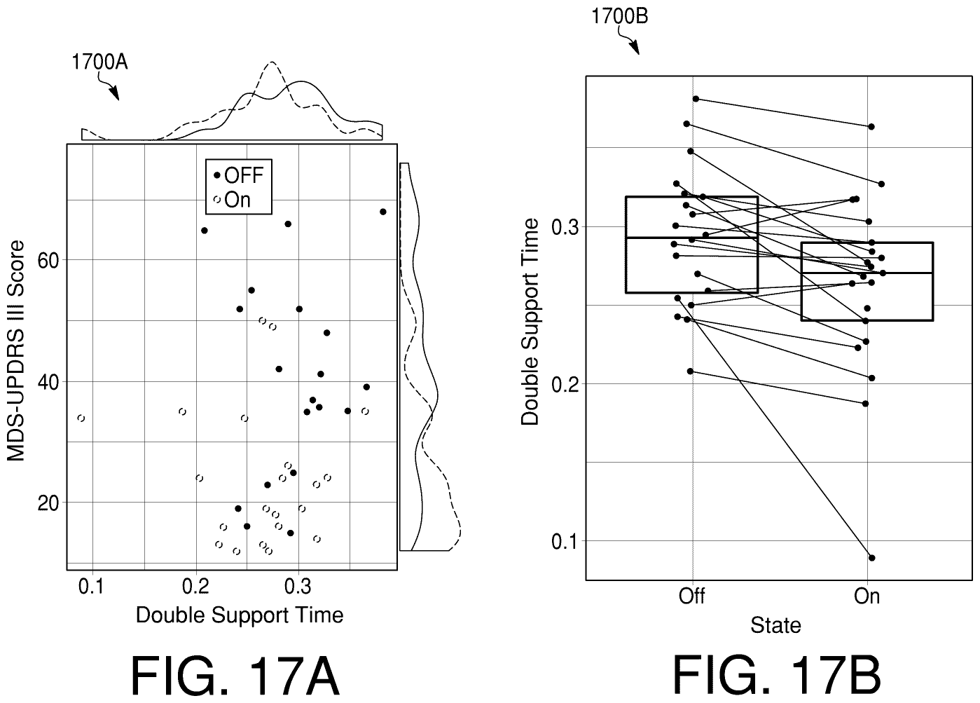

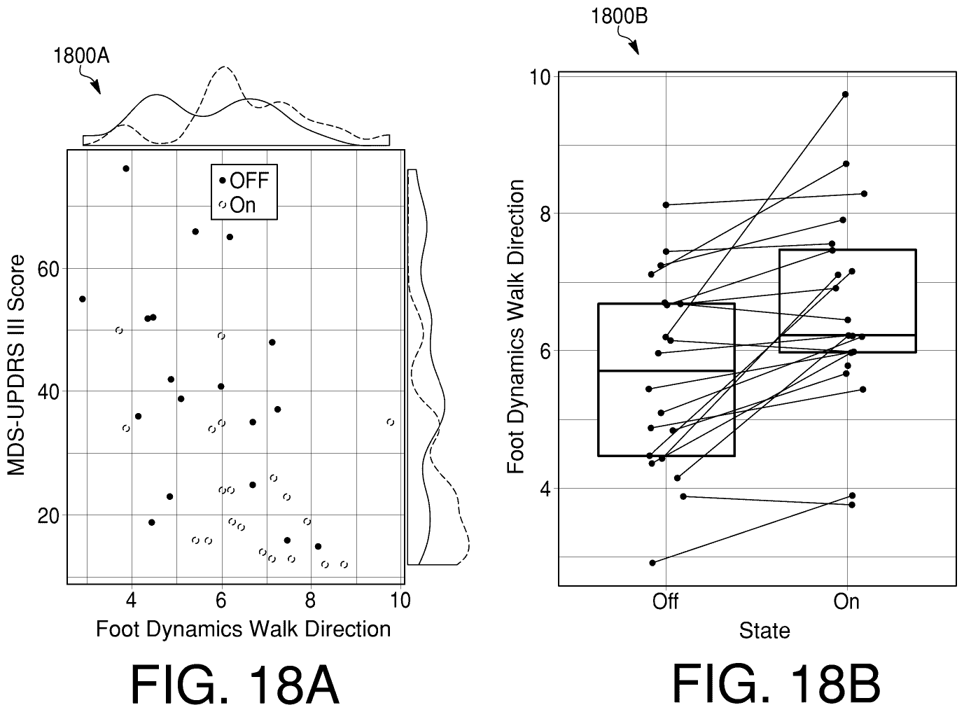

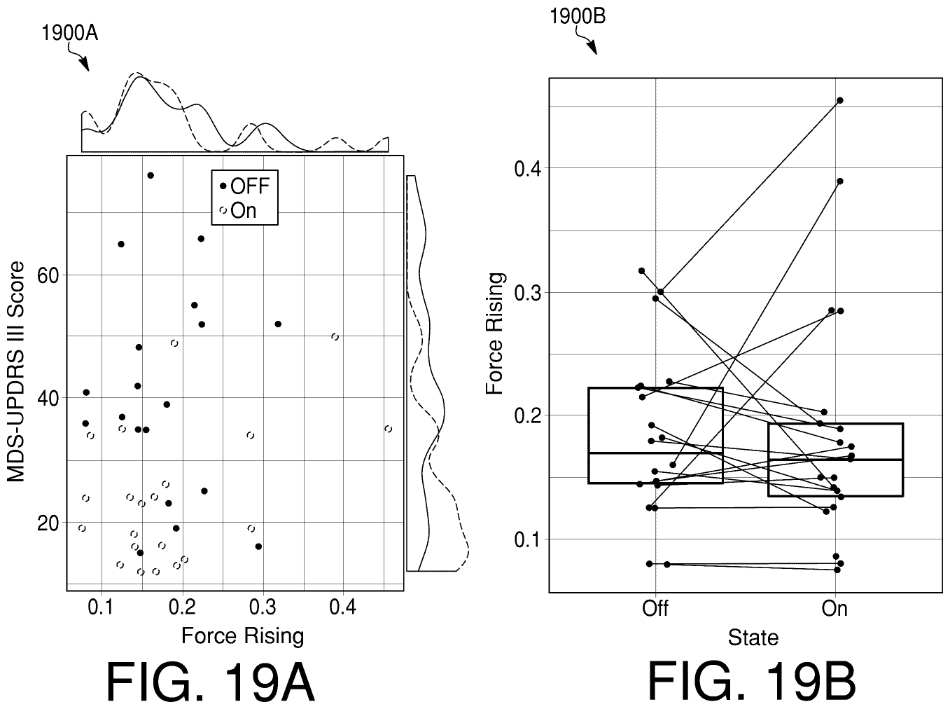

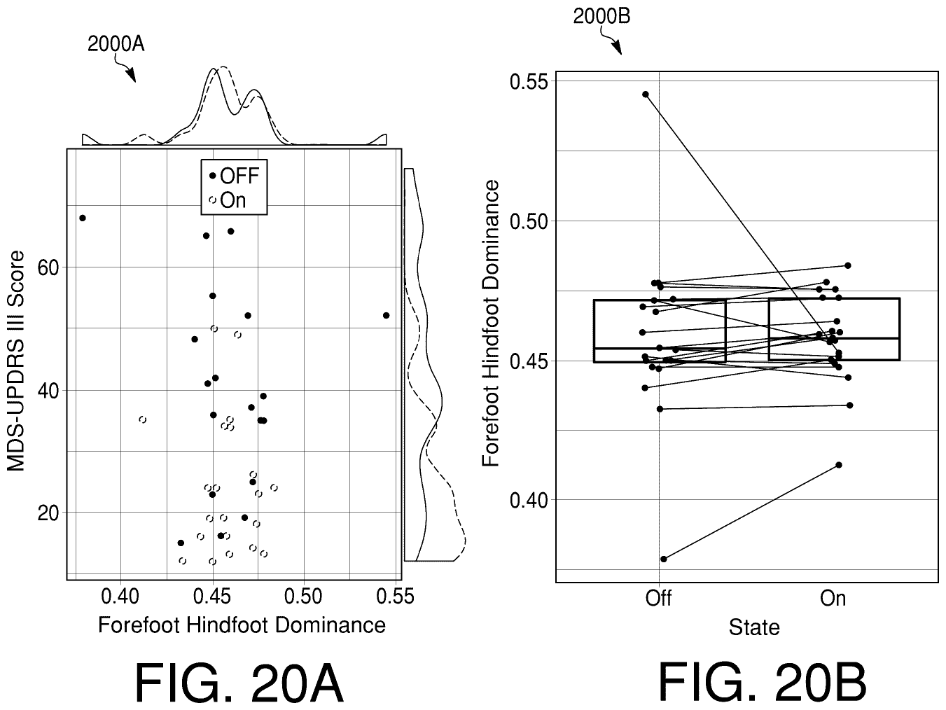

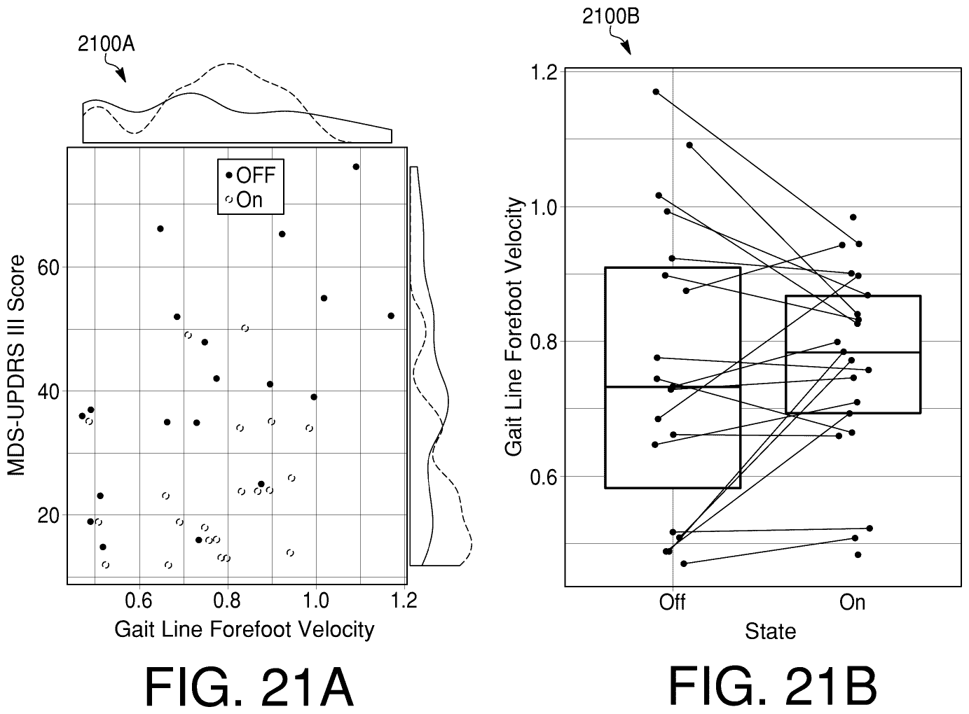

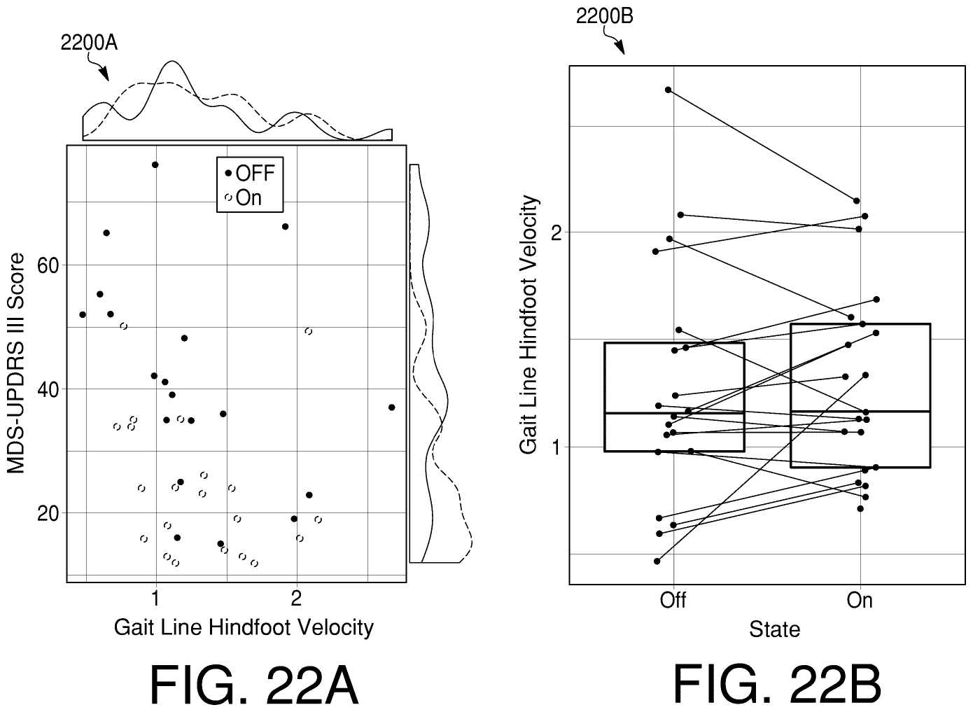

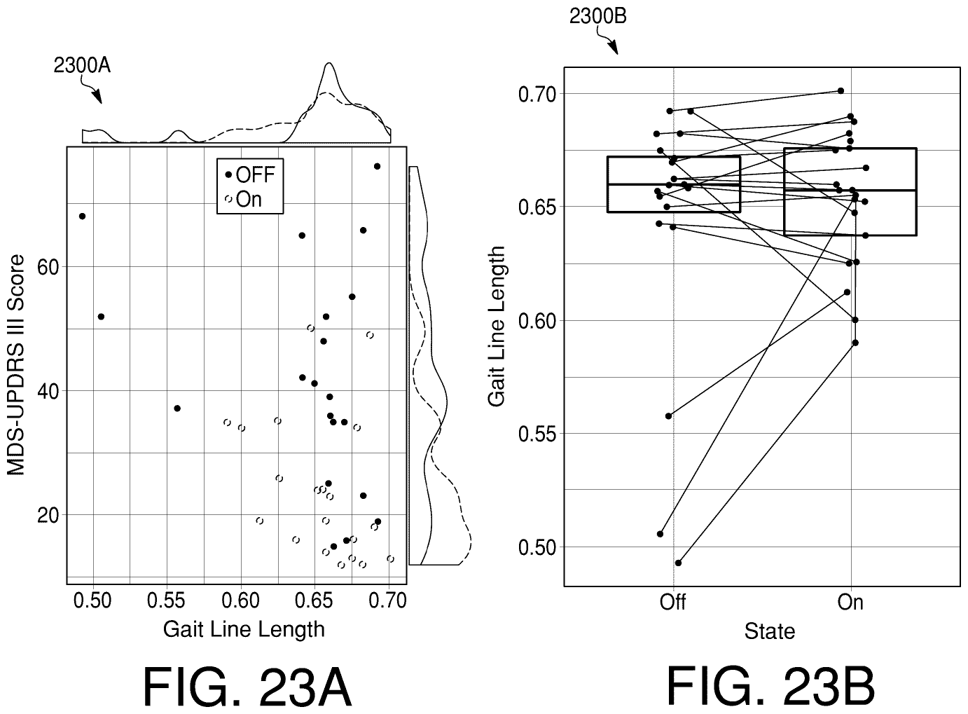

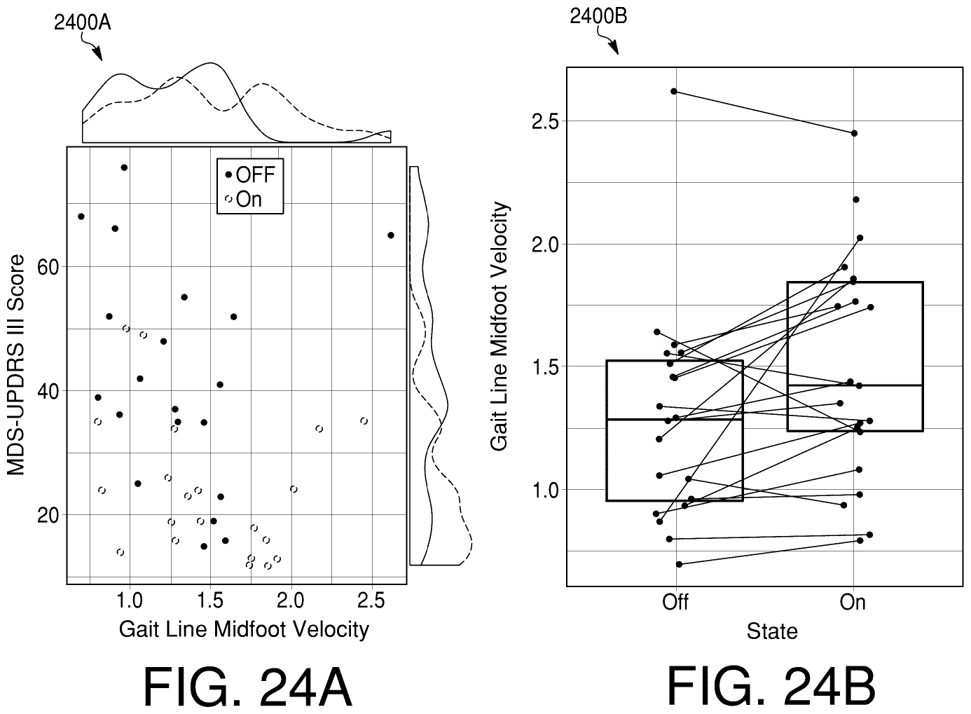

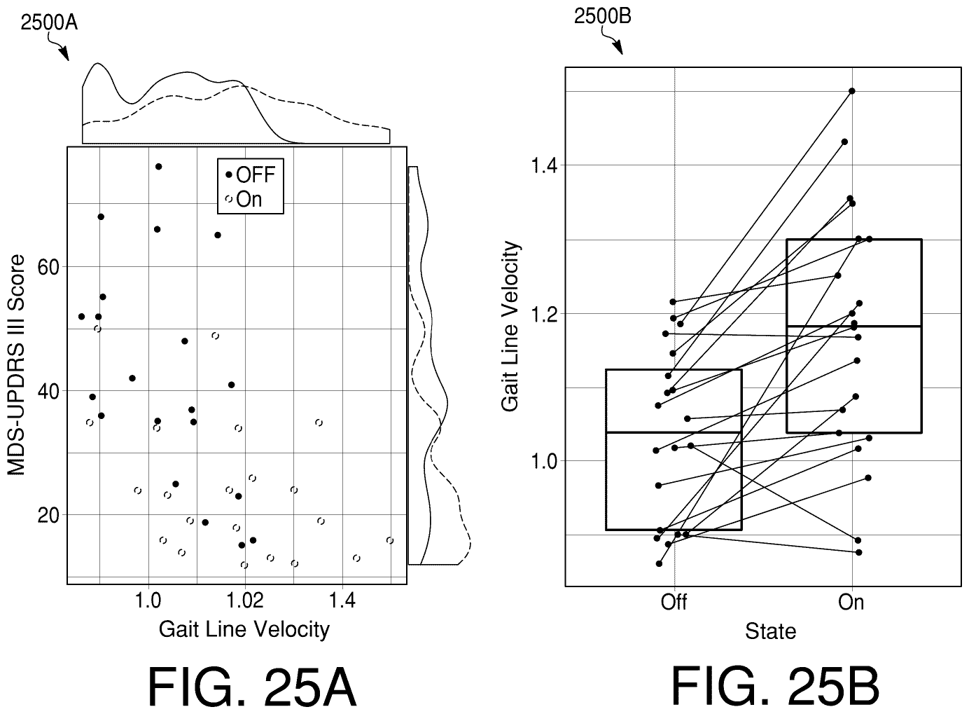

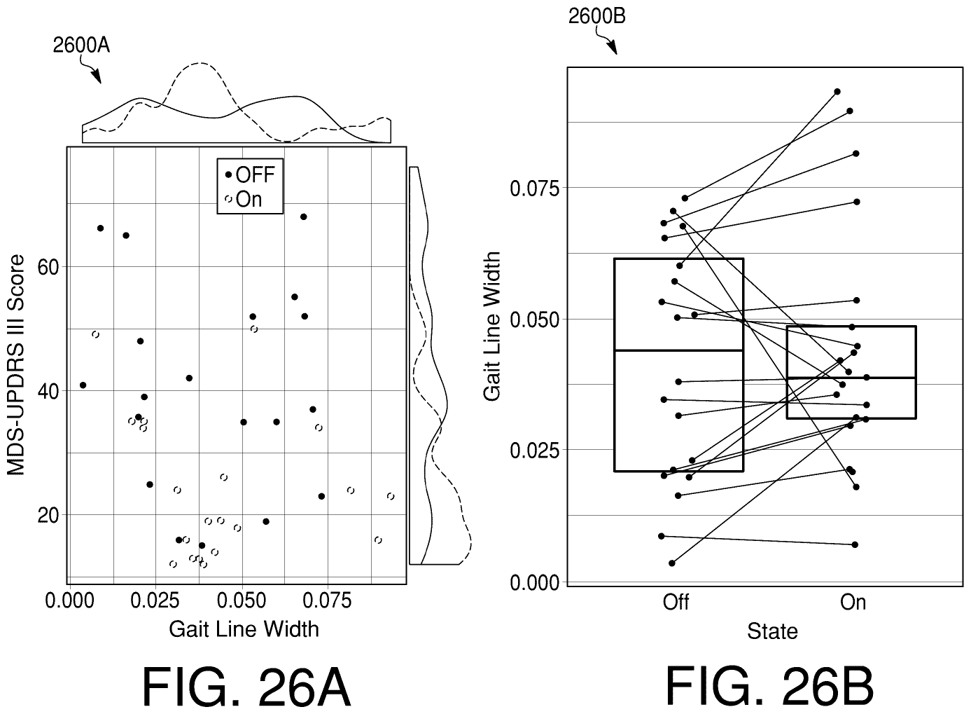

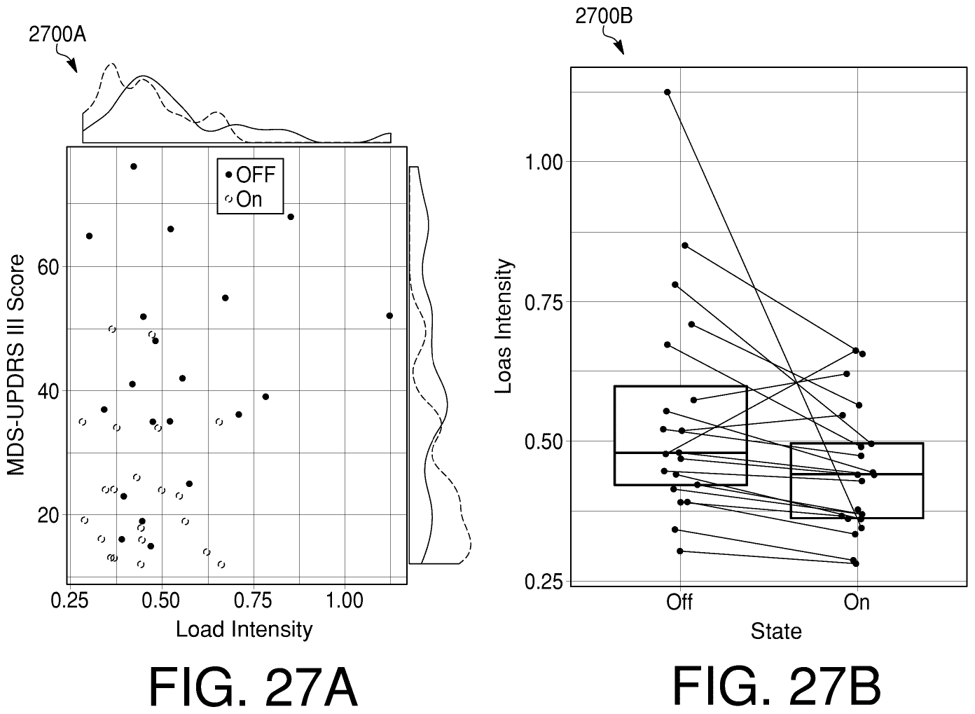

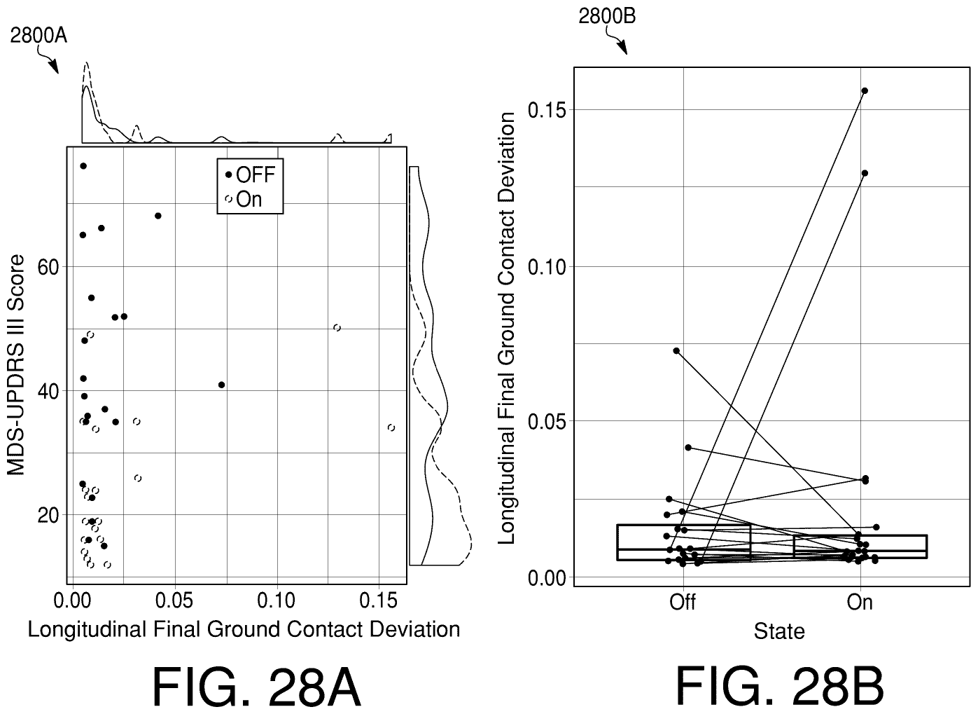

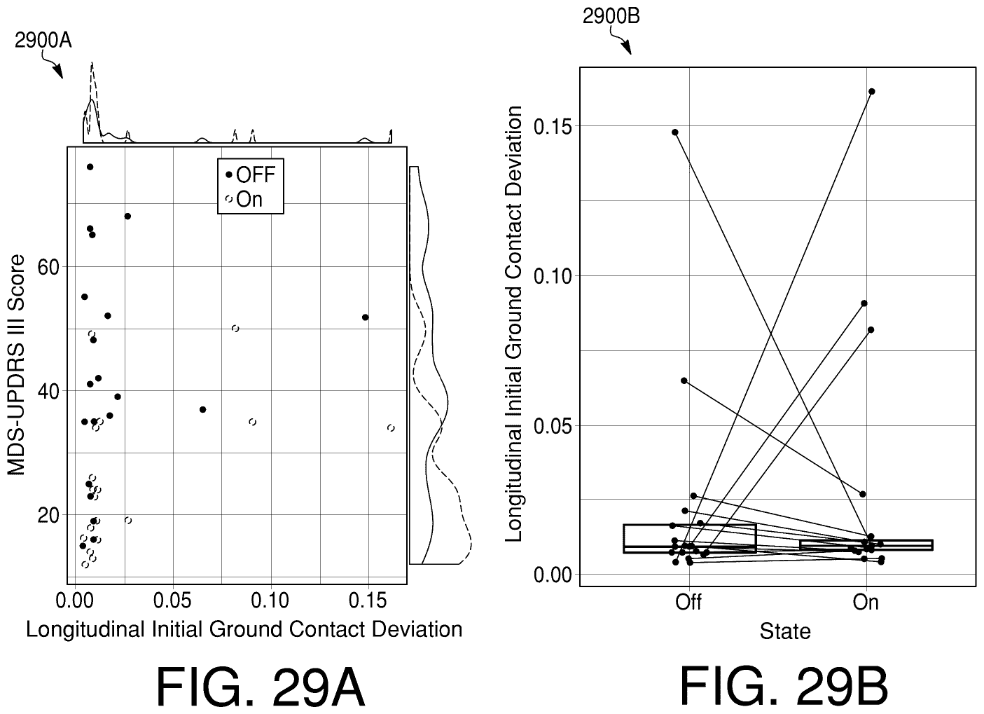

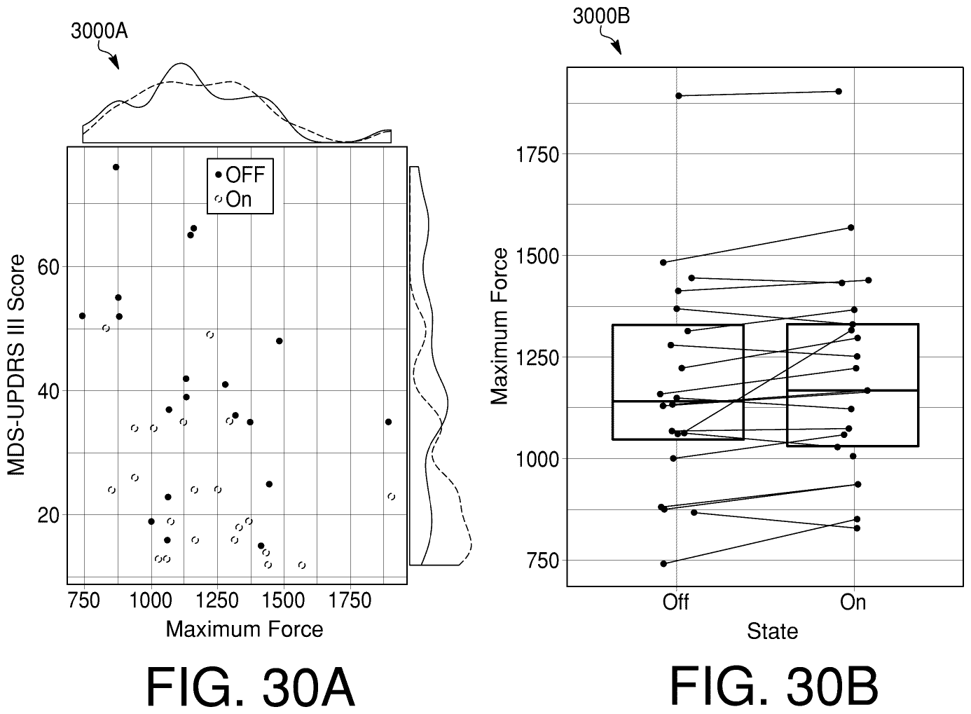

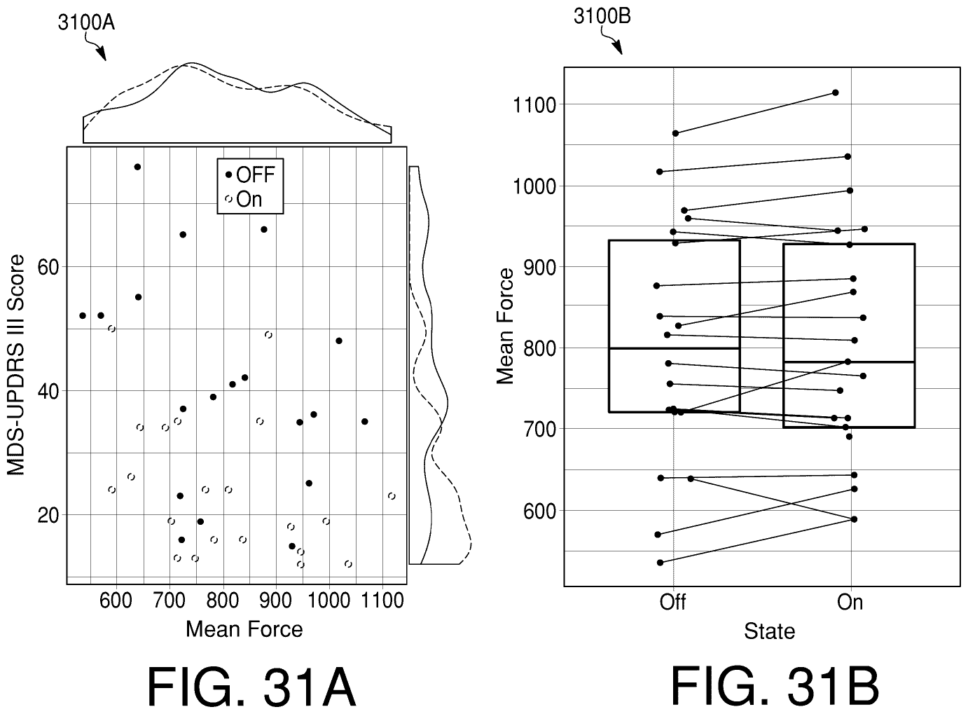

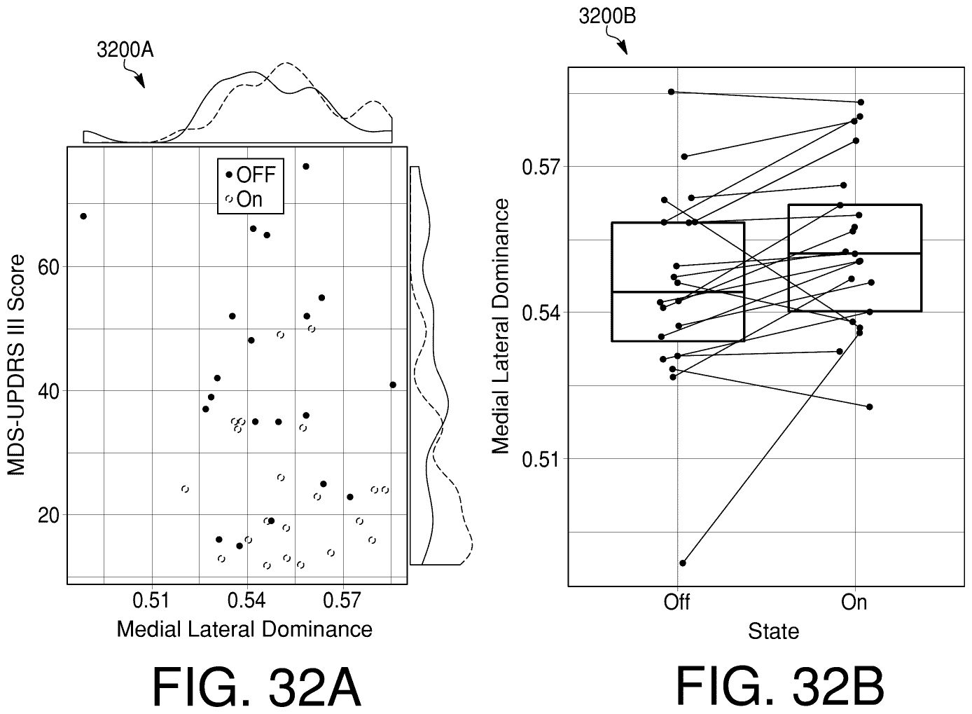

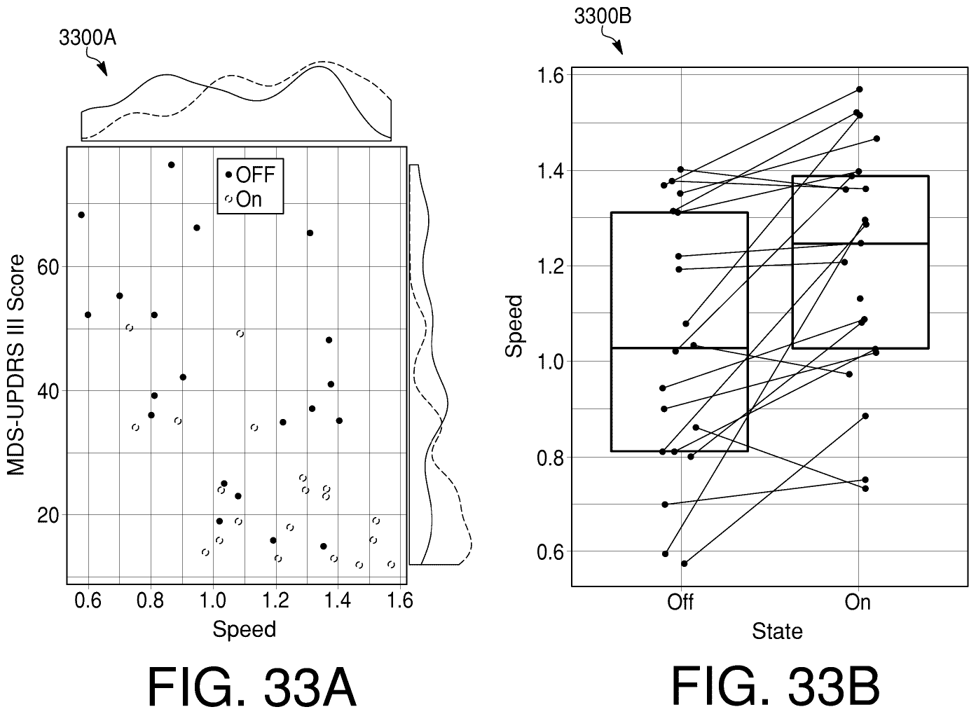

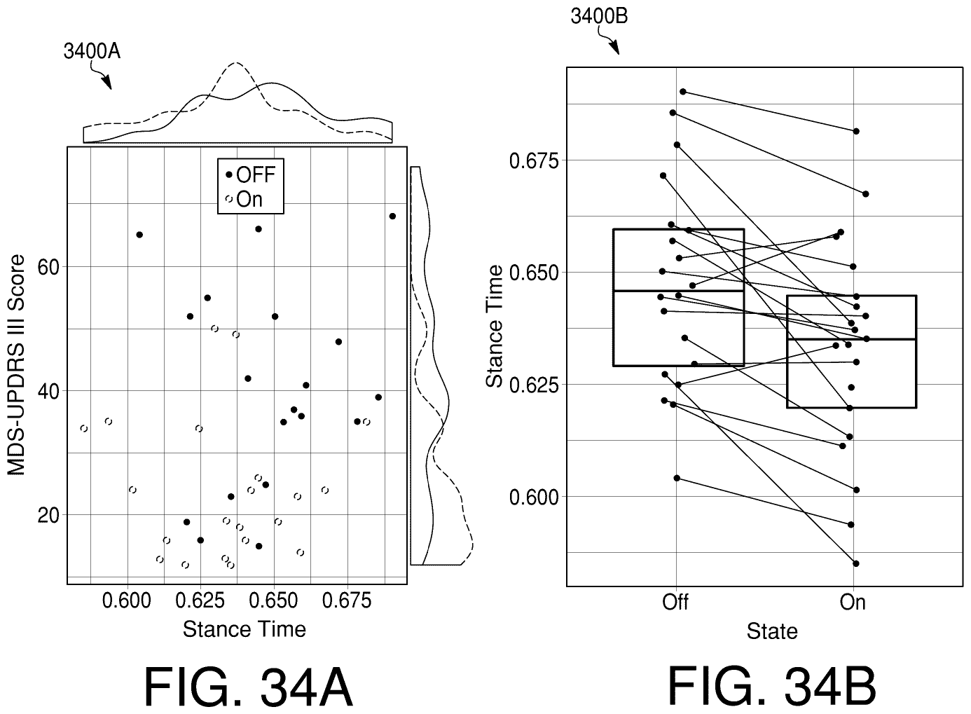

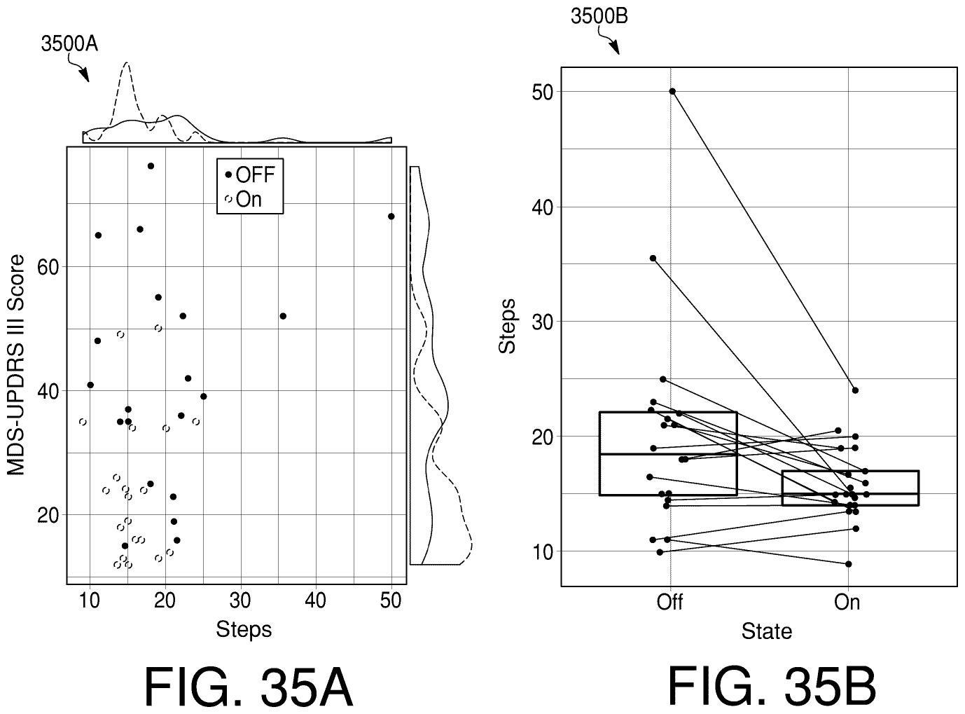

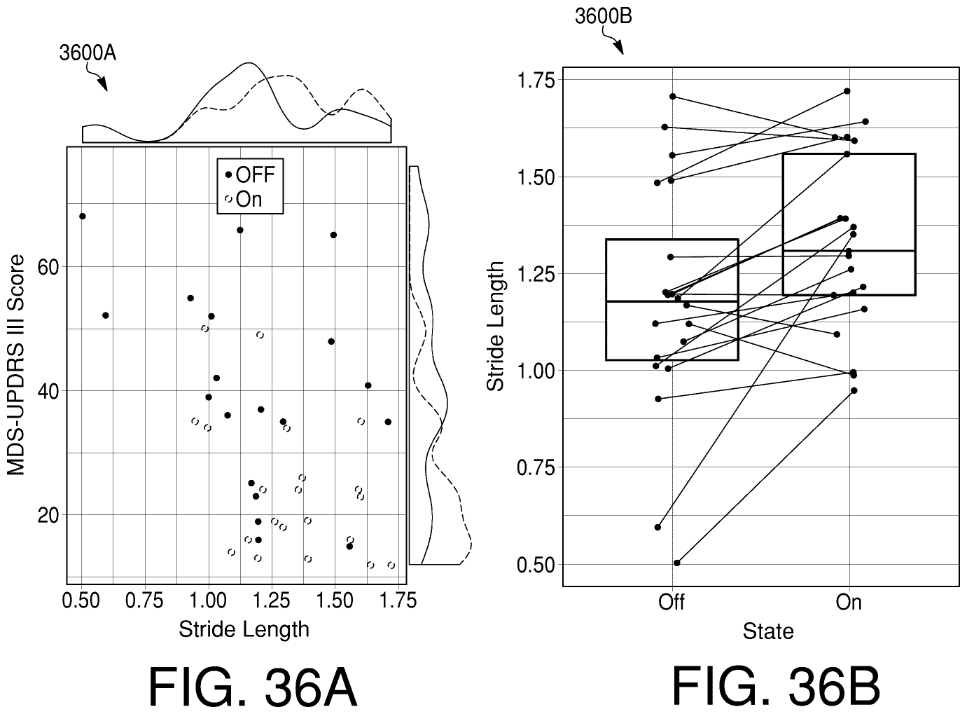

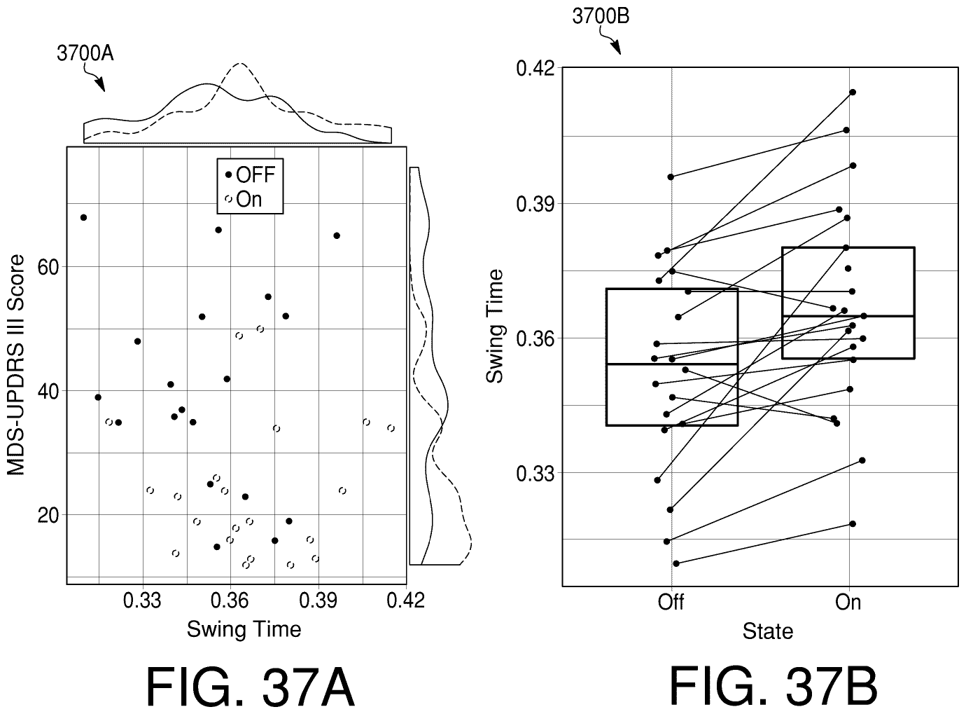

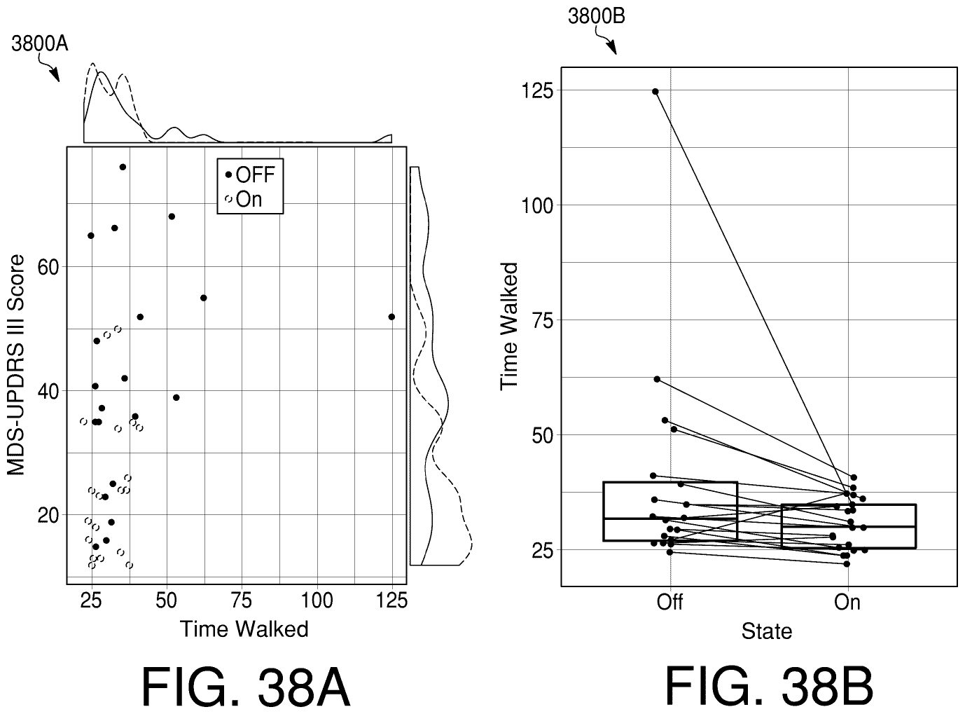

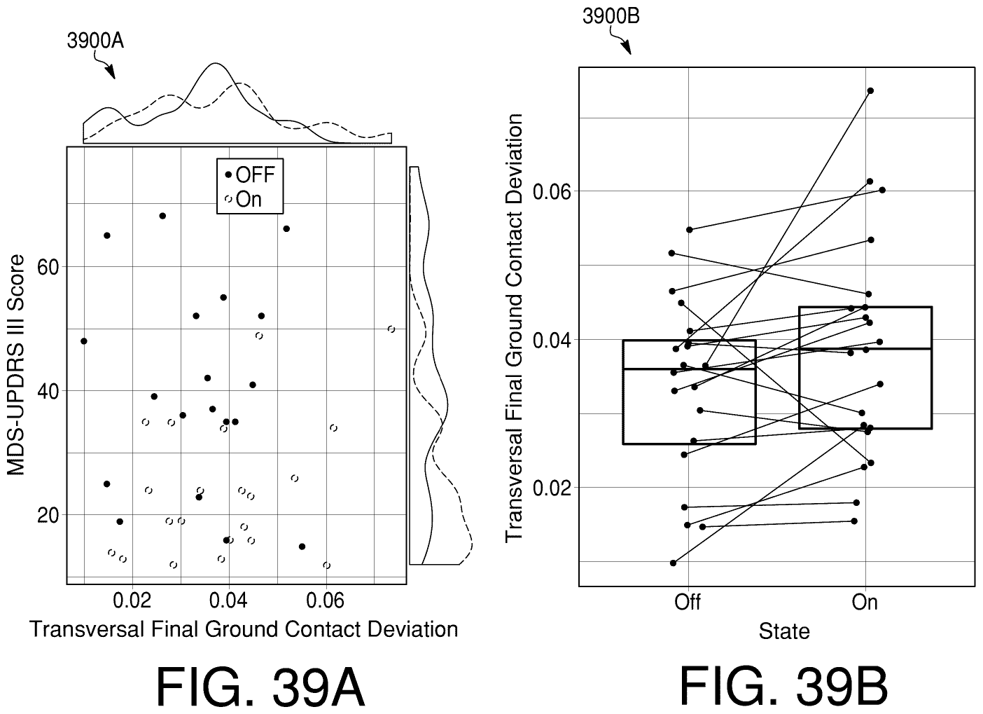

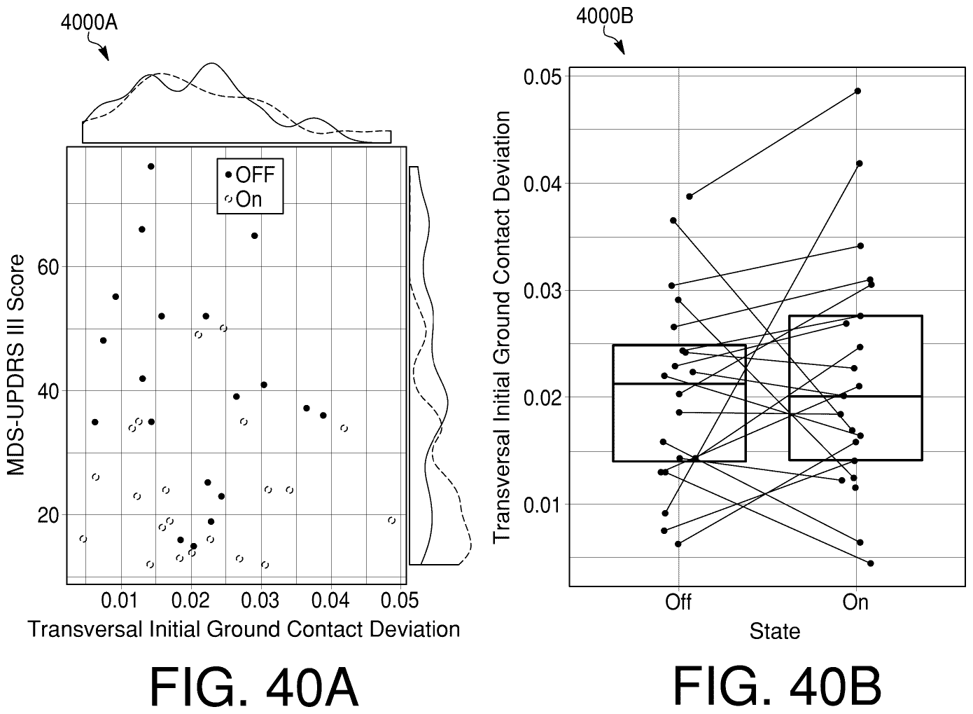

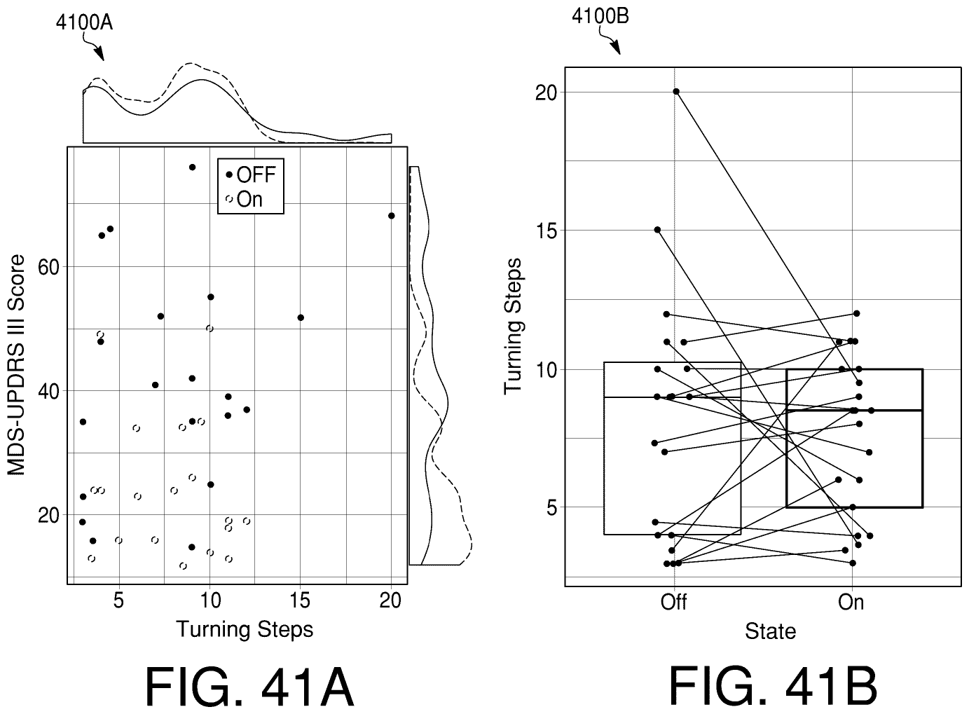

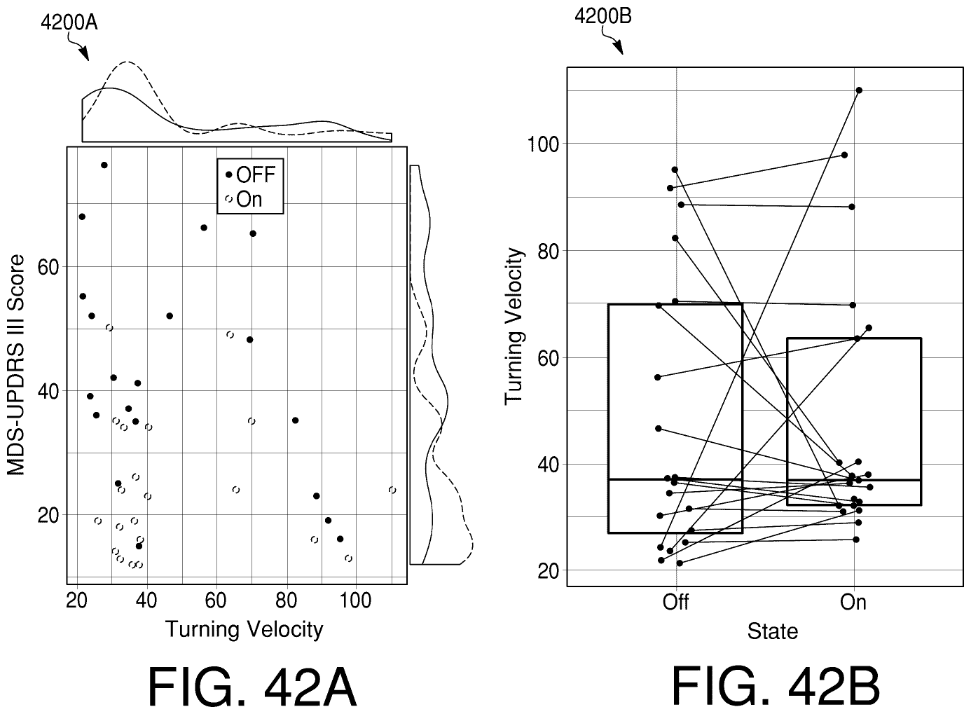

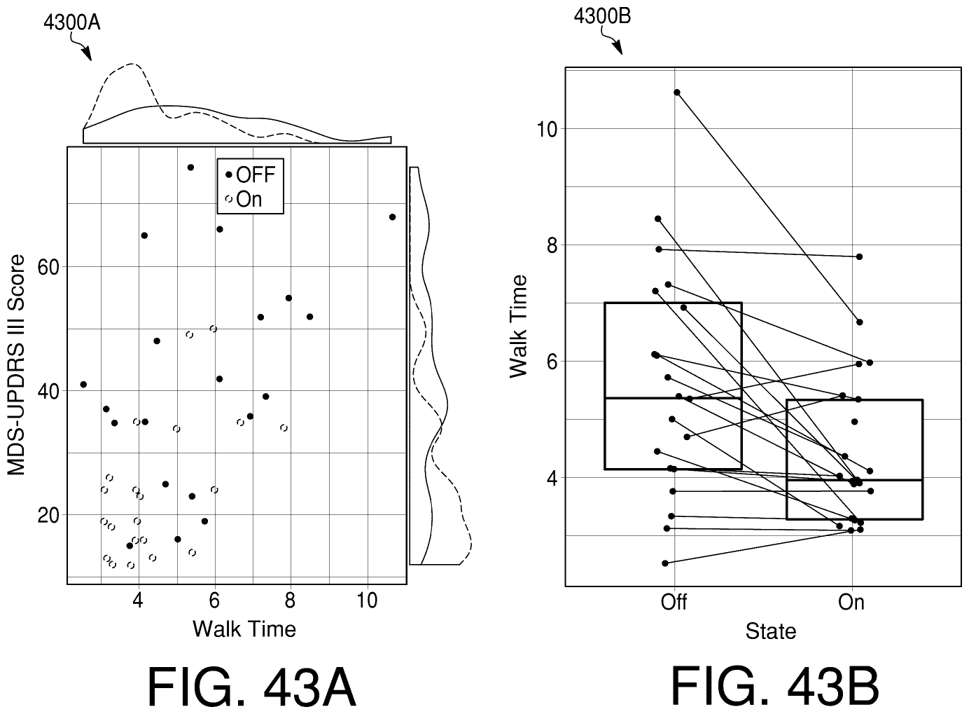

Pluralities of scatter plots between PD severity and each gait characteristic, along with differences between levodopa states, are provided for the test device in plots 1400A-4300B in FIGS. 14A-43B. More particularly, the gait characteristics represented by plots 1400A-4300B include: cadence (in plots 1400A and 1400B in FIGS. 14A and 14B), delta of walk times (in plots 1500A and 1500B in FIGS. 15A and 15B), distance (in plots 1600A and 1600B in FIGS. 16A and 16B), double support time (in plots 1700A and 1700B in FIGS. 17A and 17B), foot dynamics walk direction (in plots 1800A and 1800B in FIGS. 18A and 18B), force rising (in plots 1900A and 1900B in FIGS. 19A and 19B), forefoot hindfoot dominance (in plots 2000A and 2000B in FIGS. 20A and 20B), gait line forefoot velocity (in plots 2100A and 2100B in FIGS. 21A and 21B), gait line hindfoot velocity (in plots 2200A and 2200B in FIGS. 22A and 22B), gait line length (in plots 2300A and 2300B in FIGS. 23A and 23B), gait line midfoot velocity (in plots 2400A and 2400B in FIGS. 24A and 24B), gait line velocity (in plots 2500A and 2500B in FIGS. 25A and 25B), gait line width (in plots 2600A and 2600B in FIGS. 26A and 26B), load intensity (in plots 2700A and 2700B in FIGS. 27A and 27B), longitudinal final ground contact deviation (in plots 2800A and 2800B in FIGS. 28A and 28B), longitudinal initial ground contact deviation (in plots 2900A and 2900B in FIGS. 29A and 29B), maximum force (in plots 3000A and 3000B in FIGS. 30A and 30B), mean force (in plots 3100A and 3100B in FIGS. 31A and 31B), medial lateral dominance (in plots 3200A and 3200B in FIGS. 32A and 32B), speed (in plots 3300A and 3300B in FIGS. 33A and 33B), stance time (in plots 3400A and 3400B in FIGS. 34A and 34B), steps (in plots 3500A and 3500B in FIGS. 35A and 35B), stride length (in plots 3600A and 3600B in FIGS. 36A and 36B), swing time (in plots 3700A and 3700B in FIGS. 37A and 37B), time walked (in plots 3800A and 3800B in FIGS. 38A and 38B), transversal final ground contact deviation (in plots 3900A and 3900B in FIGS. 39A and 39B), transversal initial ground contact deviation (in plots 4000A and 4000B in FIGS. 40A and 40B), turning steps (in plots 4100A and 4100B in FIGS. 41A and 41B), turning velocity (in plots 4200A and 4200B in FIGS. 42A and 42B), and walk time (in plots 4300A and 4300B in FIGS. 43A and 43B).

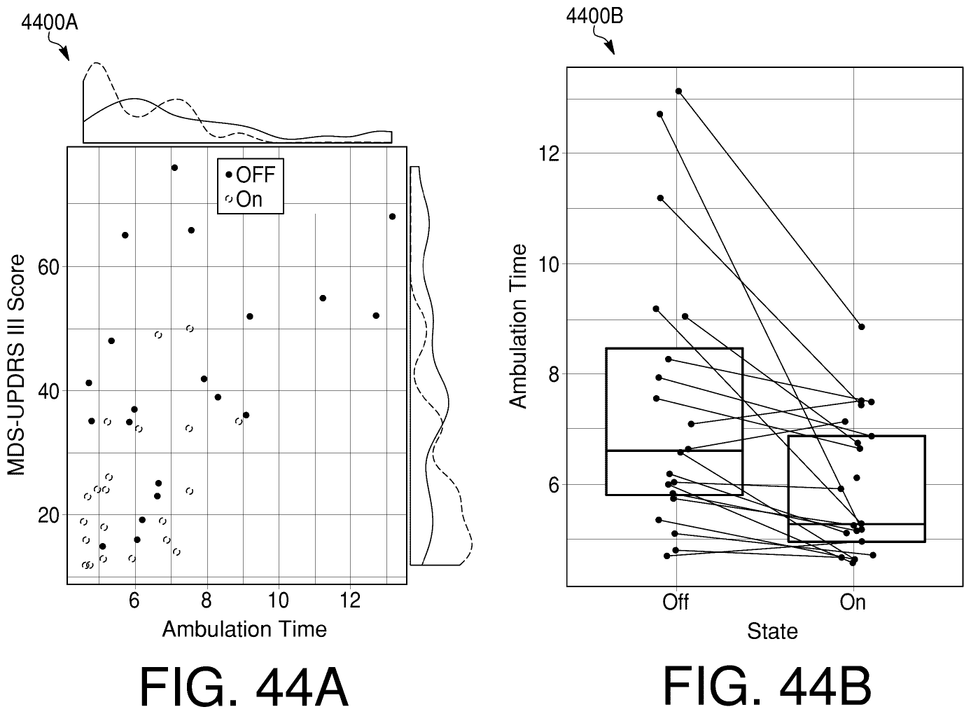

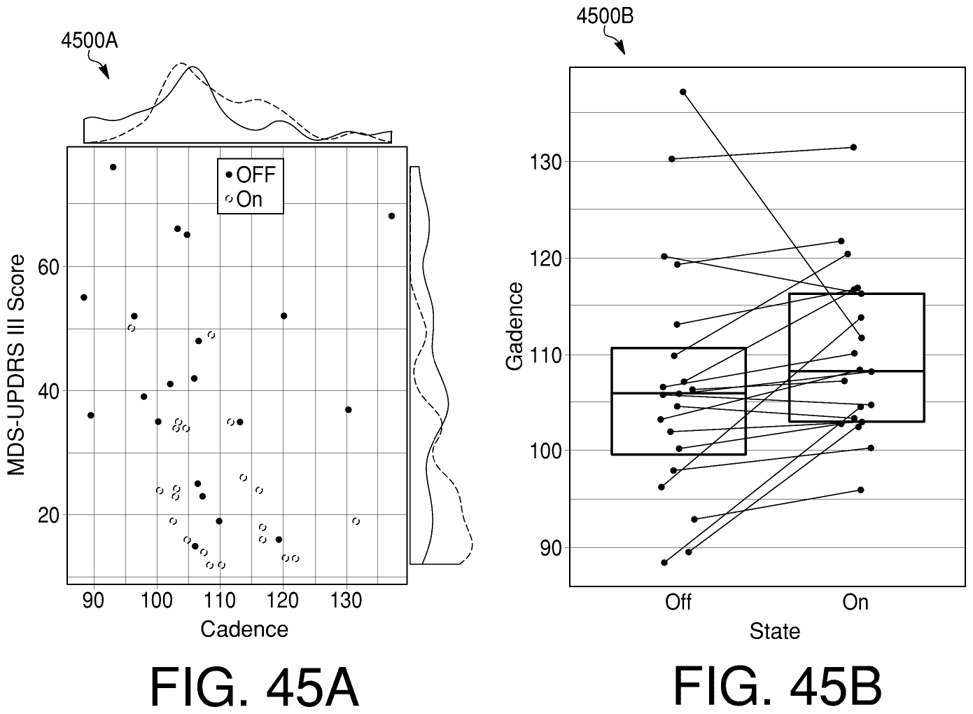

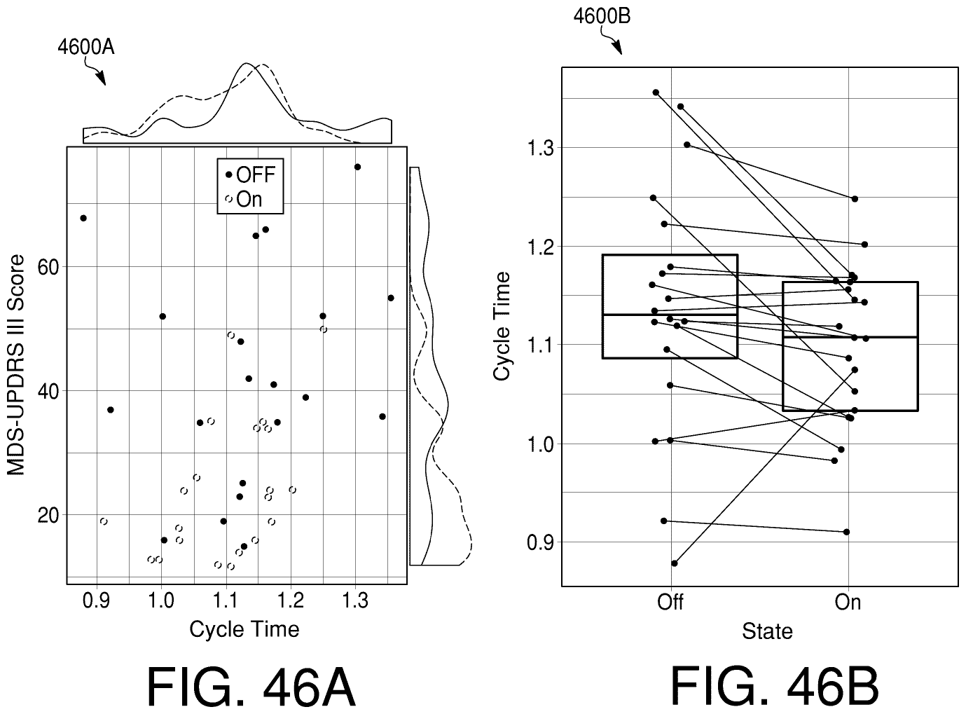

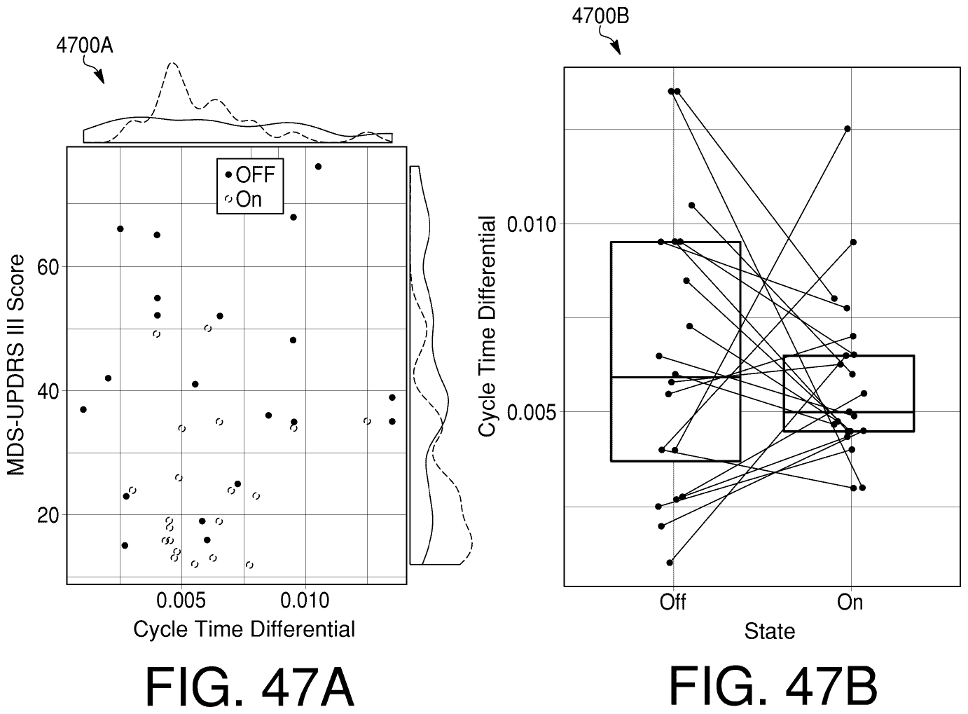

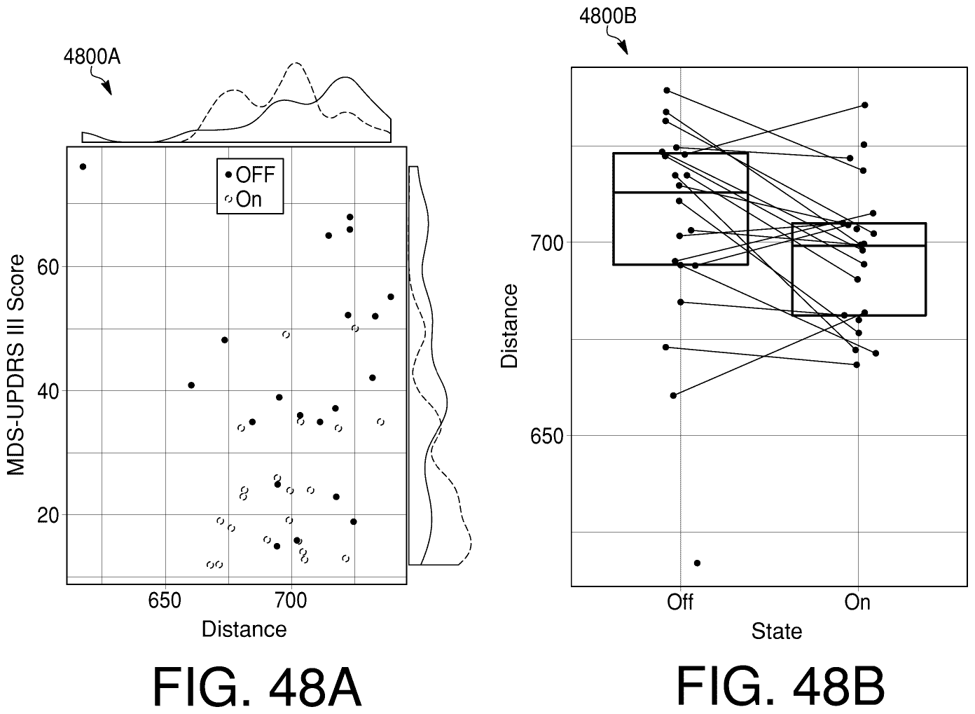

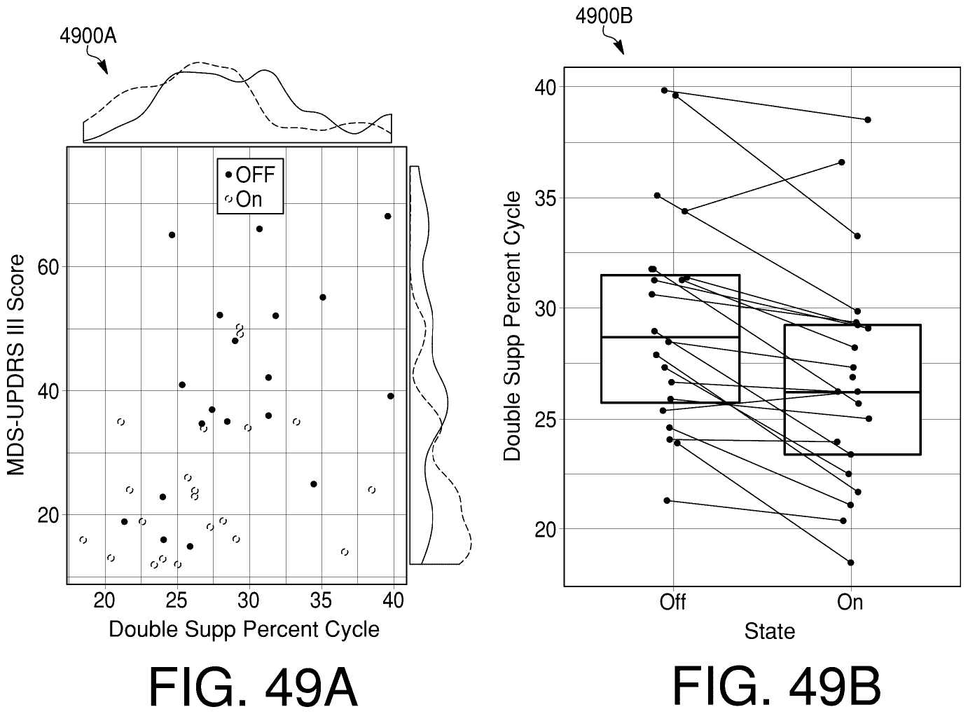

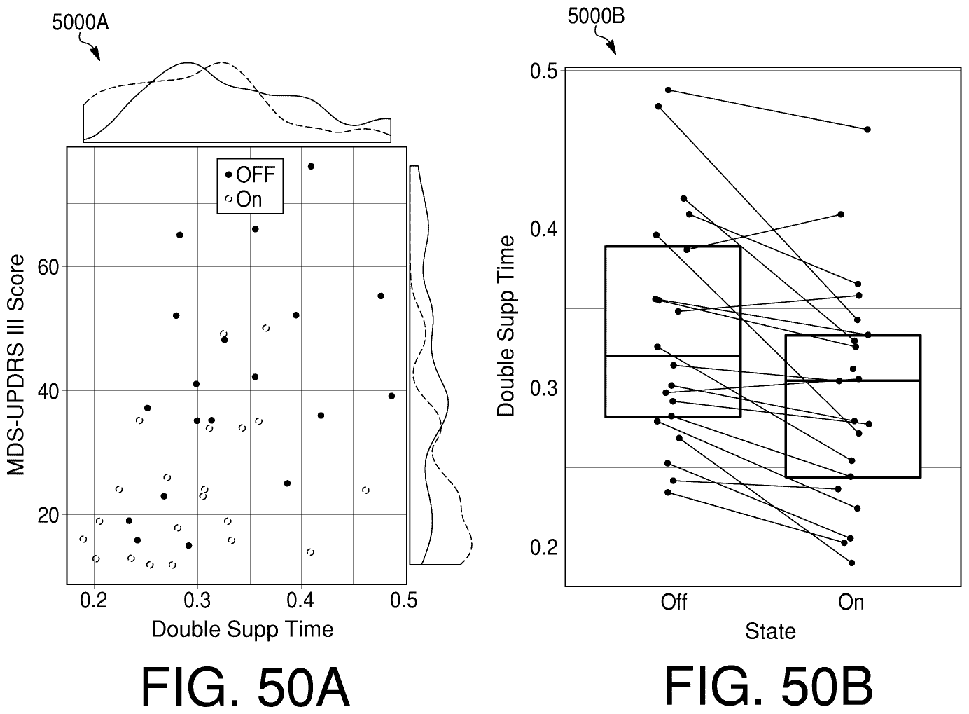

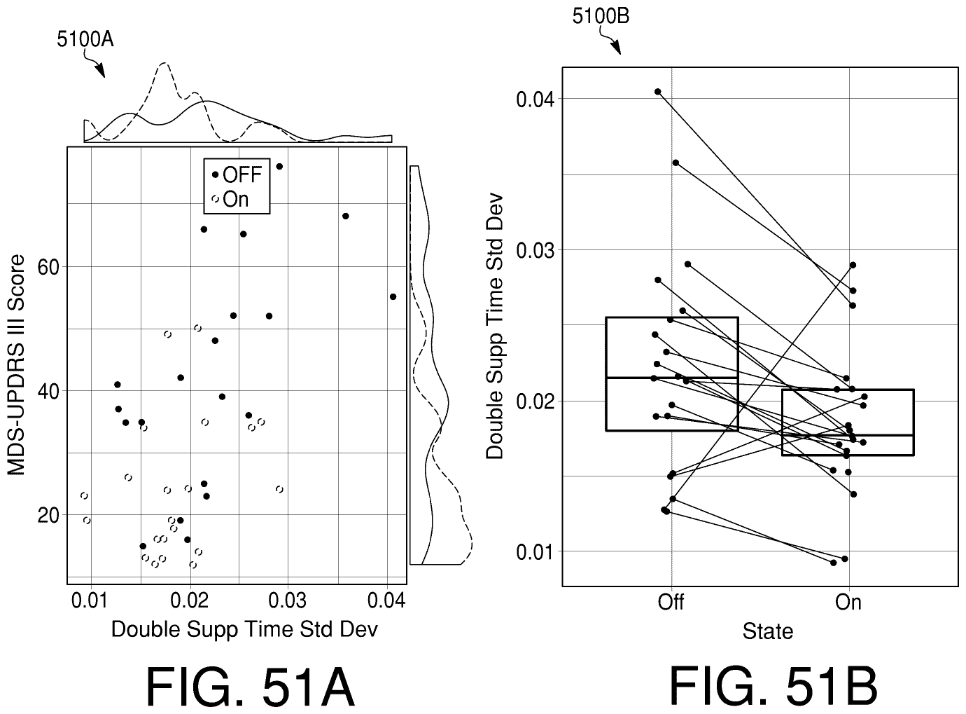

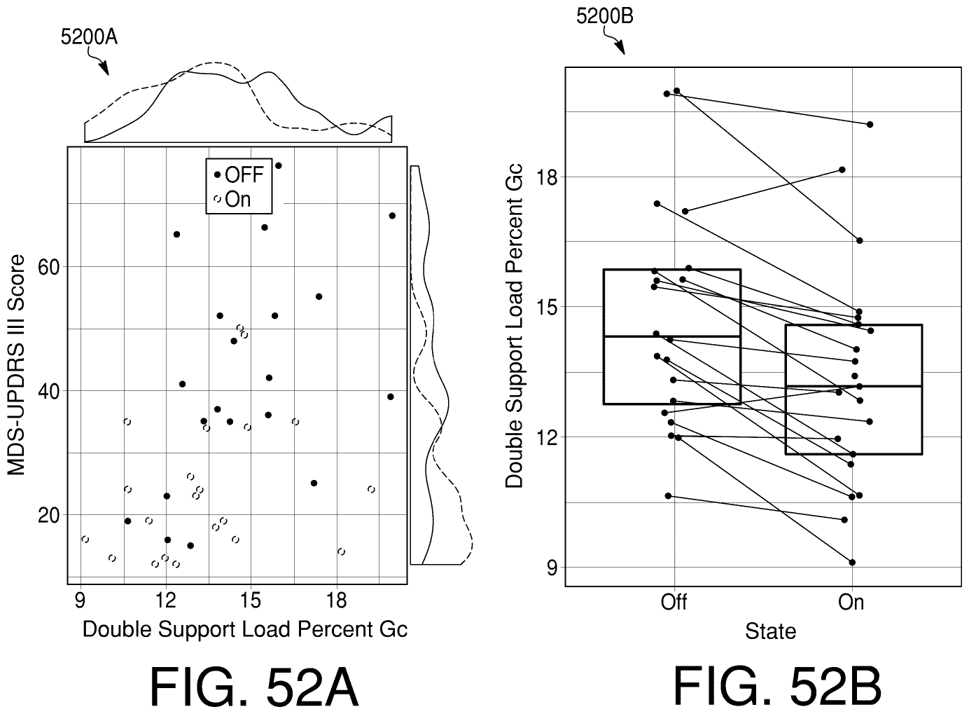

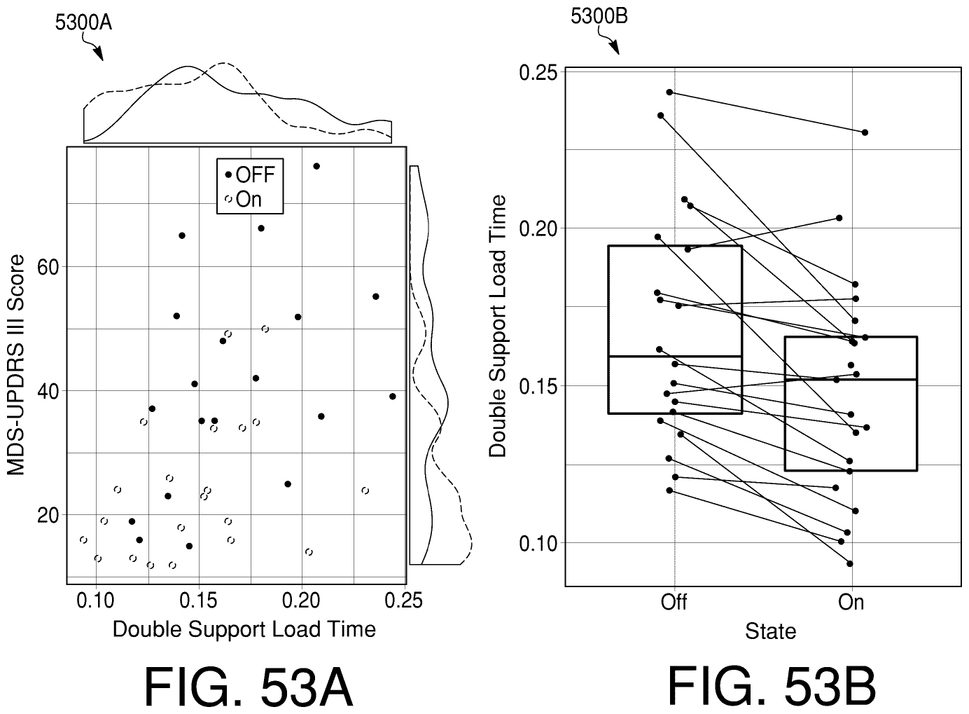

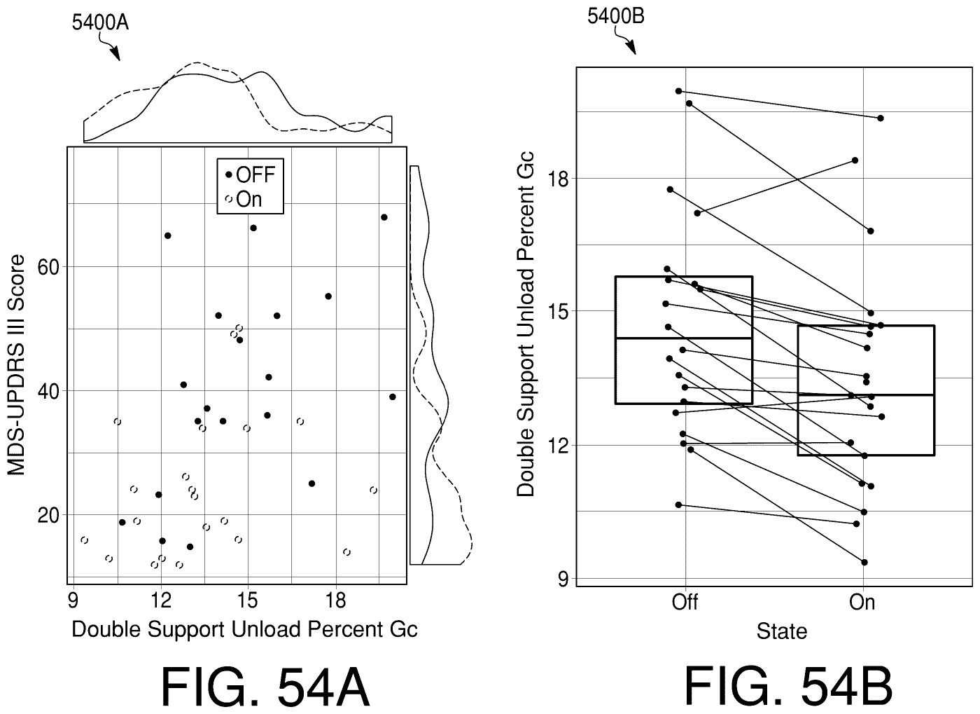

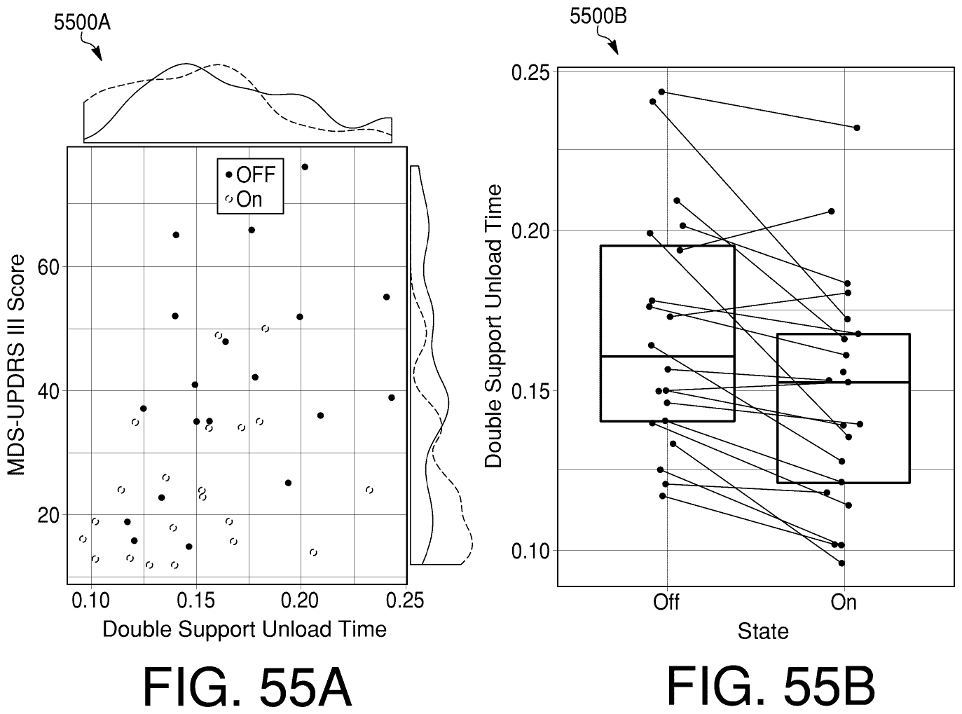

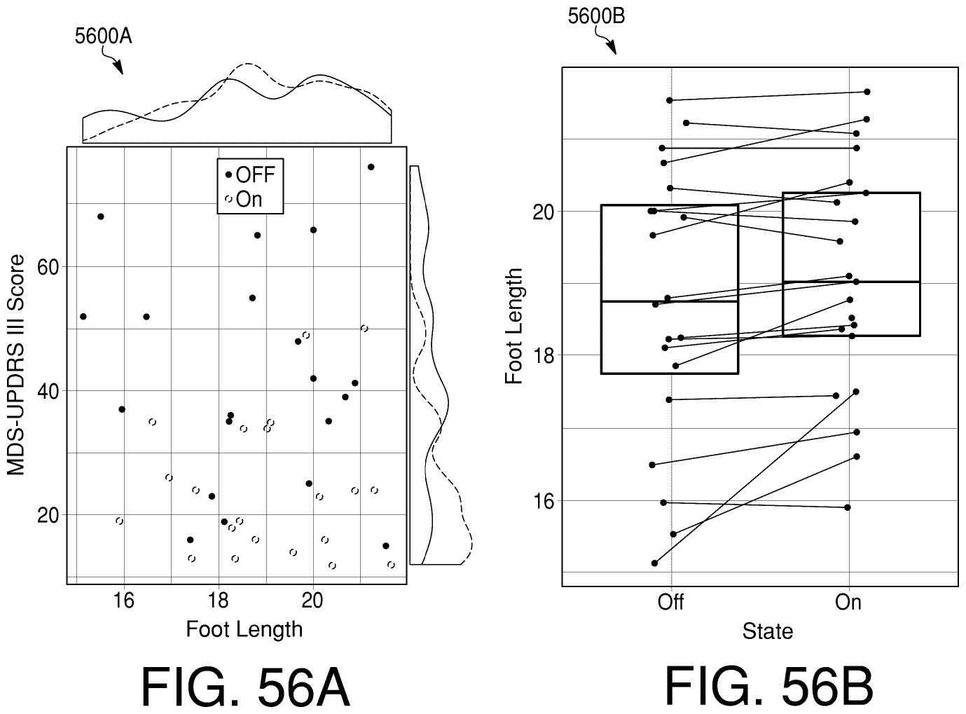

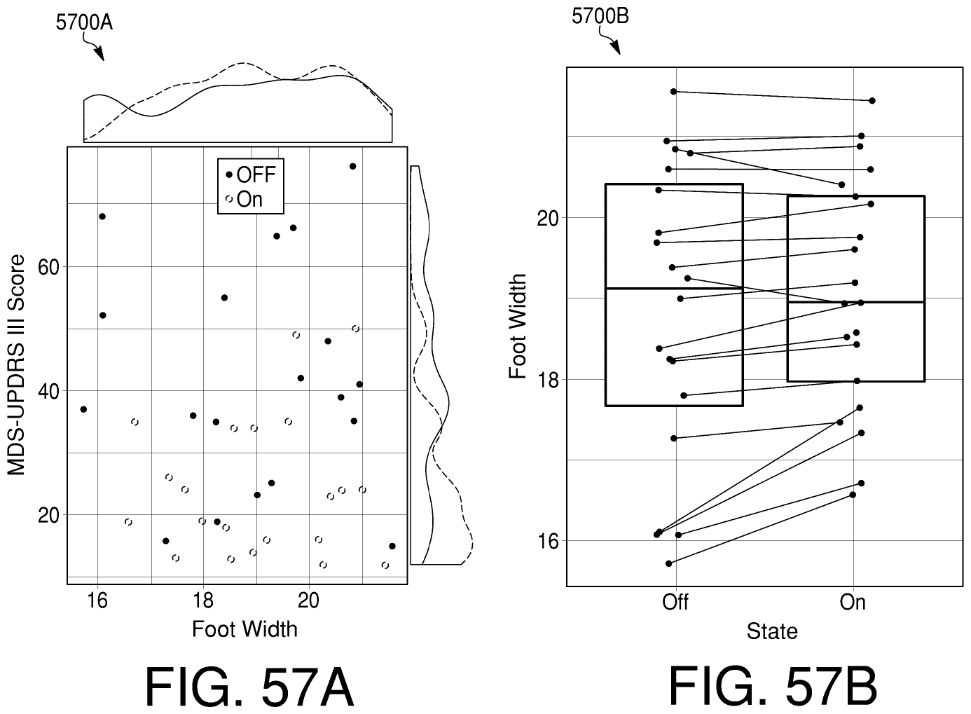

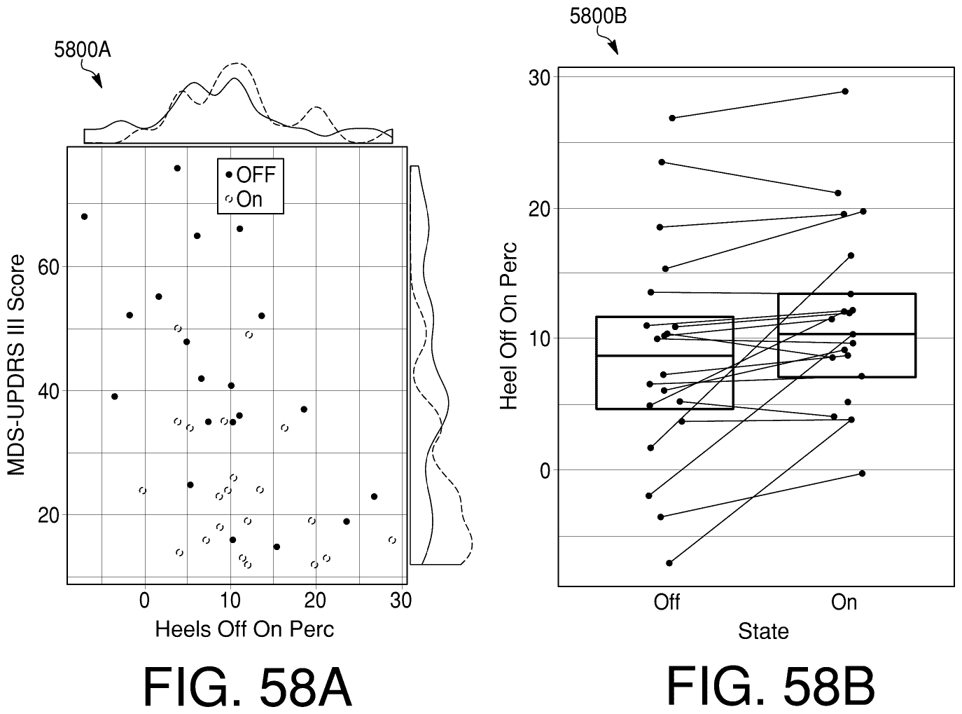

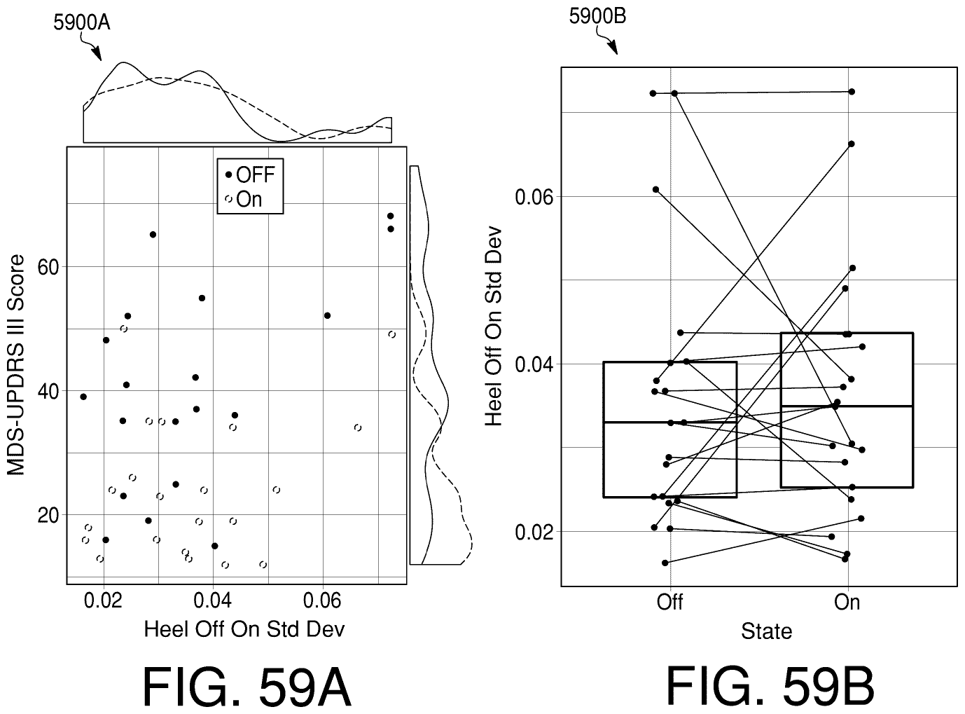

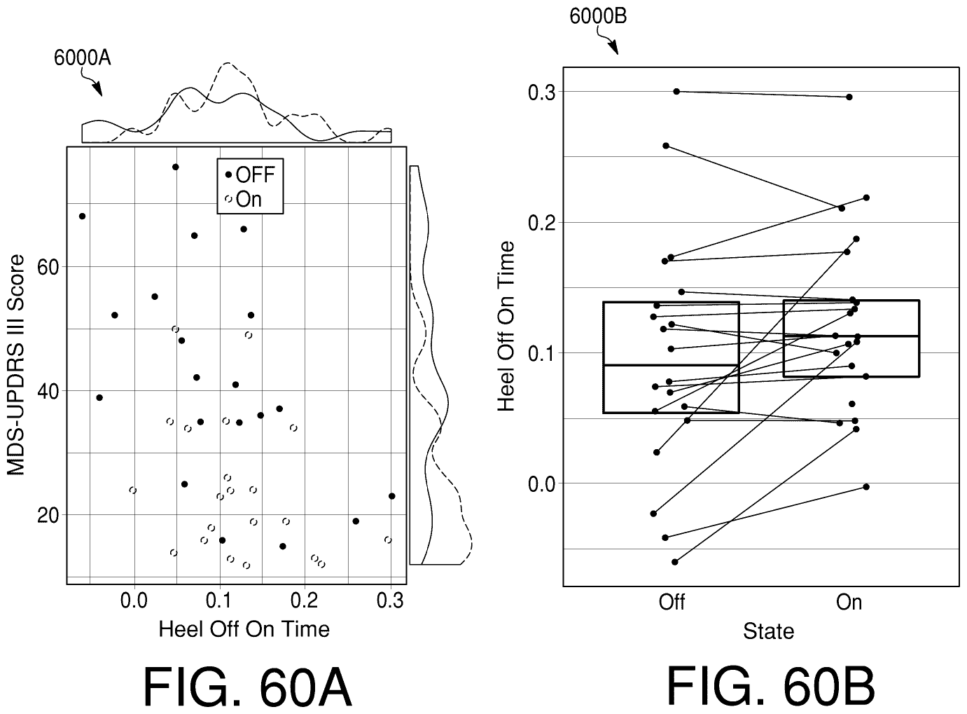

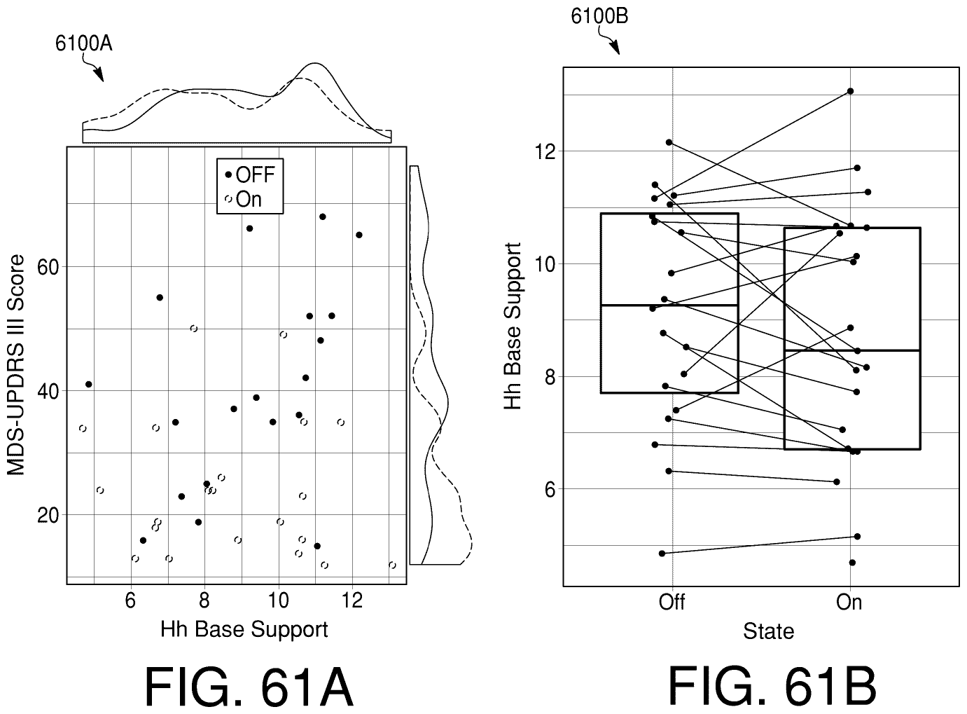

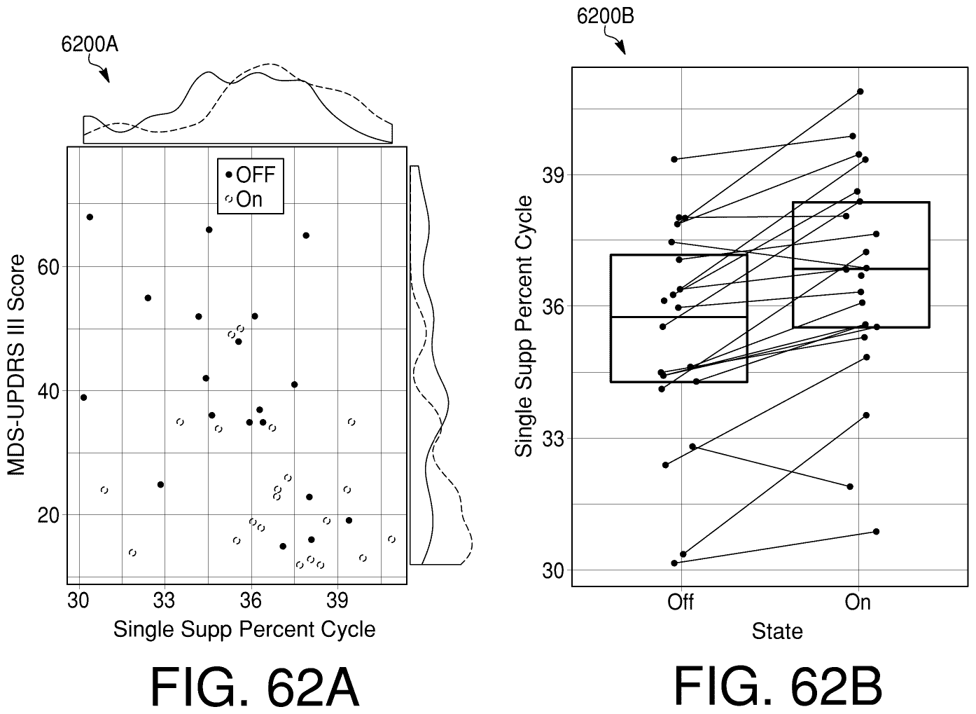

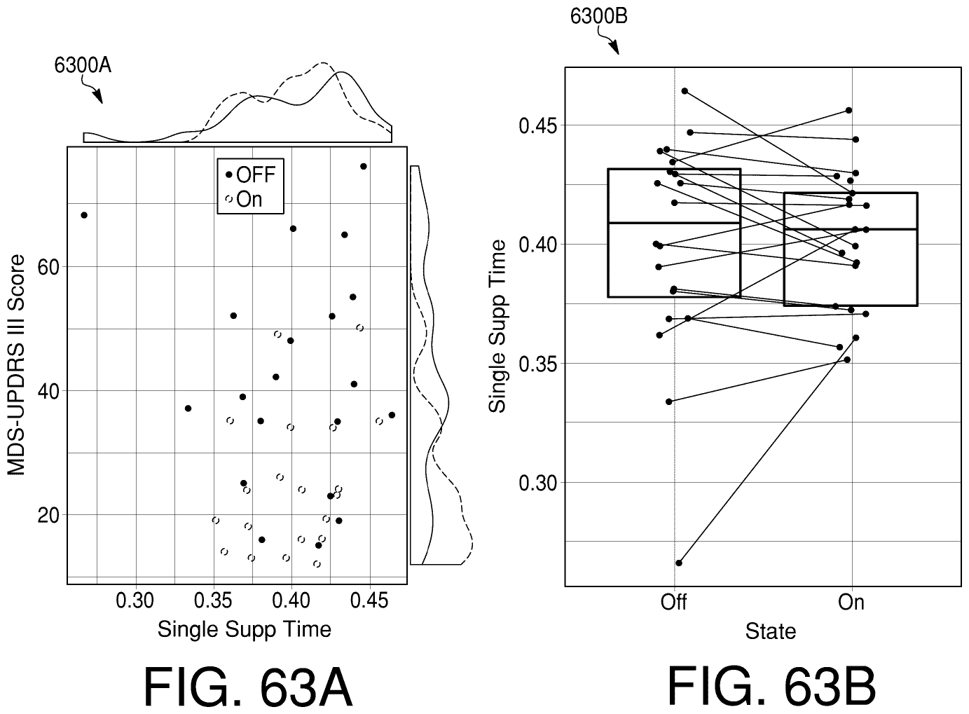

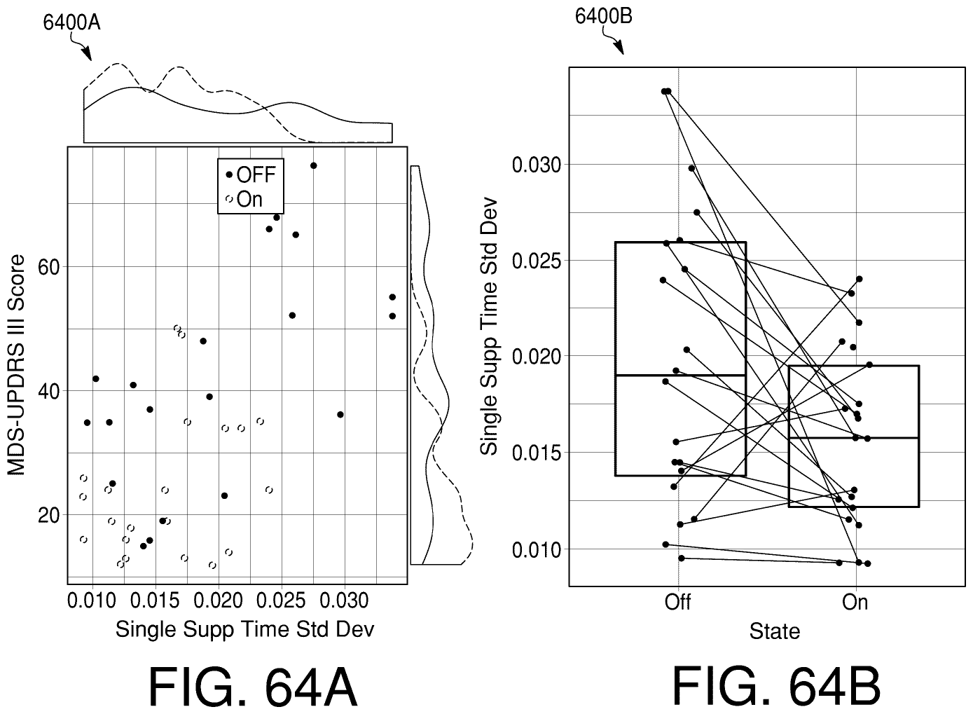

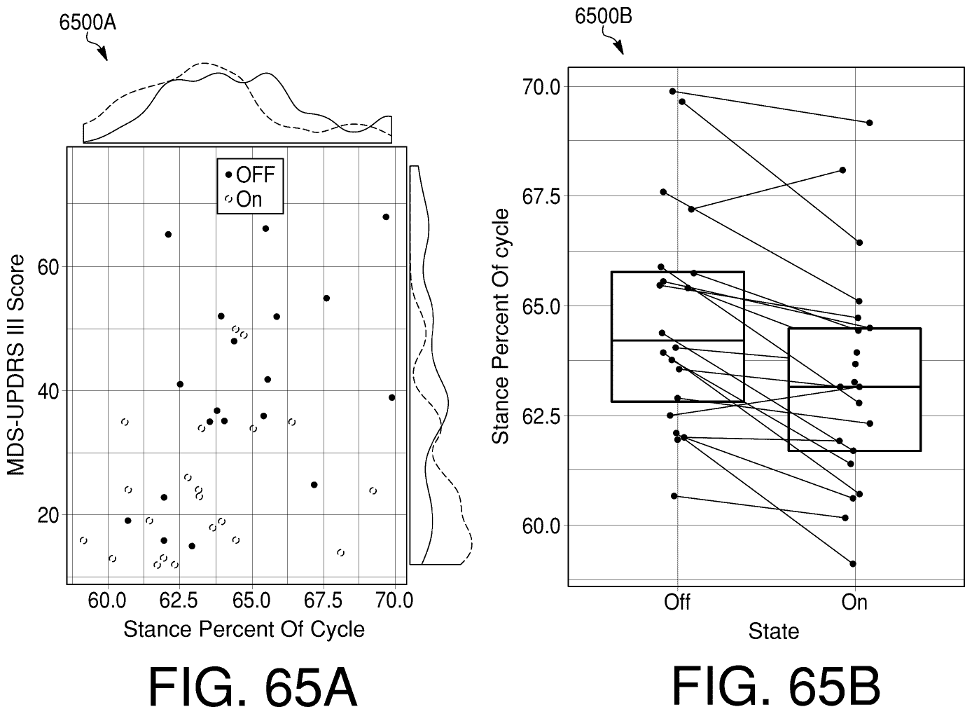

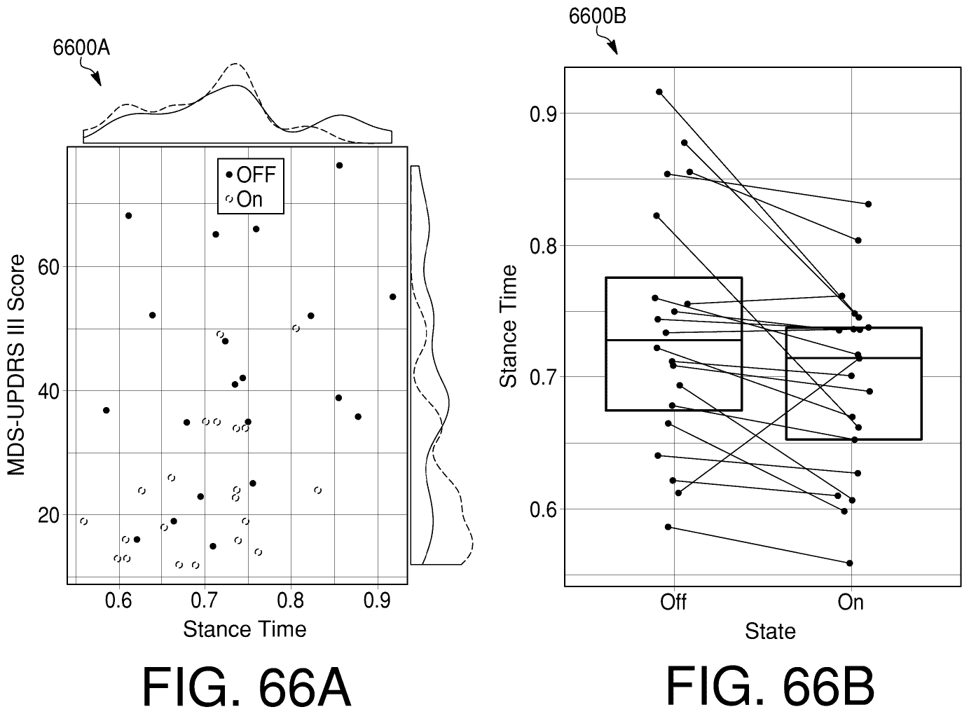

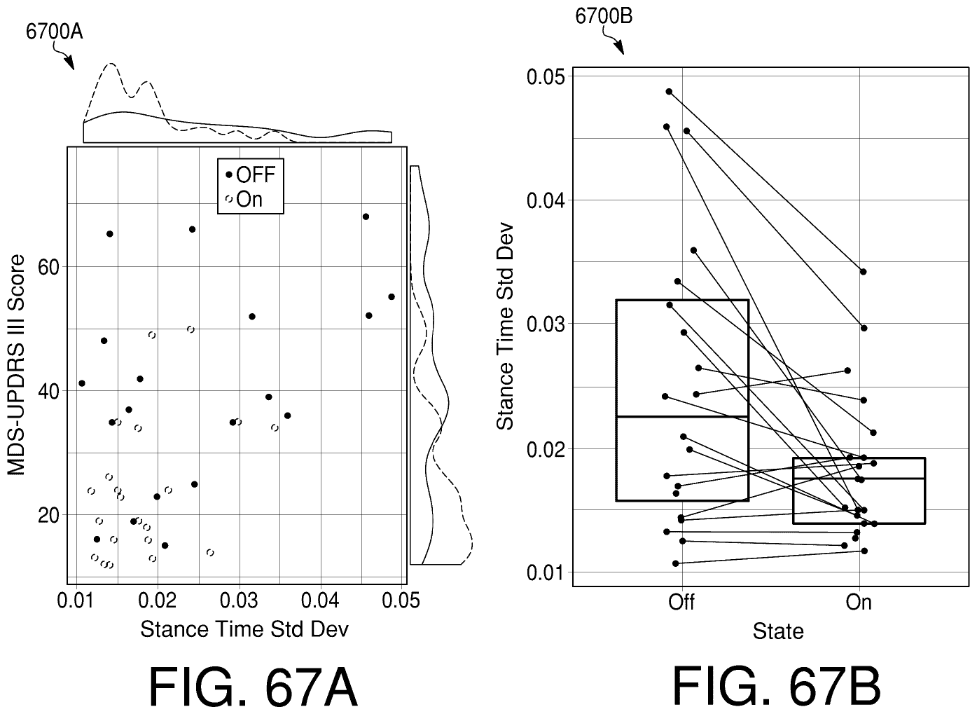

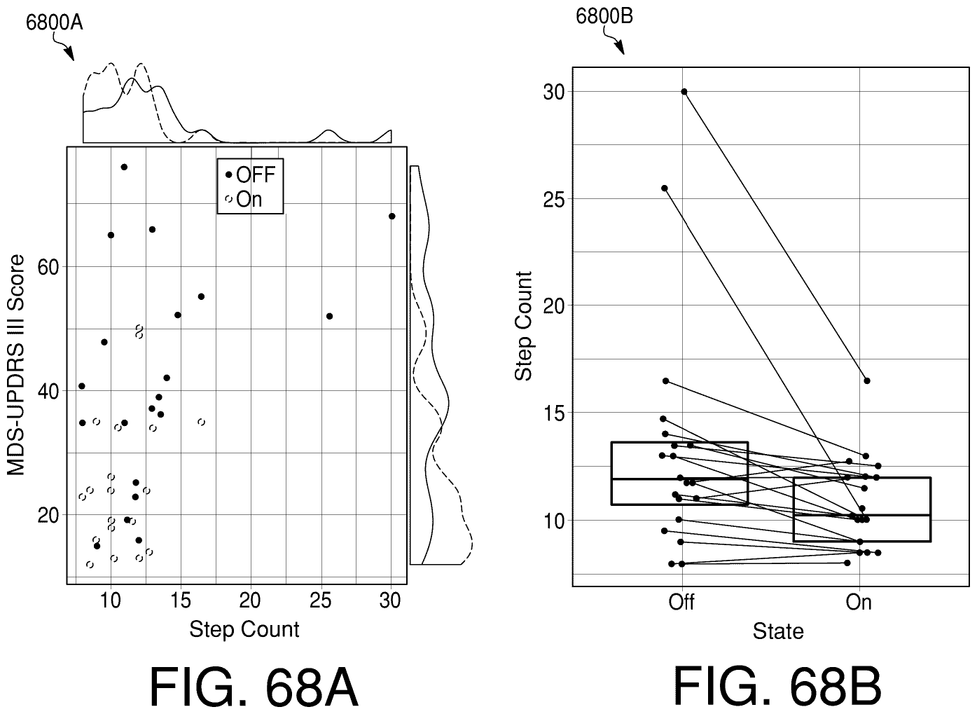

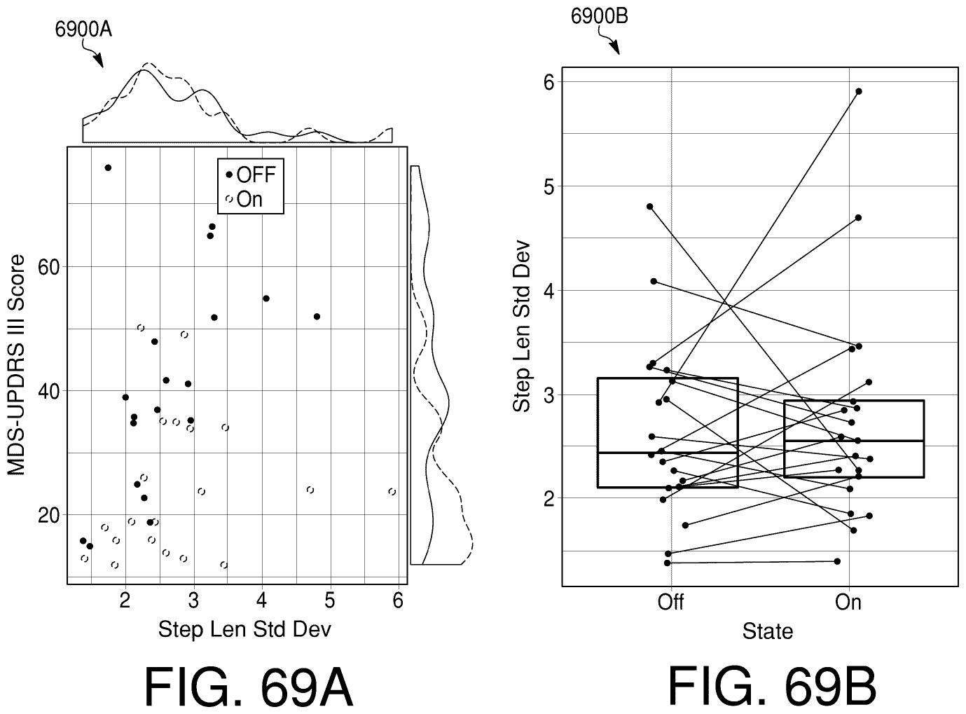

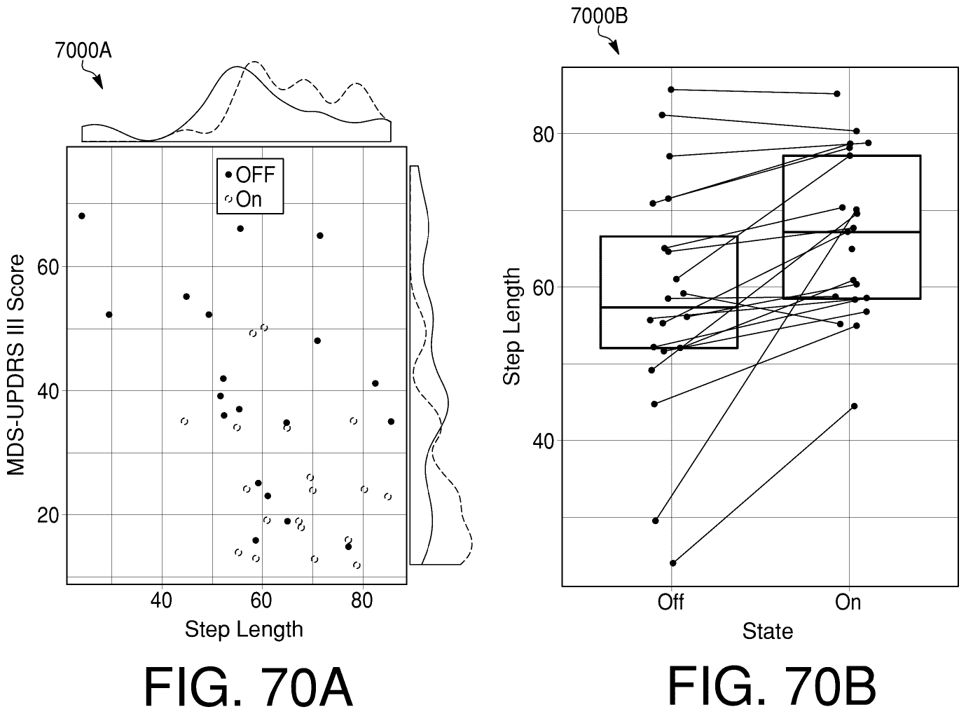

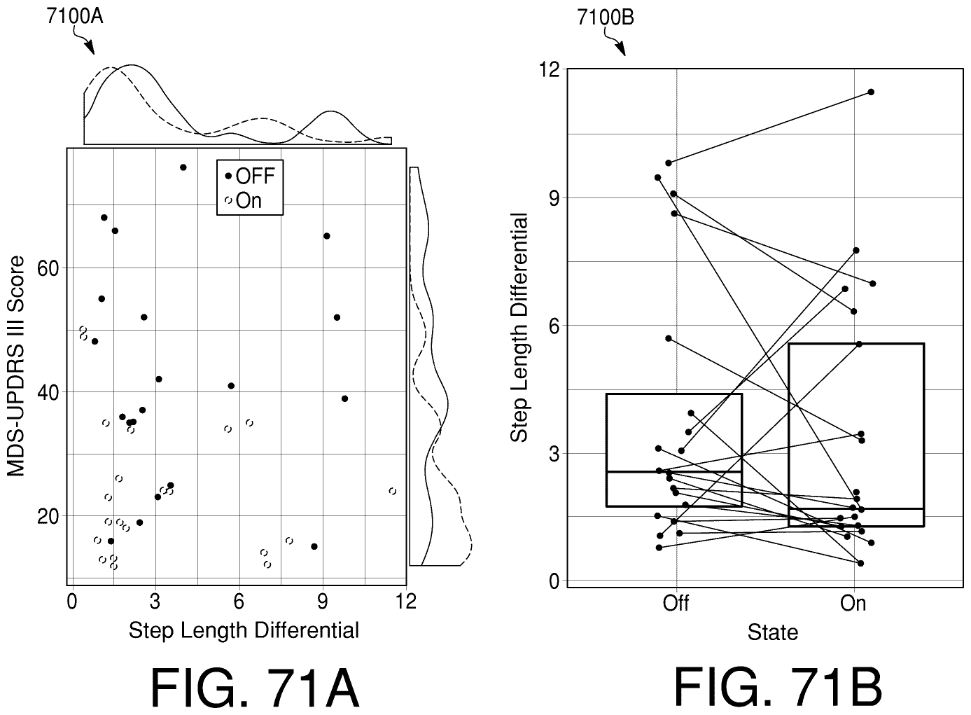

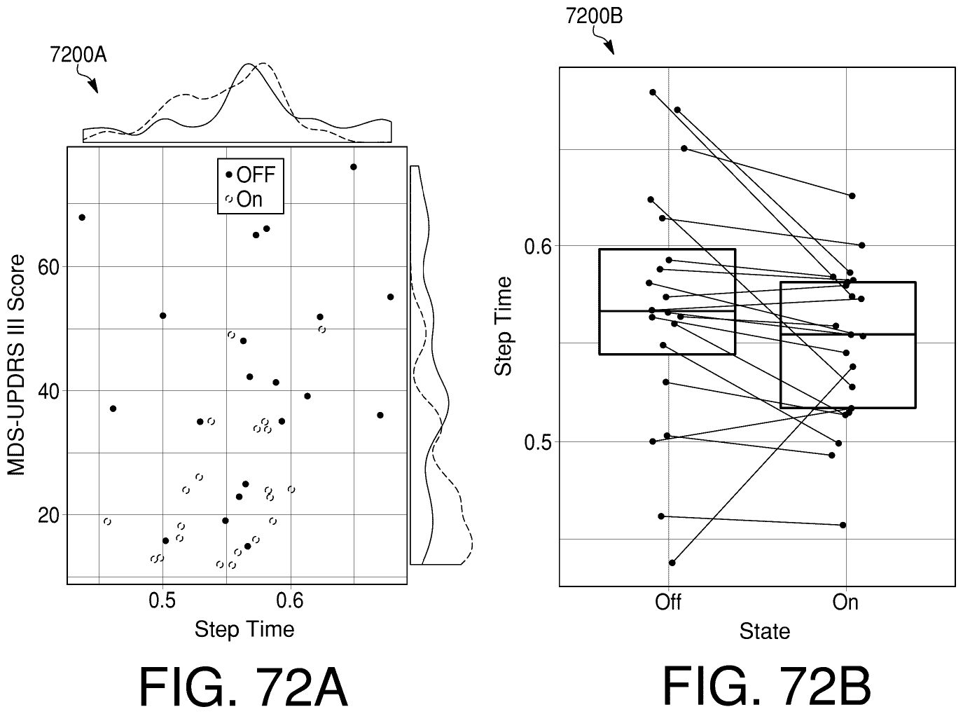

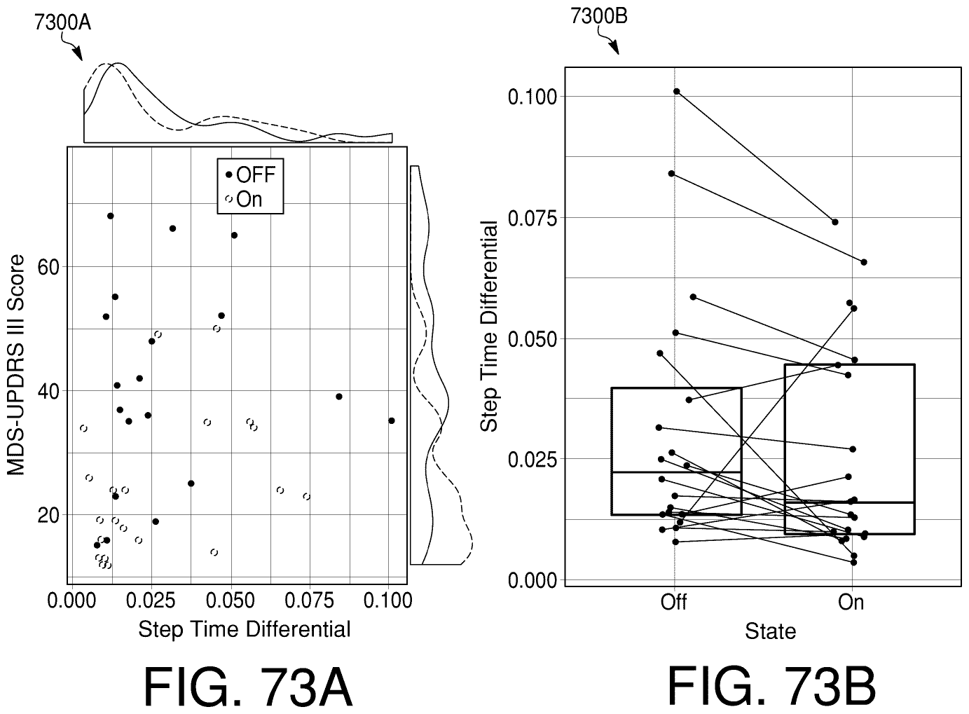

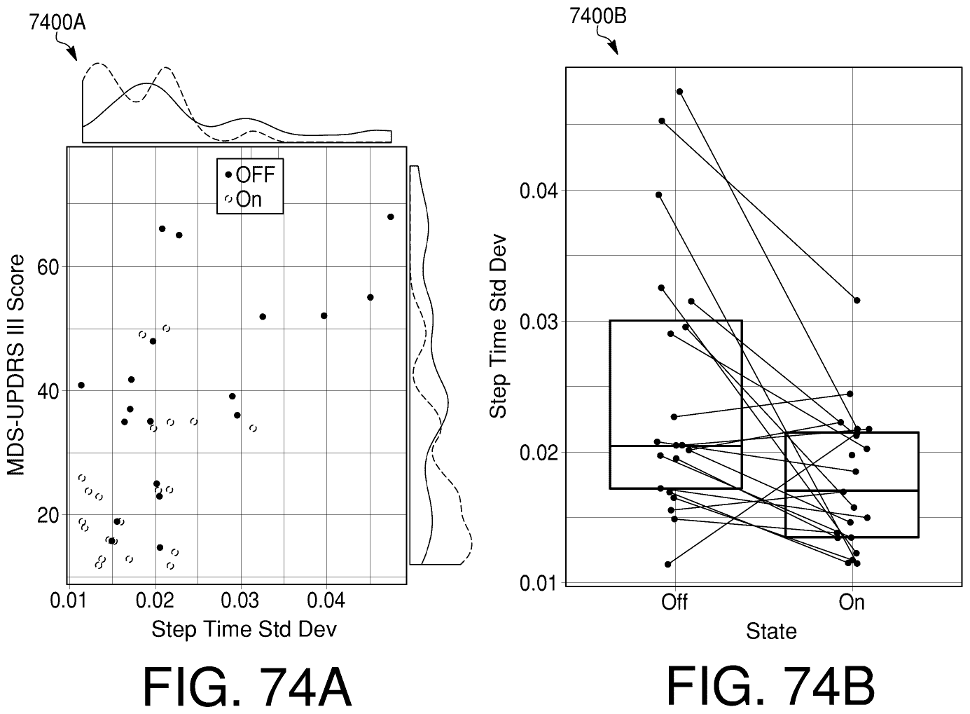

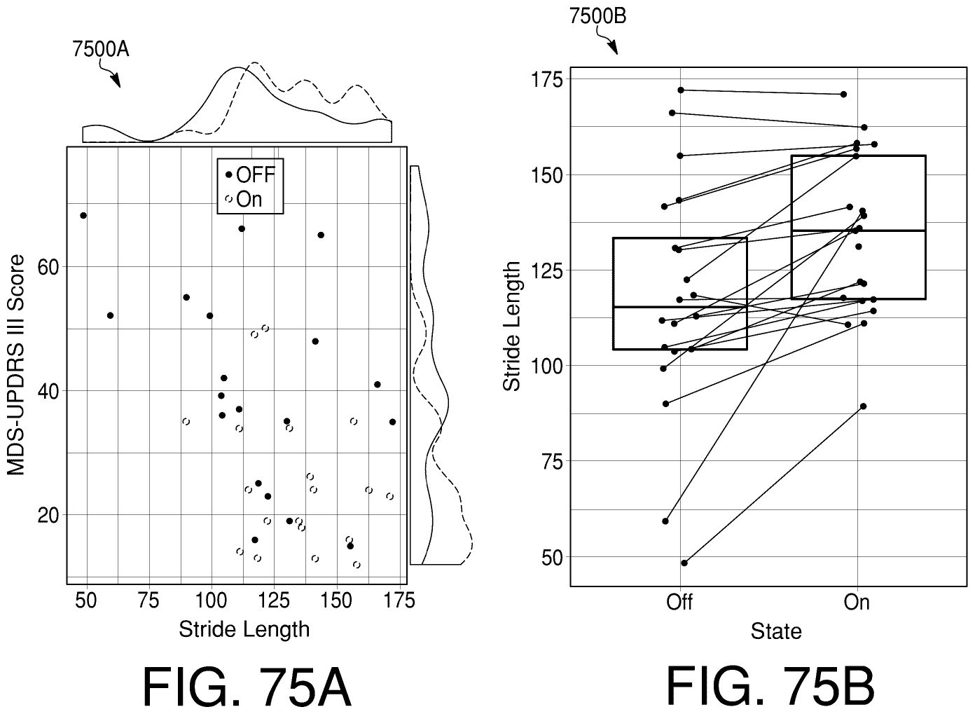

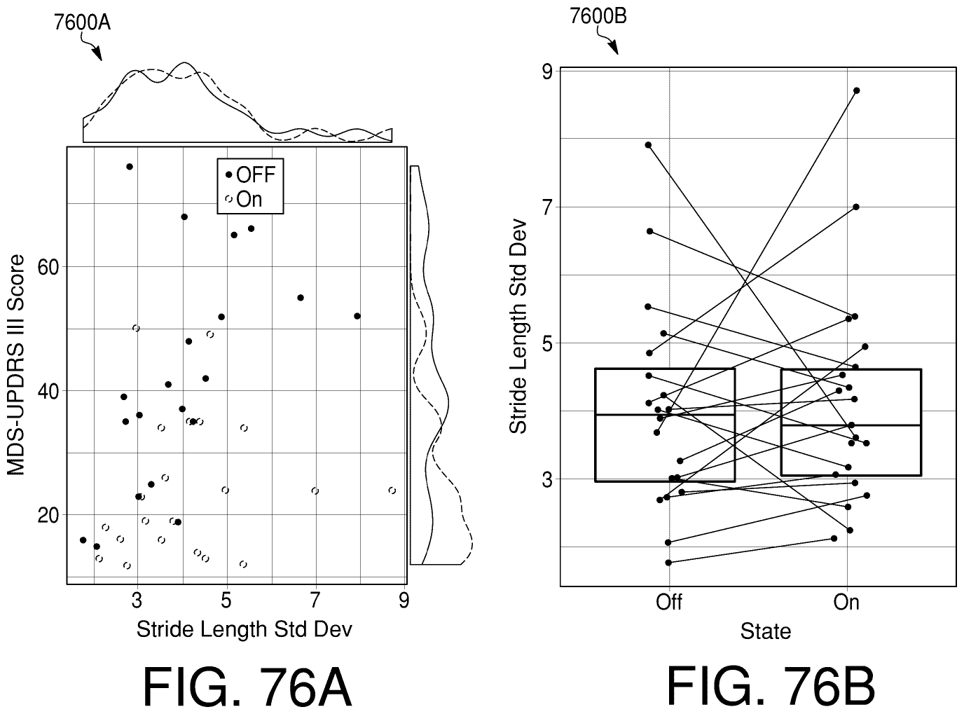

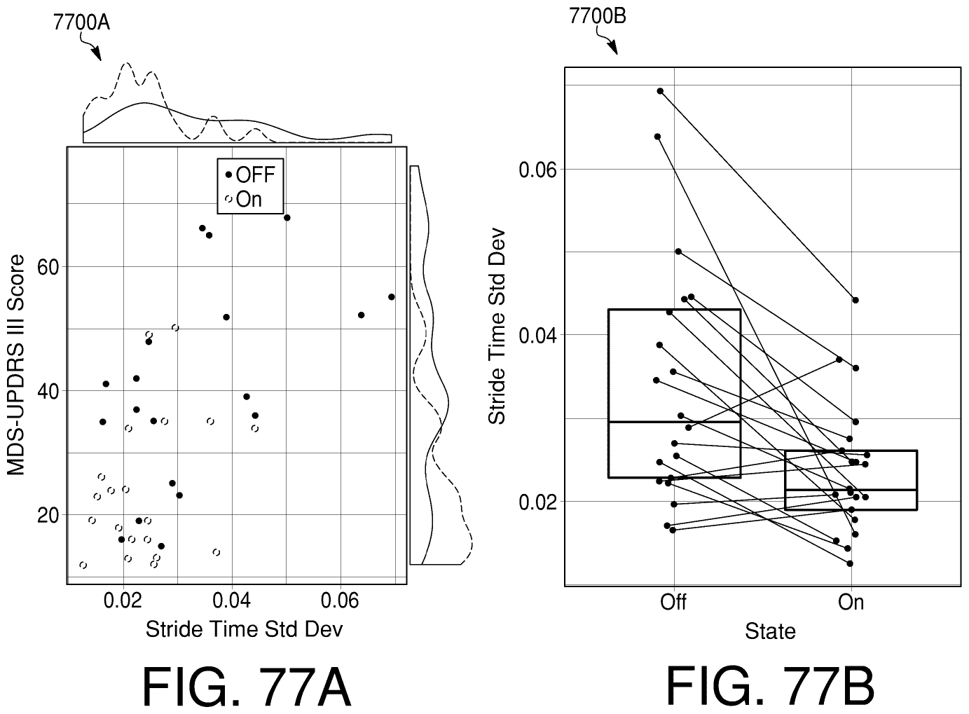

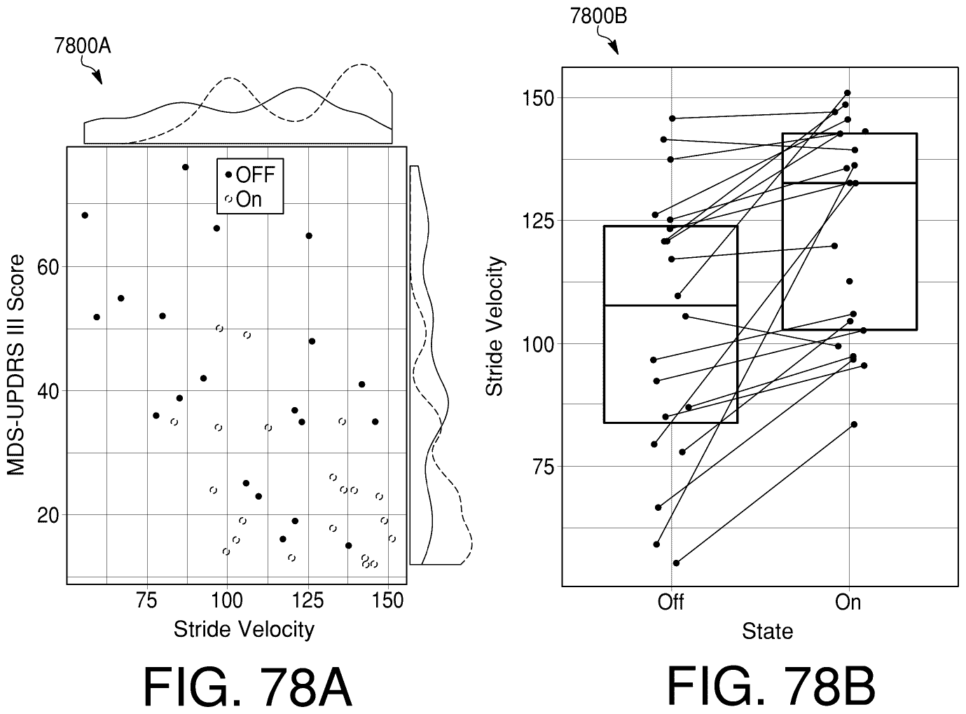

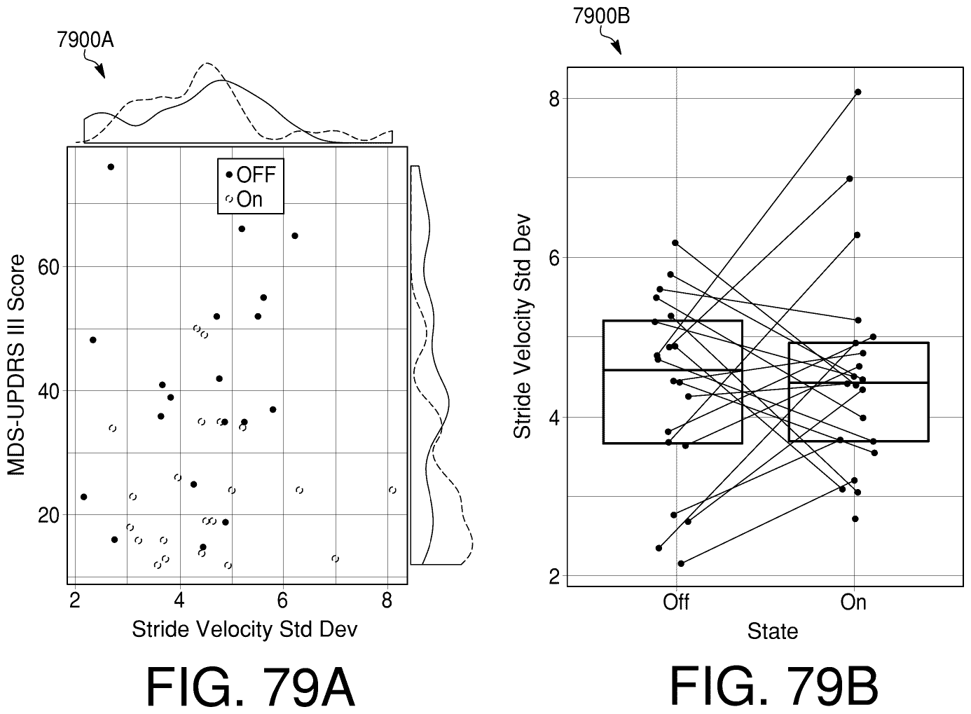

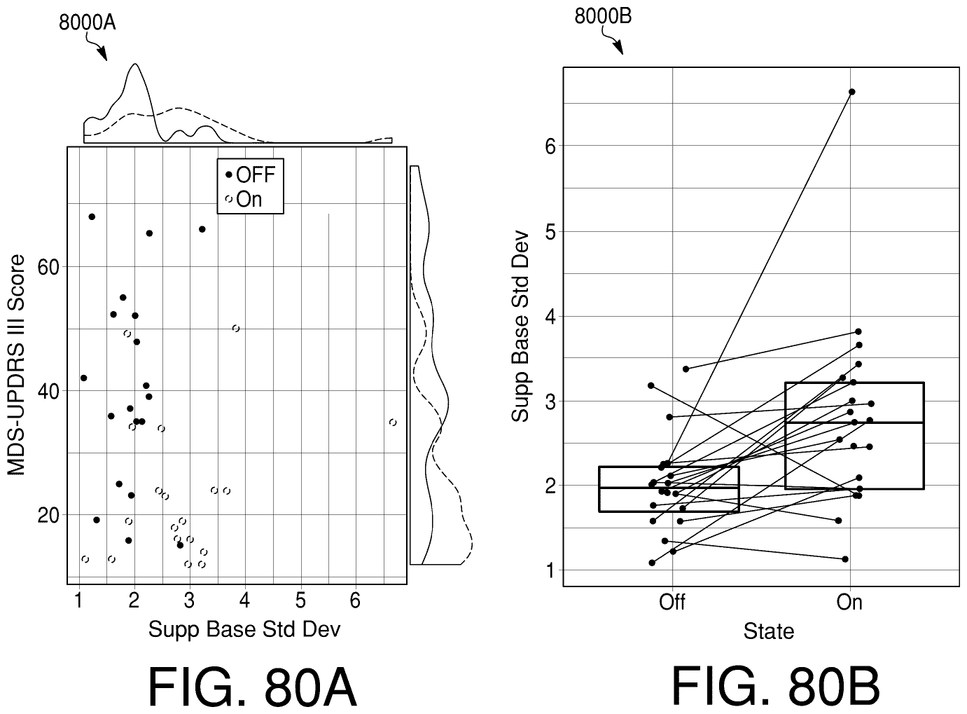

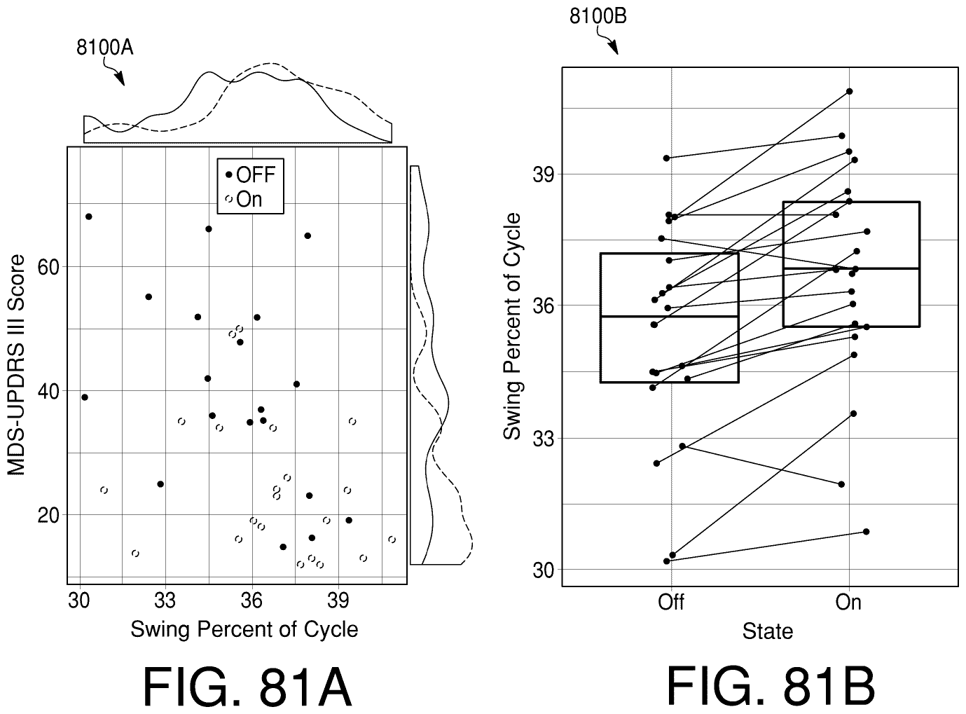

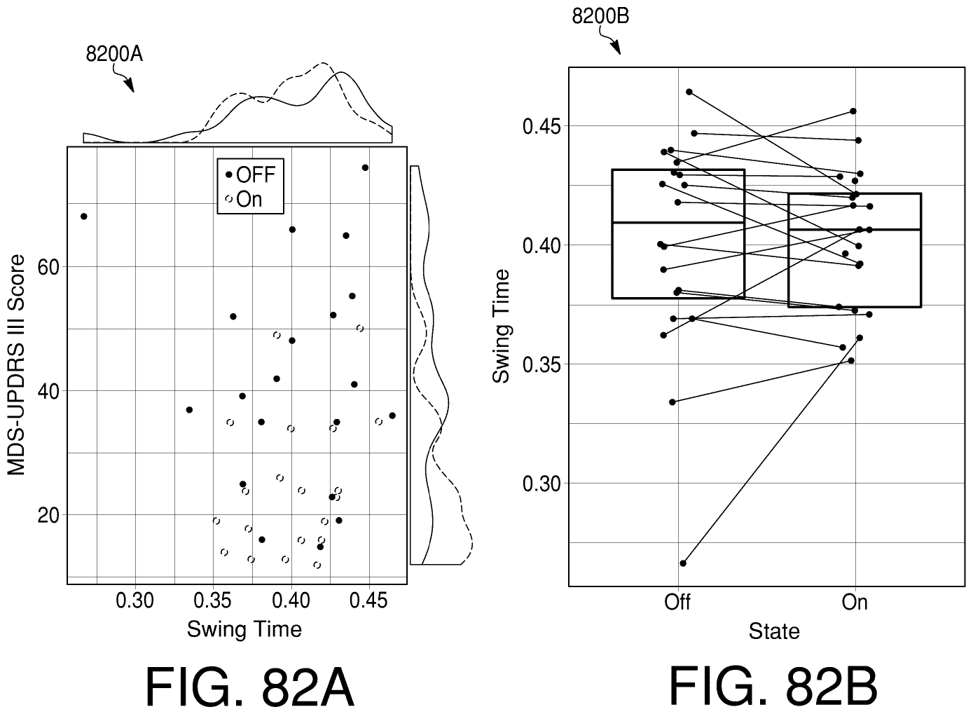

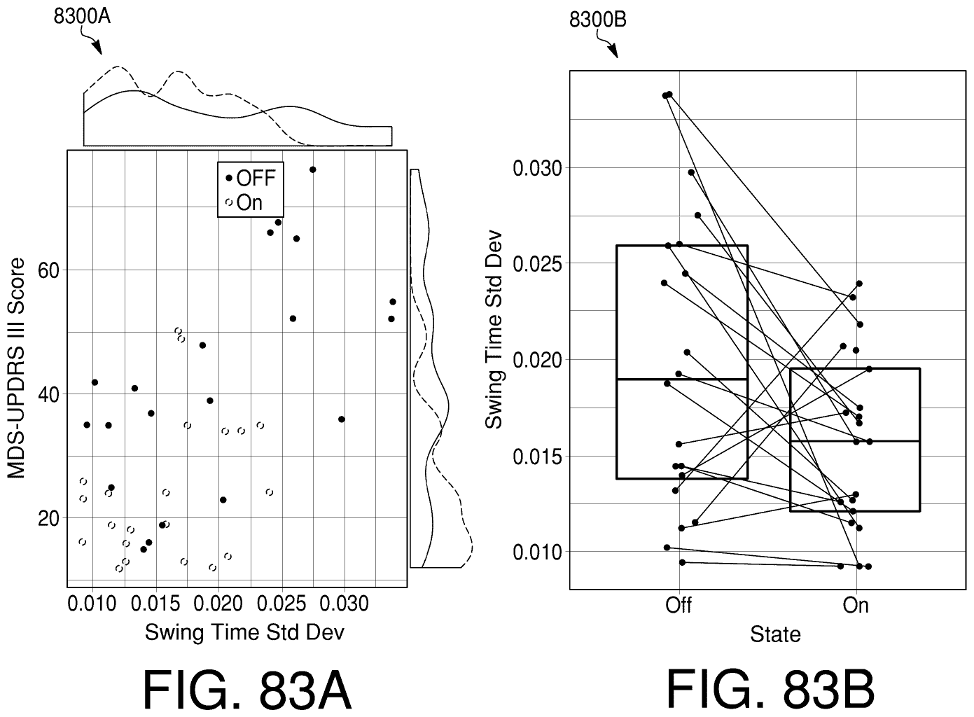

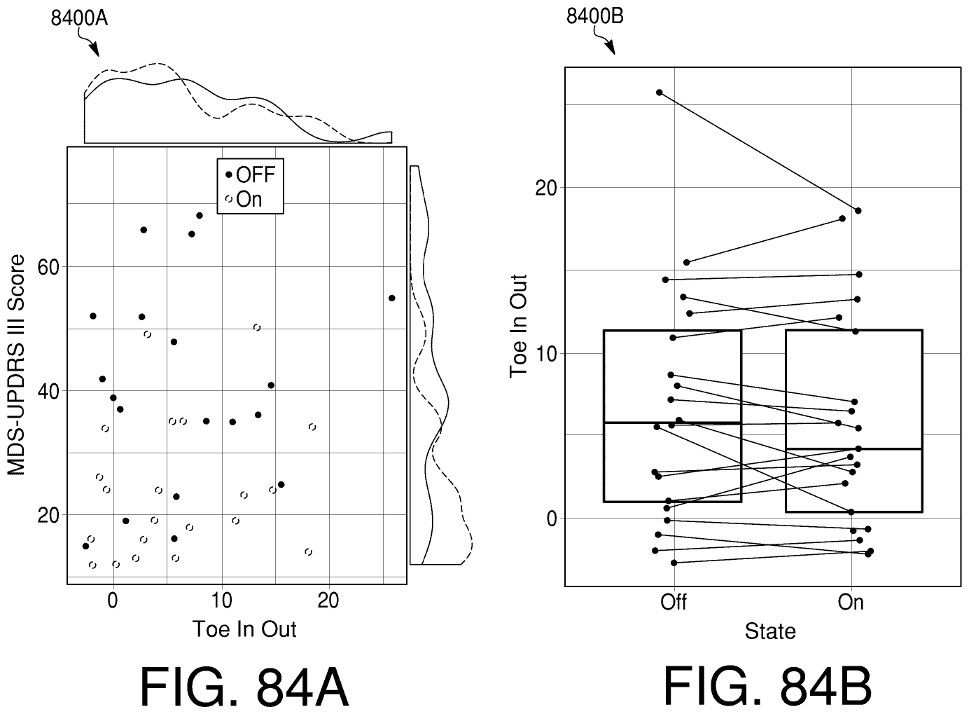

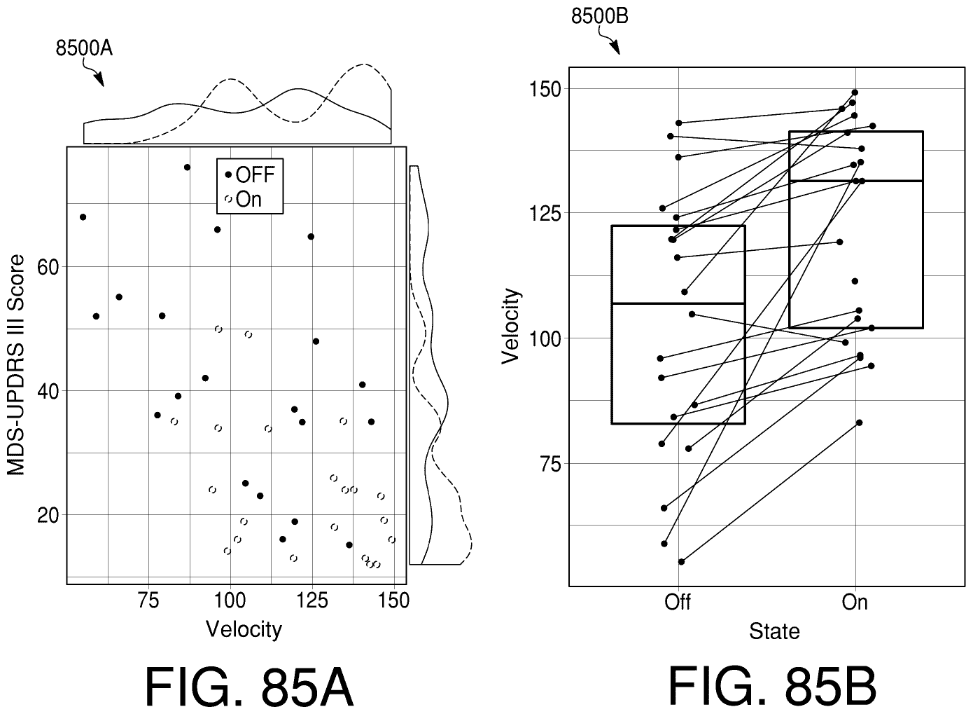

Pluralities of scatter plots between PD severity and each gait characteristic, along with differences between levodopa states, are provided for the production device in plots 4400A-8500B in FIGS. 44A-85B. More particularly, the gait characteristics represented by plots 4400A-8500B include: ambulation time (in plots 4400A and 4400B in FIGS. 44A and 44B), cadence (in plots 4500A and 4500B in FIGS. 45A and 45B), cycle time (in plots 4600A and 4600B in FIGS. 46A and 46B), cycle time differential (in plots 4700A and 4700B in FIGS. 47A and 47B), distance (in plots 4800A and 4800B in FIGS. 48A and 48B), double support percent cycle (in plots 4900A and 4900B in FIGS. 49A and 49B), double support time (in plots 5000A and 5000B in FIGS. 50A and 50B), double support time standard deviation (in plots 5100A and 5100B in FIGS. 51A and 51B), double support load percent GC (in plots 5200A and 5200B in FIGS. 52A and 52B), double support load time (in plots 5300A and 5300B in FIGS. 53A and 53B), double support unload percent GC (in plots 5400A and 5400B in FIGS. 54A and 54B), double support unload time (in plots 5500A and 5500B in FIGS. 55A and 55B), foot length (in plots 5600A and 5600B in FIGS. 56A and 56B), foot width (in plots 5700A and 5700B in FIGS. 57A and 57B), heel off on percent (in plots 5800A and 5800B in FIGS. 58A and 58B), heel off on standard deviation (in plots 5900A and 5900B in FIGS. 59A and 59B), heel off on time (in plots 6000A and 6000B in FIGS. 60A and 60B), HH base support (in plots 6100A and 6100B in FIGS. 61A and 61B), single support percent cycle (in plots 6200A and 6200B in FIGS. 62A and 62B), single support time (in plots 6300A and 6300B in FIGS. 63A and 63B), single support time standard deviation (in plots 6400A and 6400B in FIGS. 64A and 64B), stance percent of cycle (in plots 6500A and 6500B in FIGS. 65A and 65B), stance time (in plots 6600A and 6600B in FIGS. 66A and 66B), stance time standard deviation (in plots 6700A and 6700B in FIGS. 67A and 67B), step count (in plots 6800A and 6800B in FIGS. 68A and 68B), step length standard deviation (in plots 6900A and 6900B in FIGS. 69A and 69B), step length (in plots 7000A and 7000B in FIGS. 70A and 70B), step length differential (in plots 7100A and 7100B in FIGS. 71A and 71B), step time (in plots 7200A and 7200B in FIGS. 72A and 72B), step time differential (in plots 7300A and 7300B in FIGS. 73A and 73B), step time standard deviation (in plots 7400A and 7400B in FIGS. 74A and 74B), stride length (in plots 7500A and 7500B in FIGS. 75A and 75B), stride length standard deviation (in plots 7600A and 7600B in FIGS. 76A and 76B), stride time standard deviation (in plots 7700A and 7700B in FIGS. 77A and 77B), stride velocity (in plots 7800A and 7800B in FIGS. 78A and 78B), stride velocity standard deviation (in plots 7900A and 7900B in FIGS. 79A and 79B), support base standard deviation (in plots 8000A and 8000B in FIGS. 80A and 80B), swing percent of cycle (in plots 8100A and 8100B in FIGS. 81A and 81B), swing time (in plots 8200A and 8200B in FIGS. 82A and 82B), swing time standard deviation (in plots 8300A and 8300B in FIGS. 83A and 83B), toe in out (in plots 8400A and 8400B in FIGS. 84A and 84B), and velocity (in plots 8500A and 8500B in FIGS. 85A and 85B).

### MDS-UPDRS III Score and Speech Characteristics

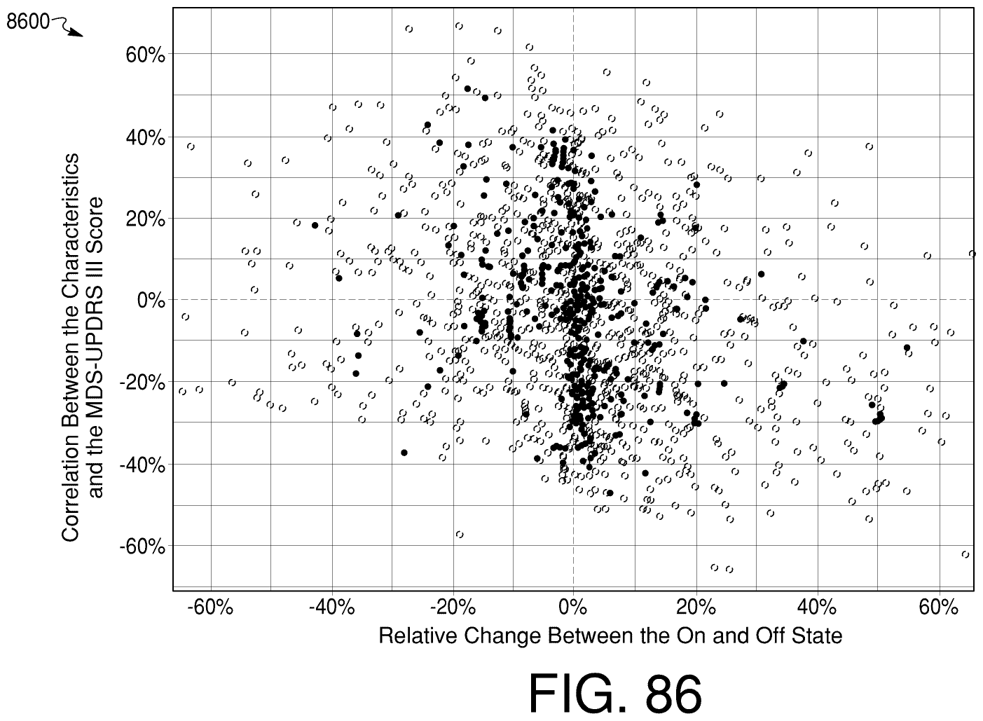

An analysis was conducted examining the relationship between speech characteristics and clinical measures of PD's severity, specifically the MDS-UPDRS III scores, and the impact of levodopa treatment. FIG. 86 presents scatter plot 8600 that provides a visual representation of both the strength of association with disease severity and the sensitivity to levodopa treatment. The y-axis contains the Spearman correlation between each speech characteristic and the MDS-UPDRS III score. Each point's position on the x-axis represents the percent difference in the speech characteristic between the ON and the OFF state. Compared to gait characteristics, the speech characteristics exhibit more variability in their correlation with the MDS-UPDRS III score and their percent change between ON and OFF states. This suggests that speech features might be more heterogeneous in how they reflect disease severity and respond to medication.

### Clinical Validation

A goal of the validation process is to answer two questions, (i) which gait/speech characteristics associate with the MDS-UPDRS III score; and (ii) which gait/speech characteristics have substantial changes between the ON and OFF levodopa state?

I. Gait Characteristics that Associate with the MDS-UPDRS III Score

In an aspect, a mixed effects model is employed to analyze the relationship between gait characteristics measured by the wearable insole and the MDS-UPDRS III score. Given that:

Yi|γi˜Poisson(μij)

In which Yij is the jth MDS-UPDRS III score for the ith subject, the following equation for the model is provided:

μij=exp{β0+γ0i+β1Xijk+β2I{Stateij=On}+β3I{Sitei=RUSH}}

In this equation, γi0˜Normal(0,σγ2). Additionally, in the model, XijkXijk is the value of the kkth gait characteristic for the jth observation of the ith subject. That is, the model above is applied to each gait characteristic measured by the wearable insole test device.

The direct association of the levodopa state ranges from a decrease in MDS-UPDRS III score between 40% to 46%, whereas the direct association of recruitment site ranges from a decrease of 52% to 56%. Out of 30 wearable insole test device gait characteristics, 5 have a nominally significant association with the MDS-UPDRS III score and most of these were negative associations.

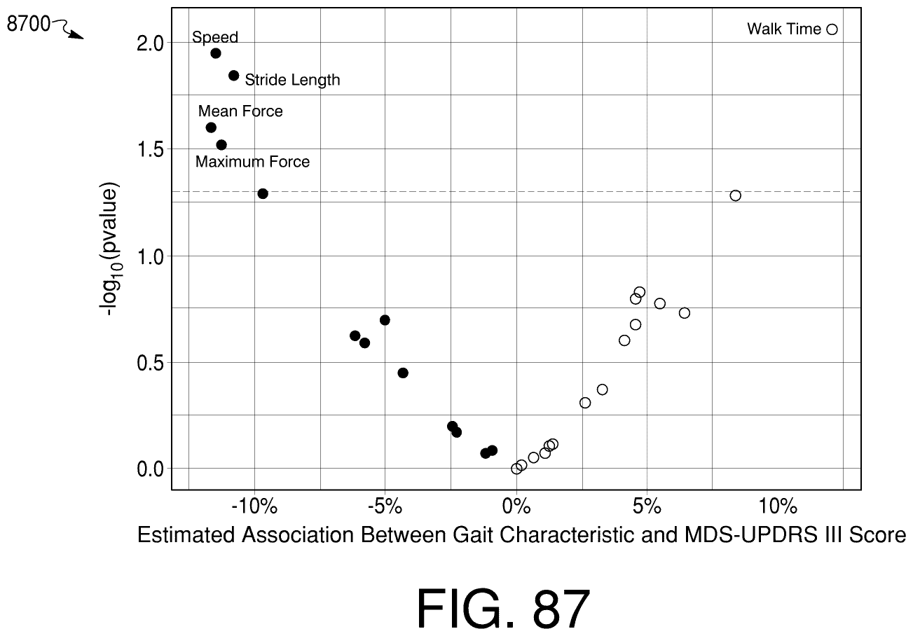

FIG. 87 provides volcano plot 8700 of wearable insole test device gait characteristic associations with MDS-UPDRS III scores. The x-axis represents the percent change in the MDS-UPDRS III score for a one standard deviation increase in the gait characteristic. The y-axis represents the p-value for the association between the gait characteristic and the MDS-UPDRS III score. The filled circles indicate negative associations (higher gait characteristic values associated with lower MDS-UPDRS III scores) whereas the empty circle indicate positive associations (higher gait characteristic values associated with higher MDS-UPDRS III scores). The horizontal dashed-gray line represents the p-value threshold for significant (0.05). Points above this line are considered nominally significant and gait characteristics with nominally significant associations are labeled for emphasis. An examination of this data reveals that the significant reduction in MDS-UPDRS III scores due to the ON state underscores the effectiveness of levodopa in mitigating PD's symptoms.

Table 3, from page 3 of Appendix A of U.S. Provisional Patent Application No. 63/695,789, which is incorporated by reference herein, shows the estimated associations and corresponding p-values of all gait characteristics measured by the wearable insole test device.

The same model described above may be implemented for the production device. Here, the direct association of the levodopa state ranges from a decrease in MDS-UPDRS III score between 39% to 44%, whereas the direct association of recruitment site ranges from a decrease of 49% to 55%. These are similar to the estimates found with the data derived by the wearable insole test device. Out of 42 gait characteristics measured by the production device, 9 have a nominally significant association with the MDS-UPDRS III score.

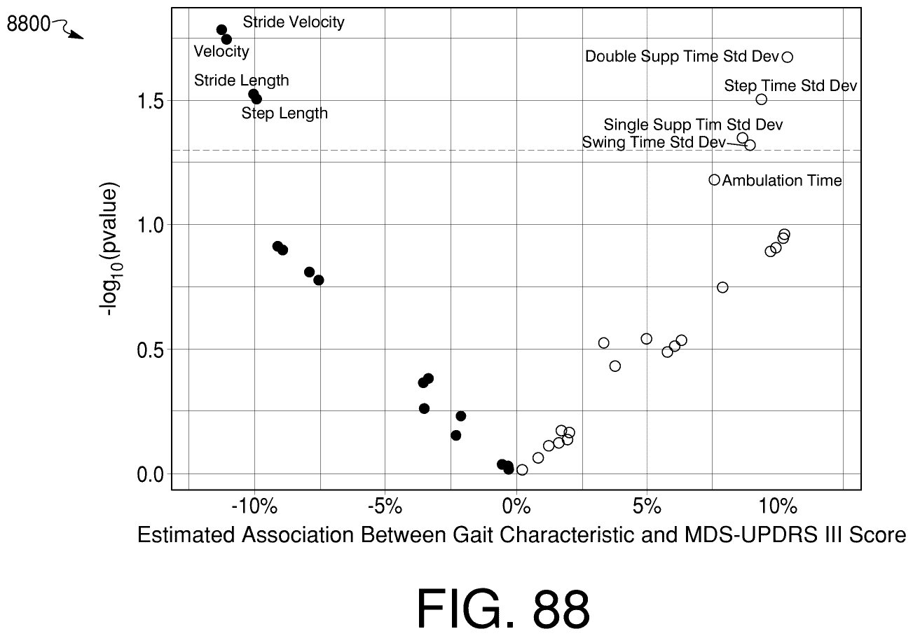

FIG. 88 provides volcano plot 8800 of production device gait characteristic associations with MDS-UPDRS III scores. The x-axis represents the percent change in the MDS-UPDRS III score for a one standard deviation increase in the gait characteristic. The y-axis represents the p-value for the association between the gait characteristic and the MDS-UPDRS III score. The solid circles indicate negative associations (higher gait characteristic values associated with lower MDS-UPDRS III scores) whereas the empty circles indicate positive associations (higher gait characteristic values associated with higher MDS-UPDRS III scores). The horizontal dashed-gray line represents the p-value threshold for significant (0.05). Points above this line are considered nominally significant and gait characteristics with nominally significant associations are labeled for emphasis. An examination of this data reveals that the significant reduction in MDS-UPDRS III scores due to the ON state underscores the effectiveness of levodopa in mitigating PD′ symptoms.

Table 4, from pages 4-5 of Appendix A of U.S. Provisional Patent Application No. 63/695,789, which is incorporated by reference herein, shows the estimated associations and corresponding p-values of all gait characteristics measured by the production device.

II. Gait Characteristics with Substantial Changes Between the ON and OFF state

In an aspect, to answer the second question regarding which gait characteristics have substantial changes between the On and Off levodopa state, a mixed effects model may be employed to analyze the relationship between gait characteristics measured by the wearable insole and the MDS-UPDRS III score. In this regard, given that Xᵢⱼₖ is the value of the kth gait characteristic for the jth observation of the ith subject, and assuming that Xijk|γi′˜Normal(uijk′, σk2), then:

$$μ {ijk}^{′} = {β0}^{′} + γ0 {i}^{′} + {β1}^{′} I \{ Stateij = Off \} + {β2}^{′} YiOff$$

In the above equation, γi0′˜Normal(0, σγ′2)γi0′˜Normal(0, σγ′2). In the model, YiOffYiOff is the MDS-UPDRS III score for the ith subject in the OFF state and is used to account for disease severity. This model is applied to each gait characteristic measured by the test device.

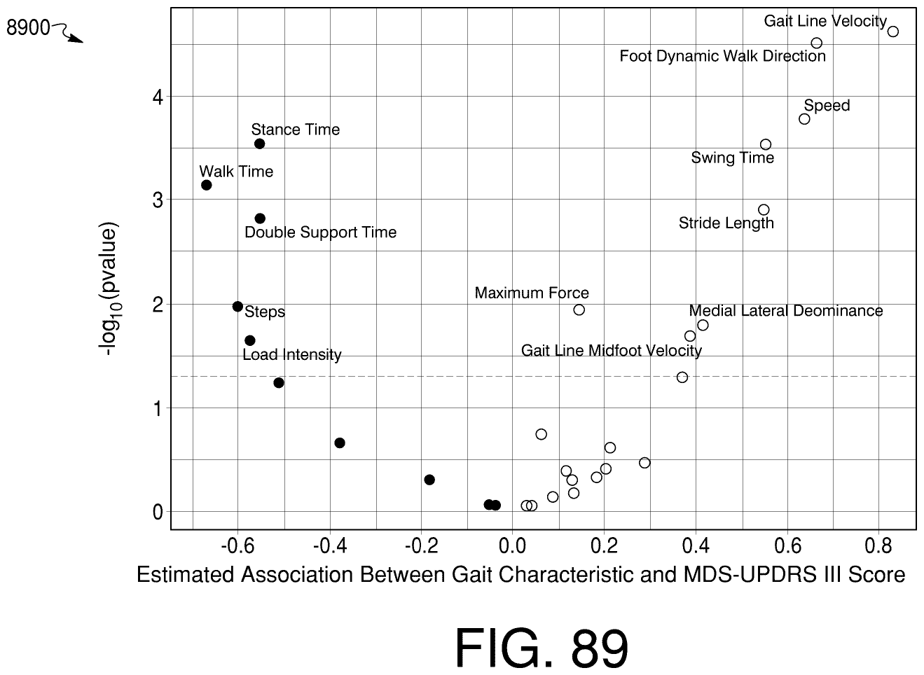

FIG. 89 provides a volcano plot 8900 of mean differences between the ON and OFF state per gait characteristic measured by the test device. The x-axis represents the expected mean difference between the ON and OFF states for each gait characteristic. The solid circles indicate a negative mean difference, meaning that the characteristic value decreases in the ON state compared to the OFF state. The empty circles indicate a positive mean difference, meaning that the characteristic value increases in the ON state compared to the OFF state. The y-axis represents the statistical significance of the mean difference. Lower p-values (positioned higher on the y-axis) indicate greater statistical significance. The horizontal dashed-gray line represents the p-value threshold for significant (0.05). Points above this line are considered nominally significant and gait characteristics with nominally significant associations are labeled for emphasis. The analysis reveals that out of 30 gait characteristics measured by the test device, 13 exhibited significant differences between the ON and OFF states.

Table 5, from page 6 of Appendix A of U.S. Provisional Patent Application No. 63/695,789, which is incorporated by reference herein, shows the estimated mean difference and corresponding p-values of all gait characteristics measured by the test device.

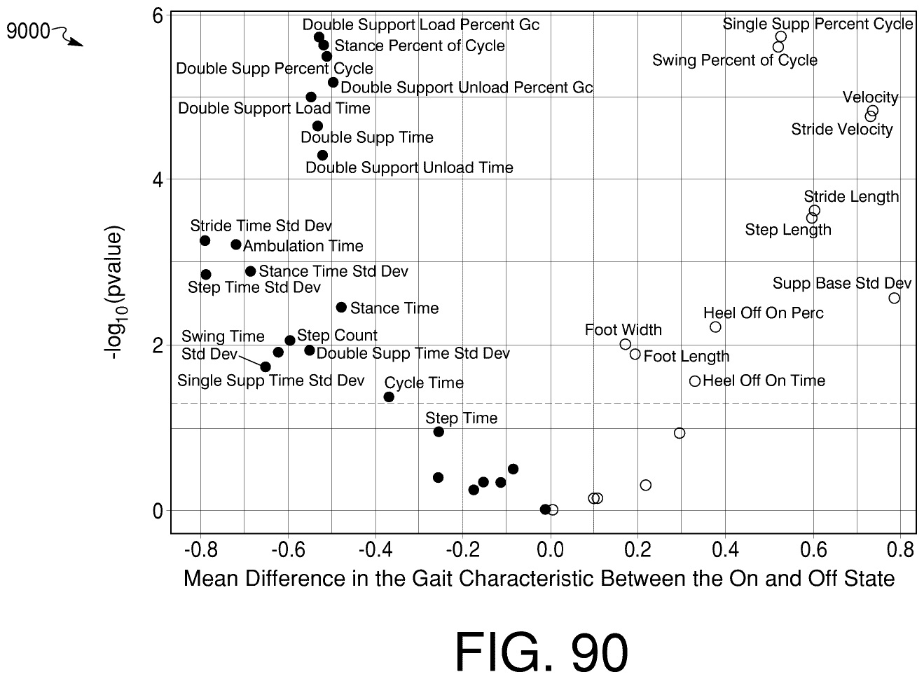

The same model described above may be implemented for the gait characteristics measured by the production device. FIG. 90 provides a volcano plot 9000 of mean differences between the ON and OFF state per gait characteristic measured by the production device. The x-axis represents the expected mean difference between the ON and OFF states for each gait characteristic. The solid circles indicate a negative mean difference and the empty circles indicate a positive mean difference. The y-axis represents the statistical significance of the mean difference. Lower p-values (positioned higher on the y-axis) indicate greater statistical significance. The horizontal dashed-gray line represents the p-value threshold for significant (0.05). Points above this line are considered nominally significant and gait characteristics with nominally significant associations are labeled for emphasis. The analysis reveals that out of 42 gait characteristics measured by the production device, 29 exhibited significant differences between the ON and OFF states.

Table 6, from page 7-8 of Appendix A of U.S. Provisional Patent Application No. 63/695,789, which is incorporated by reference herein, shows the estimated mean difference and corresponding p-values of all gait characteristics measured by the production device.

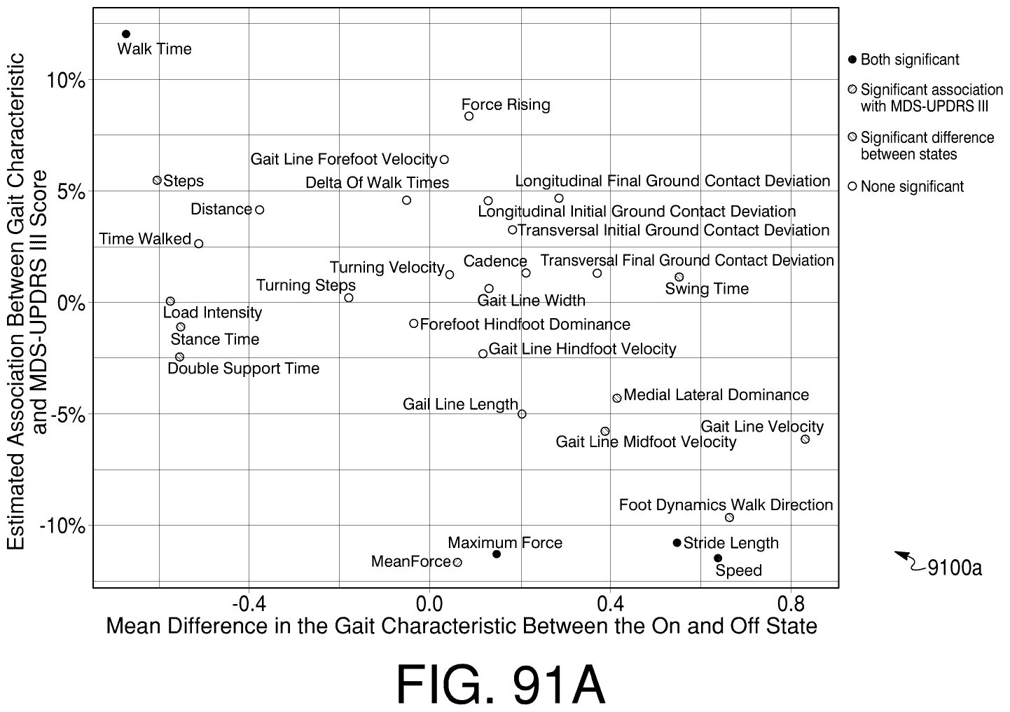

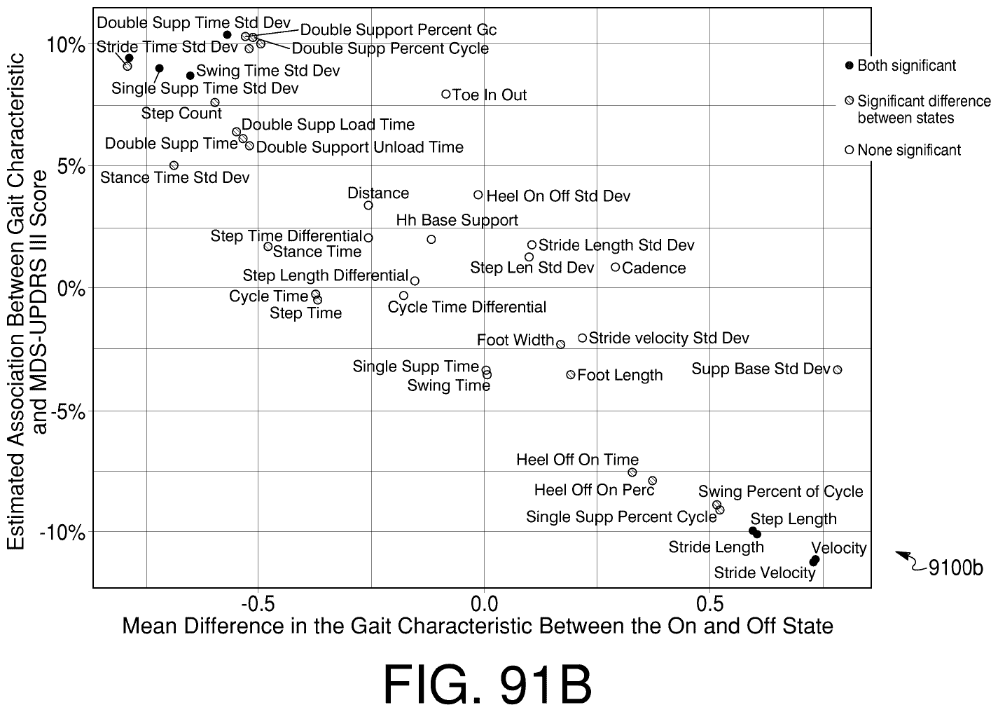

FIGS. 91A and 91B provide graphs 9100A and 9100B, respectively, that present the model estimated association between each gait characteristic measured by the test device (FIG. 9100A) and the production device (FIG. 9100B) and the MDS-UPDRS III score in the y-axis, and the mean difference in each gait characteristic between the ON and OFF state on the x-axis. Each point represents a specific gait characteristic and data points are colored based on nominal significance. For example, one color might represent characteristics with significant associations, while another might represent those without.

III. Speech Characteristics that Associate with the MDS-UPDRS III Score

The direct association of the levodopa state ranges from a decrease in MDS-UPDRS III score between 28% to 52%, whereas the direct association of recruitment site ranges from a decrease of 45% to 61%. Out of over 2,000 speech features assessed by the digital mobile speech-based platform, 166 showed a nominally significant association with the MDS-UPDRS III score.

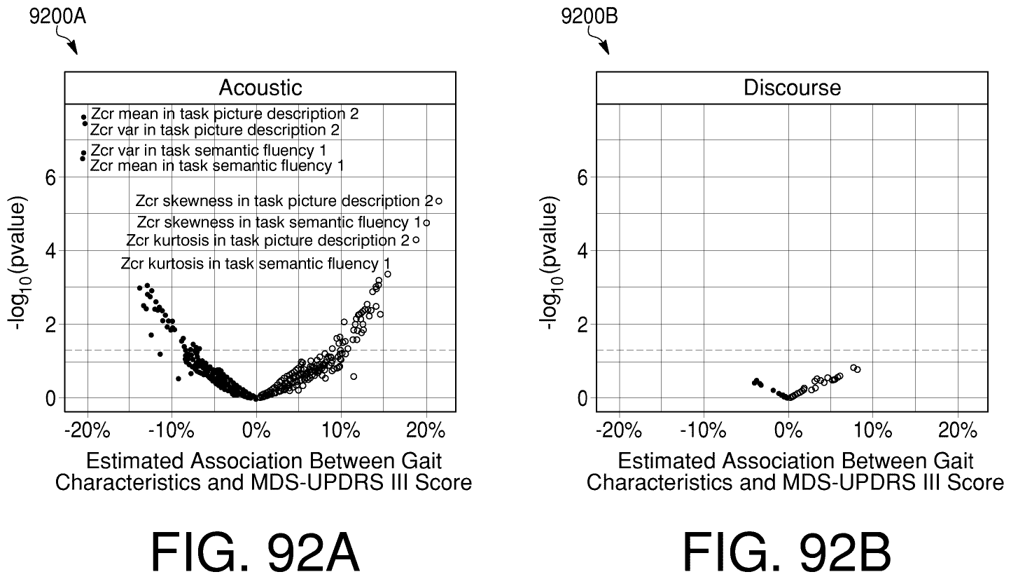

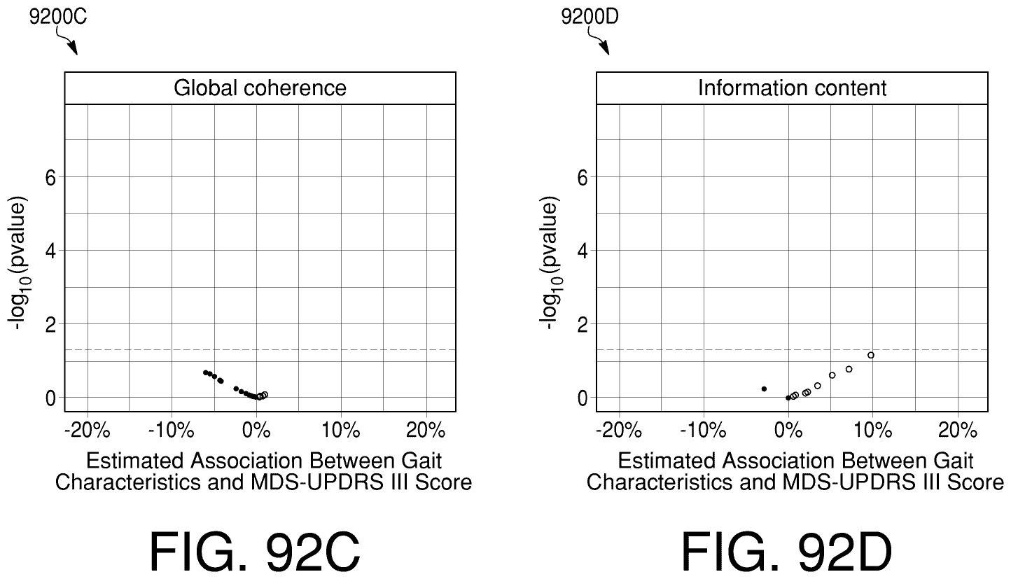

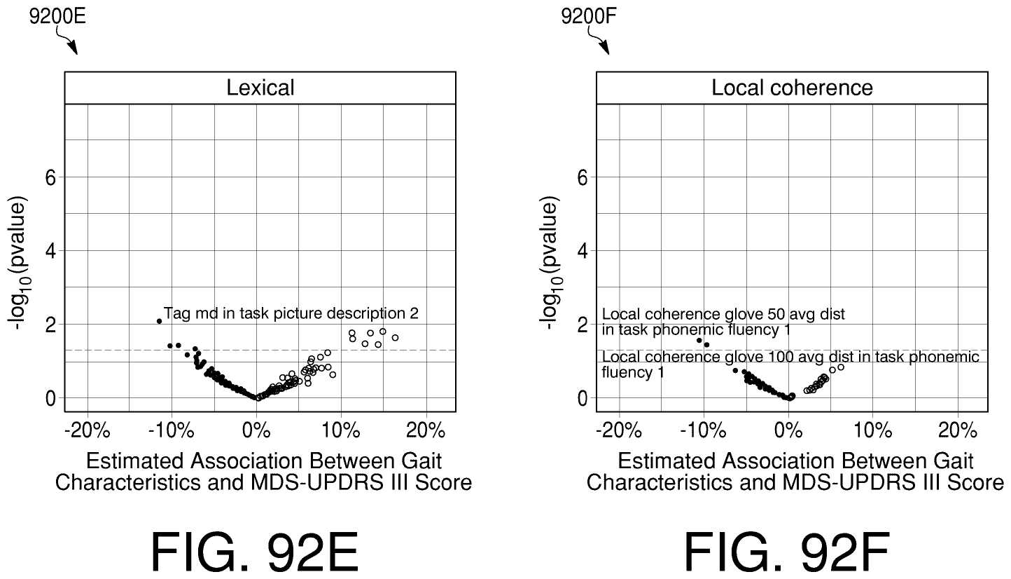

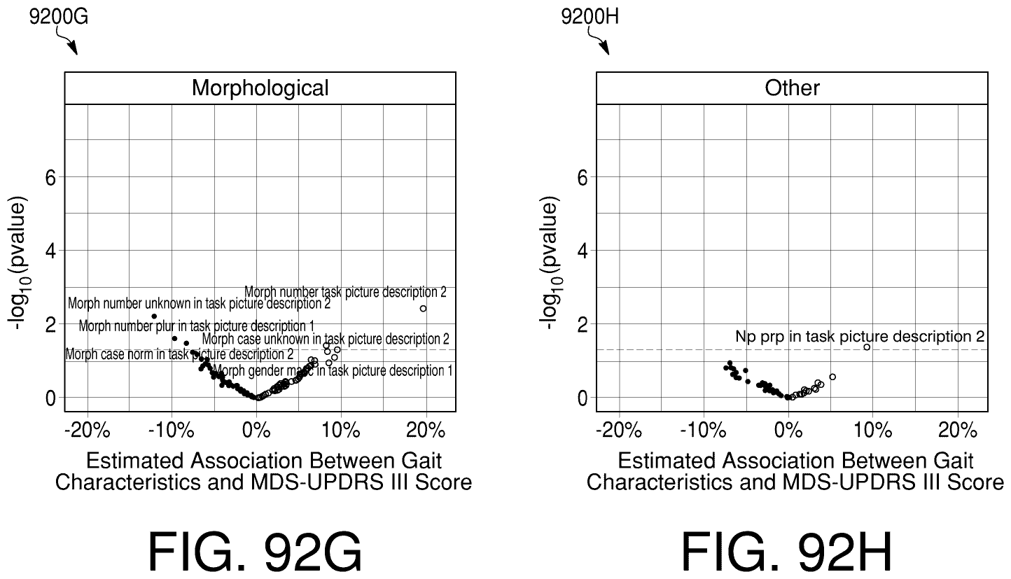

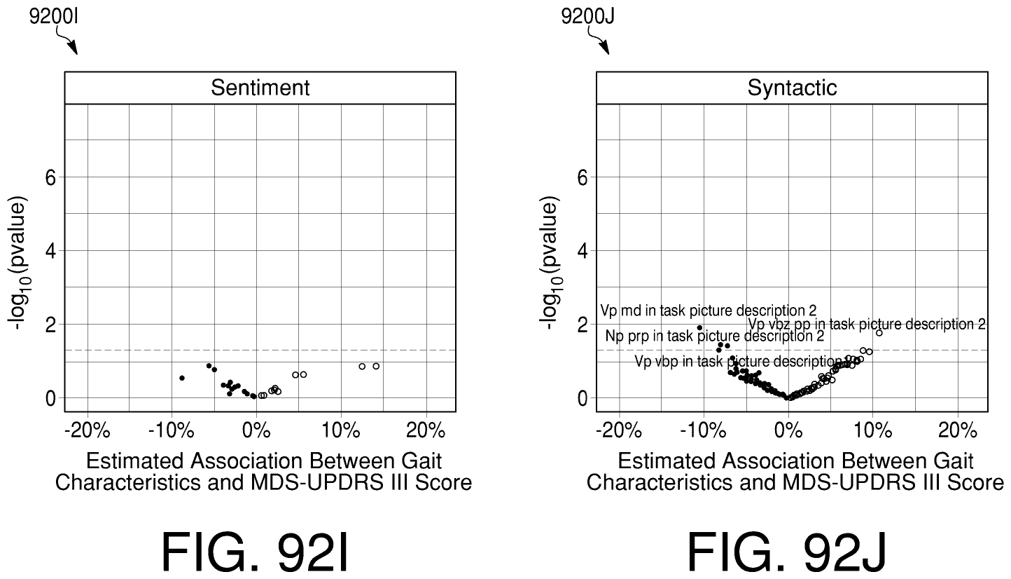

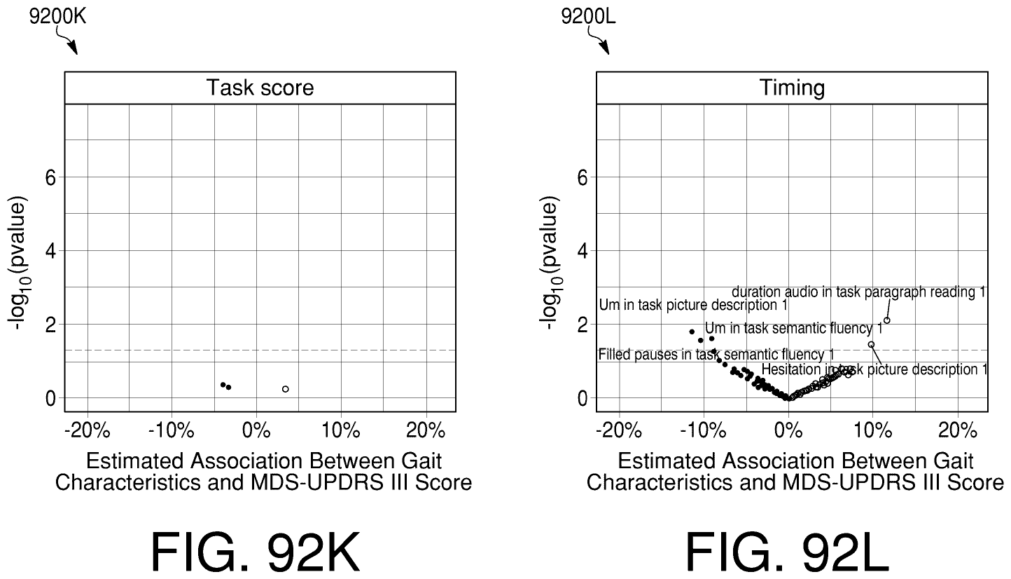

FIGS. 92A-L provide a plurality of volcano plots 9200A-9200L of different speech characteristic associations with the MDS-UPDRS III score. The speech characteristics examined include: acoustic (9200A in FIG. 92A), discourse (9200B in FIG. 92B), global coherence (9200C in FIG. 92C), information content (9200D in FIG. 92D), lexical (9200E in FIG. 92E), local coherence (9200F in FIG. 92F), morphological (9200G in FIG. 92G), other (9200H in FIG. 92H), sentiment (92001 in FIG. 921), syntactic (9200J in FIG. 92J), task score (9200K in FIG. 92K), and timing (9200L in FIG. 92L). The x-axis correspond to the percent change in MDS-UPDRS III score for a one standard deviation increase in the speech characteristic. The y-axis represents the p-value for said association. Solid circles represent a negative association and empty circles represent a positive association. The horizontal dashed-gray line represents the significance level of 0.05. Speech characteristics that have a nominally significant association with the MDS-UPDRS III score are highlighted with labels.

Table 7, from pages 16-65 of Appendix A of U.S. Provisional Patent Application No. 63/695,789, which is incorporated by reference herein, shows the estimated associations and corresponding p-values of all digital mobile speech-based platform speech characteristics.

IV. Speech Characteristics with Substantial Changes between the ON and OFF State

With respect to identifying speech characteristics with substantial changes between the ON and OFF state, out of over 2,000 speech characteristics assessed by the digital mobile speech-based platform, 449 were found to have nominally significant mean difference between the ON and OFF state.

FIGS. 93A-L provide a plurality of volcano plots 9300A-9300L of mean differences between the ON and OFF state per digital mobile speech-based platform speech characteristic. The speech characteristics examined include: acoustic (9300A in FIG. 93A), discourse (9300B in FIG. 93B), global coherence (9300C in FIG. 93C), information content (9300D in FIG. 93D), lexical (9300E in FIG. 93E), local coherence (9300F in FIG. 93F), morphological (9300G in FIG. 93G), other (9300H in FIG. 93H), sentiment (93001 in FIG. 931), syntactic (9300J in FIG. 93J), task score (9300K in FIG. 93K), and timing (9300L in FIG. 93L). The x-axis corresponds to the expected difference between the ON and OFF state per speech characteristic. The y-axis represents the p-value for said mean difference. Solid circles represent a negative difference and empty circles represent a positive difference. The horizontal dashed-gray line represents the significance level of 0.05. Speech characteristics that have a nominally significant difference between levodopa states are highlighted with labels.

Table 8, from pages 65-114 of Appendix A of U.S. Provisional Patent Application No. 63/695,789, which is incorporated by reference herein, shows the estimated mean difference and corresponding p-values of all WinterLight speech characteristics.

FIGS. 94A-L provide a plurality of volcano plots 9400A-9400L, in which the y-axis contains the model estimated association between each speech characteristic and the MDS-UPDRS III score and the x-axis contains the mean difference in each speech characteristic between the On and Off state. Each data point is colored based on the nominal significance. The speech characteristics examined include: acoustic (9400A in FIG. 94A), discourse (9400B in FIG. 94B), global coherence (9400C in FIG. 94C), information content (9400D in FIG. 94D), lexical (9400E in FIG. 94E), local coherence (9400F in FIG. 94F), morphological (9400G in FIG. 94G), other (9400H in FIG. 94H), sentiment (94001 in FIG. 941), syntactic (9400J in FIG. 94J), task score (9400K in FIG. 94K), and timing (9400L in FIG. 94L).

### Analytical Validation

### I. Bias

A mixed effects model was utilized to account for bias. In this equation, given that Xijk is the value of the kth gait characteristic for the jth observation of the ith subject and assuming that Xijklγi†˜Normal(μijk†,σk2)Xijk|γi†˜Normal(μijk†,σk2), then:

$$μ ijk † = \text{} β0† + γ0 i † + \text{} ( β1† + γ 1 i † ) I \{ Stateij = On \} + β2† I \{ Deviceij = Moticon \} + \text{} β3 † \text{} YiOff + \text{} β 4 † I \{ Stateij = On , Deviceij = Moticon \}$$

Where γi0†˜Normal(0,σγ†2). In the model, YiOff is the MDS-UPDRS III score for the ith subject in the OFF state and is used to account for disease severity. The model above is applied to every gait characteristic measured by the test and production device. This approach allows for the assessment of how different gait characteristics correlate with disease severity and treatment effects. In an aspect, to accurately compare and analyze data from the two devices, it is important to standardize the units of measurements. The necessary conversions may include: (i) converting the units of distance measurement of the production device from centimeters (cm) to meters (m) to match units of measurement of the test device, (ii) converting the units of speed measurement of the production device from (cm/s) to (m/s), (iii) converting the units of stride length measurement of the production device from (cm) to (m), and (iv) converting the units of cadence measurement of the test device from strides per minute to steps per minute.

In an aspect, seven gait characteristics were measured by both devices: cadence (e.g., the number of steps or strides taken per unit of time), distance (e.g., the total distance covered during walking), stance time (e.g., the duration of time a foot remains in contact with the ground during a gait cycle), stride length (e.g., the distance covered in one stride, typically from the heel strike of one foot to the next heel strike of the same foot), swing time (e.g., the duration of time a foot is in the air, moving forward during the gait cycle), speed (e.g., the rate at which a subject walks, measured in distance per unit of time), steps (e.g., the total number of steps taken during the walking assessment), and walk time (e.g., the total time taken to complete the walking assessment).

FIGS. 95A-F present box and whisker plots 9500A-9500F that compare the gait characteristics measured by the test and production devices by levodopa state. The gait characteristics include gait cadence (in plot 9500A in FIG. 95A), gait speed (in plot FIG. 9500B in FIG. 95B), stride length (in plot 9500C in FIG. 95C), stance percentage of the gait cycle (in plot 9500D in FIG. 95D), swing percentage of the gait cycle (in plot 9500E in FIG. 95E), and double support percentage of the gait cycle (in plot 9500F in FIG. 95F). Solid circles are production device measurements and empty circles are test device measurements.

In an aspect, the model described above may be fitted to each of the gait characteristics measured by both the test and production device. This steps involves analyzing the data to determine how each characteristic varies between the two devices and under different levodopa states (e.g., ON state and OFF state). More particularly, turning now to FIG. 96, a visual representation diagram 9600 of the analytical validation results is provided that illustrates the comparison of the measured gait characteristics and levodopa state. Upon examination, three gait characteristics (e.g., gait cadence, gait speed, and stride length) appear to be measured similarly by both devices. Specifically, the statistical analysis shows that there is no significant mean difference between the measurements from the test and production devices for these characteristics. However, it is important to note that failing to reject the null hypothesis of no mean difference does not confirm that the devices are equivalent, it simply indicates that there is no strong evidence to suggest a difference. For the remaining gait characteristics (e.g., stance percentage of gait cycle, swing percentage of gait cycle, and double support percentage of gait cycle), significant differences were found between the measurements from the two devices. These differences were anticipated for the gait characteristics of steps, walk time, and distance (e.g., based on the study design). However, substantial differences were observed between the two devices for stance time and swing time, which were unexpected.

FIGS. 97A-F present Bland-Altman plots 9700A-9700F that further assess device equivalence for analytical validation. The gait characteristics include gait cadence (in plot 9700A in FIG. 97A), gait speed (in plot 9700B in FIG. 97B), stride length (in plot 9700C in FIG. 97C), stance percentage of the gait cycle (in plot 9700D in FIG. 97D), swing percentage of the gait cycle (in plot 9700E in FIG. 97E), and double support percentage of the gait cycle (in plot 9700F in FIG. 97F). Upon examination, it is noticed that there is a strong correlation between device difference and magnitude of measurements for distance and stance time.

### II. Intraclass Correlation Coefficient (ICC)

As a validation tool, the interclass correlation coefficient (ICC) was computed between the test and production devices in both levodopa states. More particularly, for each gait characteristic measured by both devices, the absolute agreement ICC was computed to measure the consistency and agreement of the devices' readings. FIGS. 98A-F provide plots 9800A-9800F that visually represent this reliability for each gait characteristic, with short-black segments indicating excellent reliability (high agreement) and longer segments indicating poorer reliability (low agreement). The gait characteristics assessed include: gait cadence (in plot 9800A in FIG. 98A), gait speed (in plot 9800B in FIG. 98B), stride length (in plot 9800C in FIG. 98C), stance percentage of the gait cycle (in plot 9800D in FIG. 98D), swing percentage of the gait cycle (in plot 9800E in FIG. 98E), and double support percentage of the gait cycle (in plot 9800F in FIG. 98F). Solid circles are production device measurements and empty circles are test device measurements. Circles represents OFF-state observations and diamonds represent ON-state observations. This analysis helps ensure that the measurements from the two devices are reliable and consistent.

Table 9, from pages 9-11 of Appendix A of U.S. Provisional Patent Application No. 63/695,789, which is incorporated by reference herein, is provided below that combines the analytical validation results in a results table.

Upon examination, the gait characteristics of stance, swing, and double support time have moderate reliability between devices even with minute mean differences between devices. FIGS. 99A-F present plots 9900A-9900F that visually assess discrepancies between equivalency and reliability for the test and production devices in the varying OFF and ON levodopa states for different gait characteristics. Data points corresponding to the production device are represented by a solid circle whereas data points corresponding to the test device are represented by an empty circle. The gait characteristics assessed include: gait cadence (in plot 9900A in FIG. 99A), gait speed (in plot 9900B in FIG. 99B), stride length (in plot 9900C in FIG. 99C), stance percentage of the gait cycle (in plot 9900D in FIG. 99D), swing percentage of the gait cycle (in plot 9900E in FIG. 99E), and double support percentage of the gait cycle (in plot 9900F in FIG. 99F).

As discussed, one or more implementation disclosed herein include a machine learning model. A machine learning model disclosed herein may be trained using the data flow 10000 of FIG. 100. As shown in FIG. 100, training data 10012 may include one or more of stage inputs 10014 and known outcomes 10018 related to a machine learning model to be trained. The stage inputs 10014 may be from any applicable source including data input or output from a component, step, or module shown in FIGS. 1A-2B. The known outcomes 10018 may be included for machine learning models generated based on supervised or semi-supervised training. An unsupervised machine learning model may not be trained using known outcomes 10018. Known outcomes 10018 may include known or desired outputs for future inputs similar to or in the same category as stage inputs 10014 that do not have corresponding known outputs.

The training data 10012 and a training algorithm 10020 may be provided to a training component 10030 that may apply the training data 10012 to the training algorithm 10020 to generate a machine learning model. According to an implementation, the training component 10030 may be provided comparison results 10016 that compare a previous output of the corresponding machine learning model to apply the previous result to re-train the machine learning model. The comparison results 4816 may be used by the training component 10030 to update the corresponding machine learning model. The training algorithm 10020 may utilize machine learning networks and/or models including, but not limited to a deep learning network such as Deep Neural Networks (DNN), CNN, Fully Convolutional Networks (FCN) and Recurrent Neural Networks (RCN), probabilistic models such as Bayesian Networks and Graphical Models, and/or discriminative models such as Decision Forests and maximum margin methods, or the like.

FIG. 101 is a simplified functional block diagram of a computer system 10100 that may be configured as a device for executing the techniques disclosed herein, according to exemplary embodiments of the present disclosure. FIG. 101 is a simplified functional block diagram of a computer system that may generate features, statistics, analysis and/or another system according to exemplary embodiments of the present disclosure. In various embodiments, any of the systems (e.g., computer system 10100) disclosed herein may be an assembly of hardware including, for example, a data communication interface 10120 for packet data communication. The computer system 10100 also may include a central processing unit (“CPU”) 10102, in the form of one or more processors, for executing program instructions 10124. The computer system 10100 may include an internal communication bus 10108, and a storage unit 10106 (such as ROM, HDD, SDD, etc.) that may store data on a computer readable medium 10122, although the computer system 10100 may receive programming and data via network 10115 communications. The computer system 10100 may also have a memory 10104 (such as RAM) storing instructions 10124 for executing techniques presented herein, although the instructions 10124 may be stored temporarily or permanently within other modules of computer system 10100 (e.g., processor 10102 and/or computer readable medium 10122). The computer system 10100 also may include input and output ports 10112 and/or a display 10110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data, it should be appreciated that the presently disclosed embodiments may be applicable to any environment, such as a desktop or laptop computer, a mobile device, a wearable device, an application, or the like. Also, the presently disclosed embodiments may be applicable to any type of Internet protocol.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

Features enumerated above have been described within the context of particular embodiments. However, as one of ordinary skill in the art would understand, features and aspects of each embodiment may be combined, added to other embodiments, subtracted from an embodiment, etc. in any manner suitable to assist with controlled preparation and/or delivery of a drug.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

### Additional Speech Characteristic Data

Table 10, from page 12 of Appendix A of U.S. Provisional Patent Application No. 63/695,789, which is incorporated by reference herein, further summarizes the clinicodemographic information across all subjects enrolled in the study by medication state. The demographic information is substantially balanced between the ON and OFF medication states.

Table 11, from page 12 of Appendix A of U.S. Provisional Patent Application No. 63/695,789, which is incorporated by reference herein, further summarizes the clinicodemographic information across all subjects by medication state and by recruitment site. The demographic information is substantially balanced across the two testing sites.

Table 12, from pages 12-13 of Appendix A of U.S. Provisional Patent Application No. 63/695,789, which is incorporated by reference herein, further summarizes the clinicodemographic information across subjects enrolled in the study by medication and by enrollment group. The demographic information is substantially balanced across the two enrollment groups.

Upon collective examination of Tables 10-12, it can be seen that the mean difference in the MDS-UPDRS III and the LE subscore between the ON and OFF state is-19.1 (SD=10) and −5.7 (SD=4), respectively. Demographic variables are mostly balanced across the two enrollment sites. The same is true for the two enrollment groups, except for the clinical scores, which were induced by design.

FIGS. 102A-102C provide graphs 10200A-10200C that collectively present a visualization of the clinical scores by mediation state, the correlation between the clinical scores by medication state, and how the value of the clinical scores in the OFF state correlated to that in the ON state. More particularly, FIG. 102A presents graph 10200A that illustrates the change in MDS-UPDRS III and LE scores per subject overall. Graph 10200A provides an indication of subject MDS-UPDRS III scores and subject lower extremity (LE) subscores before and after levodopa administration (e.g., when the subject is in the OFF state and again when they are in the ON state). FIG. 102B presents graph 10200B that illustrates the correlation between the MDS-UPDRS III score (plotted on the y-axis) and the LE subscore (plotted on the x-axis). In general, a strong correlation was found between these two score types in both OFF-state subjects and ON-state subjects. FIG. 102C presents graph 10200C that illustrates the effects on the MDS-UPDRS III Score and the LE subscore when the subject is in the OFF state versus the ON state.

Collective examination of graphs 10200A-10200C reveals that the clinical scores may be highly correlated with each other. More particularly, in the OFF state, the MDS-UPDRS III and the LE subscore have a correlation of 0.87 [95% CI: 0.70 to 0.94]. In the ON state, the same correlation is 0.85 [95% CI: 0.66 to 0.94]. Now, the correlation between the MDS-UPDRS III in the OFF and the same score in the ON state is 0.87 [95% CI: 0.71 to 0.95], whereas for the LE subscore the same correlation is 0.83 [95% CI: 0.61 to 0.95].

Table 13, from pages 13-14 of Appendix A of U.S. Provisional Patent Application No. 63/695,789, which is incorporated by reference herein, presents, on a subject-specific basis, the high variability in clinical differences between medication state.

In an aspect, the subjects associated with the study underwent a plurality of six speech tasks: diadochokinesis (DDK), paragraph reading, phonemic fluency, picture description, semantic fluency, and sustained vowel phonation. In total, 1722 speech characteristics were measured across the six task. The picture description task has the most measured characteristics with 506.

Table 14, from page 14 of Appendix A of U.S. Provisional Patent Application No. 63/695,789, which is incorporated by reference herein, presents indications of the number of speech characteristics per task.

FIG. 103 presents graph 10300 that identifies the change in average word duration spoken by subjects in different medication states. Examination of graph 1030 reveals that, in general, there is a prevalent trend of decreasing word duration as between subjects that have not and have been administered their levodopa treatment. This may imply that levodopa administration may help to improve the speed of speech.

FIGS. 104A-104F provide a series of correlation heat maps 10400A-10400F of speech characteristics across tasks assessed using the digital mobile speech-based platform (e.g., paragraph reading in FIG. 104A, picture description in FIG. 104B, diadochokinetic rate (DDK) in FIG. 104C, sustained vowel phonation in FIG. 104D, semantic fluency in FIG. 104E, and phonemic fluency in FIG. 104F). These plots visualize the correlations between different features within each task. Similar to the gait correlation heat maps, the rows and columns are ordered such that variables that are highly correlated cluster together. Discernible clusters may be found across all six speech tasks with strong positive and pairwise correlations. Additionally, to the left and top of each heat map, annotations were added that denote the category of each speech characteristics. Most clusters are made up of acoustic speech characteristics. Acoustic features include aspects like pitch, volume, and speech rate. Upon examination, the heat maps show large clusters of highly correlated features, indicating that many acoustic features tend to vary together.

The amount of variation exhibited in the correlation heat maps of FIGS. 104A-104F may be further investigated by performing PCA of the speech characteristics by task. More particularly, scree and scatter plots illustrated in FIGS. 105A-FIG. 105F plot the first principal component (PC1) against the second principal component (PC2) by speech task (e.g., paragraph reading in plots 10500A-10515A in FIG. 105A, picture description in plots 10500B-10515B in FIG. 105A, diadochokinetic rate (DDK) in plots 10500C-10515C in FIG. 105C, sustained vowel phonation in plots 10500D-10515D in FIG. 105D, semantic fluency in plots 10500E-10515E in FIG. 105E, and phonemic fluency in plots 10500F-10515F in FIG. 105F). Examination of these scree and scatter plots may reveal important information about various speech tasks. For instance, for the DDK and sustained vowel phonation tasks (illustrates in FIGS. 105C and 105D respectively), the first PC explains over 40% of the variation in the corresponding speech characteristics. For the paragraph reading task, the first PC explains over 30% of the variation of the data, whereas for picture description, semantic fluency, and phonemic fluency, the first PC explains less than 30%. For all tasks, the vast majority of the variation is explained by a subset of the PCs. As in the PCA of gait characteristics, this is an indication that underlying data generating mechanism is of a lower dimension. When looking at scatter plots of the first two PCs is hard to discern what's driving the variation in each. At the very least, it is not immediately apparent to assess if the variation in either of the first two PCs is account for by enrollment site, biological sex, or medication state. This is true across all six speech task.

### Clinical Scores and Speech Characteristics

FIG. 106 presents heat map 10600 of scaled speech characteristic values across subjects, particularly in relation to their clinical status as measured by the MDS-UPDRS III scores. The x-axis contains subjects and is ordered by MDS-UDPRS III scores. The y-axis contains gait characteristics and is ordered by the correlation between each characteristic and the MDS-UPDRS III score. The annotations at the left, top, and bottom, map to a variable or metric of interest with corresponding legends to the right. The color of each cell corresponds to the scaled value, across subjects, of that subject and gait characteristic. Additionally, the correlation of each speech characteristic with the MDS-UPDRS III score is visualized as a function of how much the corresponding characteristics changes between the ON and OFF states. Additionally still, the rows may be ranked by the association of each speech characteristics with the MDS-UPDRS III score. ON and OFF states from each participant are organized in columns from left to right based on disease severity as measured by the MDS-UPDRS III sub score. Characteristics are separated by speech task. Examination of heat map 10600 reveals that there is a cluster of characteristics at the top that are positively correlated with the MDS-UPDRS III score, which exhibit a characteristic change left-to-right, which further validates the strong positive association with the motor score. A similar behavior is observed for those characteristics with a negative correlation with the motor score.

FIG. 107 presents scatter plot 10700 that represents the correlation of each speech characteristic with the MDS-UPDRS III score as a function of how much the corresponding characteristics change between the ON and OFF states. More particularly, in plot 10700, the y-axis contains the Spearman correlation between each speech characteristic and the MDS-UPDRS III score. The x-axis contains the scaled mean difference per speech characteristics between the ON and OFF states. Further examination of plot 10700 reveals that there is a linear association that arises, seemingly indicating that the speech characteristics that are most associated with the MDS-UDPRS III score (i.e., extremes of the y-axis) seem to be the ones that are most sensitive to levodopa treatment (i.e., extremes of the x-axis). However, speech characteristics were identified that seem to be much more sensitive to levodopa treatment than the gait characteristics.

### Methods

A goal of the study in this section is to, i) identify which speech characteristics associated with the MPS-UPDRS III score, and ii) identify which speech characteristics have substantial changes between the ON and the OFF levodopa state.

To answer the first question, a mixed effects model is leveraged where when Yᵢⱼ is the jth MDS-UPDRS III score for the ith subject and Yᵢⱼ|γᵢ˜Poisson(λᵢⱼ)Yij|γi˜Poisson(λᵢⱼ), then:

$${λ}_{ij} = exp \{ {β}_{0} + {γ}_{0 i} + {β}_{1} {X}_{ijk} + {β}_{2} I \{ {State}_{ij} = ON \} + {β}_{3} I \{ {Site}_{ij} = Rush \} \}$$

where γᵢ₀˜Normal(0,σ_{γ}²). In the model, Xᵢⱼₖ is the scaled and centered value of the kth speech characteristic for the jth observation of the ith subject. That is, the model above was applied to each speech characteristic. For each speech characteristic the null hypothesis of no association between the characteristic and the MDS-UPDRS III was evaluated.

To answer the second question, another mixed effects model was leveraged where when Xᵢⱼₖ is the scaled and centered value of the kth speech characteristic for the jth observation of the ith subject, it can be assumed that Xᵢⱼₖ|γ′ᵢ˜Normal(μ′ᵢⱼₖ,σ²k), then:

$${μ}_{ijk}^{′} = {β}_{0}^{′} + {γ}_{{0}_{i}}^{′} + {β}_{1}^{′} I \{ {State}_{ij} = OFF \} + {β}_{2}^{′} {Y}_{iOFF}$$

where γ′₀ᵢ˜Normal(0,σ′_{γ}²). In the model, Yi_{OFF} is the MDS-UDPRS III score in the OFF state. The model above may be applied to each speech characteristic. For each speech outcome, the null hypothesis of no mean difference between medication states was assessed.

### Clinical Evaluation Results

FIGS. 108A-L presents the clinical validation modeling results divided by category, e.g., across 10800A-10800L. The speech characteristics examined include: acoustic (10800A), discourse (10800B), global coherence (10800C), information content (10800D), lexical (10800E), local coherence (10800F), morphological (10800G), other (10800H), sentiment (108001), syntactic (10800J), task score (10800K), and timing (10800L). The y-axis shows the estimated association between each speech characteristic and the MDS-UPDRS III score. The x-axis shows the estimated mean differences between the ON and OFF state. Empty circles correspond to speech characteristics that are not associated with the motor score nor have nominally statistically significant mean changes between medication states. Solid circles correspond to speech characteristics that have a nominally significant mean difference between medication states but are not nominally significantly associated with the motor score. Empty squares correspond to speech characteristics that do not have a nominally significant mean difference between medication states but that are nominally significantly associated with the motor score. Solid square correspond to speech characteristics that have a nominally significant mean difference between medication states and are nominally significantly associated with the MDS-UPDRS III score. The centered and scaled mean difference for the MDS-UPDRS III and LE subscore are highlighted with vertical-dashed lines.

In an aspect, out of 722 speech characteristics, 307 had a nominally significant mean difference between the ON and OFF states and 146 are nominally statistically significantly associated with the MDS-UPDRS III score. In the context of this disclosure, nominally means that the p-value is less than zero. Adjustments were not made for multiple comparisons. Furthermore, some speech characteristics are as sensitive to levodopa treatment as the clinical scores; some of the characteristics are even more sensitive. Furthermore the empty squares in FIG. 108 represent speech characteristics that do not have a nominally significant mean difference between medication states but that are nominally significantly associated with the motor score.

Referring now to FIG. 109, plot 10900 is presented that illustrates the estimated association between each speech characteristic and the MDS-UPDRS III motor score (on the y-axis) plotted against the estimated mean differences between the ON and OFF states. The speech characteristics associated with nominally significant (i.e., p<0.05) mean changes between medication states are colored. Examination of plot 10900 reveals that within-subject comparisons between OFF vs ON states, significant differences were observed within acoustic, syntactic, and lexical domains of speech. Notably, features denoting pitch changes showed particularly strong differences between medication states. These features were also positively correlated with the MDS-UPDRS III motor subscore. Some individual features had effect sizes within the range of the clinical score.

Collectively, it can be deduced from the foregoing data that digital speech assessments captured the treatment effect of levodopa in PD patients. A variety of acoustic, linguistic, and pitch changes were particularly salient having strong clinical face validity and good correlations with existing measures.

Embodiments of the present disclosure may include the following features:

Item 1. A method for triggering an automated action, the method comprising:
receiving, at a computer system, a first set of gait characteristic data from a first device and a second set of gait characteristic data from a second device for an activity performed by a subject in an OFF-state at a first time;
receiving, at the computer system, a third set of gait characteristic data from the first device and a fourth set of gait characteristic data from the second device for the activity performed by the subject in an ON-state at a second time, wherein the second time occurs after the first time;
comparing, using a processor associated with the computer system, the first set of gait characteristic data, the second set of gait characteristic data, the third set of gait characteristic data, and the fourth set of gait characteristic data against a reference score scale associated with a disease condition; and
triggering, at the computer system, the automated action based on a result of the comparing.

Item 2. The method of Item 1, wherein the first device is a wearable insole including a plurality of sensors and wherein the second device is a pressure force mat including a plurality of sensors.

Item 3. The method of Item 1, wherein the OFF-state is a subject state when the subject is not under an effect of a dopaminergic medication, a subject state when the subject is under the effect of the dopaminergic medication at a first dosage, or a subject state when the subject is under the effect of the dopaminergic medication after a first duration of time.

Item 4. The method of Item 1, wherein the ON-state is a subject state when the subject is under an effect of a dopaminergic medication, a subject state when the subject is under the effect of the dopaminergic medication at a first dosage, or a subject state when the subject is under the effect of the dopaminergic medication before expiration of a first duration of time.

Item 5. The method of Item 1, further comprising: identifying, based on the comparing, insufficient data associated with one or more of the first set of gait characteristics, the second set of gait characteristics, the third set of gait characteristics, and/or the fourth set of gait characteristics, wherein triggering the automated action comprises activating an updated activity based on identifying the insufficient data.

Item 6. The method of Item 1, wherein triggering the automated action comprises generating, using the processor and based on the comparing, a treatment plan for the subject to ameliorate the disease condition.

Item 7. The method of Item 1, wherein triggering the automated action comprises: assessing, based on the comparing, an eligibility of the subject for enrollment in a clinical trial; and transmitting, responsive to determining that the subject is eligible for enrollment in the clinical trial, details associated with trial enrollment to the subject.

Item 8. The method of Item 1, wherein triggering the automated action comprises: identifying, using the processor and based on the comparing, a first plurality of gait characteristics measured by the first device and a second plurality of gait characteristics measured by the second device that are nominally statistically significantly associated with disease severity for the disease condition based on the reference score scale.

Item 9. The method of Item 8, wherein the first plurality of gait characteristics measured by the first device correspond to gait speed, stride length, walk time, maximum force, mean force, a first pressure-based gait characteristic, or a second pressure-based gait characteristic.

Item 10. The method of Item 8, wherein the second plurality of gait characteristics measured by the second device correspond to gait speed, stride length, stride velocity, step length, walk time, double support time standard deviation, single support time standard deviation, swing time standard deviation, step time standard deviation, a first distributional gait characteristic, a second distributional gait characteristic, a third distributional gait characteristic, or a fourth distributional gait characteristic.

Item 11. The method of Item 1, wherein triggering the automated action comprises: identifying, using the processor and based on the comparing, whether a gait characteristic measured by the first device or the second device has a nominally statistically significant mean difference between the ON-state and the OFF-state of the subject.

Item 12. The method of Item 1, wherein triggering the automated action comprises: identifying, using the processor and based on the comparing, a plurality of congruent gait characteristics measured by the first device and the second device.

Item 13. The method of Item 12, wherein the plurality of congruent gait characteristics include gait cadence, gait speed, stride length, stance, swing, or double support time.

Item 14. The method of Item 12, wherein trigger the automated action further comprises: identifying, using the processor, one or more mean differences between the plurality of congruent gait characteristics; evaluating, using the processor, the one or more mean differences to identify whether any of the one or more mean differences are significantly different from zero; and establishing the first device and the second device as an accurate assessment device for the disease condition.

Item 15. The method of Item 1, wherein the disease condition is Parkinson's Disease and wherein the reference score scale is a Movement Disorder Society Unified Parkinson's Disease Rating Scale (MDS-UPDRS).

Item 16. A method for triggering an automated action, the method comprising:
receiving, at a computer system, a first set of speech characteristic data from a speech application platform of a mobile device for at least one activity performed by a subject in an OFF-state at a first time;
receiving, at the computer system, a second set of speech characteristic data from the speech application platform for the at least one activity performed again by the subject in an ON-state at a second time, wherein the second time occurs after the first time;
comparing, using a processor associated with the computer system, the first set of speech characteristic data and the second set of speech characteristic data against a reference score scale associated with a disease condition; and
triggering, at the computer system, the automated action based on a result of the comparing.

Item 17. The method of Item 16, wherein the activity is at least one activity corresponding to: paragraph reading, picture description, object naming, rapid syllable repetition, and sustained phonation.

Item 18. The method of Item 16, wherein the triggering the automated action comprises: identifying, using the processor and based on the comparing, a plurality of speech characteristics measured by the speech application platform that are nominally statistically significantly associated with disease severity for the disease condition based on the reference score scale.

Item 19. The method of Item 16, wherein the triggering the automated action comprises: identifying, using the processor and based on the comparing, whether a speech characteristic measured by the speech application platform has a nominally statistically significant mean difference between the ON-state and the OFF-state of the subject.

Item 20. A method for triggering an automated action, the method comprising:
receiving, at a computer system, a first set of gait characteristic data from a first device and a second set of gait characteristic data from a second device for a first activity performed by a subject associated with an OFF-state at a first time;
receiving, at the computer system, a first set of speech characteristic data from a speech application platform of a third device for at least one second activity performed by the subject associated with the OFF-state;
receiving, at the computer system, a third set of gait characteristic data from the first device and a fourth set of gait characteristic data from the second device for the first activity performed again by the subject associated with an ON-state at a second time, wherein the second time occurs after the first time;
receiving, at the computer system, a second set of speech characteristic data from the speech application platform for the at least one second activity performed again by the subject associated with the ON-state;
analyzing, using a processor associated with the computer system, a data collection against a reference score scale associated with a disease condition, wherein the data collection include: the first set of gait characteristic data, the second set of gait characteristic data, the third set of gait characteristic data, the fourth set of gait characteristic data, the first set of speech characteristic data, and the second set of speech characteristic data;
assessing, using the processor and based on the analyzing, a severity of the disease condition and a fluctuation between the ON-state and the OFF-state of the subject; and
triggering, at the computer system, the automated action based on a result of the assessing.

Item 21. The method of Item 20, wherein the triggering the automated action comprises transmitting an alert to a designated device responsive to identifying that the severity of the disease is greater than a predetermined threshold.

Item 22. The method of Item 20, wherein the triggering the automated action comprises generating an alteration to a medication regimen for the subject.

Item 23. The method of Item 20, wherein the triggering the automated action comprises generating a combined therapy recommendation that addresses a gait impairment and a speech impairment associated with the disease condition.

Item 24. The method of Item 20, wherein the triggering the automated action comprises: generating a visualization that displays the data collection; and transmitting the visualization to a device associated with the computer system.

What is claimed is:

1. A method for triggering an automated action, comprising:
receiving, at a computer system, a first set of gait characteristic data from a first device and a second set of gait characteristic data from a second device for an activity performed by a subject in an OFF-state at a first time;
receiving, at the computer system, a third set of gait characteristic data from the first device and a fourth set of gait characteristic data from the second device for the activity performed by the subject in an ON-state at a second time, wherein the second time occurs after the first time;
comparing, using a processor associated with the computer system, the first set of gait characteristic data, the second set of gait characteristic data, the third set of gait characteristic data, and the fourth set of gait characteristic data against a reference score scale associated with a disease condition; and
triggering, at the computer system, the automated action based on a result of the comparing.

2. The method of claim 1, wherein the first device is a wearable insole including a plurality of sensors and wherein the second device is a pressure force mat including a plurality of sensors.

3. The method of claim 1, wherein the OFF-state is a subject state when the subject is not under an effect of a dopaminergic medication, a subject state when the subject is under the effect of the dopaminergic medication at a first dosage, or a subject state when the subject is under the effect of the dopaminergic medication after a first duration of time.

4. The method of claim 1, wherein the ON-state is a subject state when the subject is under an effect of a dopaminergic medication, a subject state when the subject is under the effect of the dopaminergic medication at a first dosage, or a subject state when the subject is under the effect of the dopaminergic medication before expiration of a first duration of time.

5. The method of claim 1, further comprising:
identifying, based on the comparing, insufficient data associated with one or more of the first set of gait characteristics, the second set of gait characteristics, the third set of gait characteristics, and/or the fourth set of gait characteristics, wherein triggering the automated action comprises activating an updated activity based on identifying the insufficient data.

6. The method of claim 1, wherein triggering the automated action comprises generating, using the processor and based on the comparing, a treatment plan for the subject to ameliorate the disease condition.

7. The method of claim 1, wherein triggering the automated action comprises:
assessing, based on the comparing, an eligibility of the subject for enrollment in a clinical trial; and
transmitting, responsive to determining that the subject is eligible for enrollment in the clinical trial, details associated with trial enrollment to the subject.

8. The method of claim 1, wherein triggering the automated action comprises:
identifying, using the processor and based on the comparing, a first plurality of gait characteristics measured by the first device and a second plurality of gait characteristics measured by the second device that are nominally statistically significantly associated with disease severity for the disease condition based on the reference score scale.

9. The method of claim 8, wherein the first plurality of gait characteristics measured by the first device correspond to gait speed, stride length, walk time, maximum force, mean force, a first pressure-based gait characteristic, or a second pressure-based gait characteristic.

10. The method of claim 8, wherein the second plurality of gait characteristics measured by the second device correspond to gait speed, stride length, stride velocity, step length, walk time, double support time standard deviation, single support time standard deviation, swing time standard deviation, step time standard deviation, a first distributional gait characteristic, a second distributional gait characteristic, a third distributional gait characteristic, or a fourth distributional gait characteristic.

11. The method of claim 1, wherein triggering the automated action comprises:
identifying, using the processor and based on the comparing, whether a gait characteristic measured by the first device or the second device has a nominally statistically significant mean difference between the ON-state and the OFF-state of the subject.

12. The method of claim 1, wherein triggering the automated action comprises:
identifying, using the processor and based on the comparing, a plurality of congruent gait characteristics measured by the first device and the second device.

13. The method of claim 12, wherein the plurality of congruent gait characteristics include gait cadence, gait speed, stride length, stance, swing, or double support time.

14. The method of claim 12, wherein triggering the automated action further comprises:
identifying, using the processor, one or more mean differences between the plurality of congruent gait characteristics;
evaluating, using the processor, the one or more mean differences to identify whether any of the one or more mean differences are significantly different from zero; and
establishing the first device and the second device as an accurate assessment device for the disease condition.

15. The method of claim 1, wherein the disease condition is Parkinson's Disease and wherein the reference score scale is a Movement Disorder Society Unified Parkinson's Disease Rating Scale (MDS-UPDRS).

16. A method for triggering an automated action, the method comprising:
receiving, at a computer system, a first set of speech characteristic data from a speech application platform of a mobile device for at least one activity performed by a subject in an OFF-state at a first time;
receiving, at the computer system, a second set of speech characteristic data from the speech application platform for the at least one activity performed again by the subject in an ON-state at a second time, wherein the second time occurs after the first time;
comparing, using a processor associated with the computer system, the first set of speech characteristic data and the second set of speech characteristic data against a reference score scale associated with a disease condition; and
triggering, at the computer system, the automated action based on a result of the comparing.

17. The method of claim 16, wherein the activity is at least one activity corresponding to:
paragraph reading, picture description, object naming, rapid syllable repetition, and sustained phonation.

18. The method of claim 16, wherein the triggering the automated action comprises:
identifying, using the processor and based on the comparing, a plurality of speech characteristics measured by the speech application platform that are nominally statistically significantly associated with disease severity for the disease condition based on the reference score scale.

19. The method of claim 16, wherein the triggering the automated action comprises:
identifying, using the processor and based on the comparing, whether a speech characteristic measured by the speech application platform has a nominally statistically significant mean difference between the ON-state and the OFF-state of the subject.

20. A method for triggering an automated action, the method comprising:
receiving, at a computer system, a first set of gait characteristic data from a first device and a second set of gait characteristic data from a second device for a first activity performed by a subject associated with an OFF-state at a first time;
receiving, at the computer system, a first set of speech characteristic data from a speech application platform of a third device for at least one second activity performed by the subject associated with the OFF-state;
receiving, at the computer system, a third set of gait characteristic data from the first device and a fourth set of gait characteristic data from the second device for the first activity performed again by the subject associated with an ON-state at a second time, wherein the second time occurs after the first time;
receiving, at the computer system, a second set of speech characteristic data from the speech application platform for the at least one second activity performed again by the subject associated with the ON-state;
analyzing, using a processor associated with the computer system, a data collection against a reference score scale associated with a disease condition, wherein the data collection include: the first set of gait characteristic data, the second set of gait characteristic data, the third set of gait characteristic data, the fourth set of gait characteristic data, the first set of speech characteristic data, and the second set of speech characteristic data;
assessing, using the processor and based on the analyzing, a severity of the disease condition and a fluctuation between the ON-state and the OFF-state of the subject; and
triggering, at the computer system, the automated action based on a result of the assessing.

21. The method of claim 20, wherein the triggering the automated action comprises transmitting an alert to a designated device responsive to identifying that the severity of the disease is greater than a predetermined threshold.

22. The method of claim 20, wherein the triggering the automated action comprises generating an alteration to a medication regimen for the subject.

23. The method of claim 20, wherein the triggering the automated action comprises generating a combined therapy recommendation that addresses a gait impairment and a speech impairment associated with the disease condition.

24. The method of claim 20, wherein the triggering the automated action comprises:
generating a visualization that displays the data collection; and
transmitting the visualization to a device associated with the computer system.
